SEQUENCE LISTING (618 sequences)

SEQ ID NO: 1 - DNA, 3667 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcctggcccttttttttttttttttttttttttttaaagagatggcatcttgctatgtcgtccaggctggtcttgaactcctgagttcaagcagtcctcctgcttcaacatacagctacaggtaccccccactatacatttttaataaggattcatggctcagagggattttctgatggttttgctgatttgtttctagtttttttgtgtttatatttaacatgaagaccaagtttatataactaggtatctgtataatgcaacaacattggaacacaataaagatgtatttttgtaaa

SEQ ID NO: 2 - DNA, 32101 bp, Homo sapiens
actccgtctctaaaataaaataaaataaaatcaccctgtaacttactacctcctcagaaattaagtttgcgctgtccaacagcaagaggaattcgcttgataagaataattaaacacataaaggcaatgcctaacctcgtaacgccaccccatattccttaatctttaccaagccactataagacggcagaaaacacaagctgcactttcgttcttttctcgtgtcccgttttcaacagaccatggatttttttttttttttttagatggagtctcgctctgtgccaggctggagtgcagtggcacgatctcagctcactgcaacctctgcctcccgggtttaagcaattctcctgcctcagccaccagagtagctgggactacaggcgtgtgccaccacgcccagctaattttttttgtattcttagtagagagggggtttcacctgttggccaggatggtctccatctctttacctcgtaatccgcccacctcggcctcccaaagtgttgggatattataggcgtgagccaccgcgcccgaccagaacatggaaatttaaagagaaaaaaataagtgacttctgatttcaaaaacacaaccacccacttttctattctattcctggtgagcacattctggagactctgcggcaaccatagcctcggccgcggcggaaggctccggggcggccacacctcagggggcagccacacctcagggggcagcgcgcacaggcgggtcaggctctgattcccatctctctccggtcttcactggaaggacccacactacatcacaaactaatggctttacaattaacttctaagtagctaaatttagtaatgggcgctctagttttaaaaaatattattcaggccgggcgcggtggctcacgcctgtaatcccagcactttgggaggccgaggctggtggatcacctgaggttaggagttcaagaccagcctggccaaaagggtgaaaacccgtctctgcaaaaatacaaaaaattagccaggcgtggtggcggacgcctgtagtccccagctactcgggaggctgaggcaggagaatcgcttgaacccgggaagcggaggttgcagtaaggcgaggtcgcgccactggactccagcctgggcaacaagagcgaaagcgaaactctgtctcaaaaaaagaatattattcaaaaactgattttcacatttttaaaaaagacccactgagggaggaagaccgaatcccacacatgcgcgcgcacagcggttccgagttctgcaaccaacctcccacgggcgccgcgccttctccgcgtcacgtgacggccgtctcccgcaccttccatactctcaatgtagggagaggacgggttccatcagcaatttaataaaaaccattggtgaagcaaaaaaaaaaagcctgccaacctcttataacgttttttctttatttttctcttttatgtggacaggaagccttaggttggctatatttaccctcaaatctctttttaaaatgatttttaagtttcagcatactcccgcgcttaatgaaatggtttcccccagcatccccagctggtgcggtctgaaccattcggaacttcccagaggggggcaccggaataggggcggtggaaaagggtaattgtcgggcagcggcccctgcttcccagtcagacctcggagttgtaggactcggactgtgttcctgggcttgcgagagctagggaggcccggctctcctcgcggtgcaggacgcgctcggacggctcccgcacccccaccgcagtgttgggtggtggtacggctcagggagtcacgcgcctgggtgtcggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcaggtaggcgggggtctgaggggttggcggaggggcatgtccgagttttcgttggtcccaggtggacgagtaatgggggagggtttagggccagagtccagggcgtcgccgagaccctgagatttgaagataggttgggggcacgcccattaaccaagtctatggggatgaggactgtgatctcatttgatgttgccatgaaacgggactgtttgccttcccttctcccaggttttgtcttcgctctgtttggaggagagggtgtgtgtcatcctcttctcccagtttggcgttcaggagggtcctctgatgcgctaatagggtagcaccgtgtcctccagggagggtggaagaccgcgcttctctccagttgagagtactgtcagtcgcgtccttgtctcctggaaagaatggattggcttgtggattgaagtccaaggtgtggtcactagcagtctcttcaagtggggacacagggcaaggaggagcttgctggaagtaggttctggctctctagcctttggccacagccagtttagtgccagattatcaccgtggtcatcaagtaattgagtaatagtggcaggtgacactgcgtttgcccttctagtgtcctatgtcagggagctctgtgaatggaaatccagggaaacggtggaaatagagccacagtgagagtggatctgaactggcagtcatcccctgcagaacttttcaccctgggcttgttgggatcccagctgctaacttaatgaggctccccagtgggagactagttatagaagctgtgtgtccactgctggttaagatccgttatagtgctttgtttatttagagtgcccttacttagtggcctctttaattctccacggtgctatgagaacaccataggaagtagggttgaaatagggccagggcgcaccacgggtggtggtgaggtcactgtccaggtctgggactcagccattgtgggagggggacatgaacagacactctaccacatcagggaatttgaatactgtctttggatatgttgatgaagagactgtcagccagggcatttcggcttcctatgtgggttctggaaagcttgttggaaacagactcgtgagacacactcaaaccagtttgttttcttcagcaaacacctggtagagattggaattgaacagtgagtgaagaatgacctcacgctgcccagaattctgattccatgtgtaatgcgaattctgagaggcctctgtttctcagcttttgggtggtattcagagaggtctagtaacacttggtgtcacctggctggtgagggcaggctccggtgaccgcaaagctaagtgctctgtagggcacctccctctctttcaggggctgcctgggcctctggtgcacatgatccatggagtcagagtcagagctctcgtctgacaagtgcttttgctttctttctttctttctttcttttttggagacagaatctcattctgtcgcccaggctggagtgcagtgatgcgatctcggctcactgccacctccgcttccaggctcaaacgattctcctgcctcagcctcctgagtagctggcactacaggcacgtgccaccaggcctggctaattttcgtattttgtattttttgaaatggagttttactcttattgcccaggctggagtgcaatggtgcgatctcagctcactgcaacctccgtctcccaggttcaagcaattctcctgcctcagattccccagtagctgggattacaggcatgcaccaccaccatgcctggctttttttttttttttttttttgaaatggagtcttgctctgtggcccaggctggagtgcagtggtgcgatcagctcactgcaagctgtgcctcctgggttcacgccattctcctccctcggcctccccagtagctggggctacaggcccccgccaccatgcccagctaattttttgtattttttagtagagacggggtttcactgtgttagccaggatggtctcaatctcctgacctcgtgatctgcccacctcggcctcccaaagtgctgggattacaggcgtgagccaccgcacccagcctaatttttgtatttttagtagagatggggtttcaatatgttggccaggctggtctcgaactcctgacctcgtggtccgcctgcctcggcctcccaaagtgctgggattacaagcatgagccaccacacccggcaatttttctatttttagtagagacagggtttcaccatgttagcctggctggtcacaaactcctgaccttaggtgatctgccctccttggcctcccaaagtgctgggattacaggcttaagccactgcacccagcgagtgcttttcattaaaaggaaaaacgaataagagtagcccatgttttggccagacatggtggcttatgcctgtaatcccagcactttgggaggccgaggaggcgggagaatcacctgaggtcaggagttcaagaccagcctggctaacatggcgaaaccccatctctactaaaaatacaaaagttggctggtcctggtggcgcacggccataattccagctactcgggaagctgaggcaggagaattgcttgaacctgggaggcggaggtggcagtgatccgagattgctgcactgcactccagcctgggcaacagaatgagacccttccgcaaaaaaaaaaacaaaccaaaaaaaaacaaaacagtgtagcccatgttggctctgggtgctgggaagtggcttgtcagcgttgcctctggtttccctctcctgcttgagaaggaaactgccaggcctatggcctgaaaagtaagttggtgtgatgtagatgctgccagagcacccccttataaccaagagtaagaaagatggggacttggctggatgcagtggctcatgcctgtaatcctagcacttagggaggctgaggtaggtggatcacctgaggtcgggagttcgagaccagcctggccaacatggtgaaactctgtctctactaaaatacaaaaattagccaggtgtggtggcagacacctgtaatcccagctactcaggaggctgaggcaggagaatcacttaaacctgggaggcagaggttgcagtgagctgagatcacgccattgcactccagcctgggtgacaagagtgaaacttggtctcaaaaaaaaaaaaaaaaaaaaaaaaggaaagatgggaacttgcagccttgacctcccaggctgaagtgacctcccaacttagtcccccaagtaacttggactagaggcatgcccggctaatttttttttttttttttttttttgagatggagtcttgctgtgtcgcccaggctggagtgcagtgccgtgatcttggctcactgcaagctctgcgtctcaggttcacgccattctcctgcctcagcctcccgagtagctgggactataggcgcctgtcaccatgcctggctgattttttttgtatttttagtagagatggggtttcaccatgttagccaggatggtctcgatctcctgacctcctgatctgcccacctcggcctcccaaagtgccggggttacaggcgtgagccaccgcgcccggcctggctaattttttttaaaaattttttgtagagacagggtctcactgtgttacccaggctggtctcaaactcatgggctgaagcaatcctcctgccttggcctcccgaagtgttgggattacaagcatgagccactgagctggcctcaagaccctatctctaaaaaaataaatacagagatccttgatcaaatactggtttagagtgcctcagaggtgcaagagttcagctaaaatgtcagctcctgctgagaaggtcagaggcctgctaagccattcagaaccctggggagtgtctacatcctcatcagacacgttcttatttgagactggctgtaagctgctcccattatttttcatgctacgaaggcctctaatcttagaaatagctctaattttttttttctcttacctagaaaacgccaggtcttcaagggtgtctgccaccaccatgcctgacccatttggcagcagcctcgtgtgtggtggtctggtgtggacggtggaagcgtgattctgctgagtgtcaggttagtgtgggtgctctgcaggtgggctgagacttggcccaggcttgctgtcctgtgcatgtcttgctagaaggaaccttatttctcttcccctgatgcctctcagagaaaggcgtggccttccttttcgcagtttaatgcctttacctttcctttgggtcctgctaccccttactccatcagtggtcgccccatctcctgttgagcagtagatttttaagccactcatcatagtgaaccctgtgggccctttattgaaactgtctgtttggttcctgtgccactagactctggctccggccttccccacagctgtggctcctgaacctctgccgcccctagattccgcccctttcttggcaacagctgtcatctttccaagggcgacttctcagtcctctcatttatctaacacgtgaatgtggcgcttcctgtgtgtcaggaaggcactttggaggcgcagttctgcgatgaggagcgctacgactcaaagcagggcaggggagggtgtcggggtggaggggagctcttctggtgggctggcaaggggtactctgagcaggggagcaagtggaggccgagtggtgaggagtgagccaggtgacggtgtgtggagcattcctgacctttgtccccagccctgccctctcctgtcctgcagcctgcatctttgctgagttctcagggccttccagctagaagttctgccatctgttaaatgcgtatgtttcctctcccgctgcctgtctgcctccctctcggagtgcacctacagagcactagccctccattccctgccagccacacccagggtctccctctctgactcccacacctgcctcactgccagcccagctaaggttctcttcaaatgtctgttttctgtctgcctctgccattcccagtgtgaccactcgtgctcagccgtatctcagcaggaggacaggtgccggagcagctcgtgcagctaagcagccaactgcagaaacgtcaggtgggtggtgcattcgcaggcatgctgaagaagcagttccaggcatgggccgccggcagagaggctggctgcagcaccccccaccaccatgcacatcgtcctttgcttcttcctagagttagcggctttggcaggccatgccttgtttgtgttcacagcctgcctgtcaggagcgaagacagaacgtctgacttttggatccgtaacaggggtgggggctccccagaagagaagaggctttgggtcctggccagtgtccactactcagtcaaacattcagaaacttacatgattcttcatcgtcccaagcaagtctgacttgggccctttattgaaactctgtttcctctcccgctgcctgtctgcctccctctcggagtgcacctactgagcaccagccctccattccctgccagccacacccagaaagcaatggggctttctgggaaggcagaaatattctgtgacctgggctattcagaggggtggcgtgagtcgtgtgtgcctagcaaacactcaggacatggccggtgagacagaagggctgagcttttgtcctagctaatatattaattaattaattcattatttattttgagacggagtctctctgtcgcccaggctggactgcagttgcgcagtcttggctcactgcaaccttcgcctcctaggttcaagcaattctcccgcctcagcctcttgagtagctgggactacaggtgtgcgccaccatgcctggctaattttttttgtacttgtagtagaaatggggttttgccatgttggccaggctggtcttgaactcctgacctcaagtgatctgcccacctcggcctcccaaagtgctgggattacaggcatgaacaaccgcactcagcctatttatttaattttttcgagacgaagtctcactcactctgtcacccaggctggaatgtagtagtacgatctcagctcactgcaacctccacctcccaggttcgagcgattctcctgcctaggcctcccaagtagttgggactacaagcgtgcgccaccactccaggctacttttttttttttttttttgagatggagtctcgccctgtcataaaggctggagtgcagtgacctagtcttggctcactgcaacctccgcctcccaggttcaagtgattctcctccctcagcctcccaagtagctgggtttataggtgaccgccaacacgcccagctaaggtttttgttttattttgttttgtttttgttgtttttggttttttttgagactgagtctcactttgttgcccaggctcgagtgccgtggtgcgatctcggctcactgcaaccttgcctcccaggttcaagtgattctcgtgcctcagcctcctgagtagctgggactaccagcgcacaccaccacacctggctaatctttgtattcttagtagaggcagggtttcactgcgtcggccaggctggtctcaaactcctgacctcaagtgatccacccgcctcagcctcccaaagtgttgggattacaggctcatgggattacaggcatgagccactgtgccggcctctcctagctaatttaaattttaaatttaagttgtcacacatagctgtggccaccgtatggacaatgtgggtctggctgcatgtgtcaggctgtgtcacatgtcaggctgttggctgtggctgtccccactgtgcacctttgcttttgcggtctcgtttccagaacttctctgccagtgagaaccccatgacacacccccattttaaagccttctctggctttcctgcattcgttaggaaggcatactgcctcatgctgccgcggccatccctctgcaatgagtgcttggtgtccctcagactttgtgcctgcagctgcccctggccttcacatggttccgccggcatcgcagctgtactgtggccaagcttggggctttccttgactccatgtccccaccaggtgtcccattgctggctctgccacggaaactctccaaagggctctctctacccactcctgtgagctttttctccaccggaaactgtgttaaggtcacctttgacttcatcattgttctgcaggttaccactcccccttttaaaaagattgtggtgaaataatacatgacctaaaatgcgccatttgaaccactgttcggcgtgcagtgccgtggcatgaagtatgttcacagtgctgtgcggccatcgccaccttccgtctccagaactgagccccgctcttccctcccagtccccggcagctgccattctgctctctgtctctatggctttgaggactctaggtacctcctatttaagtggaatcctacagtgtttatccgtttgtgaccagtgtagatcatttcgtgtaatgtccttcagtttcagccgtgccgtagcttgtggcagggtttccttcctttttaaggccatgtgacattccagtcccaccttgctaaagcagtttacctggtcactggggtgactctggagctcccctctggcctcactgcctgtgcttggtcatgttggctagctgtcctcccttctccacctctgagtctacaagggctgcactccacggaggtcccatgcgtcagccccgaccgcagcgcttggctctgctgctgtgtcccgtccccgtgaaggctcagcaggcactcactgggccttctcccctgctcactgctccgcactctgcaccccacccgcactgtgcgtcttcaccctgcctggcctgggccactgtcccttcctgctcagatcagtcagcagggtgcctcagcttgttccccacctccaccctggccccctgccatcttttctgcacatgggcatgagtgaacctattcaagtgaaattcacattttctcatgtctcatcagaacccccaaagcctccccagtctctgataacacatccagggtctttcggtggcctggaggagctataagatctgcttcctacagcctccaccctgtctgctgctgctctttggacaccggaccttacacacccctcagacagtacagcacccaccctgcccttcaactcttgcttggccccgtaagccagtccttccaggctcctgcctcctctggtcattccctgcgtgggcctctcgcaggaagcagttttcctgtgtcccccgccgaccccctgcaggaggtaagctgcatgagggcagtatcatgttgcctagcaccttggcccacatggagcaggtgccaagcagatatgtattgggtggaggaccctctccctgtggccttttatctatttgttctcacagggtttgccaggtttggctgcagttcatgtcacttgaggacattcctttccagcgccgttggctttgtggctgtggctctgtcacagctccagctgtgctatcattggtccacattgtcccctcccctatttgctgtgactcccatgggaagatcctatctctgttcctctcgcactggggcaaagcagtgctggcagggggctggggaactggcgggtggcctctcctgggcaggaagtccatggcacccaggtgctccctgtgctccagaggggctggggaggtttcatggagtcaataggcattggcaagctgacatgtcccctgtcccacgcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgaggcaagtgtctttggggtcttggttttgtgatccctgcagtaggtgtgggatggacaggaggacctgagagcagtggacggggcctcccggggtgacacagactgcttctcaggggcccttcagttctttccgctcatcctgactgtagcttttgttttgttttgttttgttttttttgagacagagttttgctcttattgcccaggctggagtgccatggtgtgatcatggctcactgcaacctctgcctcccgggttcaagcaattctcctgcctcagcctcccgaataacgaattacacttgcctgccaccacgcctggctaattttgtatttttagtagagatggggtttcaccatgttgggcaggctggtcttgaactcgtgacctcaggtgatccacctgcctcggcctcccaaagtgctgagattacaggcgtgagccaccacacccagcctatgttaacttctttaacccactctgcctccctgccgtggccttcatgtttgtattcacacactccagcctgtcctggaggcgggtccccaaagtcccgtgctcccagtggagtttcctgctcccaggacccccagtgttctcggtgggtcactccatcagctcttgaactctcgggaattagtgtgagttttccccagtgggccacttgtttttgtggaggacctgggtcagggcccaaaatcaggggtgggtgttggacctgccactgcagtgcaaacgggaacccaagcttgttcttttggttctgctgcagaagcagaggacgtgtaagagctgcatccattgctggaattgcctgtaaggaatgggggatggggagcacctcagacctgggaaggagggcaccgccaggctgctggggcatccttgagctggttttggggttgcccatggaggaatcctgcgacactgtggggagagtcttgctgacgcactgccgcacagtcctggggtaggagcagctccagggcgcacagcctcagcatcggagtggctgtggctgcagcggggcagcggggcagcagtggtgagctgccacaccaacatcctggagcttggtgctcgccctatttattgggcctctcgcctgtggctgaagccccttccaggtgttagttcagaaccaatgatggaggctgggggcagtgggtagtgttaccaccacagagcccctcaccctcgggccagctacttctcacttcagcctcagttttgtcttctgtagaatggggcttctcactgacattatctcagagggtggcacttgggattgaatgagatggagcatgcagggcccttttcatgttacctacttcgtaagtgctcagcaaatacctgttaggaacaacctctggcaatctctgcgatctctgtgttctgttttcctctttaggaaacatttaaatcttcagataagtataaatctggaagccaattatttttttacaaaaatagcttgagttttccgagctcagatttgcataaacctaagagagtttgtcgccctgcttcccaactaacagatttacttttccttttcatggacagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtgagccttgtggccacagagcctgtctctgtgggagcccacatgctcccaggtgctctgggtgggctcggagccagcattgcacagggaggcaggcactggtcctctctcagggcggacccgcagatcagcacagcacagggtacaggacgcctcccttgtccagaattgcgggaggagtcatgttggacttgctgagcattttctgattgagtgctagcttttgcactttaagcaccgaacttttattatttgtcctttctttattattattgttttcttttttccttcttttgtgtcagggtctcgctctgtcgtccagactggagtgcagtggtgccgtcatggttcactgcaacctcaaactcctgggctcaagcgatcctcccgccttagcctcccaggtggctgggaccactggtgtgtgccaccatgctcaactggggtttgttttttgttgttgttaagacagagtctcactcggttgcccaggctggagtgcagtggtgcaatctcggcttactgcaacctccaccctgggtttaagtgattcttctgtctcagctatcctgagtagctgggacaacaggcgtgtgccaccatgcccggctaattttttgtatttttagtagagaagagatttcaccatgttggccgagctggtcttgaactcctgaccttgtgatttgcccgcctcggcctcccgaagtgctgggattacaggtgggagccactgtgccccacctttttaaaatttttttgtagagatgaggtctcagtatgttgcccaggttggtctcgaactcaagcaatcctctggctcatcctcccaaagcactaggattacaggtgtgagccgccatgcccagccatgtgtgtctattcttgatggatgtattcctaagataggaagcatttctaggttaggtggttcattgcatcaacatcacctttttttattatctcagtcctcattccaagtctcagtgatgatcccgtgctgctctcagcctgctgaggtgtggatgatttcttcctaaactacgtcgctgctgctgttgctcttgactctgagctcaggtctttgttacaaagaattcgtcgttcagttactaaacttccttgtcattggtgtccacccagttcgacacaccaagctcccttactgttctcattcagtgtggctgtgtgcctgccctcaactgtcctctccttttaatttcctaccgctggtctgctagcaggcagctcggcatgtctgttagaattgtgtgttaaggaagagtaagtatgtaatgaggagggactgaaatcctcttataagaataaagtacgtggggctgggcatggtggcccactcctgtaatcccagccctttgggaggccaaggccggcagatcacctgaggtcaggagttcgagaccagcctggccaacatggtgaaaccccctctctaaaaaaaatacaaaaaaattagccaggcgtggtggcacactcttacaatcccagctactcaggaggctgaggcaggagaattgcttgaacccaggagacggaggttgcagtgggccaggatcacaccactgcattccagcctgggtgacaagagcgagactccatctcaaataaagaaagaaagaaagaataaaatgtgcggatttcagtgcagggagagtgcagtggctgctctgacctgctggttcttctgcatgctccaggtgagtaagctggtacatttgagtctgggagaggtgtgacatatgcatgggagttatttatggctcaaggtggaagaaaagcaataagccttgttaccggcccttgcagtccacaatgacaagtgttgttggaagcttgatcacaggagcagaagagtacagtcttcggctctcatggcccccactgtgccactctccccgttattaacgctggctgatttgtgccagctgactctgaaaagtctgaagaagaggctttgatttggtgtcactaagcgaggaaagggctgtctgagtcgccggtgtccctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgaattcagagatgaggtgccggggcggcagggggccctgcactgcatgagccgctctgtgctgaggttgaggcctggagaccgcgtggcgtcttcccctctgcttacagccagtgcactgtgtgaattgtagtctcttggcttcagagctggaggtcgtgaggagcctcccaggccatcccgcctgctttgctggtctctcttgtgcccgttgtcctggttctcgctcagctctggtttctttgctgcagtcagacccctggtgtctttgtttcctctggccttcatctcagggatgtctgttaatcccattgagttgcaccaccgaatgtaggcctccagctgtggagatgcagctcaggccaggccaggccaggtcgtgctctggtaacagcctcggatctcagcaccttcaagccatagggcctgtttcttactcatggtgtgtgcattgcaggagagtggggctctgcttgctgtggtcgtcctggcaggcagagcggcagccatatgaaaatgcttcccctaggctgggcgcagtggctcacgcctgtaatcccagcactttgggaggccaaggcgggcagatcacctgaggtcgggagtttgagaccagcttgaccaacatggagaaaccccgtctcttccagaaatacaaaattagctgggcatggtggtgcatgcctgtaatcccagctacttgggaggctgaggcaggagaaacgcttgaacccatgaggcagaggttgtagtgagccgagatcacaccattgcactccagcctgggcgacagagtgagactccatctcaaaaaaaaaaagaaaggaaaaaatgttgctccttctgccctccagaagcacagatggtgctgagggccttgtttgtctcagctcattgaccagagccagtcacgtgtgccttagccaagtagtgcacccagaaggggaggtggagacaggtggggagcagcgcagatggccactcacgagaatccccctgggtgccttgggtcactcattaggaaggggacaggagaagctcaaccgatttctgttctcggatagctcctgatgtagccggggcagagctatgctgttgggaatgattggactggtggcactttgtgtgctctgcatggagagagtatagtgggactgatcctccaggagtcaggtcctggagttggctgtgaacgagtaggggtccgagctgctggcagctttggcttaaagaggccatcccttccctttgcatttcaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggtaacgggcgtctctgcttttggcatatctgttaggatagagcagtcacgatacattggttggcttgtccatggatttgggagaacagtccttagccgtgtttgcttgctgcctctgtgaggagcactggcctgtgggcacttgtcagtgagcacagatctgggctgggagtccttgttctgtaggcctcttcccctgagtgtgctgaaaacatattgatgtggaactcacttcttcctgggttcaagtgattctcctgccccagcctcccaagtagctgggattacaggcgcctgtcaccacacccagctaatttttttatttttggtagagacggggtttcaccatgttggccaggctgttctcaaactcctgacctcatgtgatccacctgcctcagcctcccagagtgctgggattacaggtgtgagccaccgcgcccagcctttttttttttttttaaagcaaatctgagtcgtatcattccagccataaatataatcacaatactgtgatcatgtccgacaaaacggacagactcctgagtgttctccaggctgcaagcccttgtgcagtttaccttttgtctcacaagtgactgcattggcatcggagcagcgccccaagtctctctggtttagaacagctcccccttgcatcccctcccctccaagttcctcccatccatctattgaaggaccttggtcgttgacttgtggaactgcctgcatgcgggatctggctggttgtgccctgctggtgttcttttatacccattttttctttaaactggtaggatcaggggctgggtgagcgtcaggtttgctttttcctttggcgagaacgctcatagctggtgctgtgttctccaggctcctgaaggcgtgtggcagccggctgaccgagaagctcctggaaggtgctccgaccgaggataccttggtccagatggagaagctcgctggtgaggcaggggtgctgttgccggggccttggcccggatggccgtggggcggtaccagctgtctgctctcctggcaggaatcgctgagggagggaaacgcggctctgaatcagcccagaacgagccttcgggaagctcaccctccgatctcggtgtgattgttgtgattgttgtgatttcctgtctcgtttgccttgaccgccatgtgaaagaatctgttccccagctaggtggggaaaattcacaggtgggctgtctgtagagagaactggctgattaaaggcttctcgtcccgattttgtgatagccaagtgcttggcctggtcgacggtctttgctcctttacaaataaagtgttctgtttcagttcgtcccaagttttccatgaagggcagtggttccctgacctcccaggtgcctgggcttccccaggttcctgatctggggcttggggccctgtgtttggggatcgtggcactgtgtgcaccagcctggaagcactgggccagtcttggccaagctttccatcagggatgatttgatcttggtgctacaggtctgtggtacgaccattgttccacaccacatgtcattaataatgcttcccatgcttctgcttgcaaatgaccagccttccaaacagccagagctgtttcgaggtgtttctgcaggcaggtgcaggcgtgccctcaaataagctttgccaatggagtctcagcaagagcaaaacctggtcaggaaagacaaagcctgggaatccacccccatgccctgcaggttggctggccctggagccatttattatagtgctaatcatgtttctaggcaggtgcagatggcaagggcagtgtcttggtgagctttttagcacgaagagccaggtctgtcgaagcctttgtgagagctggaaacgcaggtgtgctgggcatgcgcagtatggggtttcgggctcagggcttgccctttggcatcagacagacctggcttcgcatcctggatttgcttctgacgtgcacccttccctttgggtctcgtgatgtgaaatggagatgttgtcatttgtgagggctccatgaagtttcgttgaaatgacaaatactaatttcttcatctgtgaaatggagataatagtgctgacctcagaacagctgagaggactaaatgaaatgatgttggatgtagccataaagaacgaagtcaggcactggtgcacgcctggaatcccagctcttgggagaccgagacaggtggattgcttgagctcaggagtttgagaccagcctgagcaacatagggaggtccagtctctacaaaaaatatgaaaagtagctgggcgtggtggcgcatgcctgtagtcccactacttggaaggcttcgttgggaggatcacttgagcccagaagattgaggctgcagtaagccgtgatcgtgccactgcattccagcctgggcaacagagcgagacactgtctcaaataaaaaagatgggaatagtagacactgggggctccagaaggagggagggagggaggaaggggaggaagggctgaaatgctttctattggatactatctgggcatattacttcctgtggttcactgtctgggtgacaggattcatagaagcccaaactttagcaccacgcagcatacccttgtaacaaagccgcacacgtacgccctcaagctaaaacaaaagtggaccgggaggccgaggtcgggggatcatgaggtcaggagtttgagaccagcctggcagataacggtgaaaccccgtctctactaaaaataccaaaaaaagttagccggacatggtggcaggtgcctgtagtcccagctacttgggaggctggggcagaagaatcgcttgaacccaggaggcggaggttgcagtgagccgagattgcgccactgcactccagcctgtgcgacagagtgagactccgtctcaaaaaaaaaaaaaaaaaaaaaagtggaaaaaaaagaaacaacgtgtagatcctcgagaactgtggccgactgggtgtgttccgtagacacgctgttggcatttttcttactgatttgagttttctcaccagcagagaacagctgacggtctttccataagtgcttctaagagagatgtgtggtaccccgggctaagtagacagcaactgagccctccctcccaccccagggctcccagagcaacagggagcagggagcataggacctggccgcagccaggaatctacactgaccggctcagcccatgaagtatcttgggctgaagtcacaggatgagactgtttgtatctgtaactgtccttgtcatctgtcttgcagatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtaggcggcaggggcacagcgcatcaggagctcaggaggagagccagtcatgggctgtccctgcccacacgccttgcctctggaccctccaccttcaaaaccctgcaggaagtgactgacagcctcctcggtgggtggctgagggcacagggcgttgggggtattagccataggatcagtgctcccctttcagtgatgacctaagtatcttcaaagaaaagcccagatagttggcatcccatggtcctctaatcctctaagaagctcctaaaggcattgaacagcactgtaattggtttggatgcagcctggaccctgcccattgtaagtgatggtctggctgggcgtggtggctctcacctgtaatcccagcactttgggaggccaagggggagcggatcacgaggtcaggagttcgagaccagcctggccaacatggtgaaaccccatctctattaatacaaaaattagctgggcatggtggcatgtgcctgtagtcccagctacttggaggctgaggcaggagaatcgcttgaacctgggaggtggaggttaacagtgagccaagatggcaccactgcactccagactggtgacagagcgagactccatctcaaaaaaaaaaaaaaaaagaaagtgatggtcattcttacaaaaaattaggaacaacttgcatatcaaaccatactagtaaacagtagccaaaccatggaatatgattaattagaagaaataaagcaggcactgtgtgttcagggtgatgtatgtacatgtgtgcctgccagacacgacaaaatctaacagtgttgtctccagttacatcgagtcagatgccgggagagaggggtgtgggttgctggattacaagggcaggctgccttcttataaaattcttctgcatgaatttttccaatgagcactgattcattagaagagagttatttgtttttgaaaatggagtgagactgatttcatggcagatgtccttggggcttctggatgtaaacacccattccatcaaaatcctgcatttggctgaaaccttccttgtccgttgcttatgttgatgaaggttggaaggggtctggaattcctctggaggtgggactactttgaggctgttggggtttggtgagacatagtattgtggcctctctcttatgttgtgtttcaaagctgggtttggcttcttagaagccgtgagacagcaggctggactctcctaaggtgtcaatgtcatggaagttaataacaggtggggtgtggggagaggctgttctaggttaaagaataaggacgtgacaactaaaagccatgatgggtcctgtagtatctcctggatccaaagaacagcagccgtcaagcaggcagcagccctgggcttgtgcacacggctggatgtcagatgcgtccgtccaggcactcctggctgcagtcatggtctcagctgtggagatgcccttattctcctgggtgctgccatgtcgagggccctacagcttgctttcaaatggttcagcagaaagtgtctgtagagagaaggtgaatattgcacaaagttaacatctttttgcttcaaatttttaaaaacaaaactgggagaaagatcttggagcaagagctttggaatctagcatttatgtgcaaacctggaacttctctctctgcccctttaaccctgggccgggcacccgcctccctgagaagataagtgtctttctcctgagccctgtctttgctctacaaagttttatgaacacacaaataacttcatgggaagggagagaaaaaccatttctactagtcacgctgaaagcactgcactctcttgtttaggtcctgggtgccccttctttccgcatgctggcctggcacgttctcatggggaaccaggtgatctggaaaagcagagacgtggacctcgtccagtcagcttttgaagtacttcgggtgagaacatcttttccttaggtgtgcggcgcagggcagtattggcctggtgcctggcgtcaggtggggatgccaaggtgcaccgactgcccagcacagaaccgctgtgcaccacacggtctccactgggggcgcagtgggcacggagacccgggtctggtctgatgatgtttaaatcatcagaaggatgcgtgtgcctgctgactgcccaggcctgagagggaacaaaaatgcatatgacacagtcttggtaaaagcttctttgtgggatgcggtaggagacaaacatctgtgcaggcaaacattaatatgagttaacaccacagagcgaggggcggtacaagctggtgtgtgactggcggtccagacaagagttagggacctggtggtggaggaccctcccccctcacccccactgtgggctgagagtctggggactcctctggcaggagggagggcttggaccgggctgcaggaggcgtgtggggtttgggtagtagagcatggatggccggggctgagggcttcctcgtgtccttggtctgagtcctgctgtcctcctcagaccatgcttcccgtgggctgcgtccgcatcatcccatacagcagccagtacgaggaggcctatcggtgcaacttcctggggctcagcccgcacgtgcagatccccccccacgtgctctcctcaggtgcgtgcccccgcgggctgtggggccatgccctgcgctgaggttccccacgcctcaggcccaaagtggaaccagatctctgtcatgggttgtggagaggaggcctcattgcagggccagcttcccccatggctctgggcctccgtgttctatcttcacacaggtagcggtgggtgagtggacctgatgggaaggttggctggcagggactgtcgtcagtgggctgcaggtcaccatcgtggggtgggaggatgtgtccctggccggaggatcctggtcgtcctggtgattgggtgcctatgagatggaggcctgctgaatgctgggtgtgagctgcctgcttctgtgggccctgccatcctctcctcactcttctactgttcccctgtcctgacttctgctctcctccagcctttttcttcacctcatgtgcccgaggtcttcccggccttcactggtgtggtcagggctcagcaaacccctggtcttttggtttggggatgtccagggatcagccccccaggctcatcagtgacttcccccagcacagcggtgaccagcagctgctgactcatggtccgagtggagctaccctgagcccccaatcagcacccaggctgggtcctgactccctgcgaggcagtgccttttttccgggtagcattggaaacacaggctctggtttcaaacagacttcaaattcttaccactacgagcaggttatttaacgtctccaggcctgtttccccatctgtagaatagggtcatggtgagaccaccctcatagggctgcttagccagaggtaaattgatgtatataaagtactttgtgattattgtaatataataaattattagtttaactgaggcaaaagttaacccttaactattcacgaataacctgaaaggttttaaataccaaagcattgatttgggccagtcttttcaccagcagccttccccactgacctgggatgagcggagtggggcccacggtgggctagcgcaggggaggtgaggagcatgggggagatgaggagccacgggccttgtgttgttacagagtttgctgtcatcgtggaggtccacgcagccgcacgttccaccctccaccctgtggggtgtgaggatgaccagtctctcagcaagtacgagtttgtggtgaccagtgggagccctgtagctgcagaccgaggtgggtgcccccaggcagagtgtcctttttgtgagaagatgcccccgccccgcgctaacgcctgcccttgctctgcccctgcccttctccccagtgggccccaccatcctgaataagattgaagcggctctgaccaaccagaacctgtctgtggatgtggtggaccagtgcctcgtctgcctcaaggaggagtggatgaagtaagcaacagtgtgggtgaggcccctttgcttgcgaccctggagaaaacctggagctgtttccaaaagaggagctgggccgtggccactgagggaggagccgagagaagaggttgggggcggggttccaactccacgtccgccactgcaaagctcaggtggcctcgggaggcggtcatcatgtctgcctccggctcctgcatggggaggtgggggtggcgcctgccttggagagcgactgaggcttgggcaagggctccaagctcacaggagggttcataatggaagctctgtcatctgctgtgcaattaacttcaaaacctaaaagcccagtaacccatgccccaccccgctttttattttattttatttttattttttattttttttgagtcagagtttggctcttgttgcccaggctggagtgcaatggcatgatcttggctcaccacaacctccgcctcccgggttcaagcgattcttctgcctcagcctctggagtagctgggattacaggcacccaccaccatgcctggctaatatttgtatttttagtagagacagggttttagcatgttggccaggctggtcttgaactcctgacttcgtgatccgcccaccttggcctcccaaagtgctgggattacaggcgtgagccactgcacctggccccccgggtttttttttttgtaagcaaatttactttatttcctcctgcctcgattcttggtaattttttgggtcaaatttattaatcaaacattgtcctgatttaaaatcatgcagtgaagcttaggagcaaccagtgtcttccacgagtttaaggaaggtttttctttcttcttggcgtcttgacaacagcttgagggagggtgtgggaagcgcacaggccccatacccatacccgacagagggccgagcccagccccgacagcttcccttccttgctgggacacagctcctttcagcagttcaggggccaagcctgctgccccaagggtgtggattccagctctgcctgcacagctcctgctggtgccaaagccgtgtcacccctggttggccccgtgaccagggctcgagggattgtgctgtggtgtctgagtgttttgttttggttttcttcagcaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcctggcccttttttttttttttttttttttttttaaagagatggcatcttgctatgtcgtccaggctggtcttgaactcctgagttcaagcagtcctcctgcttcaacatacagctacaggtaccccccactatacatttttaataaggattcatggctcagagggattttctgatggttttgctgatttgtttctagtttttttgtgtttatatttaacatgaagaccaagtttatataactaggtatctgtataatgcaacaacattggaacacaataaagatgtatttttgtaaattgttactacatggttttattggtttgatgcctactatttatttgcgctcaggcacaggaaatactatggttaaaaaataccagtgagtcagccgggcgcagtggctcatgcctgtaatctcagcactttgggaggctgaggcaggcggatcacgaggtcaggagatcgacaccatcctggctaatacggtgaaaccctgtctgtactaaaaatataaaaaattagccaggcatggtgacagatgcctgtagtcccagctactcaggaagctgaggcaggagaatcacttgaacccgggaggcagaggttgcagtgagccaagatcacaccactgcactccagcctgggcaacagagtgagactccatctcaaaaaacaaaacaaaataccagtgagtggccaattcgcacagttaagctgctataatgatcgtagacaatcttctccaagttagtcgttggagttggtgttggagagaagcaaacactcatttatcccaaggacatctgttgagtacctatgcccatgtgcttttcattttgttcagcattagaaatggctcatcccttaataatgatgatatttaaaagacatgtccatctgagaggaagatggctggaatcaccctgggtgaccgtggcaatccctaaaacagcaggttcacggtttagtcatctacctcgagtgggaggggaccccctggccaatgtgggtcaagggagagatgaccacccactgtcctccccggcttcgggtacagattcctgtctttctctcgtgcatggcattcctgggaacgtggttctatgctcagtttttgccagttgtctgccagtctagtggctgaatccacttggcaagtaggagcccagtgtaagttagctctgcattcacttcaggggcatgtgtgacctggatttcccatgtctaacagagcttgctggcgagggggcaggcaaggccctatgccgactggtggttttgtttttgtttttgttttttgagacagagtcttgctccgttgccaggctgtagtgcagtggtgtgatctcggctcactgcaagctccgactccctggttcaagcaattcagcctcagcctcccgagtaactgggattacaggtacatgccaccacacccggctaatttttgtatttttagtagagatggggtttcatcatgttggccagactggtctcgaactcctgacctcgtgatccgcccgcctcggcctcctaaagtgctggaattagaggggtgagccaccgcacccagctgccaactggtgtttctaggcagctgtgggctttttctccccacaggcatgcagttcttgaagatttttcctattggtcatgagtgcagaagagcaggaggctgagctgaacctatgccagcccaagaccaggccagactctgctgtccttgtgagggaataggggttcctgattttgtagcttttcacaaaatggaagagacagaaaacaagttccagatcttgaaccttccccatgtgtttgaaggcagtcaagggtttcctgattttacattcaagatacttacactcagggccgggcatggtggctcacgcctgtaatcccagcactttgggaggccgaggcaggaggatcacctgagcccaggagtttgataccagcctggccaacatggtgaaaccccgtctctactaaaaatacaaaaaaaaaaattagccaggctggtggtgtacgcctgtagtcccagctactcaggaggctgaggcaggagaatcacttgagctcttatatattgtggaaattaaccccttatctgataatatagtttgtataagcttttaaaaaataaattgtatatatttaaggtatacgacatgatgttaatgacatacacacacatattaaaatggttattatagccaggcactgtggctcacacctgtaatcccagcactttgggaggccaaggtgggcggatcacctgaggtcaggtgttcgagaccagcctggccaacatggtgaactcccatctctactaaaagtacaaaaattagctaggcgtggtgtcaggcacctgtaatcccagctgctcaggaggctgaggcaggagaatcgcttgaaccagggaggtggaggttgcagtgagctgagatggccccatggcactcctgtctgggcagcaaagcgagactctgtctcaaaaaataaaaaataaacacaaacggccaggcacggtggctcacacctgtaatcccagcactttgggaggccaaggtgggcggatcacctgatgtgaggtgttcgagaccagcctggccaacatggtgaaaccccgtctccactaaacatacaaaaattagccgggcgtggtggtgggtgcctgtaatcccagctactctggaggctgaggcaggagaattgcttgaatctgggagacggaggttgccgtgagccaagatcacgtcactgctgacagagcaagactccatctgaaaaataaatgaataaaaaactaggccgggcacggtggctcacgcctgtaatcccagcactttgggaggccgaggagggcagatagtgaggtcaggagatcgagaccatcctggctaacatagtgaaaccccatctctactaaaaatacgaaaaattagctgggcgtggtggcgggcacctgtagtcccagctacttgggaggctgaggcaggagaatggcgtgaacctgggaggcagagcttgcaaagtgagctgagatcgcaccactgcactccagcctgggcaacagagcaagactccgtctcaaataaataaataaataaataaactaataactaaatgttgtaacacaaaagaggataaatgcttaaggtgatggatatcctgtttaccctgatgtgattattacaaattgcatgtctgtaccaaaatatctcatgtacccgataaatatatatacctactatataccgacaaagttaaaaatagaaactggaagacaaaaaataaataaaaatcaaataaagcttctcattttgttgtctgcctttggtagatttccagccctgaagtggctgtttttgaaaactgttttcccccagttttatttttgtttatccctctctggagggatttgagtccttctttatacctccacaactccaagtccctcctctcattatcgttttagatgaggaaagtgaagctcagaagagatgaatcaatttgccctagaccatgcagctaggaagcctgggagccagcactaaactcaagtgttctttttctttgttaagaattttattcgccaggcacggtggctcacacctgtaattccagcactttgggaggccgaggcaggctgatcacgaggtcaggcgatcgagaccatcctagctaacacgatgaaaccccgtctctactaaaaatacaaaaacaaaaatttagccgatgtggtggtgtgcatttgtagtcccagctacttgggaggctgaggcaggagaattgcttgaacccaggaggtgaaggttgcagtgagccgagatcgtgccactgcactccagactgggcgacagagcgagacttcatctcaaaaaaaaaaaaaagaaggccgggtgcggtggctcacgcctgtaatcccagcactttgggaggccgaggcaggtggatcacgaggtcaggagttcgagaccagcacggccaagatggtgaaaccccatctctactaaaaatacaaaaattagccgggtgtggtggtgggcgcctgtaatcccagctgctcgggaggctgacgcagagactcacttgaaccccggaggcagaggttgcagtgagccgagatcgcaccactgcacaccagcctgggcgaaagaacgaggctccgtcttaaaaaaaaaaaaaagaattttattgacataatcactgacatacatttttagttatttacaccatacaatttgacatatgtatatacctgtgaaaccaccacagtcaacccgataagcacacatccatcactccacttccttgtggctttgttggcctccaggctacactggtctgctttccctcaatctagtttgcattttctagaattttatgtaaatggaatcatacagtatgtactctttttgtctggcttttcccactcaatattaatgaaattcatccacgtcgtagcacatgtgagtactgtttttttttttttttggtgggggggggatagggtcttgctgtgtcacccaggctggagtgcaatggcatgatcttggctcactgcaaccttagtctccagagctcaagcgattctcccacctcagcctcccgagtagctgggatcacaggcacgtcagcgcactcagctaatttttgtattttttggtagagacagtgtttcaccatgttggccaagctggtttcgaattcccgaactcaagctgtcctcctgcctcagcctcccaaagttctgggataacaggcatgaaccaccgtgccaggccacttcattctattttattgctaaatagcattcccttgtacagatatacaaaagcttatttacctgtttacctgctgacgcctttttgggttgttctaggcacatacgcctgccaggacacgtgcttgtgttaatggatcccgccctttctcctaggagtgggatagctggatcatatggaaggtgcaagtttaatattttaataaacttttaatttagaataatttgctttacagaaaagtttcgaatatagaacagagagatccatttacccccacgcagcttcctttgttaaccttttacattcctttcatacatttgtcaaagctaagaaactgacattggtacaatgcgattaagctccagactttatttggctctcaccagtttttccgtgaatgtcttctttcggttccaggatccagcccagaccaccacattgccccgtttccctatgcccccggcccagtgccctcggctgccttagtctcttcctctgtttatgcttaaagcagggcaggccttccaacaacgccggctggaaaaaacctcttgggctaggaaaaaacctctgggccgacaccgcctccagtatgctgggttccaatgctgcctccagctgtgttgattccctgattatctgtgttcccaggtcattccccagcccagcctagctgctcccgagaagacagtgaaggtgtgaccccacatttccagagcagctctgctgtgctccatgcgaggaaagccggggttgctgtggcttctagcgcggagagcggtctctggcctgggcaggtgacgggtgtgctagggcccgtg

SEQ ID NO: 3 - DNA, 3325 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctggtgaggcaggggtgctgttgccggggccttggcccggatggccgtggggcggtaccagctgtctgctctcctggcaggaatcgctgagggagggaaacgcggctctgaatcagcccagaacgagccttcgggaagctcaccctccgatctcggtgtgattgttgtgattgttgtgatttcctgtctcgtttgccttgaccgccatgtgaaagaatctgttccccagctaggtggggaaaattcacaggtgggctgtctgtagagagaactggctgattaaaggcttctcgtcccgattttgtgatagccaagtgcttggcctggtcgacggtctttgctcctttacaaataaagtgttctgtttcagttcgtcccaagttttccatgaagggcagtggttccctgacctcccaggtgcctgggcttccccaggttcctgatctggggcttggggccctgtgtttggggatcgtggcactgtgtgcaccagcctggaagcactgggccagtcttggccaagctttccatcagggatgatttgatcttggtgctacaggtctgtggtacgaccattgttccacaccacatgtcattaataatgcttcccatgcttctgcttgcaaatgaccagccttccaaacagccagagctgtttcgaggtgtttctgcaggcaggtgcaggcgtgccctcaaataagctttgccaatggagtctcagcaagagcaaaacctggtcaggaaagacaaagcctgggaatccacccccatgccctgcaggttggctggccctggagccatttattatagtgctaatcatgtttctaggcaggtgcagatggcaagggcagtgtcttggtgagctttttagcacgaagagccaggtctgtcgaagcctttgtgagagctggaaacgcaggtgtgctgggcatgcgcagtatggggtttcgggctcagggcttgccctttggcatcagacagacctggcttcgcatcctggatttgcttctgacgtgcacccttccctttgggtctcgtgatgtgaaatggagatgttgtcatttgtgagggctccatgaagtttcgttgaaatgacaaatactaatttcttcatctgtgaaatggagataatagtgctgacctcagaacagctgagaggactaaatgaaatgatgttggatgtagccataaagaacgaagtcaggcactggtgcacgcctggaatcccagctcttgggagaccgagacaggtggattgcttgagctcaggagtttgagaccagcctgagcaacatagggaggtccagtctctacaaaaaatatgaaaagtagctgggcgtggtggcgcatgcctgtagtcccactacttggaaggcttcgttgggaggatcacttgagcccagaagattgaggctgcagtaagccgtgatcgtgccactgcattccagcctgggcaacagagcgagacactgtctcaaataaaaaagatgggaatagtagacactgggggctccagaaggagggagggagggaggaaggggaggaagggctgaaatgctttctattggatactatctgggcatattacttcctgtggttcactgtctgggtgacaggattcatagaagcccaaactttagcaccacgcagcatacccttgtaacaaagccgcacacgtacgccctcaagctaaaacaaaagtggaccgggaggccgaggtcgggggatcatgaggtcaggagtttgagaccagcctggcagataacggtgaaaccccgtctctactaaaaataccaaaaaaagttagccggacatggtggcaggtgcctgtagtcccagctacttgggaggctggggcagaagaatcgcttgaacccaggaggcggaggttgcagtgagccgagattgcgccactgcactccagcctgtgcgacagagtgagactccgtctcaaaaaaaaaaaaaaaaaaaaa

SEQ ID NO: 4 - DNA, 3929 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcaggttttgtcttcgctctgtttggaggagagggtgtgtgtcatcctcttctcccagtttggcgttcaggagggtcctctgatgcgctaatagggtagcaccgtgtcctccagggagggtggaagaccgcgcttctctccagttgagagtactgtcagtcgcgtccttgtctcctggaaagaatggattggcttgtggattgaagtccaagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgagtgcagggagagtgcagtggctgctctgacctgctggttcttctgcatgctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcctggcccttttttttttttttttttttttttttaaagagatggcatcttgctatgtcgtccaggctggtcttgaactcctgagttcaagcagtcctcctgcttcaacatacagctacaggtaccccccactatacatttttaataaggattcatggctcagagggattttctgatggttttgctgatttgtttctagtttttttgtgtttatatttaacatgaagaccaagtttatataactaggtatctgtataatgcaacaacattggaacacaataaagatgtatttttgtaaa

SEQ ID NO: 5 - DNA, 3950 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcagaacccccaaagcctccccagtctctgataacacatccagggtctttcggtggcctggaggagctataagatctgcttcctacagcctccaccctgtctgctgctgctctttggacaccggaccttacacacccctcagacagtacagcacccaccctgcccttcaactcttgcttggccccgtaagccagtccttccaggctcctgcctcctctggtcattccctgcgtgggcctctcgcaggaagcagttttcctgtgtcccccgccgaccccctgcaggaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcctggcccttttttttttttttttttttttttttaaagagatggcatcttgctatgtcgtccaggctggtcttgaactcctgagttcaagcagtcctcctgcttcaacatacagctacaggtaccccccactatacatttttaataaggattcatggctcagagggattttctgatggttttgctgatttgtttctagtttttttgtgtttatatttaacatgaagaccaagtttatataactaggtatctgtataatgcaacaacattggaacacaataaagatgtatttttgtaaa

SEQ ID NO: 6 - DNA, 3866 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcagcctccaccctgtctgctgctgctctttggacaccggaccttacacacccctcagacagtacagcacccaccctgcccttcaactcttgcttggccccgtaagccagtccttccaggctcctgcctcctctggtcattccctgcgtgggcctctcgcaggaagcagttttcctgtgtcccccgccgaccccctgcaggaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcctggcccttttttttttttttttttttttttttaaagagatggcatcttgctatgtcgtccaggctggtcttgaactcctgagttcaagcagtcctcctgcttcaacatacagctacaggtaccccccactatacatttttaataaggattcatggctcagagggattttctgatggttttgctgatttgtttctagtttttttgtgtttatatttaacatgaagaccaagtttatataactaggtatctgtataatgcaacaacattggaacacaataaagatgtatttttgtaaa

SEQ ID NO: 7 - DNA, 2298 bp, Homo sapiens
gagtcacgcgcctgggtgtcggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgagtgcagggagagtgcagtggctgctctgacctgctggttcttctgcatgctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactga

SEQ ID NO: 8 - DNA, 3488 bp, Homo sapiens
tcggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggtgtcccattgctggctctgccacggaaactctccaaagggctctctctacccactcctgtgagctttttctccaccggaaactgtgttaaggtcacctttgacttcatcattgttctgcaggttaccactcccccttttaaaaagattgtggtgaaataatacatgacctaaaatgcgccatttgaaccactgttcggcgtgcagtgccgtggcatgaagtatgttcacagtgctgtgcggccatcgccaccttccgtctccagaactgagccccgctcttccctcccagtccccggcagctgccattctgctctctgtctctatggctttgaggactctaggtacctcctatttaagtggaatcctacagtgtttatccgtttgtgaccagtgtagatcatttcgtgtaatgtccttcagtttcagccgtgccgtagcttgtggcagggtttccttcctttttaaggccatgtgacattccagtcccaccttgctaaagcagtttacctggtcactggggtgactctggagctcccctctggcctcactgcctgtgcttggtcatgttggctagctgtcctcccttctccacctctgagtctacaagggctgcactccacggaggtcccatgcgtcagccccgaccgcagcgcttggctctgctgctgtgtcccgtccccgtgaaggctcagcaggcactcactgggccttctcccctgctcactgctccgcactctgcaccccacccgcactgtgcgtcttcaccctgcctggcctgggccactgtcccttcctgctcagatcagtcagcagggtgcctcagcttgttccccacctccaccctggccccctgccatcttttctgcacatgggcatgagtgaacctattcaagtgaaattcacattttctcatgtctcatcagaacccccaaagcctccccagtctctgataacacatccagggtctttcggtggcctggaggagctataagatctgcttcctacagcctccaccctgtctgctgctgctctttggacaccggaccttacacacccctcagacagtacagcacccaccctgcccttcaactcttgcttggccccgtaagccagtccttccaggctcctgcctcctctggtcattccctgcgtgggcctctcgcaggaagcagttttcctgtgtcccccgccgaccccctgcaggaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgagtgcagggagagtgcagtggctgctctgacctgctggttcttctgcatgctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactga

SEQ ID NO: 9 - DNA, 2479 bp, Homo sapiens
tcggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcagcctccaccctgtctgctgctgctctttggacaccggaccttacacacccctcagacagtacagcacccaccctgcccttcaactcttgcttggccccgtaagccagtccttccaggctcctgcctcctctggtcattccctgcgtgggcctctcgcaggaagcagttttcctgtgtcccccgccgaccccctgcaggaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgagtgcagggagagtgcagtggctgctctgacctgctggttcttctgcatgctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactga

SEQ ID NO: 10 - DNA, 2184 bp, Homo sapiens
ggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgagtgcagggagagtgcagtggctgctctgacctgctggttcttctgcatgctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactga

SEQ ID NO: 11 - DNA, 2606 bp, Homo sapiens
gtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcaggttttgtcttcgctctgtttggaggagagggtgtgtgtcatcctcttctcccagtttggcgttcaggagggtcctctgatgcgctaatagggtagcaccgtgtcctccagggagggtggaagaccgcgcttctctccagttgagagtactgtcagtcgcgtccttgtctcctggaaagaatggattggcttgtggattgaagtccaagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcagcctccaccctgtctgctgctgctctttggacaccggaccttacacacccctcagacagtacagcacccaccctgcccttcaactcttgcttggccccgtaagccagtccttccaggctcctgcctcctctggtcattccctgcgtgggcctctcgcaggaagcagttttcctgtgtcccccgccgaccccctgcaggaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactga

SEQ ID NO: 12 - DNA, 2220 bp, Homo sapiens
gagtcacgcgcctgggtgtcggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgagtgcagggagagtgcagtggctgctctgacctgctggttcttctgcatgctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaagtcattccccagcccagcctagctgctcccgagaagacagtgaaggtgtgaccccacatttccagagcagctctgctgtgctccatgcgaggaaagccggggttgctgtggcttctagcgcgga

SEQ ID NO: 13 - DNA, 3169 bp, Homo sapiens
gagtcacgcgcctgggtgtcggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcc

SEQ ID NO: 14 - DNA, 2043 bp, Homo sapiens
ccgtgtcctccagggagggtggaagaccgcgcttctctccagttgagagtactgtcagtcgcgtccttgtctcctggaaagaatggattggcttgtggattgaagtccaagaaaacgccaggtcttcaagggtgtctgccaccaccatgcctgacccatttggcagcagcctcgtgtgtggtggtctggtgtggacggtggaagcgtgattctgctgagtgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgagtgcagggagagtgcagtggctgctctgacctgctggttcttctgcatgctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactga

SEQ ID NO: 15 - DNA, 2221 bp, Homo sapiens
gagtcacgcgcctgggtgtcggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgagtgcagggagagtgcagtggctgctctgacctgctggttcttctgcatgctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggat

SEQ ID NO: 16 - DNA, 20 bp, Artificial Sequence
  Synthetic oligonucleotide
catggctgatatatcccggg

SEQ ID NO: 17 - DNA, 20 bp, Artificial Sequence
  Synthetic oligonucleotide
cagccagcgttaataacggg

SEQ ID NO: 18 - DNA, 22 bp, Artificial Sequence
  Synthetic oligonucleotide
tcctctggtgtaggaatggcgt

SEQ ID NO: 19 - DNA, 18 bp, Artificial Sequence
  Synthetic oligonucleotide
cagccagcgttaataacg

SEQ ID NO: 20 - DNA, 18 bp, Artificial Sequence
  Synthetic oligonucleotide
ctggtgtaggaatggcgt

SEQ ID NO: 21 - DNA, 18 bp, Artificial Sequence
  Synthetic oligonucleotide
tggctgatatatcccggg

SEQ ID NO: 22 - DNA, 19 bp, Artificial Sequence
  Synthetic oligonucleotide
atttaatggaggtctcttt

SEQ ID NO: 23 - DNA, 19 bp, Artificial Sequence
  Synthetic oligonucleotide
tcatggctgatatatcccg

SEQ ID NO: 24 - DNA, 19 bp, Artificial Sequence
  Synthetic oligonucleotide
aatggcgtgaaggctgtgt

SEQ ID NO: 25 - DNA, 19 bp, Artificial Sequence
  Synthetic oligonucleotide
tctggaccaaggtatcctc

SEQ ID NO: 26 - DNA, 19 bp, Artificial Sequence
  Synthetic oligonucleotide
atggtgatgatgctgtacc

SEQ ID NO: 27 - DNA, 21 bp, Artificial Sequence
  Synthetic oligonucleotide
atcatggctgatatatcccgg

SEQ ID NO: 28 - DNA, 21 bp, Artificial Sequence
  Synthetic oligonucleotide
atgaggtgtgacttgtaggtc

SEQ ID NO: 29 - DNA, 21 bp, Artificial Sequence
  Synthetic oligonucleotide
aatcttattcaggatggtggg

SEQ ID NO: 30 - DNA, 20 bp, Artificial Sequence
  Synthetic oligonucleotide
aaaaaaaaaaagggccaggc

SEQ ID NO: 31 - DNA, 20 bp, Artificial Sequence
  Synthetic oligonucleotide
tgaaattgtcttcaatcacc

SEQ ID NO: 32 - DNA, 20 bp, Artificial Sequence
  Synthetic oligonucleotide
aactgtaaccaaacgtaaat

SEQ ID NO: 33 - DNA, 20 bp, Artificial Sequence
  Synthetic oligonucleotide
tgaaagcagcaataaagaca

SEQ ID NO: 34 - DNA, 20 bp, Artificial Sequence
  Synthetic oligonucleotide
ggactccatcattaagcccc

SEQ ID NO: 35 - DNA, 20 bp, Artificial Sequence
  Synthetic oligonucleotide
accttcaccagcacctgcag

SEQ ID NO: 36 - DNA, 20 bp, Artificial Sequence
  Synthetic oligonucleotide
cacatttcagaggaccaaaa

SEQ ID NO: 37 - DNA, 20 bp, Artificial Sequence
  Synthetic oligonucleotide
gatgaggaagagatccactt

SEQ ID NO: 38 - DNA, 20 bp, Artificial Sequence
  Synthetic oligonucleotide
cttcactccccaaagtctct

SEQ ID NO: 39 - DNA, 20 bp, Artificial Sequence
  Synthetic oligonucleotide
gcggagccctaactcaatca

SEQ ID NO: 40 - DNA, 20 bp, Artificial Sequence
  Synthetic oligonucleotide
gtactgaatattcacaactg

SEQ ID NO: 41 - DNA, 20 bp, Artificial Sequence
  Synthetic oligonucleotide
gcttgaatgttaacctcggg

SEQ ID NO: 42 - DNA, 20 bp, Artificial Sequence
  Synthetic oligonucleotide
aacctcgggagcagacatgt

SEQ ID NO: 43 - DNA, 20 bp, Artificial Sequence
  Synthetic oligonucleotide
catgttattgcgactgcata

SEQ ID NO: 44 - DNA, 20 bp, Artificial Sequence
  Synthetic oligonucleotide
cctgtttctcctgcgggttt

SEQ ID NO: 45 - DNA, 20 bp, Artificial Sequence
  Synthetic oligonucleotide
agagagagcccatcatccct

SEQ ID NO: 46 - DNA, 20 bp, Artificial Sequence
  Synthetic oligonucleotide
gcgattccaacggctggagg

SEQ ID NO: 47 - DNA, 20 bp, Artificial Sequence
  Synthetic oligonucleotide
aaacctgacagggccgagcc

SEQ ID NO: 48 - DNA, 20 bp, Artificial Sequence
  Synthetic oligonucleotide
gacacagctccttccagcag

SEQ ID NO: 49 - DNA, 20 bp, Artificial Sequence
  Synthetic oligonucleotide
gtggacagccatccctgtct

SEQ ID NO: 50 - DNA, 20 bp, Artificial Sequence
  Synthetic oligonucleotide
tgcggaccgtggacatgagg

SEQ ID NO: 51 - DNA, 20 bp, Artificial Sequence
  Synthetic oligonucleotide
gacttgtaggtcttgctcag

SEQ ID NO: 52 - DNA, 20 bp, Artificial Sequence
  Synthetic oligonucleotide
caatccctcgagccctggtc

SEQ ID NO: 53 - DNA, 20 bp, Artificial Sequence
  Synthetic oligonucleotide
aggcttggcccctgaactgc

SEQ ID NO: 54 - DNA, 20 bp, Artificial Sequence
  Synthetic oligonucleotide
ggaagctgtcggggctgggc

SEQ ID NO: 55 - DNA, 20 bp, Artificial Sequence
  Synthetic oligonucleotide
ctcggccctctgtcgggtat

SEQ ID NO: 56 - DNA, 20 bp, Artificial Sequence
  Synthetic oligonucleotide
gtatgggtatggggcctgtg

SEQ ID NO: 57 - DNA, 20 bp, Artificial Sequence
  Synthetic oligonucleotide
tcaagctgttgtcaagacgc

SEQ ID NO: 58 - DNA, 20 bp, Artificial Sequence
  Synthetic oligonucleotide
cgtggaagacactggttgct

SEQ ID NO: 59 - DNA, 20 bp, Artificial Sequence
  Synthetic oligonucleotide
taccaagaatcgaggcagga

SEQ ID NO: 60 - DNA, 20 bp, Artificial Sequence
  Synthetic oligonucleotide
aaaacccggggggccaggtg

SEQ ID NO: 61 - DNA, 20 bp, Artificial Sequence
  Synthetic oligonucleotide
tgggcggatcacgaagtcag

SEQ ID NO: 62 - DNA, 20 bp, Artificial Sequence
  Synthetic oligonucleotide
cacgaagtcaggagttcaag

SEQ ID NO: 63 - DNA, 20 bp, Artificial Sequence
  Synthetic oligonucleotide
gtggtgagccaagatcatgc

SEQ ID NO: 64 - DNA, 20 bp, Artificial Sequence
  Synthetic oligonucleotide
ggggcatgggttactgggct

SEQ ID NO: 65 - DNA, 20 bp, Artificial Sequence
  Synthetic oligonucleotide
cctcagtcgctctccaaggc

SEQ ID NO: 66 - DNA, 20 bp, Artificial Sequence
  Synthetic oligonucleotide
ctctccaaggcaggcgccac

SEQ ID NO: 67 - DNA, 20 bp, Artificial Sequence
  Synthetic oligonucleotide
gaggccacctgagctttgca

SEQ ID NO: 68 - DNA, 20 bp, Artificial Sequence
  Synthetic oligonucleotide
gtggcggacgtggagttgga

SEQ ID NO: 69 - DNA, 20 bp, Artificial Sequence
  Synthetic oligonucleotide
ggagttggaaccccgccccc

SEQ ID NO: 70 - DNA, 20 bp, Artificial Sequence
  Synthetic oligonucleotide
aggggcagagcaagggcagg

SEQ ID NO: 71 - DNA, 20 bp, Artificial Sequence
  Synthetic oligonucleotide
cctcacctcccctgcgctag

SEQ ID NO: 72 - DNA, 20 bp, Artificial Sequence
  Synthetic oligonucleotide
ccctgcgctagcccaccgtg

SEQ ID NO: 73 - DNA, 20 bp, Artificial Sequence
  Synthetic oligonucleotide
ctcgtagtggtaagaatttg

SEQ ID NO: 74 - DNA, 20 bp, Artificial Sequence
  Synthetic oligonucleotide
ccaatgctacccggaaaaaa

SEQ ID NO: 75 - DNA, 20 bp, Artificial Sequence
  Synthetic oligonucleotide
gcctcgcagggagtcaggac

SEQ ID NO: 76 - DNA, 20 bp, Artificial Sequence
  Synthetic oligonucleotide
ttgggggctcagggtagctc

SEQ ID NO: 77 - DNA, 20 bp, Artificial Sequence
  Synthetic oligonucleotide
ccactcggaccatgagtcag

SEQ ID NO: 78 - DNA, 20 bp, Artificial Sequence
  Synthetic oligonucleotide
gtttgctgagccctgaccac

SEQ ID NO: 79 - DNA, 20 bp, Artificial Sequence
  Synthetic oligonucleotide
cacaccagtgaaggccggga

SEQ ID NO: 80 - DNA, 20 bp, Artificial Sequence
  Synthetic oligonucleotide
cgggaagacctcgggcacat

SEQ ID NO: 81 - DNA, 20 bp, Artificial Sequence
  Synthetic oligonucleotide
gcattcagcaggcctccatc

SEQ ID NO: 82 - DNA, 20 bp, Artificial Sequence
  Synthetic oligonucleotide
agtccctgccagccaacctt

SEQ ID NO: 83 - DNA, 20 bp, Artificial Sequence
  Synthetic oligonucleotide
ccaaccttcccatcaggtcc

SEQ ID NO: 84 - DNA, 20 bp, Artificial Sequence
  Synthetic oligonucleotide
gcacgcacctgaggagagca

SEQ ID NO: 85 - DNA, 20 bp, Artificial Sequence
  Synthetic oligonucleotide
acgtggggggggatctgcac

SEQ ID NO: 86 - DNA, 20 bp, Artificial Sequence
  Synthetic oligonucleotide
gagccccaggaagttgcacc

SEQ ID NO: 87 - DNA, 20 bp, Artificial Sequence
  Synthetic oligonucleotide
ctccaccaccaggtccctaa

SEQ ID NO: 88 - DNA, 20 bp, Artificial Sequence
  Synthetic oligonucleotide
tccctaactcttgtctggac

SEQ ID NO: 89 - DNA, 20 bp, Artificial Sequence
  Synthetic oligonucleotide
ccgccagtcacacaccagct

SEQ ID NO: 90 - DNA, 20 bp, Artificial Sequence
  Synthetic oligonucleotide
agcttgtaccgcccctcgct

SEQ ID NO: 91 - DNA, 20 bp, Artificial Sequence
  Synthetic oligonucleotide
gcttttaccaagactgtgtc

SEQ ID NO: 92 - DNA, 20 bp, Artificial Sequence
  Synthetic oligonucleotide
tatgcatttttgttccctct

SEQ ID NO: 93 - DNA, 20 bp, Artificial Sequence
  Synthetic oligonucleotide
cccccagtggagaccgtgtg

SEQ ID NO: 94 - DNA, 20 bp, Artificial Sequence
  Synthetic oligonucleotide
cagcggttctgtgctgggca

SEQ ID NO: 95 - DNA, 20 bp, Artificial Sequence
  Synthetic oligonucleotide
aagatgttctcacccgaagt

SEQ ID NO: 96 - DNA, 20 bp, Artificial Sequence
  Synthetic oligonucleotide
cttcaaaagctgactggacg

SEQ ID NO: 97 - DNA, 20 bp, Artificial Sequence
  Synthetic oligonucleotide
gaggtccacgtctctgcttt

SEQ ID NO: 98 - DNA, 20 bp, Artificial Sequence
  Synthetic oligonucleotide
ccaggccagcatgcggaaag

SEQ ID NO: 99 - DNA, 20 bp, Artificial Sequence
  Synthetic oligonucleotide
ggaaagaaggggcacccagg

SEQ ID NO: 100 - DNA, 20 bp, Artificial Sequence
  Synthetic oligonucleotide
gcacccaggacctaaacaag

SEQ ID NO: 101 - DNA, 20 bp, Artificial Sequence
  Synthetic oligonucleotide
agtgctttcagcgtgactag

SEQ ID NO: 102 - DNA, 20 bp, Artificial Sequence
  Synthetic oligonucleotide
ggctcaggagaaagacactt

SEQ ID NO: 103 - DNA, 20 bp, Artificial Sequence
  Synthetic oligonucleotide
atcttctcagggaggcgggt

SEQ ID NO: 104 - DNA, 20 bp, Artificial Sequence
  Synthetic oligonucleotide
agggttaaaggggcagagag

SEQ ID NO: 105 - DNA, 20 bp, Artificial Sequence
  Synthetic oligonucleotide
gagagaagttccaggtttgc

SEQ ID NO: 106 - DNA, 20 bp, Artificial Sequence
  Synthetic oligonucleotide
tgcacataaatgctagattc

SEQ ID NO: 107 - DNA, 20 bp, Artificial Sequence
  Synthetic oligonucleotide
ccaagatctttctcccagtt

SEQ ID NO: 108 - DNA, 20 bp, Artificial Sequence
  Synthetic oligonucleotide
tattcaccttctctctacag

SEQ ID NO: 109 - DNA, 20 bp, Artificial Sequence
  Synthetic oligonucleotide
tttctgctgaaccatttgaa

SEQ ID NO: 110 - DNA, 20 bp, Artificial Sequence
  Synthetic oligonucleotide
atttgaaagcaagctgtagg

SEQ ID NO: 111 - DNA, 20 bp, Artificial Sequence
  Synthetic oligonucleotide
ggccctcgacatggcagcac

SEQ ID NO: 112 - DNA, 20 bp, Artificial Sequence
  Synthetic oligonucleotide
tactacaggacccatcatgg

SEQ ID NO: 113 - DNA, 20 bp, Artificial Sequence
  Synthetic oligonucleotide
cagcctctccccacacccca

SEQ ID NO: 114 - DNA, 20 bp, Artificial Sequence
  Synthetic oligonucleotide
tccagcctgctgtctcacgg

SEQ ID NO: 115 - DNA, 20 bp, Artificial Sequence
  Synthetic oligonucleotide
ttctaagaagccaaacccag

SEQ ID NO: 116 - DNA, 20 bp, Artificial Sequence
  Synthetic oligonucleotide
agaggccacaatactatgtc

SEQ ID NO: 117 - DNA, 20 bp, Artificial Sequence
  Synthetic oligonucleotide
caaagtagtcccacctccag

SEQ ID NO: 118 - DNA, 20 bp, Artificial Sequence
  Synthetic oligonucleotide
ggaattccagaccccttcca

SEQ ID NO: 119 - DNA, 20 bp, Artificial Sequence
  Synthetic oligonucleotide
caaccttcatcaacataagc

SEQ ID NO: 120 - DNA, 20 bp, Artificial Sequence
  Synthetic oligonucleotide
taagcaacggacaaggaagg

SEQ ID NO: 121 - DNA, 20 bp, Artificial Sequence
  Synthetic oligonucleotide
caaggaaggtttcagccaaa

SEQ ID NO: 122 - DNA, 20 bp, Artificial Sequence
  Synthetic oligonucleotide
tcagccaaatgcaggatttt

SEQ ID NO: 123 - DNA, 20 bp, Artificial Sequence
  Synthetic oligonucleotide
gtgtttacatccagaagccc

SEQ ID NO: 124 - DNA, 20 bp, Artificial Sequence
  Synthetic oligonucleotide
aagccccaaggacatctgcc

SEQ ID NO: 125 - DNA, 20 bp, Artificial Sequence
  Synthetic oligonucleotide
attttataagaaggcagcct

SEQ ID NO: 126 - DNA, 20 bp, Artificial Sequence
  Synthetic oligonucleotide
gcagcctgcccttgtaatcc

SEQ ID NO: 127 - DNA, 20 bp, Artificial Sequence
  Synthetic oligonucleotide
ccagcaacccacacccctct

SEQ ID NO: 128 - DNA, 20 bp, Artificial Sequence
  Synthetic oligonucleotide
tctctcccggcatctgactc

SEQ ID NO: 129 - DNA, 20 bp, Artificial Sequence
  Synthetic oligonucleotide
ggagacaacactgttagatt

SEQ ID NO: 130 - DNA, 20 bp, Artificial Sequence
  Synthetic oligonucleotide
caccctgaacacacagtgcc

SEQ ID NO: 131 - DNA, 20 bp, Artificial Sequence
  Synthetic oligonucleotide
acacagtgcctgctttattt

SEQ ID NO: 132 - DNA, 20 bp, Artificial Sequence
  Synthetic oligonucleotide
cttctaattaatcatattcc

SEQ ID NO: 133 - DNA, 20 bp, Artificial Sequence
  Synthetic oligonucleotide
tattccatggtttggctact

SEQ ID NO: 134 - DNA, 20 bp, Artificial Sequence
  Synthetic oligonucleotide
gtttactagtatggtttgat

SEQ ID NO: 135 - DNA, 20 bp, Artificial Sequence
  Synthetic oligonucleotide
gttcctaattttttgtaaga

SEQ ID NO: 136 - DNA, 20 bp, Artificial Sequence
  Synthetic oligonucleotide
ctcagcctccaagtagctgg

SEQ ID NO: 137 - DNA, 20 bp, Artificial Sequence
  Synthetic oligonucleotide
cagccagaccatcacttaca

SEQ ID NO: 138 - DNA, 20 bp, Artificial Sequence
  Synthetic oligonucleotide
ccaggctgcatccaaaccaa

SEQ ID NO: 139 - DNA, 20 bp, Artificial Sequence
  Synthetic oligonucleotide
gcctttaggagcttcttaga

SEQ ID NO: 140 - DNA, 20 bp, Artificial Sequence
  Synthetic oligonucleotide
ctatctgggcttttctttga

SEQ ID NO: 141 - DNA, 20 bp, Artificial Sequence
  Synthetic oligonucleotide
gaagatacttaggtcatcac

SEQ ID NO: 142 - DNA, 20 bp, Artificial Sequence
  Synthetic oligonucleotide
gagcactgatcctatggcta

SEQ ID NO: 143 - DNA, 20 bp, Artificial Sequence
  Synthetic oligonucleotide
ggctaatacccccaacgccc

SEQ ID NO: 144 - DNA, 20 bp, Artificial Sequence
  Synthetic oligonucleotide
cagccacccaccgaggaggc

SEQ ID NO: 145 - DNA, 20 bp, Artificial Sequence
  Synthetic oligonucleotide
ctgcagggttttgaaggtgg

SEQ ID NO: 146 - DNA, 20 bp, Artificial Sequence
  Synthetic oligonucleotide
cagaggcaaggcgtgtgggc

SEQ ID NO: 147 - DNA, 20 bp, Artificial Sequence
  Synthetic oligonucleotide
cctgagctcctgatgcgctg

SEQ ID NO: 148 - DNA, 20 bp, Artificial Sequence
  Synthetic oligonucleotide
ctacctgcctcatgtgccgg

SEQ ID NO: 149 - DNA, 20 bp, Artificial Sequence
  Synthetic oligonucleotide
gccggagggacttgaagact

SEQ ID NO: 150 - DNA, 20 bp, Artificial Sequence
  Synthetic oligonucleotide
tcagctcccgcccttctgta

SEQ ID NO: 151 - DNA, 20 bp, Artificial Sequence
  Synthetic oligonucleotide
ctctctggcaacacaggggc

SEQ ID NO: 152 - DNA, 20 bp, Artificial Sequence
  Synthetic oligonucleotide
cctcctcttcagcctcagag

SEQ ID NO: 153 - DNA, 20 bp, Artificial Sequence
  Synthetic oligonucleotide
cctcagagttgtcccagctt

SEQ ID NO: 154 - DNA, 20 bp, Artificial Sequence
  Synthetic oligonucleotide
cccagctttctgattcctct

SEQ ID NO: 155 - DNA, 20 bp, Artificial Sequence
  Synthetic oligonucleotide
tgacaaggacagttacagat

SEQ ID NO: 156 - DNA, 20 bp, Artificial Sequence
  Synthetic oligonucleotide
acagatacaaacagtctcat

SEQ ID NO: 157 - DNA, 20 bp, Artificial Sequence
  Synthetic oligonucleotide
tgagccggtcagtgtagatt

SEQ ID NO: 158 - DNA, 20 bp, Artificial Sequence
  Synthetic oligonucleotide
gtgtagattcctggctgcgg

SEQ ID NO: 159 - DNA, 20 bp, Artificial Sequence
  Synthetic oligonucleotide
ggccaggtcctatgctccct

SEQ ID NO: 160 - DNA, 20 bp, Artificial Sequence
  Synthetic oligonucleotide
gctgtctacttagcccgggg

SEQ ID NO: 161 - DNA, 20 bp, Artificial Sequence
  Synthetic oligonucleotide
gccaacagcgtgtctacgga

SEQ ID NO: 162 - DNA, 20 bp, Artificial Sequence
  Synthetic oligonucleotide
ggatctacacgttgtttctt

SEQ ID NO: 163 - DNA, 20 bp, Artificial Sequence
  Synthetic oligonucleotide
gttgtttcttttttttccac

SEQ ID NO: 164 - DNA, 20 bp, Artificial Sequence
  Synthetic oligonucleotide
cgattcttctgccccagcct

SEQ ID NO: 165 - DNA, 20 bp, Artificial Sequence
  Synthetic oligonucleotide
tgtccggctaactttttttg

SEQ ID NO: 166 - DNA, 20 bp, Artificial Sequence
  Synthetic oligonucleotide
ccggctaactttttttggta

SEQ ID NO: 167 - DNA, 20 bp, Artificial Sequence
  Synthetic oligonucleotide
acctcggcctcccggtccac

SEQ ID NO: 168 - DNA, 20 bp, Artificial Sequence
  Synthetic oligonucleotide
cttgagggcgtacgtgtgcg

SEQ ID NO: 169 - DNA, 20 bp, Artificial Sequence
  Synthetic oligonucleotide
gtacgtgtgcggctttgtta

SEQ ID NO: 170 - DNA, 20 bp, Artificial Sequence
  Synthetic oligonucleotide
tggtgctaaagtttgggctt

SEQ ID NO: 171 - DNA, 20 bp, Artificial Sequence
  Synthetic oligonucleotide
atccaatagaaagcatttca

SEQ ID NO: 172 - DNA, 20 bp, Artificial Sequence
  Synthetic oligonucleotide
aagcatttcagcccttcctc

SEQ ID NO: 173 - DNA, 20 bp, Artificial Sequence
  Synthetic oligonucleotide
caagtgatcctcccaacgaa

SEQ ID NO: 174 - DNA, 20 bp, Artificial Sequence
  Synthetic oligonucleotide
caacgaagccttccaagtag

SEQ ID NO: 175 - DNA, 20 bp, Artificial Sequence
  Synthetic oligonucleotide
ttccaggcgtgcaccagtgc

SEQ ID NO: 176 - DNA, 20 bp, Artificial Sequence
  Synthetic oligonucleotide
tgcaccagtgcctgacttcg

SEQ ID NO: 177 - DNA, 20 bp, Artificial Sequence
  Synthetic oligonucleotide
ttatggctacatccaacatc

SEQ ID NO: 178 - DNA, 20 bp, Artificial Sequence
  Synthetic oligonucleotide
ttgtcatttcaacgaaactt

SEQ ID NO: 179 - DNA, 20 bp, Artificial Sequence
  Synthetic oligonucleotide
aacgaaacttcatggagccc

SEQ ID NO: 180 - DNA, 20 bp, Artificial Sequence
  Synthetic oligonucleotide
ccctcacaaatgacaacatc

SEQ ID NO: 181 - DNA, 20 bp, Artificial Sequence
  Synthetic oligonucleotide
caacatctccatttcacatc

SEQ ID NO: 182 - DNA, 20 bp, Artificial Sequence
  Synthetic oligonucleotide
gagacccaaagggaagggtg

SEQ ID NO: 183 - DNA, 20 bp, Artificial Sequence
  Synthetic oligonucleotide
gaagggtgcacgtcagaagc

SEQ ID NO: 184 - DNA, 20 bp, Artificial Sequence
  Synthetic oligonucleotide
gaagcaaatccaggatgcga

SEQ ID NO: 185 - DNA, 20 bp, Artificial Sequence
  Synthetic oligonucleotide
cttcgtgctaaaaagctcac

SEQ ID NO: 186 - DNA, 20 bp, Artificial Sequence
  Synthetic oligonucleotide
gctcaccaagacactgccct

SEQ ID NO: 187 - DNA, 20 bp, Artificial Sequence
  Synthetic oligonucleotide
gccatctgcacctgcctaga

SEQ ID NO: 188 - DNA, 20 bp, Artificial Sequence
  Synthetic oligonucleotide
tgcctagaaacatgattagc

SEQ ID NO: 189 - DNA, 20 bp, Artificial Sequence
  Synthetic oligonucleotide
tataataaatggctccaggg

SEQ ID NO: 190 - DNA, 20 bp, Artificial Sequence
  Synthetic oligonucleotide
cagggccagccaacctgcag

SEQ ID NO: 191 - DNA, 20 bp, Artificial Sequence
  Synthetic oligonucleotide
ctgcagggcatgggggtgga

SEQ ID NO: 192 - DNA, 20 bp, Artificial Sequence
  Synthetic oligonucleotide
ctttgtctttcctgaccagg

SEQ ID NO: 193 - DNA, 20 bp, Artificial Sequence
  Synthetic oligonucleotide
ctgagactccattggcaaag

SEQ ID NO: 194 - DNA, 20 bp, Artificial Sequence
  Synthetic oligonucleotide
gcatgggaagcattattaat

SEQ ID NO: 195 - DNA, 20 bp, Artificial Sequence
  Synthetic oligonucleotide
ttaatgacatgtggtgtgga

SEQ ID NO: 196 - DNA, 20 bp, Artificial Sequence
  Synthetic oligonucleotide
ggtgtggaacaatggtcgta

SEQ ID NO: 197 - DNA, 20 bp, Artificial Sequence
  Synthetic oligonucleotide
accacagacctgtagcacca

SEQ ID NO: 198 - DNA, 20 bp, Artificial Sequence
  Synthetic oligonucleotide
tggtgcacacagtgccacga

SEQ ID NO: 199 - DNA, 20 bp, Artificial Sequence
  Synthetic oligonucleotide
aaacacagggccccaagccc

SEQ ID NO: 200 - DNA, 20 bp, Artificial Sequence
  Synthetic oligonucleotide
caagccccagatcaggaacc

SEQ ID NO: 201 - DNA, 20 bp, Artificial Sequence
  Synthetic oligonucleotide
cctgggaggtcagggaacca

SEQ ID NO: 202 - DNA, 20 bp, Artificial Sequence
  Synthetic oligonucleotide
gaacactttatttgtaaagg

SEQ ID NO: 203 - DNA, 20 bp, Artificial Sequence
  Synthetic oligonucleotide
tcgaccaggccaagcacttg

SEQ ID NO: 204 - DNA, 20 bp, Artificial Sequence
  Synthetic oligonucleotide
ccaagcacttggctatcaca

SEQ ID NO: 205 - DNA, 20 bp, Artificial Sequence
  Synthetic oligonucleotide
gcctttaatcagccagttct

SEQ ID NO: 206 - DNA, 20 bp, Artificial Sequence
  Synthetic oligonucleotide
ctacagacagcccacctgtg

SEQ ID NO: 207 - DNA, 20 bp, Artificial Sequence
  Synthetic oligonucleotide
ctttcacatggcggtcaagg

SEQ ID NO: 208 - DNA, 20 bp, Artificial Sequence
  Synthetic oligonucleotide
caaggcaaacgagacaggaa

SEQ ID NO: 209 - DNA, 20 bp, Artificial Sequence
  Synthetic oligonucleotide
aggaaatcacaacaatcaca

SEQ ID NO: 210 - DNA, 20 bp, Artificial Sequence
  Synthetic oligonucleotide
cacaacaatcacaccgagat

SEQ ID NO: 211 - DNA, 20 bp, Artificial Sequence
  Synthetic oligonucleotide
gagatcggagggtgagcttc

SEQ ID NO: 212 - DNA, 20 bp, Artificial Sequence
  Synthetic oligonucleotide
gaaggctcgttctgggctga

SEQ ID NO: 213 - DNA, 20 bp, Artificial Sequence
  Synthetic oligonucleotide
ctgattcagagccgcgtttc

SEQ ID NO: 214 - DNA, 20 bp, Artificial Sequence
  Synthetic oligonucleotide
cctcagcgattcctgccagg

SEQ ID NO: 215 - DNA, 20 bp, Artificial Sequence
  Synthetic oligonucleotide
gtaccgccccacggccatcc

SEQ ID NO: 216 - DNA, 20 bp, Artificial Sequence
  Synthetic oligonucleotide
tcaccagcgagcttctccat

SEQ ID NO: 217 - DNA, 20 bp, Artificial Sequence
  Synthetic oligonucleotide
ctggaccaaggtatcctcgg

SEQ ID NO: 218 - DNA, 20 bp, Artificial Sequence
  Synthetic oligonucleotide
gagcttctcggtcagccggc

SEQ ID NO: 219 - DNA, 20 bp, Artificial Sequence
  Synthetic oligonucleotide
gccggctgccacacgccttc

SEQ ID NO: 220 - DNA, 20 bp, Artificial Sequence
  Synthetic oligonucleotide
cacacgccttcaggagcctg

SEQ ID NO: 221 - DNA, 20 bp, Artificial Sequence
  Synthetic oligonucleotide
ggagaacacagcaccagcta

SEQ ID NO: 222 - DNA, 20 bp, Artificial Sequence
  Synthetic oligonucleotide
tgagcgttctcgccaaagga

SEQ ID NO: 223 - DNA, 20 bp, Artificial Sequence
  Synthetic oligonucleotide
acctgacgctcacccagccc

SEQ ID NO: 224 - DNA, 20 bp, Artificial Sequence
  Synthetic oligonucleotide
ggcacaaccagccagatccc

SEQ ID NO: 225 - DNA, 20 bp, Artificial Sequence
  Synthetic oligonucleotide
gccagatcccgcatgcaggc

SEQ ID NO: 226 - DNA, 20 bp, Artificial Sequence
  Synthetic oligonucleotide
gctccgatgccaatgcagtc

SEQ ID NO: 227 - DNA, 20 bp, Artificial Sequence
  Synthetic oligonucleotide
aggtaaactgcacaagggct

SEQ ID NO: 228 - DNA, 20 bp, Artificial Sequence
  Synthetic oligonucleotide
gcagcctggagaacactcag

SEQ ID NO: 229 - DNA, 20 bp, Artificial Sequence
  Synthetic oligonucleotide
gtgattatatttatggctgg

SEQ ID NO: 230 - DNA, 20 bp, Artificial Sequence
  Synthetic oligonucleotide
atggctggaatgatacgact

SEQ ID NO: 231 - DNA, 20 bp, Artificial Sequence
  Synthetic oligonucleotide
cgactcagatttgctttaaa

SEQ ID NO: 232 - DNA, 20 bp, Artificial Sequence
  Synthetic oligonucleotide
aaaaaaaaaggctgggcgcg

SEQ ID NO: 233 - DNA, 20 bp, Artificial Sequence
  Synthetic oligonucleotide
gaagaagtgagttccacatc

SEQ ID NO: 234 - DNA, 20 bp, Artificial Sequence
  Synthetic oligonucleotide
tccacatcaatatgttttca

SEQ ID NO: 235 - DNA, 20 bp, Artificial Sequence
  Synthetic oligonucleotide
ttcagcacactcaggggaag

SEQ ID NO: 236 - DNA, 20 bp, Artificial Sequence
  Synthetic oligonucleotide
cctacagaacaaggactccc

SEQ ID NO: 237 - DNA, 20 bp, Artificial Sequence
  Synthetic oligonucleotide
ccagatctgtgctcactgac

SEQ ID NO: 238 - DNA, 20 bp, Artificial Sequence
  Synthetic oligonucleotide
ctcactgacaagtgcccaca

SEQ ID NO: 239 - DNA, 20 bp, Artificial Sequence
  Synthetic oligonucleotide
ccagtgctcctcacagaggc

SEQ ID NO: 240 - DNA, 20 bp, Artificial Sequence
  Synthetic oligonucleotide
cagaggcagcaagcaaacac

SEQ ID NO: 241 - DNA, 20 bp, Artificial Sequence
  Synthetic oligonucleotide
gctaaggactgttctcccaa

SEQ ID NO: 242 - DNA, 20 bp, Artificial Sequence
  Synthetic oligonucleotide
ccaaatccatggacaagcca

SEQ ID NO: 243 - DNA, 20 bp, Artificial Sequence
  Synthetic oligonucleotide
gactgctctatcctaacaga

SEQ ID NO: 244 - DNA, 20 bp, Artificial Sequence
  Synthetic oligonucleotide
caaaagcagagacgcccgtt

SEQ ID NO: 245 - DNA, 20 bp, Artificial Sequence
  Synthetic oligonucleotide
cccgttaccaggcaaaggag

SEQ ID NO: 246 - DNA, 20 bp, Artificial Sequence
  Synthetic oligonucleotide
agcgatgtcagcgagcgggc

SEQ ID NO: 247 - DNA, 20 bp, Artificial Sequence
  Synthetic oligonucleotide
ggcgttgccgttcctctggt

SEQ ID NO: 248 - DNA, 20 bp, Artificial Sequence
  Synthetic oligonucleotide
catcctctgagcacgctgtg

SEQ ID NO: 249 - DNA, 20 bp, Artificial Sequence
  Synthetic oligonucleotide
agcagctcggacccctactc

SEQ ID NO: 250 - DNA, 20 bp, Artificial Sequence
  Synthetic oligonucleotide
ctcgttcacagccaactcca

SEQ ID NO: 251 - DNA, 20 bp, Artificial Sequence
  Synthetic oligonucleotide
caactccaggacctgactcc

SEQ ID NO: 252 - DNA, 20 bp, Artificial Sequence
  Synthetic oligonucleotide
gactcctggaggatcagtcc

SEQ ID NO: 253 - DNA, 20 bp, Artificial Sequence
  Synthetic oligonucleotide
gagcacacaaagtgccacca

SEQ ID NO: 254 - DNA, 20 bp, Artificial Sequence
  Synthetic oligonucleotide
attcccaacagcatagctct

SEQ ID NO: 255 - DNA, 20 bp, Artificial Sequence
  Synthetic oligonucleotide
gctctgccccggctacatca

SEQ ID NO: 256 - DNA, 20 bp, Artificial Sequence
  Synthetic oligonucleotide
acatcaggagctatccgaga

SEQ ID NO: 257 - DNA, 20 bp, Artificial Sequence
  Synthetic oligonucleotide
gctatccgagaacagaaatc

SEQ ID NO: 258 - DNA, 20 bp, Artificial Sequence
  Synthetic oligonucleotide
gagcttctcctgtccccttc

SEQ ID NO: 259 - DNA, 20 bp, Artificial Sequence
  Synthetic oligonucleotide
ttcctaatgagtgacccaag

SEQ ID NO: 260 - DNA, 20 bp, Artificial Sequence
  Synthetic oligonucleotide
ttctcgtgagtggccatctg

SEQ ID NO: 261 - DNA, 20 bp, Artificial Sequence
  Synthetic oligonucleotide
tggccatctgcgctgctccc

SEQ ID NO: 262 - DNA, 20 bp, Artificial Sequence
  Synthetic oligonucleotide
gtctccacctccccttctgg

SEQ ID NO: 263 - DNA, 20 bp, Artificial Sequence
  Synthetic oligonucleotide
gactggctctggtcaatgag

SEQ ID NO: 264 - DNA, 20 bp, Artificial Sequence
  Synthetic oligonucleotide
ggtcaatgagctgagacaaa

SEQ ID NO: 265 - DNA, 20 bp, Artificial Sequence
  Synthetic oligonucleotide
ttcaagcgtttctcctgcct

SEQ ID NO: 266 - DNA, 20 bp, Artificial Sequence
  Synthetic oligonucleotide
tatttctggaagagacgggg

SEQ ID NO: 267 - DNA, 20 bp, Artificial Sequence
  Synthetic oligonucleotide
agcattttcatatggctgcc

SEQ ID NO: 268 - DNA, 20 bp, Artificial Sequence
  Synthetic oligonucleotide
ctgcctgccaggacgaccac

SEQ ID NO: 269 - DNA, 20 bp, Artificial Sequence
  Synthetic oligonucleotide
ggacgaccacagcaagcaga

SEQ ID NO: 270 - DNA, 20 bp, Artificial Sequence
  Synthetic oligonucleotide
ccccactctcctgcaatgca

SEQ ID NO: 271 - DNA, 20 bp, Artificial Sequence
  Synthetic oligonucleotide
cacaccatgagtaagaaaca

SEQ ID NO: 272 - DNA, 20 bp, Artificial Sequence
  Synthetic oligonucleotide
gcttgaaggtgctgagatcc

SEQ ID NO: 273 - DNA, 20 bp, Artificial Sequence
  Synthetic oligonucleotide
gttaccagagcacgacctgg

SEQ ID NO: 274 - DNA, 20 bp, Artificial Sequence
  Synthetic oligonucleotide
ctggcctgagctgcatctcc

SEQ ID NO: 275 - DNA, 20 bp, Artificial Sequence
  Synthetic oligonucleotide
agctggaggcctacattcgg

SEQ ID NO: 276 - DNA, 20 bp, Artificial Sequence
  Synthetic oligonucleotide
acattcggtggtgcaactca

SEQ ID NO: 277 - DNA, 20 bp, Artificial Sequence
  Synthetic oligonucleotide
gagatgaaggccagaggaaa

SEQ ID NO: 278 - DNA, 20 bp, Artificial Sequence
  Synthetic oligonucleotide
gccagaggaaacaaagacac

SEQ ID NO: 279 - DNA, 20 bp, Artificial Sequence
  Synthetic oligonucleotide
gaaaccagagctgagcgaga

SEQ ID NO: 280 - DNA, 20 bp, Artificial Sequence
  Synthetic oligonucleotide
gagcgagaaccaggacaacg

SEQ ID NO: 281 - DNA, 20 bp, Artificial Sequence
  Synthetic oligonucleotide
gacaacgggcacaagagaga

SEQ ID NO: 282 - DNA, 20 bp, Artificial Sequence
  Synthetic oligonucleotide
gaagacgccacgcggtctcc

SEQ ID NO: 283 - DNA, 20 bp, Artificial Sequence
  Synthetic oligonucleotide
tgaattcaccttgagcgcct

SEQ ID NO: 284 - DNA, 20 bp, Artificial Sequence
  Synthetic oligonucleotide
gccctggagctcatcgatga

SEQ ID NO: 285 - DNA, 20 bp, Artificial Sequence
  Synthetic oligonucleotide
ttgatgaggtagatccggtc

SEQ ID NO: 286 - DNA, 20 bp, Artificial Sequence
  Synthetic oligonucleotide
agatccggtccatcatgatg

SEQ ID NO: 287 - DNA, 20 bp, Artificial Sequence
  Synthetic oligonucleotide
gatgatgctgtaccagcgct

SEQ ID NO: 288 - DNA, 20 bp, Artificial Sequence
  Synthetic oligonucleotide
ccaggctgtccttgatgaag

SEQ ID NO: 289 - DNA, 20 bp, Artificial Sequence
  Synthetic oligonucleotide
cttcacggccagggcagacc

SEQ ID NO: 290 - DNA, 20 bp, Artificial Sequence
  Synthetic oligonucleotide
cagggcagacctggagggac

SEQ ID NO: 291 - DNA, 20 bp, Artificial Sequence
  Synthetic oligonucleotide
ctggagggacaccggcgact

SEQ ID NO: 292 - DNA, 20 bp, Artificial Sequence
  Synthetic oligonucleotide
cagacagccctttcctcgct

SEQ ID NO: 293 - DNA, 20 bp, Artificial Sequence
  Synthetic oligonucleotide
tttcctcgcttagtgacacc

SEQ ID NO: 294 - DNA, 20 bp, Artificial Sequence
  Synthetic oligonucleotide
accaaatcaaagcctcttct

SEQ ID NO: 295 - DNA, 20 bp, Artificial Sequence
  Synthetic oligonucleotide
tcttcagacttttcagagtc

SEQ ID NO: 296 - DNA, 20 bp, Artificial Sequence
  Synthetic oligonucleotide
tcagctggcacaaatcagcc

SEQ ID NO: 297 - DNA, 20 bp, Artificial Sequence
  Synthetic oligonucleotide
gcgttaataacggggagagt

SEQ ID NO: 298 - DNA, 20 bp, Artificial Sequence
  Synthetic oligonucleotide
gagtggcacagtgggggcca

SEQ ID NO: 299 - DNA, 20 bp, Artificial Sequence
  Synthetic oligonucleotide
cagactcaaatgtaccagct

SEQ ID NO: 300 - DNA, 20 bp, Artificial Sequence
  Synthetic oligonucleotide
cttactcacctggagcatgc

SEQ ID NO: 301 - DNA, 20 bp, Artificial Sequence
  Synthetic oligonucleotide
cagaagaaccagcaggtcag

SEQ ID NO: 302 - DNA, 20 bp, Artificial Sequence
  Synthetic oligonucleotide
ccacgcctggctaatttttt

SEQ ID NO: 303 - DNA, 20 bp, Artificial Sequence
  Synthetic oligonucleotide
agccccacgtactttattct

SEQ ID NO: 304 - DNA, 20 bp, Artificial Sequence
  Synthetic oligonucleotide
aagaggatttcagtccctcc

SEQ ID NO: 305 - DNA, 20 bp, Artificial Sequence
  Synthetic oligonucleotide
gagaggacagttgagggcag

SEQ ID NO: 306 - DNA, 20 bp, Artificial Sequence
  Synthetic oligonucleotide
gccacactgaatgagaacag

SEQ ID NO: 307 - DNA, 20 bp, Artificial Sequence
  Synthetic oligonucleotide
gagaacagtaagggagcttg

SEQ ID NO: 308 - DNA, 20 bp, Artificial Sequence
  Synthetic oligonucleotide
tgtcgaactgggtggacacc

SEQ ID NO: 309 - DNA, 20 bp, Artificial Sequence
  Synthetic oligonucleotide
gacaccaatgacaaggaagt

SEQ ID NO: 310 - DNA, 20 bp, Artificial Sequence
  Synthetic oligonucleotide
gacaaggaagtttagtaact

SEQ ID NO: 311 - DNA, 20 bp, Artificial Sequence
  Synthetic oligonucleotide
gtaactgaacgacgaattct

SEQ ID NO: 312 - DNA, 20 bp, Artificial Sequence
  Synthetic oligonucleotide
gtaacaaagacctgagctca

SEQ ID NO: 313 - DNA, 20 bp, Artificial Sequence
  Synthetic oligonucleotide
ctcagagtcaagagcaacag

SEQ ID NO: 314 - DNA, 20 bp, Artificial Sequence
  Synthetic oligonucleotide
atccacacctcagcaggctg

SEQ ID NO: 315 - DNA, 20 bp, Artificial Sequence
  Synthetic oligonucleotide
gcaggctgagagcagcacgg

SEQ ID NO: 316 - DNA, 20 bp, Artificial Sequence
  Synthetic oligonucleotide
tgagacttggaatgaggact

SEQ ID NO: 317 - DNA, 20 bp, Artificial Sequence
  Synthetic oligonucleotide
ggactgagataataaaaaaa

SEQ ID NO: 318 - DNA, 20 bp, Artificial Sequence
  Synthetic oligonucleotide
gtgatgttgatgcaatgaac

SEQ ID NO: 319 - DNA, 20 bp, Artificial Sequence
  Synthetic oligonucleotide
catggcggctcacacctgta

SEQ ID NO: 320 - DNA, 20 bp, Artificial Sequence
  Synthetic oligonucleotide
gaggatgagccagaggattg

SEQ ID NO: 321 - DNA, 20 bp, Artificial Sequence
  Synthetic oligonucleotide
ccagaggattgcttgagttc

SEQ ID NO: 322 - DNA, 20 bp, Artificial Sequence
  Synthetic oligonucleotide
agttcgagaccaacctgggc

SEQ ID NO: 323 - DNA, 20 bp, Artificial Sequence
  Synthetic oligonucleotide
ggccgaggcgggcaaatcac

SEQ ID NO: 324 - DNA, 20 bp, Artificial Sequence
  Synthetic oligonucleotide
caaaaaacaaaccccagttg

SEQ ID NO: 325 - DNA, 20 bp, Artificial Sequence
  Synthetic oligonucleotide
acctgggaggctaaggcggg

SEQ ID NO: 326 - DNA, 20 bp, Artificial Sequence
  Synthetic oligonucleotide
cagagcgagaccctgacaca

SEQ ID NO: 327 - DNA, 20 bp, Artificial Sequence
  Synthetic oligonucleotide
cctgacacaaaagaaggaaa

SEQ ID NO: 328 - DNA, 20 bp, Artificial Sequence
  Synthetic oligonucleotide
gaaaatgctcagcaagtcca

SEQ ID NO: 329 - DNA, 20 bp, Artificial Sequence
  Synthetic oligonucleotide
cagcaagtccaacatgactc

SEQ ID NO: 330 - DNA, 20 bp, Artificial Sequence
  Synthetic oligonucleotide
tcctcccgcaattctggaca

SEQ ID NO: 331 - DNA, 20 bp, Artificial Sequence
  Synthetic oligonucleotide
aggcgtcctgtaccctgtgc

SEQ ID NO: 332 - DNA, 20 bp, Artificial Sequence
  Synthetic oligonucleotide
cgggtccgccctgagagagg

SEQ ID NO: 333 - DNA, 20 bp, Artificial Sequence
  Synthetic oligonucleotide
aggaccagtgcctgcctccc

SEQ ID NO: 334 - DNA, 20 bp, Artificial Sequence
  Synthetic oligonucleotide
ctgcctccctgtgcaatgct

SEQ ID NO: 335 - DNA, 20 bp, Artificial Sequence
  Synthetic oligonucleotide
gtgcaatgctggctccgagc

SEQ ID NO: 336 - DNA, 20 bp, Artificial Sequence
  Synthetic oligonucleotide
gctccgagcccacccagagc

SEQ ID NO: 337 - DNA, 20 bp, Artificial Sequence
  Synthetic oligonucleotide
ggccacaaggctcacctcac

SEQ ID NO: 338 - DNA, 20 bp, Artificial Sequence
  Synthetic oligonucleotide
gctcaggctccggacacagg

SEQ ID NO: 339 - DNA, 20 bp, Artificial Sequence
  Synthetic oligonucleotide
ggcctggcggacaatgctga

SEQ ID NO: 340 - DNA, 20 bp, Artificial Sequence
  Synthetic oligonucleotide
aagagctgggggtggctggg

SEQ ID NO: 341 - DNA, 20 bp, Artificial Sequence
  Synthetic oligonucleotide
ggtgctggtggctgacgtat

SEQ ID NO: 342 - DNA, 20 bp, Artificial Sequence
  Synthetic oligonucleotide
tatcatggctgatatatccc

SEQ ID NO: 343 - DNA, 20 bp, Artificial Sequence
  Synthetic oligonucleotide
ggtgccctgcagcaagtgac

SEQ ID NO: 344 - DNA, 20 bp, Artificial Sequence
  Synthetic oligonucleotide
catgaaaaggaaaagtaaat

SEQ ID NO: 345 - DNA, 20 bp, Artificial Sequence
  Synthetic oligonucleotide
gtaaatctgttagttgggaa

SEQ ID NO: 346 - DNA, 20 bp, Artificial Sequence
  Synthetic oligonucleotide
gacaaactctcttaggttta

SEQ ID NO: 347 - DNA, 20 bp, Artificial Sequence
  Synthetic oligonucleotide
cagaacacagagatcgcaga

SEQ ID NO: 348 - DNA, 20 bp, Artificial Sequence
  Synthetic oligonucleotide
ggttgttcctaacaggtatt

SEQ ID NO: 349 - DNA, 20 bp, Artificial Sequence
  Synthetic oligonucleotide
gagcacttacgaagtaggta

SEQ ID NO: 350 - DNA, 20 bp, Artificial Sequence
  Synthetic oligonucleotide
aggtaacatgaaaagggccc

SEQ ID NO: 351 - DNA, 20 bp, Artificial Sequence
  Synthetic oligonucleotide
ctgcatgctccatctcattc

SEQ ID NO: 352 - DNA, 20 bp, Artificial Sequence
  Synthetic oligonucleotide
ccctctgagataatgtcagt

SEQ ID NO: 353 - DNA, 20 bp, Artificial Sequence
  Synthetic oligonucleotide
tctacagaagacaaaactga

SEQ ID NO: 354 - DNA, 20 bp, Artificial Sequence
  Synthetic oligonucleotide
gaagtagctggcccgagggt

SEQ ID NO: 355 - DNA, 20 bp, Artificial Sequence
  Synthetic oligonucleotide
gtaacactacccactgcccc

SEQ ID NO: 356 - DNA, 20 bp, Artificial Sequence
  Synthetic oligonucleotide
ctccatcattggttctgaac

SEQ ID NO: 357 - DNA, 20 bp, Artificial Sequence
  Synthetic oligonucleotide
acctggaaggggcttcagcc

SEQ ID NO: 358 - DNA, 20 bp, Artificial Sequence
  Synthetic oligonucleotide
agccacaggcgagaggccca

SEQ ID NO: 359 - DNA, 20 bp, Artificial Sequence
  Synthetic oligonucleotide
tagggcgagcaccaagctcc

SEQ ID NO: 360 - DNA, 20 bp, Artificial Sequence
  Synthetic oligonucleotide
agccactccgatgctgaggc

SEQ ID NO: 361 - DNA, 20 bp, Artificial Sequence
  Synthetic oligonucleotide
gaggctgtgcgccctggagc

SEQ ID NO: 362 - DNA, 20 bp, Artificial Sequence
  Synthetic oligonucleotide
gctgctcctaccccaggact

SEQ ID NO: 363 - DNA, 20 bp, Artificial Sequence
  Synthetic oligonucleotide
ctgtgcggcagtgcgtcagc

SEQ ID NO: 364 - DNA, 20 bp, Artificial Sequence
  Synthetic oligonucleotide
agactctccccacagtgtcg

SEQ ID NO: 365 - DNA, 20 bp, Artificial Sequence
  Synthetic oligonucleotide
cctccatgggcaaccccaaa

SEQ ID NO: 366 - DNA, 20 bp, Artificial Sequence
  Synthetic oligonucleotide
aaccagctcaaggatgcccc

SEQ ID NO: 367 - DNA, 20 bp, Artificial Sequence
  Synthetic oligonucleotide
gcagcctggcggtgccctcc

SEQ ID NO: 368 - DNA, 20 bp, Artificial Sequence
  Synthetic oligonucleotide
ccctccttcccaggtctgag

SEQ ID NO: 369 - DNA, 20 bp, Artificial Sequence
  Synthetic oligonucleotide
tgctccccatcccccattcc

SEQ ID NO: 370 - DNA, 20 bp, Artificial Sequence
  Synthetic oligonucleotide
ccccattccttacaggcaat

SEQ ID NO: 371 - DNA, 20 bp, Artificial Sequence
  Synthetic oligonucleotide
tccagcaatggatgcagctc

SEQ ID NO: 372 - DNA, 20 bp, Artificial Sequence
  Synthetic oligonucleotide
gctcttacacgtcctctgct

SEQ ID NO: 373 - DNA, 20 bp, Artificial Sequence
  Synthetic oligonucleotide
gaacaagcttgggttcccgt

SEQ ID NO: 374 - DNA, 20 bp, Artificial Sequence
  Synthetic oligonucleotide
ggcaggtccaacacccaccc

SEQ ID NO: 375 - DNA, 20 bp, Artificial Sequence
  Synthetic oligonucleotide
gattttgggccctgacccag

SEQ ID NO: 376 - DNA, 20 bp, Artificial Sequence
  Synthetic oligonucleotide
gacccaggtcctccacaaaa

SEQ ID NO: 377 - DNA, 20 bp, Artificial Sequence
  Synthetic oligonucleotide
cactaattcccgagagttca

SEQ ID NO: 378 - DNA, 20 bp, Artificial Sequence
  Synthetic oligonucleotide
ggagtgacccaccgagaaca

SEQ ID NO: 379 - DNA, 20 bp, Artificial Sequence
  Synthetic oligonucleotide
gggagcaggaaactccactg

SEQ ID NO: 380 - DNA, 20 bp, Artificial Sequence
  Synthetic oligonucleotide
gacccgcctccaggacaggc

SEQ ID NO: 381 - DNA, 20 bp, Artificial Sequence
  Synthetic oligonucleotide
ccaggacaggctggagtgtg

SEQ ID NO: 382 - DNA, 20 bp, Artificial Sequence
  Synthetic oligonucleotide
acaaacatgaaggccacggc

SEQ ID NO: 383 - DNA, 20 bp, Artificial Sequence
  Synthetic oligonucleotide
ccacggcagggaggcagagt

SEQ ID NO: 384 - DNA, 20 bp, Artificial Sequence
  Synthetic oligonucleotide
gttaaagaagttaacatagg

SEQ ID NO: 385 - DNA, 20 bp, Artificial Sequence
  Synthetic oligonucleotide
gttaacataggctgggtgtg

SEQ ID NO: 386 - DNA, 20 bp, Artificial Sequence
  Synthetic oligonucleotide
cgtggtggcaggcaagtgta

SEQ ID NO: 387 - DNA, 20 bp, Artificial Sequence
  Synthetic oligonucleotide
aattcgttattcgggaggct

SEQ ID NO: 388 - DNA, 20 bp, Artificial Sequence
  Synthetic oligonucleotide
ttgcctcgcacatgtccgac

SEQ ID NO: 389 - DNA, 20 bp, Artificial Sequence
  Synthetic oligonucleotide
tgtccgactttttgggcccc

SEQ ID NO: 390 - DNA, 20 bp, Artificial Sequence
  Synthetic oligonucleotide
ccccgggctgctggactcga

SEQ ID NO: 391 - DNA, 20 bp, Artificial Sequence
  Synthetic oligonucleotide
acgctggccccctctgcggg

SEQ ID NO: 392 - DNA, 20 bp, Artificial Sequence
  Synthetic oligonucleotide
cacctccgtgcagaagagag

SEQ ID NO: 393 - DNA, 20 bp, Artificial Sequence
  Synthetic oligonucleotide
gagagtgcgggggccgtgga

SEQ ID NO: 394 - DNA, 20 bp, Artificial Sequence
  Synthetic oligonucleotide
gggccgtggagctcgcagaa

SEQ ID NO: 395 - DNA, 20 bp, Artificial Sequence
  Synthetic oligonucleotide
ggacaggggacatgtcagct

SEQ ID NO: 396 - DNA, 20 bp, Artificial Sequence
  Synthetic oligonucleotide
ggtgccatggacttcctgcc

SEQ ID NO: 397 - DNA, 20 bp, Artificial Sequence
  Synthetic oligonucleotide
agaggccacccgccagttcc

SEQ ID NO: 398 - DNA, 20 bp, Artificial Sequence
  Synthetic oligonucleotide
cagttccccagccccctgcc

SEQ ID NO: 399 - DNA, 20 bp, Artificial Sequence
  Synthetic oligonucleotide
ctgctttgccccagtgcgag

SEQ ID NO: 400 - DNA, 20 bp, Artificial Sequence
  Synthetic oligonucleotide
agataggatcttcccatggg

SEQ ID NO: 401 - DNA, 20 bp, Artificial Sequence
  Synthetic oligonucleotide
agtcacagcaaataggggag

SEQ ID NO: 402 - DNA, 20 bp, Artificial Sequence
  Synthetic oligonucleotide
ggggaggggacaatgtggac

SEQ ID NO: 403 - DNA, 20 bp, Artificial Sequence
  Synthetic oligonucleotide
accaatgatagcacagctgg

SEQ ID NO: 404 - DNA, 20 bp, Artificial Sequence
  Synthetic oligonucleotide
gccacaaagccaacggcgct

SEQ ID NO: 405 - DNA, 20 bp, Artificial Sequence
  Synthetic oligonucleotide
cggcgctggaaaggaatgtc

SEQ ID NO: 406 - DNA, 20 bp, Artificial Sequence
  Synthetic oligonucleotide
gaactgcagccaaacctggc

SEQ ID NO: 407 - DNA, 20 bp, Artificial Sequence
  Synthetic oligonucleotide
acaaatagataaaaggccac

SEQ ID NO: 408 - DNA, 20 bp, Artificial Sequence
  Synthetic oligonucleotide
gagggtcctccacccaatac

SEQ ID NO: 409 - DNA, 20 bp, Artificial Sequence
  Synthetic oligonucleotide
acccaatacatatctgcttg

SEQ ID NO: 410 - DNA, 20 bp, Artificial Sequence
  Synthetic oligonucleotide
cttacggggccaagcaagag

SEQ ID NO: 411 - DNA, 20 bp, Artificial Sequence
  Synthetic oligonucleotide
gcaagagttgaagggcaggg

SEQ ID NO: 412 - DNA, 20 bp, Artificial Sequence
  Synthetic oligonucleotide
gcagggtgggtgctgtactg

SEQ ID NO: 413 - DNA, 20 bp, Artificial Sequence
  Synthetic oligonucleotide
gagcagcagcagacagggtg

SEQ ID NO: 414 - DNA, 20 bp, Artificial Sequence
  Synthetic oligonucleotide
gaggctgtaggaagcagatc

SEQ ID NO: 415 - DNA, 20 bp, Artificial Sequence
  Synthetic oligonucleotide
agcagatcttatagctcctc

SEQ ID NO: 416 - DNA, 20 bp, Artificial Sequence
  Synthetic oligonucleotide
tccaggccaccgaaagaccc

SEQ ID NO: 417 - DNA, 20 bp, Artificial Sequence
  Synthetic oligonucleotide
ggttctgatgagacatgaga

SEQ ID NO: 418 - DNA, 20 bp, Artificial Sequence
  Synthetic oligonucleotide
gagacatgagaaaatgtgaa

SEQ ID NO: 419 - DNA, 20 bp, Artificial Sequence
  Synthetic oligonucleotide
tgaatttcacttgaataggt

SEQ ID NO: 420 - DNA, 20 bp, Artificial Sequence
  Synthetic oligonucleotide
ggttcactcatgcccatgtg

SEQ ID NO: 421 - DNA, 20 bp, Artificial Sequence
  Synthetic oligonucleotide
tggcagggggccagggtgga

SEQ ID NO: 422 - DNA, 20 bp, Artificial Sequence
  Synthetic oligonucleotide
ggggaacaagctgaggcacc

SEQ ID NO: 423 - DNA, 20 bp, Artificial Sequence
  Synthetic oligonucleotide
gatctgagcaggaagggaca

SEQ ID NO: 424 - DNA, 20 bp, Artificial Sequence
  Synthetic oligonucleotide
caggccaggcagggtgaaga

SEQ ID NO: 425 - DNA, 20 bp, Artificial Sequence
  Synthetic oligonucleotide
agggtgaagacgcacagtgc

SEQ ID NO: 426 - DNA, 20 bp, Artificial Sequence
  Synthetic oligonucleotide
gtgcgggtggggtgcagagt

SEQ ID NO: 427 - DNA, 20 bp, Artificial Sequence
  Synthetic oligonucleotide
gagaaggcccagtgagtgcc

SEQ ID NO: 428 - DNA, 20 bp, Artificial Sequence
  Synthetic oligonucleotide
gagtgcctgctgagccttca

SEQ ID NO: 429 - DNA, 20 bp, Artificial Sequence
  Synthetic oligonucleotide
ggacgggacacagcagcaga

SEQ ID NO: 430 - DNA, 20 bp, Artificial Sequence
  Synthetic oligonucleotide
agcagcagagccaagcgctg

SEQ ID NO: 431 - DNA, 20 bp, Artificial Sequence
  Synthetic oligonucleotide
cggtcggggctgacgcatgg

SEQ ID NO: 432 - DNA, 20 bp, Artificial Sequence
  Synthetic oligonucleotide
atgggacctccgtggagtgc

SEQ ID NO: 433 - DNA, 20 bp, Artificial Sequence
  Synthetic oligonucleotide
agcccttgtagactcagagg

SEQ ID NO: 434 - DNA, 20 bp, Artificial Sequence
  Synthetic oligonucleotide
agggaggacagctagccaac

SEQ ID NO: 435 - DNA, 20 bp, Artificial Sequence
  Synthetic oligonucleotide
gctagccaacatgaccaagc

SEQ ID NO: 436 - DNA, 20 bp, Artificial Sequence
  Synthetic oligonucleotide
ccaagcacaggcagtgaggc

SEQ ID NO: 437 - DNA, 20 bp, Artificial Sequence
  Synthetic oligonucleotide
gtgaggccagaggggagctc

SEQ ID NO: 438 - DNA, 20 bp, Artificial Sequence
  Synthetic oligonucleotide
gctttagcaaggtgggactg

SEQ ID NO: 439 - DNA, 20 bp, Artificial Sequence
  Synthetic oligonucleotide
gtcacatggccttaaaaagg

SEQ ID NO: 440 - DNA, 20 bp, Artificial Sequence
  Synthetic oligonucleotide
gccacaagctacggcacggc

SEQ ID NO: 441 - DNA, 20 bp, Artificial Sequence
  Synthetic oligonucleotide
ggcacggctgaaactgaagg

SEQ ID NO: 442 - DNA, 20 bp, Artificial Sequence
  Synthetic oligonucleotide
gaaggacattacacgaaatg

SEQ ID NO: 443 - DNA, 20 bp, Artificial Sequence
  Synthetic oligonucleotide
ccacttaaataggaggtacc

SEQ ID NO: 444 - DNA, 20 bp, Artificial Sequence
  Synthetic oligonucleotide
gtcctcaaagccatagagac

SEQ ID NO: 445 - DNA, 20 bp, Artificial Sequence
  Synthetic oligonucleotide
agagagcagaatggcagctg

SEQ ID NO: 446 - DNA, 20 bp, Artificial Sequence
  Synthetic oligonucleotide
tgccggggactgggagggaa

SEQ ID NO: 447 - DNA, 20 bp, Artificial Sequence
  Synthetic oligonucleotide
ggggctcagttctggagacg

SEQ ID NO: 448 - DNA, 20 bp, Artificial Sequence
  Synthetic oligonucleotide
gacggaaggtggcgatggcc

SEQ ID NO: 449 - DNA, 20 bp, Artificial Sequence
  Synthetic oligonucleotide
gcgatggccgcacagcactg

SEQ ID NO: 450 - DNA, 20 bp, Artificial Sequence
  Synthetic oligonucleotide
gtgaacatacttcatgccac

SEQ ID NO: 451 - DNA, 20 bp, Artificial Sequence
  Synthetic oligonucleotide
ggcactgcacgccgaacagt

SEQ ID NO: 452 - DNA, 20 bp, Artificial Sequence
  Synthetic oligonucleotide
cagtggttcaaatggcgcat

SEQ ID NO: 453 - DNA, 20 bp, Artificial Sequence
  Synthetic oligonucleotide
cattttaggtcatgtattat

SEQ ID NO: 454 - DNA, 20 bp, Artificial Sequence
  Synthetic oligonucleotide
aatctttttaaaagggggag

SEQ ID NO: 455 - DNA, 20 bp, Artificial Sequence
  Synthetic oligonucleotide
ggagtggtaacctgcagaac

SEQ ID NO: 456 - DNA, 20 bp, Artificial Sequence
  Synthetic oligonucleotide
caaaggtgaccttaacacag

SEQ ID NO: 457 - DNA, 20 bp, Artificial Sequence
  Synthetic oligonucleotide
gaaaaagctcacaggagtgg

SEQ ID NO: 458 - DNA, 20 bp, Artificial Sequence
  Synthetic oligonucleotide
gtttccgtggcagagccagc

SEQ ID NO: 459 - DNA, 20 bp, Artificial Sequence
  Synthetic oligonucleotide
cagagccagcaatgggacac

SEQ ID NO: 460 - DNA, 20 bp, Artificial Sequence
  Synthetic oligonucleotide
ggggacatggagtcaaggaa

SEQ ID NO: 461 - DNA, 20 bp, Artificial Sequence
  Synthetic oligonucleotide
tggccacagtacagctgcga

SEQ ID NO: 462 - DNA, 20 bp, Artificial Sequence
  Synthetic oligonucleotide
accatgtgaaggccaggggc

SEQ ID NO: 463 - DNA, 20 bp, Artificial Sequence
  Synthetic oligonucleotide
cagctgcaggcacaaagtct

SEQ ID NO: 464 - DNA, 20 bp, Artificial Sequence
  Synthetic oligonucleotide
agcactcattgcagagggat

SEQ ID NO: 465 - DNA, 20 bp, Artificial Sequence
  Synthetic oligonucleotide
ccgcggcagcatgaggcagt

SEQ ID NO: 466 - DNA, 20 bp, Artificial Sequence
  Synthetic oligonucleotide
acgaatgcaggaaagccaga

SEQ ID NO: 467 - DNA, 20 bp, Artificial Sequence
  Synthetic oligonucleotide
gaaggctttaaaatgggggt

SEQ ID NO: 468 - DNA, 20 bp, Artificial Sequence
  Synthetic oligonucleotide
gttctcactggcagagaagt

SEQ ID NO: 469 - DNA, 20 bp, Artificial Sequence
  Synthetic oligonucleotide
gaagttctggaaacgagacc

SEQ ID NO: 470 - DNA, 20 bp, Artificial Sequence
  Synthetic oligonucleotide
cctgacacatgcagccagac

SEQ ID NO: 471 - DNA, 20 bp, Artificial Sequence
  Synthetic oligonucleotide
cattgtccatacggtggcca

SEQ ID NO: 472 - DNA, 20 bp, Artificial Sequence
  Synthetic oligonucleotide
ggccacagctatgtgtgaca

SEQ ID NO: 473 - DNA, 20 bp, Artificial Sequence
  Synthetic oligonucleotide
aatttaaattagctaggaga

SEQ ID NO: 474 - DNA, 20 bp, Artificial Sequence
  Synthetic oligonucleotide
taggagaggccggcacagtg

SEQ ID NO: 475 - DNA, 20 bp, Artificial Sequence
  Synthetic oligonucleotide
tcccatgagcctgtaatccc

SEQ ID NO: 476 - DNA, 20 bp, Artificial Sequence
  Synthetic oligonucleotide
cacttgaggtcaggagtttg

SEQ ID NO: 477 - DNA, 20 bp, Artificial Sequence
  Synthetic oligonucleotide
tggccgacgcagtgaaaccc

SEQ ID NO: 478 - DNA, 20 bp, Artificial Sequence
  Synthetic oligonucleotide
cacttgaacctgggaggcaa

SEQ ID NO: 479 - DNA, 20 bp, Artificial Sequence
  Synthetic oligonucleotide
gatcgcaccacggcactcga

SEQ ID NO: 480 - DNA, 20 bp, Artificial Sequence
  Synthetic oligonucleotide
ccaaaaacaacaaaaacaaa

SEQ ID NO: 481 - DNA, 20 bp, Artificial Sequence
  Synthetic oligonucleotide
acaaaataaaacaaaaacct

SEQ ID NO: 482 - DNA, 20 bp, Artificial Sequence
  Synthetic oligonucleotide
acaaaaaccttagctgggcg

SEQ ID NO: 483 - DNA, 20 bp, Artificial Sequence
  Synthetic oligonucleotide
gcgtgttggcggtcacctat

SEQ ID NO: 484 - DNA, 20 bp, Artificial Sequence
  Synthetic oligonucleotide
atcacttgaacctgggaggc

SEQ ID NO: 485 - DNA, 20 bp, Artificial Sequence
  Synthetic oligonucleotide
gcctttatgacagggcgaga

SEQ ID NO: 486 - DNA, 20 bp, Artificial Sequence
  Synthetic oligonucleotide
aaaaaaagtagcctggagtg

SEQ ID NO: 487 - DNA, 20 bp, Artificial Sequence
  Synthetic oligonucleotide
cctggagtggtggcgcacgc

SEQ ID NO: 488 - DNA, 20 bp, Artificial Sequence
  Synthetic oligonucleotide
gctgagtgcggttgttcatg

SEQ ID NO: 489 - DNA, 20 bp, Artificial Sequence
  Synthetic oligonucleotide
atattagctaggacaaaagc

SEQ ID NO: 490 - DNA, 20 bp, Artificial Sequence
  Synthetic oligonucleotide
tcaccggccatgtcctgagt

SEQ ID NO: 491 - DNA, 20 bp, Artificial Sequence
  Synthetic oligonucleotide
tgctaggcacacacgactca

SEQ ID NO: 492 - DNA, 20 bp, Artificial Sequence
  Synthetic oligonucleotide
agcccaggtcacagaatatt

SEQ ID NO: 493 - DNA, 20 bp, Artificial Sequence
  Synthetic oligonucleotide
gtgtggctggcagggaatgg

SEQ ID NO: 494 - DNA, 20 bp, Artificial Sequence
  Synthetic oligonucleotide
agggctggtgctcagtaggt

SEQ ID NO: 495 - DNA, 20 bp, Artificial Sequence
  Synthetic oligonucleotide
tccgagagggaggcagacag

SEQ ID NO: 496 - DNA, 20 bp, Artificial Sequence
  Synthetic oligonucleotide
cagagtttcaataaagggcc

SEQ ID NO: 497 - DNA, 20 bp, Artificial Sequence
  Synthetic oligonucleotide
cttgcttgggacgatgaaga

SEQ ID NO: 498 - DNA, 20 bp, Artificial Sequence
  Synthetic oligonucleotide
tcatgtaagtttctgaatgt

SEQ ID NO: 499 - DNA, 20 bp, Artificial Sequence
  Synthetic oligonucleotide
ctgagtagtggacactggcc

SEQ ID NO: 500 - DNA, 20 bp, Artificial Sequence
  Synthetic oligonucleotide
tcttctcttctggggagccc

SEQ ID NO: 501 - DNA, 20 bp, Artificial Sequence
  Synthetic oligonucleotide
ccccacccctgttacggatc

SEQ ID NO: 502 - DNA, 20 bp, Artificial Sequence
  Synthetic oligonucleotide
tgtcttcgctcctgacaggc

SEQ ID NO: 503 - DNA, 20 bp, Artificial Sequence
  Synthetic oligonucleotide
gtgaacacaaacaaggcatg

SEQ ID NO: 504 - DNA, 20 bp, Artificial Sequence
  Synthetic oligonucleotide
cctgccaaagccgctaactc

SEQ ID NO: 505 - DNA, 20 bp, Artificial Sequence
  Synthetic oligonucleotide
tctaggaagaagcaaaggac

SEQ ID NO: 506 - DNA, 20 bp, Artificial Sequence
  Synthetic oligonucleotide
cgatgtgcatggtggtgggg

SEQ ID NO: 507 - DNA, 20 bp, Artificial Sequence
  Synthetic oligonucleotide
gcagccagcctctctgccgg

SEQ ID NO: 508 - DNA, 20 bp, Artificial Sequence
  Synthetic oligonucleotide
cagcatgcctgcgaatgcac

SEQ ID NO: 509 - DNA, 20 bp, Artificial Sequence
  Synthetic oligonucleotide
gcaccacccacctgacgttt

SEQ ID NO: 510 - DNA, 20 bp, Artificial Sequence
  Synthetic oligonucleotide
gacgtttctgcagttggctg

SEQ ID NO: 511 - DNA, 20 bp, Artificial Sequence
  Synthetic oligonucleotide
agctgcacgagctgctccgg

SEQ ID NO: 512 - DNA, 20 bp, Artificial Sequence
  Synthetic oligonucleotide
gcacgagtggtcacactggg

SEQ ID NO: 513 - DNA, 20 bp, Artificial Sequence
  Synthetic oligonucleotide
gggctggcagtgaggcaggt

SEQ ID NO: 514 - DNA, 20 bp, Artificial Sequence
  Synthetic oligonucleotide
gcaggtgtgggagtcagaga

SEQ ID NO: 515 - DNA, 20 bp, Artificial Sequence
  Synthetic oligonucleotide
gagagggagaccctgggtgt

SEQ ID NO: 516 - DNA, 20 bp, Artificial Sequence
  Synthetic oligonucleotide
gggtgtggctggcagggaat

SEQ ID NO: 517 - DNA, 20 bp, Artificial Sequence
  Synthetic oligonucleotide
tgtaggtgcactccgagagg

SEQ ID NO: 518 - DNA, 20 bp, Artificial Sequence
  Synthetic oligonucleotide
tgctcagagtaccccttgcc

SEQ ID NO: 519 - DNA, 20 bp, Artificial Sequence
  Synthetic oligonucleotide
caccagaagagctcccctcc

SEQ ID NO: 520 - DNA, 20 bp, Artificial Sequence
  Synthetic oligonucleotide
ctttgagtcgtagcgctcct

SEQ ID NO: 521 - DNA, 20 bp, Artificial Sequence
  Synthetic oligonucleotide
ctcctcatcgcagaactgcg

SEQ ID NO: 522 - DNA, 20 bp, Artificial Sequence
  Synthetic oligonucleotide
gaactgcgcctccaaagtgc

SEQ ID NO: 523 - DNA, 20 bp, Artificial Sequence
  Synthetic oligonucleotide
cggaatctaggggcggcaga

SEQ ID NO: 524 - DNA, 20 bp, Artificial Sequence
  Synthetic oligonucleotide
gaaccaaacagacagtttca

SEQ ID NO: 525 - DNA, 20 bp, Artificial Sequence
  Synthetic oligonucleotide
acagtttcaataaagggccc

SEQ ID NO: 526 - DNA, 20 bp, Artificial Sequence
  Synthetic oligonucleotide
cccacagggttcactatgat

SEQ ID NO: 527 - DNA, 20 bp, Artificial Sequence
  Synthetic oligonucleotide
gagtggcttaaaaatctact

SEQ ID NO: 528 - DNA, 20 bp, Artificial Sequence
  Synthetic oligonucleotide
caggagatggggcgaccact

SEQ ID NO: 529 - DNA, 20 bp, Artificial Sequence
  Synthetic oligonucleotide
gtaaggggtagcaggaccca

SEQ ID NO: 530 - DNA, 20 bp, Artificial Sequence
  Synthetic oligonucleotide
gcgaaaaggaaggccacgcc

SEQ ID NO: 531 - DNA, 20 bp, Artificial Sequence
  Synthetic oligonucleotide
gccacgcctttctctgagag

SEQ ID NO: 532 - DNA, 20 bp, Artificial Sequence
  Synthetic oligonucleotide
taaggttccttctagcaaga

SEQ ID NO: 533 - DNA, 20 bp, Artificial Sequence
  Synthetic oligonucleotide
tctagcaagacatgcacagg

SEQ ID NO: 534 - DNA, 20 bp, Artificial Sequence
  Synthetic oligonucleotide
aagtctcagcccacctgcag

SEQ ID NO: 535 - DNA, 20 bp, Artificial Sequence
  Synthetic oligonucleotide
cacgcttccaccgtccacac

SEQ ID NO: 536 - DNA, 20 bp, Artificial Sequence
  Synthetic oligonucleotide
gacacccttgaagacctggc

SEQ ID NO: 537 - DNA, 20 bp, Artificial Sequence
  Synthetic oligonucleotide
ggcgttttctaggtaagaga

SEQ ID NO: 538 - DNA, 20 bp, Artificial Sequence
  Synthetic oligonucleotide
aaataatgggagcagcttac

SEQ ID NO: 539 - DNA, 20 bp, Artificial Sequence
  Synthetic oligonucleotide
gtagacactccccagggttc

SEQ ID NO: 540 - DNA, 20 bp, Artificial Sequence
  Synthetic oligonucleotide
gggttctgaatggcttagca

SEQ ID NO: 541 - DNA, 20 bp, Artificial Sequence
  Synthetic oligonucleotide
ttagctgaactcttgcacct

SEQ ID NO: 542 - DNA, 20 bp, Artificial Sequence
  Synthetic oligonucleotide
tcaaggatctctgtatttat

SEQ ID NO: 543 - DNA, 20 bp, Artificial Sequence
  Synthetic oligonucleotide
gtcttgaggccagctcagtg

SEQ ID NO: 544 - DNA, 20 bp, Artificial Sequence
  Synthetic oligonucleotide
aaatacaaaaaaaatcagcc

SEQ ID NO: 545 - DNA, 20 bp, Artificial Sequence
  Synthetic oligonucleotide
tcagccaggcatggtgacag

SEQ ID NO: 546 - DNA, 20 bp, Artificial Sequence
  Synthetic oligonucleotide
tgagacgcagagcttgcagt

SEQ ID NO: 547 - DNA, 20 bp, Artificial Sequence
  Synthetic oligonucleotide
agtagctgggattacaggtg

SEQ ID NO: 548 - DNA, 20 bp, Artificial Sequence
  Synthetic oligonucleotide
tgcatccagccaagtcccca

SEQ ID NO: 549 - DNA, 20 bp, Artificial Sequence
  Synthetic oligonucleotide
gaggcaacgctgacaagcca

SEQ ID NO: 550 - DNA, 20 bp, Artificial Sequence
  Synthetic oligonucleotide
ccacttcccagcacccagag

SEQ ID NO: 551 - DNA, 20 bp, Artificial Sequence
  Synthetic oligonucleotide
caacatgggctacactgttt

SEQ ID NO: 552 - DNA, 20 bp, Artificial Sequence
  Synthetic oligonucleotide
tttttttttgcggaagggtc

SEQ ID NO: 553 - DNA, 20 bp, Artificial Sequence
  Synthetic oligonucleotide
tgcagcaatctcggatcact

SEQ ID NO: 554 - DNA, 20 bp, Artificial Sequence
  Synthetic oligonucleotide
gtagctggaattatggccgt

SEQ ID NO: 555 - DNA, 20 bp, Artificial Sequence
  Synthetic oligonucleotide
cgtgcgccaccaggaccagc

SEQ ID NO: 556 - DNA, 20 bp, Artificial Sequence
  Synthetic oligonucleotide
agtttgtgaccagccaggct

SEQ ID NO: 557 - DNA, 20 bp, Artificial Sequence
  Synthetic oligonucleotide
ccaggctaacatggtgaaac

SEQ ID NO: 558 - DNA, 20 bp, Artificial Sequence
  Synthetic oligonucleotide
aggcaggcggaccacgaggt

SEQ ID NO: 559 - DNA, 20 bp, Artificial Sequence
  Synthetic oligonucleotide
aaattaggctgggtgcggtg

SEQ ID NO: 560 - DNA, 20 bp, Artificial Sequence
  Synthetic oligonucleotide
catggtggcgggggcctgta

SEQ ID NO: 561 - DNA, 20 bp, Artificial Sequence
  Synthetic oligonucleotide
aggcacagcttgcagtgagc

SEQ ID NO: 562 - DNA, 20 bp, Artificial Sequence
  Synthetic oligonucleotide
gcttgaacctgggagacgga

SEQ ID NO: 563 - DNA, 20 bp, Artificial Sequence
  Synthetic oligonucleotide
gggagacggaggttgcagtg

SEQ ID NO: 564 - DNA, 20 bp, Artificial Sequence
  Synthetic oligonucleotide
ctgtagtgccagctactcag

SEQ ID NO: 565 - DNA, 20 bp, Artificial Sequence
  Synthetic oligonucleotide
tgtagtgccagctactcagg

SEQ ID NO: 566 - DNA, 20 bp, Artificial Sequence
  Synthetic oligonucleotide
tttgagcctggaagcggagg

SEQ ID NO: 567 - DNA, 20 bp, Artificial Sequence
  Synthetic oligonucleotide
ttgtcagacgagagctctga

SEQ ID NO: 568 - DNA, 20 bp, Artificial Sequence
  Synthetic oligonucleotide
gagagctctgactctgactc

SEQ ID NO: 569 - DNA, 20 bp, Artificial Sequence
  Synthetic oligonucleotide
actccatggatcatgtgcac

SEQ ID NO: 570 - DNA, 20 bp, Artificial Sequence
  Synthetic oligonucleotide
gcaccagaggcccaggcagc

SEQ ID NO: 571 - DNA, 20 bp, Artificial Sequence
  Synthetic oligonucleotide
cagcccctgaaagagaggga

SEQ ID NO: 572 - DNA, 20 bp, Artificial Sequence
  Synthetic oligonucleotide
gagagggaggtgccctacag

SEQ ID NO: 573 - DNA, 20 bp, Artificial Sequence
  Synthetic oligonucleotide
gagcacttagctttgcggtc

SEQ ID NO: 574 - DNA, 20 bp, Artificial Sequence
  Synthetic oligonucleotide
tgccctcaccagccaggtga

SEQ ID NO: 575 - DNA, 20 bp, Artificial Sequence
  Synthetic oligonucleotide
gacctctctgaataccaccc

SEQ ID NO: 576 - DNA, 20 bp, Artificial Sequence
  Synthetic oligonucleotide
ctgggcagcgtgaggtcatt

SEQ ID NO: 577 - DNA, 20 bp, Artificial Sequence
  Synthetic oligonucleotide
aatctctaccaggtgtttgc

SEQ ID NO: 578 - DNA, 20 bp, Artificial Sequence
  Synthetic oligonucleotide
tgagtgtgtctcacgagtct

SEQ ID NO: 579 - DNA, 20 bp, Artificial Sequence
  Synthetic oligonucleotide
acaagctttccagaacccac

SEQ ID NO: 580 - DNA, 20 bp, Artificial Sequence
  Synthetic oligonucleotide
taggaagccgaaatgccctg

SEQ ID NO: 581 - DNA, 20 bp, Artificial Sequence
  Synthetic oligonucleotide
ccctgatgtggtagagtgtc

SEQ ID NO: 582 - DNA, 20 bp, Artificial Sequence
  Synthetic oligonucleotide
tgtctgttcatgtccccctc

SEQ ID NO: 583 - DNA, 20 bp, Artificial Sequence
  Synthetic oligonucleotide
tggctgagtcccagacctgg

SEQ ID NO: 584 - DNA, 20 bp, Artificial Sequence
  Synthetic oligonucleotide
gacctcaccaccacccgtgg

SEQ ID NO: 585 - DNA, 20 bp, Artificial Sequence
  Synthetic oligonucleotide
ctggccctatttcaacccta

SEQ ID NO: 586 - DNA, 20 bp, Artificial Sequence
  Synthetic oligonucleotide
cctacttcctatggtgttct

SEQ ID NO: 587 - DNA, 20 bp, Artificial Sequence
  Synthetic oligonucleotide
tgttctcatagcaccgtgga

SEQ ID NO: 588 - DNA, 20 bp, Artificial Sequence
  Synthetic oligonucleotide
caccgtggagaattaaagag

SEQ ID NO: 589 - DNA, 20 bp, Artificial Sequence
  Synthetic oligonucleotide
cactaagtaagggcactcta

SEQ ID NO: 590 - DNA, 20 bp, Artificial Sequence
  Synthetic oligonucleotide
caaagcactataacggatct

SEQ ID NO: 591 - DNA, 20 bp, Artificial Sequence
  Synthetic oligonucleotide
aaccagcagtggacacacag

SEQ ID NO: 592 - DNA, 20 bp, Artificial Sequence
  Synthetic oligonucleotide
aacaagcccagggtgaaaag

SEQ ID NO: 593 - DNA, 20 bp, Artificial Sequence
  Synthetic oligonucleotide
gtgaaaagttctgcagggga

SEQ ID NO: 594 - DNA, 20 bp, Artificial Sequence
  Synthetic oligonucleotide
aggggatgactgccagttca

SEQ ID NO: 595 - DNA, 20 bp, Artificial Sequence
  Synthetic oligonucleotide
tctcactgtggctctatttc

SEQ ID NO: 596 - DNA, 20 bp, Artificial Sequence
  Synthetic oligonucleotide
tgacataggacactagaagg

SEQ ID NO: 597 - DNA, 20 bp, Artificial Sequence
  Synthetic oligonucleotide
ctagaagggcaaacgcagtg

SEQ ID NO: 598 - DNA, 20 bp, Artificial Sequence
  Synthetic oligonucleotide
gtgtcacctgccactattac

SEQ ID NO: 599 - DNA, 20 bp, Artificial Sequence
  Synthetic oligonucleotide
tactcaattacttgatgacc

SEQ ID NO: 600 - DNA, 20 bp, Artificial Sequence
  Synthetic oligonucleotide
tggcactaaactggctgtgg

SEQ ID NO: 601 - DNA, 20 bp, Artificial Sequence
  Synthetic oligonucleotide
gtggccaaaggctagagagc

SEQ ID NO: 602 - DNA, 20 bp, Artificial Sequence
  Synthetic oligonucleotide
agcaagctcctccttgccct

SEQ ID NO: 603 - DNA, 20 bp, Artificial Sequence
  Synthetic oligonucleotide
cccacttgaagagactgcta

SEQ ID NO: 604 - DNA, 20 bp, Artificial Sequence
  Synthetic oligonucleotide
caccttggacttcaatccac

SEQ ID NO: 605 - DNA, 20 bp, Artificial Sequence
  Synthetic oligonucleotide
ctggagagaagcgcggtctt

SEQ ID NO: 606 - DNA, 20 bp, Artificial Sequence
  Synthetic oligonucleotide
ctccctggaggacacggtgc

SEQ ID NO: 607 - DNA, 20 bp, Artificial Sequence
  Synthetic oligonucleotide
gaacgccaaactgggagaag

SEQ ID NO: 608 - DNA, 20 bp, Artificial Sequence
  Synthetic oligonucleotide
ctcccccattactcgtccac

SEQ ID NO: 609 - DNA, 20 bp, Artificial Sequence
  Synthetic oligonucleotide
tcagacccccgcctacctgc

SEQ ID NO: 610 - DNA, 20 bp, Artificial Sequence
  Synthetic oligonucleotide
ccccgcccctgactgcgctg

SEQ ID NO: 611 - DNA, 20 bp, Artificial Sequence
  Synthetic oligonucleotide
gcgctgggtaggtggtcgct

SEQ ID NO: 612 - DNA, 20 bp, Artificial Sequence
  Synthetic oligonucleotide
ggaccggcggaatcacaccc

SEQ ID NO: 613 - DNA, 18 bp, Artificial Sequence
  Synthetic oligonucleotide
gcgttgccgttcctctgg

SEQ ID NO: 614 - DNA, 18 bp, Artificial Sequence
  Synthetic oligonucleotide
tgcggaccgtggacatga

SEQ ID NO: 615 - DNA, 18 bp, Artificial Sequence
  Synthetic oligonucleotide
gccgtggagctcgcagaa

SEQ ID NO: 616 - DNA, 18 bp, Artificial Sequence
  Synthetic oligonucleotide
gcctggcggacaatgctg

SEQ ID NO: 617 - DNA, 18 bp, Artificial Sequence
  Synthetic oligonucleotide
gtgctggtggctgacgta

SEQ ID NO: 618 - DNA, 18 bp, Artificial Sequence
  Synthetic oligonucleotide
cgggtgccctgcagcaag

(19) United States
(12) Patent Application Publication
(10) Pub. No.: US 2023/0272401 A1
(43) Pub. Date: Aug. 31, 2023

(54) COMPOSITIONS FOR FLCN GENE MODULATION AND METHODS THEREOF

(71) Applicant: GENETIC INTELLIGENCE, INC, New York, NY (US)

(72) Inventors: Bertrand ADANVE, New York, NY (US); Yao Zong NG, Singapore (SG); Jonathan Smithtown, Smithtown, NY (US); David Y. YOUNG, Boston, MA (US); Michael CLARK, New York, NY (US)

(21) Appl. No.: 18/009,499

(22) PCT Filed: Jun. 11, 2021
(86) PCT No.: PCT/US2021/037022

§ 371 Date: Dec. 9, 2022

Related U.S. Application Data
(60) Provisional application No. 63/037,881, filed on Jun. 11, 2020.

(51) Int. Cl. C12N 15/113; A61K 31/713; A61P 25/28

(52) U.S. Cl. CPC C12N 15/1135; A61K 31/713; A61P 25/28; C12N 2310/11; C12N 2310/341; C12N 2310/322; C12N 2310/315; C12N 2310/31; C12N 2310/3341; C12N 2310/14

(57) ABSTRACT

Compositions, systems, kits, and methods are described herein for the modulation, and in particular the reduction or inhibition, of expression of the FLCN gene or the activity of FLCN protein in a cell, animal, or human subject. Compositions, systems, and methods disclosed herein can be used to prevent, ameliorate, or treat diseases, particularly neuromuscular or neurodegenerative diseases, retinal degeneration diseases, or other TDP-43 proteinopathies. Methods are described for the modulation, and in particular the reduction or inhibition of FLCN expression or activity, comprising the use of a modulator to regulate FLCN expression or activity. Methods are also described for the development, synthesis, and production of modulators, and for therapeutic treatment of TDP-43 proteinopathies such as ALS and other related disorders. Furthermore, methods for diagnostics and testing comprising detecting FLCN associated variants, or FLCN expression or activity levels, as well as compositions comprising kits for diagnostics and testing, are described herein.

### CROSS-REFERENCE TO RELATED APPLICATIONS

The instant application claims priority to U.S. Provisional Application No. 63/037,881 (filed on Jun. 11, 2020), the entire contents of which is incorporated by reference in its entirety.

### SEQUENCE LISTING

The instant application contains a Sequence Listing, which has been filed electronically in ASCII format and is hereby incorporated by reference in its entirety. Said ASCII copy, created Jun. 8, 2021, is named Sequence_ListingST25.txt and is 218,047 bytes in size.

### FIELD OF THE INVENTION

This invention relates to compositions, systems, and methods for modulating, in particular reducing or inhibiting, the expression or activity of FLCN in a cell, an animal or human subject. Such compositions, systems, and methods can be useful to treat, prevent, or ameliorate diseases, particularly neuromuscular or neurodegenerative diseases, such as amyotrophic lateral sclerosis (ALS), frontotemporal lobar degeneration (FTLD), Alzheimer's disease, retinal degeneration diseases such as age-related macular degeneration (AMD), and other TDP-43 proteinopathies. Such compositions, systems, and methods can also be useful to treat, prevent, or ameliorate diseases, particularly oxidative stress, obesity, anemia and ischemic diseases, such as cardiovascular disease, myocardial ischemia and peripheral vascular disease. This invention also relates to compositions, systems, and methods for modulating, in particular increasing, the expression or activity of FLCN in a cell, an animal or human subject, which can be useful to treat, prevent, or ameliorate diseases, particularly Birt-Hogg-Dube (BHD) syndrome, fibrofolliculomas, lung cysts, spontaneous pneumothorax, kidney tumors, and other diseases that are linked to loss-of-function of FLCN. Such compositions, systems, and methods can also be useful to treat, prevent or ameliorate diseases, particularly inflammatory diseases, von Hippel-Lindau (VHL) disease, B cell deficiency, cardiomyopathy, as well as other cancers.

### BACKGROUND

In the following discussion certain articles and processes will be described for background and introductory purposes. Nothing contained herein is to be construed as an “admission” of prior art. Applicant expressly reserves the right to demonstrate, where appropriate, that the articles and processes referenced herein do not constitute prior art under the applicable statutory provisions.

Amyotrophic lateral sclerosis (ALS), also known as Lou Gehrig's disease, is characterized by the progressive degeneration and loss of survival of both upper motor neurons in the brain and lower motor neurons in the spinal cord. This results in a lack of muscle stimulation, leading to muscle atrophy and fasciculations. Around 10% of ALS patients can survive for 10 or more years. However, most patients (around 90%) ultimately die from respiratory failure within 3 to 5 years from the onset of symptoms. ALS affects over 30,000 in the US alone, with over 5,600 new cases annually. There is currently a lack of accurate diagnostics and effective prophylaxis or therapies for ALS (Khairoalsindi and Abuzinadah, Neurology Research International, Article ID 6534150 (2018)). The drug Riluzole, which was approved by the FDA in 1995, can in most cases only extend the survival of ALS patients by 2-3 months. Edaravone (Radicava), which was approved by the FDA in 2017, slows the decline in physical function in ALS patients but is only effective for a subset of patients (around 7%), and long-term data on any survival benefit is still lacking. The lack of a long-term, effective therapy for ALS highlights the urgent need for the development of novel ALS therapies.

It is important to identify the genetic features in patients that have ALS, as an understanding of the molecular basis of the disease will aid the development of effective modulators that can be used as prophylactics or therapeutics to prevent or treat the disease, respectively. ALS is a complex disease that arises from the interplay of multiple genetic factors that are still poorly understood. 90-95% of ALS cases are sporadic, occurring apparently at random in individuals without a family history of ALS. In contrast, about 5-10% of ALS cases are familial, occurring in individuals with a family history of ALS (Renton et al., Nature Neuroscience, 17(1): 17-23 (2014)). To date, studies of familial and sporadic ALS cases have uncovered mutations in over 30 genes (e.g., C9orf72, SOD1, STMN2, NEK1, TARDBP, FUS, VCP, OPTN, SQSTM1, UBQLN2, hnRNPA1, MATR3, and others) that collectively account for less than 17% of all ALS cases (Renton et al., Nature Neuroscience, 17(1): 17-23 (2014)). However, the majority (>83%) of ALS cases have unknown causes.

In 2006, TAR DNA-binding protein 43 (TDP-43) was discovered to be a key component of insoluble and highly ubiquitinated aggregates in the brains of patients suffering from ALS and frontotemporal lobar dementia (FTLD). Despite the potentially diverse genetic etiology of ALS, strikingly, over 97% of all ALS cases (both sporadic and familial) and around 45% of frontotemporal lobar degeneration (FTLD; also called frontotemporal dementia (FTD) which is a type of FTLD) cases display TDP-43 positive aggregates in the cytoplasm of affected neurons (Prasad et al., Frontiers in Molecular Neuroscience, 12:25 (2019)). Furthermore, mutations in TARDBP, the gene that encodes for TDP-43, have been associated with familial cases of ALS, thus cementing its central role in ALS. In general, once initiated, the aggregation of TDP-43 in the cytoplasm can proceed in a self-propagating manner, involving polymerization of RNA and protein molecules to form toxic products that are resistant to proteolysis. In healthy cells, TDP-43 is predominantly localized to the nucleus and carries out multiple important RNA processing functions there, including regulating RNA transcription, RNA splicing, RNA transport, and stability. Its multi-domain structure allows it to be a central modulator of multiple processes. For example, it comprises two RNA recognition motifs (RRM1 and RRM2) that mediate interactions with RNA and DNA, a C-terminal glycine-rich domain that mediates interactions with other proteins, as well as a nuclear localization signal (NLS) and nuclear export signal (NES) that regulates its shuttling between the nucleus and cytoplasm. Thus, TDP-43's involvement in ALS pathogenesis can be caused by a toxic gain-of-function mechanism due to increased levels of pathological TDP-43 aggregates in the cytoplasm, or a loss-of-function mechanism due to a decrease of functional TDP-43 levels in the nucleus, or a combination of both. The role of TDP-43 in ALS is described by Prasad et al. (Prasad et al., Frontiers in Molecular Neuroscience, 12:25 (2019)) and Scotter et al. (Scotter et al., Neurotherapeutics, 12(2): 352-363 (2015)), the disclosures of which, along with their references, are incorporated herein in its entirety.

Scotter et al. (Scotter et al., Neurotherapeutics, 12(2): 352-363 (2015)), discloses that “several animal studies have found that mutant TDP-43 causes toxicity in the absence of visible aggregates” and that “visible aggregates are only the endpoint of an aggregation pathway that includes a range of TDP-43 species from misfolded monomer to oligomer to mature aggregates”. Thus, the term TDP-43 aggregates can refer to a range of TDP-43 species from misfolded monomer to oligomer to mature aggregates, whether visible or not.

Consequently, a decrease in the levels of pathological TDP-43 aggregates in the cytoplasm, or an increase in the levels of normal TDP-43 in the nucleus, or a combination of both, are likely to be effective to treat, ameliorate, or prevent ALS or represent an indication thereof as a biomarker. Likewise, decreasing TDP-43 aggregates in the cytoplasm are likely to be effective to treat, ameliorate, or prevent other diseases, particularly neuromuscular or neurodegenerative diseases, involving either an increase in levels of pathological TDP-43 aggregates in the cytoplasm, or a decrease of functional TDP-43 levels in the nucleus, or a combination thereof. Such diseases are termed TDP-43 proteinopathies, examples of which include but are not limited to, ALS, Alzheimer's disease, argyrophilic grain disease, vascular dementia, frontotemporal dementia (FTD, FTD-TDP-43, and FTD-tau) and the greater group of frontotemporal lobar degeneration (FTLD), semantic dementia, dementia with Lewy bodies, polyglutamine diseases, Huntington's disease, spinocerebellar ataxia, inclusion body myopathy, inclusion body myositis, hippocampal sclerosis, parkinsonism, Parkinson's disease (PD), Perry syndrome, ALS-parkinsonism dementia complex of Guam, primary lateral sclerosis (PLS), hereditary spastic paraplegia (HSP), pseudobulbar palsy, Mills' syndrome, monomelic amyotrophy, post-polio syndrome (PPS), madras motor neuron disease (MMND), progressive muscular atrophy (PMA), spinal muscular atrophy (SMA), spinal and bulbar muscular atrophy (SBMA), progressive bulbar palsy (PBP), retinal degeneration diseases such as age-related macular degeneration (AMD), retinitis pigmentosa (RP), and glaucoma, wherein in each case at least a sub-population of patients can exhibit an increase in levels of TDP-43 aggregates in the cytoplasm. TDP-43 proteinopathies are further described in Gendron and Josephs (Gendron and Josephs, Neuropathol. Appl. Neurobiol. 36:97-112 (2010)), Lagier-Tourenne et al. (Lagier-Tourenne et al., Hum. Mol. Gen. 19(1):R46-R64 (2010)), and Matsukawa et al. (Matsukawa et al., Journal of Biological Chemistry, 291(45): 23464-23476 (2016)), the disclosures of which, together with references cited therein, are incorporated herein in its entirety.

### SUMMARY

Compositions, systems, and methods are described herein for the modulation, and in particular the reduction or inhibition, of expression of the FLCN gene or the activity of FLCN protein in a cell, animal or human subject. Compositions, systems, and methods disclosed herein can be used to prevent, ameliorate, or treat diseases, particularly neuromuscular or neurodegenerative diseases, such as ALS, FTLD, Alzheimer's disease, retinal degeneration diseases such as age-related macular degeneration (AMD), or other TDP-43 proteinopathies. Methods are described for the modulation, and in particular the reduction or inhibition of FLCN expression or activity, comprising the use of a modulator to regulate FLCN expression or activity. Included also herein are compositions for modulators used to regulate FLCN expression or activity. Related pharmaceutical compositions, kits, and methods of delivery of compositions used in modulating, and in particular reducing or inhibiting FLCN expression or activity are also described. Methods are also described for the development, synthesis, and production of modulators, as well as for therapeutic treatment of ALS and other related disorders such as other TDP-43 proteinopathies. Furthermore, methods for diagnostics and testing comprising detecting FLCN associated variants, or FLCN expression or activity levels, as well as compositions comprising kits for diagnostics and testing, are described herein.

In some embodiments, the above compositions, systems, and methods can also be useful to treat, prevent, or ameliorate diseases, particularly oxidative stress, obesity, anemia, and ischemic diseases, such as cardiovascular disease, myocardial ischemia and peripheral vascular disease.

In some embodiments, provided herein are antisense modulators to modulate FLCN expression or activity. In one embodiment, provided herein are antisense modulators to inhibit or reduce FLCN expression or activity. In one embodiment, antisense modulators comprise antisense oligonucleotides (ASOs). In one embodiment, the ASO is between 12-30 nucleobases in length. In certain embodiments, the ASO is at least 70% complementary, alternatively at least 80% complementary, alternatively at least 85% complementary, alternatively at least 90% complementary, alternatively at least 95% complementary, alternatively at least 98% complementary, alternatively 100% complementary to at least one target sequence of identical length described in SEQ ID NOs: 1-15; wherein at least one target sequence of identical length described in SEQ ID NOs: 1-15 shall be construed by a reasonable person skilled in the art as referring to an equal length portion of at least one target sequence described in SEQ ID NOs: 1-15. In certain embodiments, the ASO is at least 70% identical, alternatively at least 80% identical, alternatively at least 85% identical, alternatively at least 90% identical, alternatively at least 95% identical, alternatively at least 98% identical, alternatively 100% identical to at least one sequence described in SEQ ID NOs: 16-618. In one embodiment, the ASO includes at least one modification to an internucleoside linkage, a sugar, or a nucleobase component. In one embodiment, all of the internucleoside linkages of the ASO comprise phosphorothioate modifications. In another embodiment, all of the sugar components of the ASO comprise the 2′-MOE modification. In another embodiment, the ASO comprises a gapped sequence consisting of a central sequence of oligonucleotides without sugar modifications, which are flanked on both sides by wing sequences consisting of 2′-MOE modified nucleotides. In another embodiment, the ASO comprises a gapped sequence consisting of a central sequence of deoxynucleotides, which are flanked on both sides by wing sequences consisting of 2′-MOE modified nucleotides, and wherein the second nucleotide of the central sequence from the 5′ end of the ASO contains a 2′-OMe sugar modification. In one embodiment, all cytosine nucleobases of the ASO comprise 5-methylcytosine modifications. In certain embodiments, the ASO is conjugated to one or more molecules, such as a peptide or polypeptide, lipid, sugar, nucleotide or oligonucleotide, other polymer, cleavage agent, transport agent, intercalating agent, molecular beacon, hybridization-triggered crosslinking agent, lipophilic agent, or hydrophilic agent. In one embodiment, the ASO is conjugated to one or more N-acetyl galactosamine (GalNAc) residue or other such conjugates or complexes. In some embodiments, the ASO comprises one of the modified sequences in Table 17.

In some embodiments, antisense modulators comprise modulators used in RNA interference (RNAi), such as siRNAs, miRNAs and shRNAs. In one embodiment, the antisense modulator is a siRNA. In one embodiment, the antisense region of the siRNA is 19 to 29 nucleotides in length. In certain embodiments, the antisense region of the siRNA is at least 70% complementary, alternatively at least 80% complementary, alternatively at least 85% complementary, alternatively at least 90% complementary, alternatively at least 95% complementary, alternatively at least 98% complementary, alternatively 100% complementary to at least one target sequence of identical length described in SEQ ID NOs: 1-15; wherein at least one target sequence of identical length described in SEQ ID NOs: 1-15 shall be construed by a reasonable person skilled in the art as referring to an equal length portion of at least one target sequence described in SEQ ID NOs: 1-15. In certain embodiments, the antisense region of the siRNA is at least 70% identical, alternatively at least 80% identical, alternatively at least 85% identical, alternatively at least 90% identical, alternatively at least 95% identical, alternatively at least 98% identical, alternatively 100% identical to at least one sequence described in SEQ ID NOs: 16-618, wherein thymine is replaced by uracil. In one embodiment, the first two nucleotides at the 5′ end of the sense strand, as well as the first two nucleotides at the 5′ end of the antisense strand, of the siRNA are modified with a 2′-O-alkyl group, such as a 2′-OMe group.

In other embodiments, provided herein are modulators other than antisense modulators, for example other oligonucleotide modulators (e.g., ribozyme, deoxyribozyme, or aptamers), antibody modulators, peptide modulators, small molecule modulators, and nucleic acid vectors, for modulating, and in particular reducing or inhibiting, the expression or activity of FLCN in a cell, an animal or human subject. In certain embodiments, the antibody modulator is chosen from the set of modulators described in Table 16. In some embodiments, the antibody, antibody fragment, monobody or peptide modulator binds to the same epitope as at least one antibody modulator described in Table 16. In other embodiments, the antibody, antibody fragment, monobody or peptide modulator binds to a different epitope to that of the modulators described in Table 16. In some embodiments, the antibody, antibody fragment, monobody, or peptide modulator comprises a complementarity-determining region (CDR) that is at least 50% similar to the CDR of at least one antibody modulator described in Table 16, as assessed by sequence alignment or other scoring methods known in the art. In some embodiments, the antibody, antibody fragment, monobody, or peptide modulator comprises a complementarity-determining region (CDR) that is at least 30%, 40%, 50%, 60%, 70%, 75%, 80%, 85%, 90%, 95%, 96%, 97%, 98%, 99%, or 100% similar to the CDR of at least one antibody modulator described in Table 16, as assessed by sequence alignment or other scoring methods known in the art. In certain embodiments, provided herein are small molecule modulators comprising at least one exemplar small molecule modulator described in Table 15. In certain embodiments, the small molecule modulator comprises at least one scaffold described in Table 15. In some embodiments, the small molecule modulators, or part thereof, have a Tanimoto index of at least 0.20, 0.30, 0.40, 0.50, 0.60, 0.70, 0.75, 0.80, 0.85, 0.90, 0.95, 0.96, 0.97, 0.98, 0.99, or 1.00 compared to at least one exemplar or scaffold described in Table 15.

In certain embodiments, provided herein are methods and compositions for the delivery of antisense modulators into a cell, an animal, or a human subject, in order to modulate the expression or activity of FLCN, in particular to reduce or inhibit the expression or activity of FLCN. In one embodiment, delivery methods and compositions comprise a transfection reagent, such as a liposomal-based or amine-based transfection reagent. In one embodiment, the antisense modulators are delivered naked without a transfection reagent. In one embodiment, the antisense modulators are delivered via a nucleic acid vector. In one embodiment, the method of delivery includes one or more of the common delivery methods used to deliver drugs, such as intrathecal injection.

One set of embodiments provide for pharmaceutical compositions, comprising a modulator and a pharmaceutically acceptable carrier or diluent, which can be administered to a cell, an animal, or a human subject to modulate, and in particular to reduce or inhibit, the expression or activity of FLCN in the cell, animal or human subject. In one embodiment, the pharmaceutical composition is administered intrathecally. Such treatment methods can be used to treat, ameliorate, or prevent ALS and other diseases, such as other TDP-43 proteinopathies. In another embodiment, such treatment methods can also be used to treat, prevent, or ameliorate diseases, particularly oxidative stress, obesity, anemia, and ischemic diseases, such as cardiovascular disease, myocardial ischemia, and peripheral vascular disease. In one embodiment, a pharmaceutical composition described herein is co-administered with one or more other pharmaceutical agents, such as for example, Riluzole (Rilutek), Dexpramipexole, Edaravone, Tofersen, Baclofen (Lioresal), or other drug that is typically administered to treat, ameliorate, or manage symptoms in ALS. In other embodiments, a pharmaceutical composition described herein is co-administered with one or more pharmaceutical agents or other drug that is typically administered to treat, ameliorate, or manage symptoms in oxidative stress, obesity, anemia or ischemic diseases; as well as inflammatory diseases, von Hippel-Lindau (VHL) disease, Birt-Hogg-Dube (BHD) syndrome, spontaneous pneumothorax, B cell deficiency, cardiomyopathy, and cancers.

Some embodiments provide for the testing and monitoring of FLCN levels or activity in a cell, an animal, or a human subject. In some embodiments, these tests can be used for the purposes of diagnosing ALS. In other embodiments, these tests can be used for the purposes of determining risk or susceptibility to ALS. In some embodiments, these tests can be used for the purposes of monitoring ALS progression or response to a treatment.

In other embodiments, provided herein are methods of determining risk or susceptibility, methods of diagnosis, methods of predicting prognosis, or methods of assessing a human individual for a probability of a response to a therapeutic method and/or modulator for neuromuscular or neurodegenerative diseases, such as, for example, FTLD, Alzheimer's Disease, retinal degeneration diseases such as age-related macular degeneration (AMD), and other TDP-43 proteinopathies disclosed herein. In other embodiments, provided herein are methods of determining risk or susceptibility, methods of diagnosis, methods of predicting prognosis, or methods of assessing a human individual for a probability of a response to a therapeutic method and/or modulator for oxidative stress, obesity, anemia, or ischemic disease; as well as inflammatory disease, von Hippel-Lindau (VHL) disease, Birt-Hogg-Dube (BHD) syndrome, spontaneous pneumothorax, B cell deficiency, cardiomyopathy, and cancer.

In certain embodiments, also provided herein are compositions, systems, and methods for modulating, in particular increasing or upregulating, the expression or activity of FLCN in a cell, animal or human subject. Such compositions, systems, and methods can be used to prevent, ameliorate, or treat diseases, particularly Birt-Hogg-Dube (BHD) syndrome, fibrofolliculomas, lung cysts, spontaneous pneumothorax, kidney tumors, and other diseases that are linked to loss-of-function of FLCN.

In some embodiments, provided herein are compositions, systems, and methods for modulating, in particular increasing or upregulating, the expression or activity of FLCN in a cell, animal or human subject, which can be used to treat inflammatory diseases, von Hippel-Lindau (VHL) disease, B cell deficiency, cardiomyopathy, as well as other cancers.

### DETAILED DESCRIPTION

The following description is presented to enable one of ordinary skill in the art to make and use the invention and is provided in the context of a patent application and its requirements. Various modifications to the example embodiments and the genetic principles and features described herein will be readily apparent. The example embodiments are mainly described in terms of particular processes and systems provided in particular implementations. However, the processes and systems will operate effectively in other implementations. Phrases such as “example embodiment”, “one embodiment”, and “another embodiment” can refer to the same or different embodiments.

The example embodiments will be described with respect to methods and compositions having certain components. However, the methods and compositions can include more or less components than those shown, and variations in the arrangement and type of the components can be made without departing from the scope of the invention.

The example embodiments will also be described in the context of methods having certain steps. However, the methods and compositions operate effectively with additional steps and steps in different orders that are not inconsistent with the example embodiments. Thus, the present invention is not intended to be limited to the embodiments shown but is to be accorded the widest scope consistent with the principles and features described herein and as limited only by appended claims.

It should be noted that as used herein and in the appended claims, the singular forms “a,” “and,” and “the” include plural referents unless the context clearly dictates otherwise. Thus, for example, reference to the effect of “a neuron” can refer to the effect of one or a combination of neurons, and reference to “a method” includes reference to equivalent steps and processes known to those skilled in the art, and so forth.

Where a range of values is provided, it is to be understood that each intervening value between the upper and lower limit of that range—and any other stated or intervening value in that stated range—is encompassed within the invention. Where the stated range includes upper and lower limits, ranges excluding either of those limits are also included in the invention.

Unless expressly stated, the terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated. All publications mentioned herein are incorporated by reference for the purpose of describing and disclosing the formulations and processes that are described in the publication and which might be used in connection with the presently described invention.

Those skilled in the art will recognize, or be able to ascertain using no more than routine experimentation, many equivalents to the specific embodiments of the invention described herein in the detailed description and figures. Such equivalents are intended to be encompassed by the claims.

For simplicity, in the present document certain embodiments are described with respect to use of certain methods. It will become apparent to one skilled in the art upon reading this disclosure that the invention is not intended to be limited to a specific use, and can be used in a wide array of implementations.

Throughout this specification, the oligonucleotides and polypeptides referred to include all enantiomers, stereoisomers, racemic mixtures, optically pure isomer forms, complementary sequences, modified or analog forms, and both deoxyribonucleotide (or DNA) and ribonucleotide (or RNA) forms.

### Definitions

Unless defined otherwise, all technical and scientific terms used herein have the same meaning as commonly understood by the ordinary person skilled in the art to which the embodiments pertain.

“Nucleic acid sequence data” as used herein refers to any sequence data obtained from nucleic acids from an individual. Such data includes, but is not limited to, deoxyribonucleotide (DNA) data, ribonucleotide (RNA) data, whole genome sequencing data, exome sequencing data, genotyping data, transcriptome sequencing data, complementary DNA or cDNA library sequencing data, and the like. Nucleic acid sequences are written in the 5′ to 3′ direction.

“Nucleobases”, or “bases”, are used interchangeably and refer to nitrogen-containing compounds that form nucleosides, which in turn are components of nucleotides. The five primary or natural nucleobases are adenine (A), cytosine (C), guanine (G), thymine (T), and uracil (U). Other nucleobases, such as synthetic or modified nucleobases, are included herein and detailed below.

“Nucleotide” refers to a compound comprising a nucleoside and a linkage group, commonly a phosphate linkage group. Nucleotides include both natural and modified nucleotides.

“Sugar” or “sugar component” can mean a natural or modified sugar, which includes ribose sugars and deoxyribose sugars found in RNA and DNA, respectively, as well as other modified sugars detailed below.

“Nucleoside” refers to a compound comprising both a nucleobase and sugar component. Nucleosides can be natural or modified.

“Nucleoside linkage” or “internucleoside” linkage refers to the covalent linkages of adjacent nucleosides. Nucleoside linkages comprise the primary linkages between nucleotides in an oligonucleotide.

“Chimeric compound” refers to a compound, most commonly an oligonucleotide, that comprises at least one nucleotide having at least one nucleobase, nucleoside linkage, or sugar component that differs from at least one other nucleotide within the same compound. This difference can originate from variations in how components of nucleotides within the same compound are modified or in some cases left unmodified. In some embodiments, similar or identical modifications to nucleotides in chimeric compounds can be grouped together spatially in regions. Any modification or combination of modifications described herein or elsewhere, including those modifications known to persons skilled in the art, can be included in a chimeric compound.

“Motif” refers to a region or subsequence within the sequence of an oligonucleotide, or polypeptide, that has a specific functional or biological significance. Examples of motifs include nucleobase sequences within an oligonucleotide, such as DNA or RNA, which are recognized by a DNA or RNA-binding protein. Other examples of motifs include amino acid sequences within a polypeptide that is responsible for a specific function of the polypeptide.

“Nucleic acid sequence”, “nucleobase sequence”, “nucleotide sequence”, or simply “sequence” are used interchangeably and refer to the sequence of nucleobases on a nucleic acid molecule or oligonucleotide.

“Coding DNA” refers to a DNA sequence that is transcribed to messenger RNA (mRNA) and subsequently translated to a polypeptide or protein.

“Non-coding DNA” refers to a DNA sequence that does not encode a polypeptide, including, but not limited to, a DNA sequence that is transcribed to a functional RNA (e.g., non-coding RNA (ncRNA), transfer RNA (tRNA), ribosomal RNA (rRNA), regulatory RNA, microRNA (miRNA), small interfering RNA (siRNA), Piwi interacting RNA (piRNA) or long noncoding RNA (lncRNA)); a DNA sequence that contains a regulatory element such as a promoter, enhancer, terminator, insulator or silencer that affects the expression of one or more genes; a DNA sequence that performs a structural function (e.g., centromere, telomere, satellite); a DNA sequence that serves as a replication origin; a DNA sequence that is located within a protein-coding gene but is removed before a protein is made, otherwise known as an intron; or otherwise any DNA sequence with unknown function.

The term “mRNA” refers to messenger RNA, the message derived by the transcription of coding DNA to form precursor mRNA (pre-mRNA). Pre-mRNA is subsequently processed into mature mRNA by splicing to remove introns, and addition of a 5′ cap and poly-A tail. Mature mRNA is used as a template by ribosomes for translation into polypeptides. The term “mRNA” as used herein includes pre-mRNA, sometimes also referred to as hnRNA (heterogeneous nuclear RNA), mature mRNA, as well as mRNA in any stage of processing.

The term “gene” as used herein, refers to a DNA sequence that is transcribed to mRNA and subsequently translated to a polypeptide, and/or a DNA sequence that is transcribed to a functional RNA.

“Polypeptide”, “oligopeptide”, “peptide” and “protein” are used interchangeably, and refer to a polymer of two or more amino acids.

“Oligonucleotide” refers to a polymer comprising two or more nucleotides, and can also refer to a modified oligonucleotide. “Modified oligonucleotide” refers to an oligonucleotide comprising at least one modification to a nucleobase, nucleoside linkage, or sugar component.

An “allele”, also referred to as a “variant”, or “polymorphism”, refers to one of at least two different nucleotide sequence variations at a given position (locus) in the genome. Thus, a specific allele of a polymorphic site refers to a specific version of the sequence with respect to a polymorphic site. A “variant” or “polymorphism” can also refer to a specific allele of a polymorphic site that differs from a reference genome.

“Polymorphic marker”, also referred to as “polymorphic site” or simply as “marker”, refers to a genomic site with at least two sequence variants, or at least two alleles. Thus, genetic association with a polymorphic marker, refers to association with at least one specific allele of that polymorphic marker. “A marker” can also refer to a specific allele of a polymorphic marker. A polymorphic marker can refer to any type of sequence variation found in the genome, including but not limited to single nucleotide polymorphisms (SNPs), curated SNPs (cSNPs), insertions, deletions, copy number variations (CNVs), codon expansions, methylation status, translocations, duplications, repeat expansions, rearrangements, multi-base polymorphisms, splice variants, microsatellite polymorphisms etc. A “marker” can also refer to a “biomarker”.

A “single nucleotide polymorphism” or “SNP” is a type of variation of DNA where a single nucleotide at a specific location in a genome differs between two or more individuals, or two or more populations. Most SNPs have two alleles; in such cases, an individual is either homozygous for one allele at the polymorphic site, or heterozygous for both alleles.

An “insertion” or “deletion” is a variant with additional nucleotides or fewer nucleotides respectively compared to a reference DNA sequence.

A “microsatellite” is a type of polymorphic marker where there are multiple small repeats of bases that are 2-8 nucleotides in length.

A “haplotype” refers to a segment of genomic DNA containing a specific combination of alleles along the segment that tends to be inherited together in human evolution.

“Linkage disequilibrium” refers to the non-random association of alleles at different loci in a given population.

The term “associated with” refers to and can be used interchangeably with “within”, or “correlated with”, or “in linkage disequilibrium with”, or “functionally related with”, or any combination of the terms.

“Susceptibility” refers to the tendency, propensity or risk of an individual to develop a particular phenotype (e.g., a trait or a disease), or to being more or less able to resist developing a particular phenotype. The term encompasses decreased susceptibility to, or decreased risk of, or a protection against a disease. The term also encompasses an increased susceptibility to, or increased risk of developing, a disease.

The term “and/or” indicates “one or the other or both”. In other words, the term indicates that both or either of the items are involved.

The term “biomarker” refers to a biological molecule such as a protein, a polypeptide, a small molecule, a metabolite or a nucleic acid sequence that is associated with a phenotype such as a disease, and whose measurement can be used for determining a susceptibility to the disease, or prognosis for the disease, or diagnosis for the disease, or determining a response to a therapy for the disease.

The term “look-up table” is a table that links one form of data to another, or one or more forms of data to a predicted outcome (e.g., a trait, a disease, or other phenotype). Look-up tables can contain information about one or more polymorphic markers, one or more alleles at each polymorphic marker, and a correlation between alleles for a polymorphic marker and a particular phenotype (e.g., a trait or a disease).

A “computer-readable medium” is a medium for storage of information that is accessible by a computer interface that is custom-built or available commercially. Some examples of computer-readable media include, but are not limited to, optical storage media, magnetic storage media, memory, punch cards, or other commercially available media.

A “nucleic acid sample” refers to a DNA and/or RNA sample obtained from an individual. In certain embodiments (e.g., in detecting specific polymorphic markers and/or haplotypes), the nucleic acid sample comprises genomic DNA. Genomic DNA samples can be obtained from any source that contains genomic DNA, such as blood, saliva, tissue sample, cerebrospinal fluid, amniotic fluid etc.

A “sample” in general refers to any sample, such as a biological sample, obtained from an individual.

A “subject”, a “patient” or an “individual” refers to a living multi-cellular vertebrate organism, which includes both human and non-human mammals, unless otherwise indicated.

The term “therapeutic agent” refers to an agent that can be used for preventing, treating, or ameliorating symptoms associated with a disease.

The term “response to a therapeutic method”, “response to a therapy”, or “response to administration of a modulator” refers to the result of any kind of treatment on an individual, and includes beneficial, neutral, and adverse effects.

The term “therapeutically effective amount” refers to an amount of a therapeutic agent, which when administered alone or together with one or more additional therapeutic agents, induces the desired response, such as decreasing signs and symptoms associated with disease. Often, the therapeutically effective amount provides the desired response without causing a significant side effects to the administered subject.

The term “disease-associated nucleic acid” refers to a nucleic acid that has been found to be associated or correlated with the disease. This includes markers and haplotypes described herein, and/or markers and haplotypes in strong linkage disequilibrium therewith.

The term “modulator” refers to a compound that affects the signaling, activity or expression of polypeptides or nucleic acid sequences (also referred to as “modulates”), and includes both activators and inhibitors. A modulator that increases or upregulates the signaling, activity or expression of polypeptides or nucleic acid sequences is referred to as an “activator”. A modulator that inhibits, reduces, decreases or downregulates the signaling, activity or expression of polypeptides or nucleic acid sequences is referred to as an “inhibitor”. “Modulation” refers to the act of modulating as defined above and can be performed with a modulator. Unless specified otherwise, “modulate” or “modulation” refers to the act of modulating as defined above, and includes both increasing or upregulating the signaling, activity or expression of polypeptides or nucleic acid sequences, as well as inhibiting, reducing or downregulating the signaling, activity or expression of polypeptides or nucleic acid sequences.

The term “antisense modulator” refers to a modulator that affects the signaling, activity or expression of at least one nucleic acid sequence through some form of complementary binding or hybridization to the nucleic acid molecule. Common forms of antisense modulators include ASOs as well as nucleic acids used in the RNAi mechanism for gene modulation, including, but not limited to, miRNA, siRNA, and short hairpin RNA (shRNA).

The term “amplification” or to “amplify” refers to increasing the number of copies of a sequence of nucleotides. An example of amplification is the “polymerase chain reaction”, in which a sample containing sequences of nucleotides is contacted with a pair of oligonucleotide primers. The primers hybridize with a nucleotide sequence, are extended under suitable conditions, and then are dissociated from the nucleotide sequence. This process is repeated to increase the number of copies of a sequence of nucleotides. Other methods can be used for amplification and are known to a person with ordinary skill in the art.

The term “complementary” refers to complementary nucleobase pairing between two nucleotide sequences on either two different nucleotide strands or two regions of the same nucleotide strand. It is known in the art that adenine bases form complementary pairing with thymine or with uracil bases through the formation of specific hydrogen bonds. Likewise, cytosine bases form complementary pairing with guanine bases through the formation of specific hydrogen bonds. Other descriptions of complementary pairing are detailed herein.

The term “composition” refers to a compound that comprises one or more molecules. The composition can contain oligonucleotides, polypeptides, small molecules, other types of molecules, or a combination thereof. A “pharmaceutical composition” refers to a composition that includes a modulator, or at least one molecule considered to be a pharmaceutical agent.

The term “isolated” refers to a purified, enriched or concentrated population of molecules. “Isolated” also refers to the act of enriching or concentrating a particular molecule, compound or complex such that its purity is increased.

The term “tissue” refers to an aggregate of cells that form a specific physiological function in an organism.

The term “delivery”, when used in the context of drugs, agents, or pharmaceutical compositions, refers to the administration of a drug, agent, or pharmaceutical composition to an assay mixture, a cell in culture, an animal, or a human subject or patient.

A “carrier”, when used in the context of drugs, agents, or pharmaceutical composition is one or more molecules that is used to aid the delivery of one or more other molecules.

ALS is part of a broader spectrum of disorders known as “motor neuron disease” (MND) that includes primary lateral sclerosis (PLS), hereditary spastic paraplegia (HSP), pseudobulbar palsy, Mills' syndrome, monomelic amyotrophy, post-polio syndrome (PPS), madras motor neuron disease (MMND), progressive muscular atrophy (PMA), spinal muscular atrophy (SMA), spinal and bulbar muscular atrophy (SBMA), and progressive bulbar palsy (PBP). In one embodiment, the term ALS refers to the broader category of MND, and includes but is not limited to, PLS, HSP, pseudobulbar palsy, Mills' syndrome, monomelic amyotrophy, PPS, MMND, PMA, SMA, SBMA, and PBP.

ALS can share common mechanisms with other “neurodegenerative diseases”, “neuromuscular diseases” and “TDP-43 proteinopathies”, including frontotemporal lobar degeneration (FTLD) and frontotemporal dementia (FTD), Alzheimer's disease, Parkinson's disease, and retinal degeneration diseases such as age-related macular degeneration (AMD). In one embodiment, the term ALS refers to the broader category of neurodegenerative and neuromuscular diseases. In another embodiment, the term ALS refers to the broader category of diseases involving TDP-43 proteinopathy.

“Proteinopathies” refer to a class of diseases that can result from, in part or in whole, abnormal protein function or protein aggregates, which are caused by structural or configurational abnormalities, modifications to the protein sequence (e.g., post-translational modifications) or localization, leading to aggregation of those proteins as a consequence. Such abnormal protein function or aggregates can interrupt or alter normal cellular, tissue, or organ function.

“TDP-43 proteinopathies” refers to diseases wherein a sub-population of patients can exhibit an increase in levels of TDP-43 aggregates in the cytoplasm. Examples of which include but are not limited to, ALS, Alzheimer's disease, argyrophilic grain disease, vascular dementia, frontotemporal dementia (FTD, FTD-TDP-43, and FTD-tau) and the greater group of frontotemporal lobar degeneration (FTLD), semantic dementia, dementia with Lewy bodies, polyglutamine diseases, Huntington's disease, spinocerebellar ataxia, inclusion body myopathy, inclusion body myositis, hippocampal sclerosis, parkinsonism, Parkinson's disease (PD), Perry syndrome, ALS-parkinsonism dementia complex of Guam, primary lateral sclerosis (PLS), hereditary spastic paraplegia (HSP), pseudobulbar palsy, Mills' syndrome, monomelic amyotrophy, post-polio syndrome (PPS), madras motor neuron disease (MMND), progressive muscular atrophy (PMA), spinal muscular atrophy (SMA), spinal and bulbar muscular atrophy (SBMA), progressive bulbar palsy (PBP), retinal degeneration diseases such as age-related macular degeneration (AMD), retinitis pigmentosa (RP), and glaucoma. TDP-43 proteinopathies are further described in Gendron and Josephs (Gendron and Josephs, Neuropathol. Appl. Neurobiol. 36:97-112 (2010)), Lagier-Tourenne et al. (Lagier-Tourenne et al., Hum. Mol. Gen. 19(1):R46-R64 (2010)), and Matsukawa et al. (Matsukawa et al., Journal of Biological Chemistry, 291(45): 23464-23476 (2016)), the disclosures of which, together with references cited therein, are incorporated herein in their entirety.

“Retinal degeneration diseases” refer to a class of diseases that can result from, in part or in whole, damage to photoreceptor cells of the retina, resulting in a continuous decline in vision. The term “retinal degeneration diseases” can include diseases such as, age-related macular degeneration (AMD), retinitis pigmentosa (RP), glaucoma or vision loss associated with photoreceptor degeneration.

“Oxidative stress” refers to a condition where there is an excess production of reactive oxygen species (ROS) relative to antioxidants. The term “oxidative stress” includes diseases such as attention deficit hyperactivity disorder (ADHD), autism, Asperger syndrome, atherosclerosis, cancer, depression, myocardial infarction, cardiovascular disease, chronic fatigue syndrome, diabetes, fragile X syndrome, neurodegenerative diseases such as ALS, Huntington's disease, Parkinson's disease, Alzheimer's disease and multiple sclerosis; ophthalmological diseases such as glaucoma, cataract formation and macular degeneration; as well as liver injury, osteoporosis, autoimmune diseases, inflammatory diseases, stroke and sickle cell disease.

“Anemia” refers to a condition where there are insufficient healthy red blood cells to transport oxygen to the body's tissues, resulting in symptoms such as fatigue, weakness, and dizziness. Anemia can be an acute or chronic condition. There are different potential causes of anemia, including iron deficiency, vitamin deficiency, inflammation, aplastic anemia, bone marrow disease, hemolytic anemia or sickle cell anemia.

“Ischemic diseases” refer to vascular diseases involving an interruption or reduction in the supply of arterial blood to a tissue, or organ, resulting in tissue or organ damage. The term “ischemic disease” includes cardiovascular diseases such as cardiac ischemia, myocardial ischemia, ischemic cardiomyopathy, coronary artery disease, or myocardial infarction. The term “ischemic disease” also includes ischemic colitis, mesenteric ischemia, brain ischemia, stroke, renal ischemia, limb ischemia, peripheral vascular disease or cutaneous ischemia.

“Inflammatory diseases” refer to diseases that are characterized by inflammation. The term “inflammatory disease” includes but is not limited to, allergy, asthma, atherosclerosis, autoimmune diseases, coeliac disease, glomerulonephritis, hepatitis, inflammatory bowel disease, non-alcoholic steatohepatitis (NASH), psoriasis, renal fibrosis, reperfusion injury, rheumatoid arthritis, transplant rejection, tubular ischemia-reperfusion damage or vascular inflammation. The term “inflammatory disease” also refers to neuroinflammatory diseases that are characterized by neurological damage caused by immune responses, including but not limited to, neurodegenerative diseases such as Alzheimer's disease, Parkinson's disease, amyotrophic lateral sclerosis, multiple sclerosis, or other TDP-43 proteinopathies.

“Cancer” refers to an abnormal growth of cells which tend to proliferate in an uncontrolled way and, in some cases, to metastasize. The term “cancer” or “cancers” include but are not limited to oral, salivary, laryngeal, esophangeal, head and neck, lung, gastric, gallbladder, pancreatic, urothelial, bladder, renal, cervical, ovarian, prostate, breast or colorectal cancer; as well as fibrofolliculomas, clear cell renal cell carcinoma, multilocular clear cell renal carcinoma, chromophobe renal cell carcinoma, renal oncocytic hybrid carcinoma, uterine corpus endometrioid cancer, interdigitating dendritic cell sarcoma, hemangioblastomas (slow-growing tumors of the central nervous system), pancreatic neuroendocrine tumors, pheochromocytomas (noncancerous tumors of the adrenal glands), endolymphatic sac tumors, kidney cysts, or lung cysts.

“Ameliorating” is the lessening of severity of a disease, as measured by at least one indicator of that disease. Indicators can be symptoms of that disease or a marker associated with the disease and can be objectively or subjectively evaluated. In certain embodiments, to “ameliorate” can mean to slow, halt, or reverse the progression of a disease.

A “dose” is a specified unit of a pharmaceutical composition that is provided for administration. In some embodiments, dose can refer to a specified amount of a pharmaceutical composition that is administered over a period of time. The dose can refer to the total amount of the pharmaceutical composition administered, or the amount of pharmaceutical composition administered per unit of time.

### Inhibition of FLCN

In certain embodiments, provided herein are compositions, systems, and methods for modulating the expression or activity of FLCN in a cell, an animal, or human subject. In certain embodiments, provided herein are compositions, systems and methods for reducing or inhibiting the expression or activity of FLCN in a cell, an animal, or human subject, in order to treat, prevent, or ameliorate a disease, particularly neuromuscular or neurodegenerative diseases, such as for example, ALS, FTLD, Alzheimer's disease, retinal degeneration diseases such as age-related macular degeneration (AMD), and other TDP-43 proteinopathies.

In other embodiments, provided herein are compositions, systems and methods for reducing or inhibiting the expression or activity of FLCN in a cell, an animal, or human subject, in order to treat, prevent, or ameliorate a disease including oxidative stress, obesity, anemia or ischemic diseases, such as cardiovascular disease, myocardial ischemia and peripheral vascular disease.

The FLCN gene encodes for a protein named folliculin, which is also represented herein as FLCN. FLCN is also known as BHD, DENND8B, FLCL, MGC17998, MGC23445, BHD skin lesion fibrofolliculoma protein and Birt-Hogg-Dube syndrome protein. The FLCN protein or polypeptide, referenced herein, includes any polymorphs of the FLCN protein, for example, different protein products obtained from the translation of different FLCN RNA transcripts, such as described in SEQ ID NOs: 1-15. FLCN, as used herein, also refers to FLCN genes or transcripts harboring one or more mutations, or FLCN proteins obtained from the expression of such mutant genes or transcripts. FLCN, as used herein, refers to FLCN genes or transcripts, such as described in SEQ ID NOs: 1-15.

FLCN is expressed in most tissues, for example, the brain, the skin and its appendages, the lungs, and the kidney, etc. One known function of FLCN is as a tumor suppressor. Loss-of-function mutations in FLCN have been linked to kidney, lung and skin tumors, and Birt-Hogg-Dube (BHD) syndrome. Other functions of FLCN include roles in the adenosine-monophosphate-activated protein kinase (AMPK) and mTOR pathways. Schmidt et al. (Schmidt et al., Gene 640: 28-42 (2018)) describes the structure of FLCN, its normal roles in the cell and associated pathways, as well as its roles in diseases such as BHD, fibrofolliculomas, lung cysts, spontaneous pneumothorax and kidney tumors, the disclosures of which, along with its references, are incorporated herein in its entirety.

FLCN can dimerize with FNIP1 or FNIP2 to form a FLCN-FNIP1 or FLCN-FNIP2 complex respectively. The FLCN-FNIP1 and/or FLCN-FNIP2 complexes play important roles in regulating several pathways such as the Rag-mediated nutrient sensing pathway, the VHL-HIF-VEGF pathway, the TGF-β pathway, the autophagy pathway, the cell cycle, and RhoA signaling (Hasumi et al., PNAS 112(13): E1624-E1631 (2015)). Loss-of-function of FNIP1 and/or FNIP2, thereby leading to reduced activity of FLCN-FNIP1 or FLCN-FNIP2 complexes respectively, have been associated with cancers, such as for example, fibrofolliculomas, kidney tumors, clear cell renal cell carcinoma, multilocular clear cell renal carcinoma, chromophobe renal cell carcinoma, renal oncocytic hybrid carcinoma, bladder cancer, uterine corpus endometrioid cancer, interdigitating dendritic cell sarcoma, hemangioblastomas, pancreatic neuroendocrine tumors, pheochromocytomas, endolymphatic sac tumors, kidney cysts and lung cysts. Furthermore, loss-of-function mutation of FNIP1, thereby leading to reduced activity of the FLCN-FNIP1 complex, in mice leads to B cell deficiency and the development of cardiomyopathy, which is similar to the effects of upregulating AMPK in mice and humans (Siggs et al., PNAS 113(26): E3706-E3715 (2016)). In addition, loss of FNIP1, thereby leading to reduced activity of the FLCN-FNIP1 complex, in mice is associated with increased expression of inflammatory markers (Centini et al., PLoS One 13(6): e0197973 (2018)). Previously, Bastola et al. reported that VHL is a positive regulator of FLCN mRNA levels either by transcriptional induction, or stabilization of FLCN mRNA by repression of miRNAs that inhibit FLCN mRNA, or a combination of both (Bastola et al., PLoS ONE 8(7): e70030 (2013)). Loss-of-function mutations in VHL, thereby leading to a decrease of FLCN expression and/or activity, have been linked to von Hippel-Lindau (VHL) disease, hemangioblastomas (slow-growing tumors of the central nervous system), kidney cysts, clear cell renal cell carcinoma, pancreatic neuroendocrine tumors, pheochromocytomas (noncancerous tumors of the adrenal glands) and endolymphatic sac tumors. Gossage et al. (Gossage et al., Nature Reviews Cancer 15: 55-64 (2015)) describes the structure of VHL, its normal roles in the cell and associated pathways, as well as its roles in VHL disease and cancers, such as hemangioblastomas, kidney cysts, clear cell renal cell carcinoma, pancreatic neuroendocrine tumors, pheochromocytomas and endolymphatic sac tumors, the disclosures of which, along with its references, are incorporated herein in its entirety.

In some embodiments, provided herein are compositions, systems, and methods for increasing or upregulating the expression or activity of FLCN in a cell, animal, or human subject, which can be used to treat, prevent or ameliorate diseases such as inflammatory diseases, von Hippel-Lindau (VHL) disease, B cell deficiency, cardiomyopathy, as well as other cancers.

The present invention is, in part, related to the novel discovery that instead of upregulating FLCN (which can be effective in diseases such as BHD, fibrofulliculomas, lung cysts, spontaneous pneumothorax, and kidney tumors), perhaps counterintuitively, reducing or inhibiting the expression or activity of FLCN can be used to treat a unique set of diseases, particularly neuromuscular or neurodegenerative diseases, such as for example, ALS, FTLD, Alzheimer's disease, retinal degeneration diseases such as age-related macular degeneration (AMD), and other TDP-43 proteinopathies.

The amino acids 202-299 of FLCN can interact directly with the RRM1 and RRM2 domains of TDP-43 in human embryonic kidney (HEK293) cells, leading to FLCN-mediated shuttling of TDP-43 from the nucleus into the cytoplasm and accumulation of TDP-43 in the cytoplasm (Xia et al., Human Molecular Genetics, 25(1): 83-96 (2016)). Given that the experiments in Xia et al. were performed in kidney cells, and that cellular function and disease mechanisms are typically unique across different tissues or cell types, there remains a need for investigations into the relationship of FLCN to cytoplasmic TDP-43 accumulation in neuronal cell types. Experiments have thus been performed by the inventors in disease-relevant cells (e.g. motor neuron cells), which has led to the novel discovery that inhibiting or reducing the expression or activity of FLCN in cells in the central nervous system (CNS), which are implicated in neuromuscular or neurodegenerative diseases such as ALS and other TDP-43 proteinopathies, lead to a decrease in cytoplasmic TDP-43 aggregates (see Example 6) and an increase in cell survival (see Example 5).

Further support for this discovery can also be found in studies showing that loss of FLCN-FNIP1 or FLCN-FNIP2 in BHD patients lead to the constitutive activation of AMPK, which results in PGC-1α mediated mitochondrial biogenesis, induction of HIF-1α transcriptional activity and increased transcription of VEGF (Preston et al., Oncogene 30: 1159-1173 (2011); Yan et al., J Clin Invest. 124(6): 2640-2650 (2014)). In certain embodiments, reducing or inhibiting the expression or activity of FLCN stimulates mitochondrial biogenesis and angiogenesis via the activation of the VEGF and HIF-1α pathways, which can be used to treat, prevent or ameliorate anemia or ischemic diseases, such as cardiovascular disease, myocardial ischemia and peripheral vascular disease. In addition, FLCN-deficient mice exhibit reduced inflammation when subject to a NASH-inducing diet, while FLCN-deficient nematodes develop increased resistance to oxidative stress and pathogens (Paquette et al., bioRxiv DOI: 10.1101/2020.09.10.291617 (2020)). Thus, in certain embodiments, reducing or inhibiting the expression or activity of FLCN can be used to treat, prevent, or ameliorate inflammatory diseases and oxidative stress. Furthermore, knockout of FLCN in mice adipocytes results in resistance to obesity induced by a high-fat diet (Paquette et al., bioRxiv DOI: 10.1101/2020.09.10.291617 (2020)). Thus, in certain embodiments, reducing or inhibiting the expression or activity of FLCN can be used to treat, prevent, or ameliorate obesity.

Thus, in certain embodiments, provided herein are modulators to reduce or inhibit the expression or activity of FLCN. In some embodiments, the inhibition of FLCN expression or activity can lead to either a decrease in the level of pathological TDP-43 aggregates in the cytoplasm, or an increase in levels of functional TDP-43 in the nucleus, or a combination thereof, in order to treat, prevent or ameliorate ALS or other TDP-43 proteinopathies. In certain embodiments, provided herein are modulators that disrupt the interaction between FLCN and TDP-43. In some embodiments, the modulators target the RRM1 and RRM2 domains of TDP-43, thereby blocking its interaction with FLCN. In other embodiments, the modulators target the region between amino acids 202-299 of FLCN, thereby blocking its interaction with TDP-43. Such modulators that specifically disrupt the interaction between FLCN and TDP-43 can lead to beneficial outcomes such as either a decrease in the level of TDP-43 aggregates in the cytoplasm, or an increase in levels of functional TDP-43 in the nucleus, or a combination thereof, while allowing for the non-targeted domains in FLCN or TDP-43 to perform their normal functions, thus reducing undesired side effects. In certain embodiments, the modulators provided herein include, but are not limited to antisense modulators, oligonucleotide modulators, peptide modulators, antibody modulators, and small molecule modulators. In one embodiment, the modulators provided herein can be used as prophylaxis to prevent ALS. In one embodiment, the modulators provided herein can be used as therapeutics to treat or ameliorate the symptoms of ALS.

### Inhibition of FLCN Via Associated Genes or Pathways

In certain embodiments, the inhibition or downregulation of FLCN mRNA or FLCN protein can be achieved by targeting FLCN-associated genes or pathways. In certain embodiments, provided herein are modulators that reduce or inhibit the expression, activity or signaling of FLCN by targeting and inhibiting at least one gene or pathway that positively regulates or increases the expression, activity or signaling of FLCN respectively. In another embodiment, provided herein are modulators that reduce or inhibit the expression of FLCN by targeting and increasing the expression or activity of at least one gene or pathway that negatively regulates or inhibits the expression of FLCN. In one embodiment, provided herein are modulators that reduce or inhibit the activity of FLCN, such as the activity of shuttling TDP-43 from the nucleus to the cytoplasm, by targeting and inhibiting another gene or pathway that is responsible for positively regulating the activity of FLCN. In yet another embodiment, provided herein are modulators that reduce or inhibit the activity of FLCN by increasing the expression or activity of at least one gene or pathway that is responsible for negatively regulating the activity of FLCN.

In yet other embodiments, provided herein are modulators that affect the nucleocytoplasmic distribution of FLCN. In a preferred embodiment, provided herein are modulators that enhance the nuclear distribution of FLCN and reduce its localization to the cytoplasm. In one embodiment, provided herein is a modulator that targets, removes or interferes with the nuclear export signal of TDP-43 located between amino acids 239-250, which can reduce or prevent FLCN-mediated shuttling of TDP-43 from the nucleus into the cytoplasm, thereby leading to either a decrease in pathological TDP-43 aggregates in the cytoplasm, or an increase in levels of functional TDP-43 in the nucleus, or a combination thereof. In one embodiment, provided herein is a modulator that targets, removes or interferes with a domain of the FLCN gene or protein that is responsible for the shuttling of FLCN and TDP-43 from the nucleus into the cytoplasm. FNIP1 and FNIP2 interact with FLCN via their C terminal domains and have been shown to promote the cytoplasmic localization of FLCN. When FLCN is expressed on its own, it is mostly localized to the nucleus. However, when FNIP1 or FNIP2 is co-expressed with FLCN, FNIP1/FLCN or FNIP2/FLCN complexes are observed in the cytoplasm. The regulation of FLCN nucleocytoplasmic shuttling by FNIP1 and FNIP2 is described in Takagi et al. (Takagi et al., Oncogene, 27: 5339-5347 (2008)), Baba et al. (Baba et al., PNAS, 103(42): 15552-15557 (2006)) and Hasumi et al. (Hasumi et al., Gene, 31: 415(1-2), 60-67 (2008)), the disclosures of which, along with their references, are incorporated herein in its entirety. Thus, in certain embodiments, provided herein are modulators that targets, removes or interferes with the C terminal domains of either FNIP1, FNIP2, or FLCN, in order to reduce or prevent the cytoplasmic shuttling of FLCN, thereby leading to either a decrease in pathological TDP-43 aggregates in the cytoplasm, or an increase in levels of functional TDP-43 in the nucleus, or a combination thereof

### Antisense Modulators to Inhibit Genes

In one embodiment, the modulator used to modulate, and in particular to inhibit or reduce, the expression or activity of FLCN, is an antisense modulator. In one embodiment, the antisense modulator is an antisense oligonucleotide (ASO). ASO in various embodiments can be in any format well known to a person skilled in the art. ASOs comprise an oligonucleotide sequence that is complementary to the coding sequence, otherwise known as the sense strand, of a targeted gene. When a targeted gene is transcribed into pre-mRNA or mRNA, the ASO binds to the mRNA, forming a double-stranded RNA molecule or an RNA/DNA complex. The double-stranded nature of the resulting RNA molecule or RNA/DNA complex prevents effective translation, thereby reducing or preventing expression of the resulting polypeptide. Furthermore, double-stranded RNA or RNA/DNA complexes are subject to degradation and digestion by a collection of enzymes known as endonucleases, such as RNase H, thereby reducing or preventing expression of the resulting polypeptide. In certain embodiments, the ASO can effect a change in splicing patterns of the mRNA such that one exon is exchanged for another (i.e., splice-switching). In other embodiments, the ASO can effect a change in splicing patterns of the mRNA such that one or more exons, or portion thereof, is removed (i.e., exon-skipping). In one embodiment, exon skipping results in a shift in the reading frame during translation, leading to premature stop codons and a truncated protein that is degraded by nonsense-mediated decay (NMD). In another embodiment, the ASO can effect a change in splicing patterns of the mRNA such that one or more introns, or portion thereof, is retained (i.e., intron retention), which can lead to a decrease in protein expression or activity. In yet other embodiments, the ASO can modulate the stability and rate of degradation of the mRNA. Rinaldi & Wood (2018) (Rinaldi, C. and Wood M. J. A. Nature Review Genetics 14:19-21 (2018)) describe in more detail the functions and uses of ASO and chemical modifications for ASOs to promote effectiveness and stability in the therapeutic context. The Rinaldi & Wood (2018) reference, including all references cited therein, are incorporated herein in its entirety.

In one embodiment, the ASO inhibits the expression of a targeted polypeptide or nucleotide sequence in part or in its entirety. In another embodiment, the ASO inhibits an enzyme that affects the function of a targeted polypeptide. In yet another embodiment, the ASO inhibits the activity of an RNA molecule. In another embodiment, the ASO modulator reduces the level of an RNA molecule, such as a noncoding RNA molecule, thereby affecting the expression of a targeted nucleic acid or polypeptide.

ASOs can be produced by any number of methods known to a person who is skilled in the art (see below for examples of some specific methods).

### RNAi to Inhibit Genes

In one embodiment, the modulator can be a therapeutic oligonucleotide used in the RNAi process. The therapeutic RNAi oligonucleotide can include miRNA, siRNA, or shRNA.

The oligonucleotides used in the RNAi process can be in any form well known to a person skilled in the art. The RNAi process comprises several steps. First, a double-stranded oligonucleotide is introduced into the cell either exogenously or through the introduction of a viral vector that transcribes it. Second, the double-stranded oligonucleotide is cleaved by the ribonuclease protein Dicer into siRNA, which are short oligonucleotide fragments of around 20-25 base pairs. This step is not needed if synthetic siRNAs, which resemble the products of Dicer, are used. Third, the siRNA is separated by RISC into single-stranded oligonucleotide fragments, comprising the sense and antisense strands to a target RNA, and integrated into the RISC to form a RISC-siRNA complex. Fourth, the RISC-siRNA complex containing the antisense strand hybridizes to a target RNA that is complementary to it and cleaves the target RNA, thereby inhibiting translation of the target RNA into a polypeptide. In some embodiments, the oligonucleotides used in RNAi comprises preformed double-stranded (duplex) RNA, and which are preferably 19-29 nucleotides in length. In one embodiment, the oligonucleotide used in RNAi comprise a single-stranded, short hairpin RNA (shRNA), which consists of two complementary RNA sequences that are preferably 19-22 nucleotides each in length, and which are linked by a short loop of 4-11 nucleotides. The shRNA can be encoded in the form of DNA and delivered into cells via a nucleic acid vector, wherein it is transcribed to form the shRNA. miRNA are non-coding RNA sequences found endogenously that use the same RISC pathway to target and inhibit other RNA molecules. One difference between miRNA and siRNA is that miRNAs can operate by imperfect base pairing and typically affects multiple target RNAs, whereas siRNAs usually operate by perfect base pairing leading to specific knockdown of a target RNA. RNAi processes and their therapeutic applications are described in more detail by Aagaard and Rossi (Aagaard L and Rossi J J, Advanced Drug Delivery Reviews 59:75-86 (2007)), and Lam et al. (Lam et al., Molecular Therapy, Nucleic Acids 4, e252 (2015)), the disclosures of which, along with their references, are incorporated herein in their entirety.

In one embodiment, the RNAi oligonucleotide modulator inhibits the expression of a targeted polypeptide in part or in its entirety. In another embodiment, the RNAi oligonucleotide modulator inhibits an enzyme that affects the function of a targeted polypeptide or nucleotide sequence. In another embodiment, the RNAi oligonucleotide modulator inhibits another nucleic acid, such as a noncoding RNA, which affects the expression of the target polypeptide or nucleic acid sequence.

RNAi oligonucleotides can be produced by any number of methods known to a person who is skilled in the art (see below for specific methods).

### Hybridization

In certain embodiments, the antisense modulators disclosed herein can hybridize with a target nucleic acid encoding FLCN. The most common mechanism of hybridization involves hydrogen bonding between complementary nucleobases of the antisense modulator and target nucleic acid, such as, for example, Watson-Crick, Hoogsteen, or reversed Hoogsteen hydrogen bonding. The conditions under which an antisense modulator can hybridize with a target nucleic acid molecule can vary. Under stringent conditions, an antisense modulator hybridizes in a sequence-dependent manner determined by the nature and composition of the nucleic acid molecules to be hybridized. Methods of determining whether an antisense modulator sequence can hybridize specifically to a target nucleic acid are well known in the art.

### Target Nucleic Acids, Target Regions, Target Segments and Nucleobase Sequences

In certain embodiments, an antisense modulator comprises an oligonucleotide or portions thereof that can hybridize to a target nucleic acid, wherein the target nucleic acid encodes FLCN.

In certain embodiments, target nucleic acids can comprise nucleobase sequences encoding FLCN, including but not limited to the following: RefSeq Accession No: NM_144997.7 (incorporated herein as SEQ ID NO: 1), the reverse complement of RefSeq Accession No. NC_000017.11 truncated from nucleotides 17206900 to 17239000 (incorporated herein as SEQ ID NO: 2), RefSeq Accession No. NM_144606.7 (incorporated herein as SEQ ID NO: 3), RefSeq Accession No. NM_001353229.2 (incorporated herein as SEQ ID NO: 4), RefSeq Accession No. NM_001353230.2 (incorporated herein as SEQ ID NO: 5), RefSeq Accession No. NM_001353231.2 (incorporated herein as SEQ ID NO: 6), RefSeq Accession No. XM_011523714.3 (incorporated herein as SEQ ID NO: 7), RefSeq Accession No. XM_024450635.1 (incorporated herein as SEQ ID NO: 8), RefSeq Accession No. XM_017024305.2 (incorporated herein as SEQ ID NO: 9), RefSeq Accession No. XM_011523718.3 (incorporated herein as SEQ ID NO: 10), RefSeq Accession No. XM_017024308.1 (incorporated herein as SEQ ID NO: 11), RefSeq Accession No. XM_011523719.3 (incorporated herein as SEQ ID NO: 12), RefSeq Accession No. XM_017024309.2 (incorporated herein as SEQ ID NO: 13), RefSeq Accession No. XM_011523721.3 (incorporated herein as SEQ ID NO: 14), RefSeq Accession No. XR_001752445.2 (incorporated herein as SEQ ID NO: 15). In some embodiments, antisense modulators can also target other nucleobase sequences encoding FLCN (e.g. other DNA sequences, cDNA sequences, scaffolds, or mRNA transcript variants), which can be found by accession number in databases such as NCBI and GENBANK, and which are incorporated herein by reference. In other embodiments, previous and future versions of nucleobase sequences encoding FLCN, which can be found by accession number in databases such as NCBI and GENBANK are also incorporated herein by reference.

The nucleobase sequence set forth in each SEQ ID NO contained herein is independent of any modification to a nucleobase, a sugar moiety, or an internucleoside linkage. In some embodiments, antisense modulators or portions thereof that are defined by a percent complementarity or percent identity to a nucleobase sequence set forth in a SEQ ID NO or sample reference number (GI ID #) described herein can comprise, independently, one or more modifications to a nucleobase, one or more modifications to a sugar moiety, or one of more modifications to an internucleoside linkage.

In certain embodiments, an antisense modulator can hybridize to at least one target region within the target nucleic acid. A target region is a structurally defined region of the target nucleic acid. Examples of a target region include but are not limited to an exon, an intron, an exon-intron junction, an intron-exon junction, an exon-exon junction, a 3′ untranslated region (3′ UTR), a 5′ untranslated region (5′ UTR), a translation initiation region, a translation termination region, a 5′ donor splice site, a 3′ acceptor splice site, a start codon, an upstream open reading frame (ORF), a repeat region, a hexanucleotide repeat expansion, a splice enhancer region, an exonic splicing enhancer (ESE), a splice suppressor region, an exonic splicing silencer (ESS), an RNA destabilization motif, a miRNA binding site, or other defined nucleic acid region. The structurally defined regions for FLCN can be obtained by accession number from sequence databases such as NCBI and GENBANK, and such information is incorporated herein by reference.

In some embodiments, a target region can contain one or more target segments. In some embodiments, multiple target segments within a target region can be non-overlapping. In certain embodiments, target segments within a target region are separated by less than 5000, 2500, 1000, 500, 250, 100, 50, 40, 30, 20, 10, or 5 nucleotides. In other embodiments, target segments within a target region are overlapping or contiguous. In certain embodiments, an antisense modulator can hybridize to a 5′ target segment within a target region and a 3′ target segment within the same target region. In other embodiments, an antisense modulator can hybridize to a 5′ target segment within a target region and a 3′ target segment within a different target region.

A suitable target segment can specifically exclude a certain structurally defined target region, such as, for example, a start codon or a stop codon. The determination of suitable target segments can include a comparison of the nucleobase sequence of the target segment to other sequences throughout the genome. For example, the BLAST algorithm can be used to identify regions of similarity amongst different sequences. This comparison can enable the selection of antisense modulator sequences that have increased specificity for a target segment and a corresponding reduced likelihood of hybridizing in a non-specific manner to non-target or off-target sequences.

In some embodiments, targeting includes determination of at least one target segment within a target nucleic acid to which an antisense modulator or portion thereof can hybridize in order to produce a desired effect. The desired effect can be a decrease or increase in mRNA levels of a target nucleic acid. The desired effect can also be a decrease or increase in levels of a protein encoded by the target nucleic acid. The desired effect can also be a phenotypic change associated with a change in mRNA levels of a target nucleic acid or change in protein levels encoded by the target nucleic acid. In certain embodiments, the desired effect of using an antisense modulator to target at least one target segment within a target nucleic acid encoding FLCN to which it hybridizes, is a reduction in FLCN mRNA levels. In other embodiments, the desired effect of using an antisense modulator to target at least one target segment within a target nucleic acid encoding FLCN to which it hybridizes is a reduction in FLCN protein levels. In yet other embodiments, the desired effect of using an antisense modulator to target at least one target segment within a target nucleic acid encoding FLCN to which it hybridizes is a phenotypic change associated with the reduction of FLCN mRNA or protein levels.

### Targeting FLCN

In certain embodiments, the antisense modulators described herein or portion thereof can hybridize to any target nucleic acid comprising nucleotide sequences encoding FLCN. In some embodiments, the antisense modulators can hybridize to target nucleic acids at any stage of RNA processing within the cell, for example, pre-mRNA or mature mRNA. In yet other embodiments, antisense modulators can hybridize to any target region(s) within the target nucleic acid, for example, an exon, an intron, a 5′ UTR, a 3′ UTR, a repeat region, a hexanucleotide repeat expansion, a miRNA binding site, a splice junction, an exon-exon junction, an exon-intron junction, an intron-exon junction, an exonic splicing silencer (ESS), an exonic splicing enhancer (ESE), exon 1, exon 2, exon 3, exon 4, exon 5, exon 6, exon 7, exon 8, exon 9, exon 10, exon 11, exon 12, exon 13, exon 14, intron 1, intron 2, intron 3, intron 4, intron 5, intron 6, intron 7, intron 8, intron 9, intron 10, intron 11, intron 12, or intron 13, etc. In one embodiment, antisense modulators can hybridize to at least one exon described in Tables 1-5. In other embodiments, antisense modulators can hybridize to target regions other than exons, where such regions are described in databases such as NCBI and GENBANK, which are incorporated herein by reference.

In certain embodiments, the antisense modulators described herein can hybridize to all RNA transcript variants of FLCN. In other embodiments, the antisense modulators described herein hybridize selectively to at least one RNA transcript variant of FLCN. Transcript variants of FLCN can include mRNA transcripts generated by differential splicing, or mRNA transcripts containing mutations (e.g., SNPs, INDELs etc.) when compared to a reference sequence. In certain embodiments, the antisense modulators described herein inhibit the expression of all transcript variants of FLCN. In certain embodiments, the antisense modulators described herein inhibit expression of all transcript variants of FLCN equally. In certain embodiments, the antisense modulator described herein preferentially inhibits the expression of certain transcript variants of FLCN. In certain embodiments, antisense modulators described herein are useful for reducing cytoplasmic TDP-43 aggregates, or increasing the levels of functional TDP-43 in the nucleus, or a combination thereof. In certain embodiments, antisense modulators described herein are useful for normalizing the expression of various mis-regulated genes.

In certain embodiments, provided herein are antisense modulator sequences designed to target various regions of FLCN transcripts produced by the FLCN gene (the reverse complement of RefSeq Accession No. NC_000017.11 truncated from nucleotides 17206900 to 17239000, incorporated herein as SEQ ID NO: 2). The nucleotide sequence, target start site, target stop site, target region, and description of each antisense modulator sequence are specified in Table 6. The predicted binding energy of each antisense modulator to the target sequence, as calculated using software known in the art, such as RNAstructure, are also described under “Binding Score” in Table 6 (see SEQ ID NOs: 16-612). Antisense modulators with greater binding energy (more negative binding score) are predicted to hybridize better to the target sequence and are preferred.

In some embodiments, the antisense modulator comprises one of the modified sequences in Table 17.

### Complementarity

An antisense modulator is said to be complementary to a target nucleic acid, for example a target nucleic acid encoding FLCN, when one or more nucleobases of the antisense modulator can hydrogen bond with the corresponding complementary nucleobases of the target nucleic acid, such that the antisense modulator can hybridize in a sequence-dependent manner to the target nucleic acid. In certain embodiments, an antisense modulator can comprise one or more non-complementary nucleobases to the target nucleic acid, provided that the antisense modulator retains its ability to hybridize to the target nucleic acid. In certain embodiments, when two or more non-complementary nucleobases are present, they can be contiguous (i.e., linked) or clustered together. In other embodiments, when one or more non-complementary nucleobases are present, they can be interspersed with complementary nucleobases and need not be contiguous to each other or to complementary nucleobases. In certain embodiments, one or more non-complementary nucleobases can be located either at the 5′ end, 3′ end, internal region, or a mix of any regions of the antisense modulator. In one embodiment, a non-complementary nucleobase is present in the wing region of an antisense oligonucleotide modulator comprising a gapped sequence, as described in more detail below. An antisense modulator can also hybridize to one or more target segments in the target nucleic acid, such that adjacent or intervening segments do not take part in the hybridization event (e.g., forming a hairpin or loop structure, or mismatch).

In certain embodiments, provided herein are antisense modulators or specified portions thereof, that are at least 40%, 50%, 60%, 70%, 75%, 80%, 85%, 90%, 95%, 96%, 97%, 98%, 99%, or 100% complementary to a target nucleic acid, a target region, or a target segment encoding or associated with FLCN, such as described in SEQ ID NOs: 1-15. By way of example, an antisense modulator in which 16 of its 20 nucleobases are complementary to a target nucleic acid encoding FLCN, and would therefore specifically hybridize to it, would represent 80% complementarity. Methods to determine percent complementarity, percent homology, or sequence identity of an antisense modulator to a target nucleic acid, a target region, or a target segment, are well known in the art, for example, using basic local alignment search tools (BLAST) and PowerBLAST programs (Altschul et al., Journal of Molecular Biology, 215: 403-410 (1990); Zhang and Madden, Genome Research, 7: 649-656 (1997)), and the Gap program (Wisconsin Sequence Analysis Package) that relies on the Smith and Waterman algorithm (Smith and Waterman, Advances in Applied Mathematics, 2: 482 489 (1981)). These methods, along with their respective publications and those references cited within, are incorporated herein in their entirety.

In certain embodiments, provided herein are antisense modulators or specified portions thereof, that are 100% complementary (i.e., fully complementary) to a target nucleic acid, a target region, or a target segment, for example, encoding FLCN. As used herein, “fully complementary” means each nucleobase of an antisense modulator is capable of specific base pairing with complementary nucleobases of a target nucleic acid. By way of example, an antisense modulator that is 18 nucleobases long is said to be fully complementary to a target nucleic acid that is 3667 nucleobases long if all 18 nucleobases of the antisense modulator is capable of specific base pairing with complementary nucleobases of the target nucleic acid.

In some embodiments, complementarity can be determined for a specified portion of an antisense modulator. By way of example, a 20 nucleobase portion of a 35 nucleobase antisense modulator can be said to be fully complementary to a target nucleic acid that is 3667 nucleobases long if the 20 nucleobase portion can undergo specific base pairing with complementary nucleobases of the target nucleic acid. At the same time, in some embodiments, the entire 35 nucleobase antisense modulator can be fully complementary to the target nucleic acid sequence if the remaining 15 nucleobases of the antisense modulator are also complementary to the target sequence. In other embodiments, the 35 nucleobase antisense modulator is not fully complementary to the target nucleic acid sequence if the remaining 15 nucleobases of the antisense modulator are not fully complementary to the target sequence.

In certain embodiments, antisense modulators that are up to 10, 15, 20, 25, 30, or 35 nucleobases in length comprise no more than 1, no more than 5, no more than 10, no more than 15, no more than 20, or no more than 25 non-complementary nucleobase(s) respectively, relative to a target nucleic acid, such as a target nucleic acid encoding FLCN or a specified portion thereof.

In certain embodiments, provided herein are antisense modulators that are complementary to a specified portion of a target nucleic acid, as defined by a number of contiguous (i.e. linked) nucleobases within a target region or target segment of a target nucleic acid. In some embodiments, the antisense modulator is complementary to at least 8, 10, 12, 14, 16, 18, 20, 22, 24, 26, 28, 30, 32, 34, or more contiguous nucleobases of a target nucleic acid, for example encoding FLCN, or a range defined by any two of these values.

### Identity

In certain embodiments, the antisense modulators provided herein can have a defined percent identity to a particular nucleotide sequence, SEQ ID NO, or oligonucleotide represented by a specific sample reference number (e.g., GI ID #), or portion thereof disclosed herein. An antisense modulator is identical to a sequence disclosed herein if both possess the same nucleobase pairing ability to a complementary target nucleotide sequence, regardless of other modifications to the antisense modulator. For example, a DNA or other antisense modulator with nucleobase thymine at given position(s) is identical to an RNA sequence disclosed herein with nucleobase uracil at those equivalent position(s), since both thymine and uracil base pair with adenine. In another example, an RNA or other antisense modulator with nucleobase uracil at given position(s) is identical to a DNA sequence disclosed herein with nucleobase thymine at the equivalent positions(s). Other examples include the use of synthetic nucleobases that have the same nucleobase pairing ability as standard nucleobases, such as that of adenine, guanine, thymine, cytosine, and uracil. In certain embodiments, antisense modulators that are lengthened and shortened versions of the oligonucleotides and nucleotide sequences disclosed herein are contemplated. In certain embodiments, antisense modulators that have non-identical nucleobases relative to the oligonucleotides and nucleotide sequences disclosed herein are also contemplated. The non-identical nucleobases can be contiguous (i.e. linked or adjacent) with each other or dispersed throughout the antisense modulator. The percent identity of an antisense modulator relative to a sequence disclosed herein is derived by calculating the percentage of nucleobases of the antisense modulator that can undergo identical base pairing as compared with the sequence disclosed herein.

In certain embodiments, antisense modulators or portions thereof, are at least 40%, 50%, 60%, 70%, 75%, 80%, 85%, 90%, 95%, 96%, 97%, 98%, 99%, or 100% identical to one or more particular nucleotide sequence described in SEQ ID NOs: 16-612, or oligonucleotide represented by a GI ID #, or portion thereof disclosed herein.

In some embodiments, the antisense modulators or portions thereof, are at least 40%, 50%, 60%, 70%, 75%, 80%, 85%, 90%, 95%, 96%, 97%, 98%, 99%, or 100% identical to one or more particular nucleotide sequences described in SEQ ID NOs: 613-618, or oligonucleotide represented by a GI ID #, or portion thereof disclosed herein.

In certain embodiments, provided herein are antisense modulators that are identical to a specified portion of a nucleobase sequence, as defined by a number of contiguous (i.e. linked) nucleobases in the nucleobase sequence. In some embodiments, the antisense modulator consists of at least 8 consecutive nucleobases with at least 70%, at least 80%, at least 85%, at least 90%, at least 95%, or 100% identity to any of the nucleobase sequences of SEQ ID NOs: 16-618.

### Modifications

Modifications to an antisense modulator can be made to increase its efficacy, stability, and/or ease of administration, as well as decrease toxicity or other harmful side effects. Certain embodiments of the invention can include such modifications, which include, but are not limited to, modifications of the backbone or internucleoside linkages, modifications of the sugar component, modifications of the nucleobase component, or other modifications of the structure or chemistry of the nucleotide. All modulators and their modifications mentioned herein include salts, mixed salts, esters, salts of esters, and free acid or base forms. Certain embodiments can include a combination of one or more of the modifications mentioned below. Certain embodiments can include a combination of one or more of the modifications mentioned below with one or more unmodified nucleotides. All modifications to nucleotides mentioned herein that remove or otherwise modify naturally occurring components of nucleotides are still considered nucleotides, respectively. By way of example, a nucleotide that is missing a phosphorus or phosphate component, or that has its natural phosphate internucleoside linkage replaced by a phosphorothioate linkage for instance, or that has a ribose or deoxyribose component replaced by a modified sugar component, is still considered a nucleotide. Likewise, an oligonucleotide comprising a chain of at least two nucleotides, including the modified nucleotides described herein, is still considered an oligonucleotide, even if the component nucleotides in the oligonucleotide are covalently bonded in a manner that is modified from that of the naturally found phosphodiester bond. By way of example, a compound formed by two modified nucleotides covalently bonded with a phosphorothioate bond, is considered an oligonucleotide.

Modifications to an antisense modulator can be made along the backbone (i.e., linkages between different nucleosides). In one embodiment, the modification is the inclusion of a modified phosphoester group. In one embodiment, the modification is the inclusion of a phosphorothioate group or linkage. Phosphorothioate linkages have been shown to increase the resistance of antisense modulators to nucleases and thus increase their overall stability, while also able to maintain cleavability by certain ribonucleases (RNases), such as RNase H, once paired with a complementary or near-complementary oligonucleotide strand, thus increasing effectiveness in certain antisense applications (Eckstein F, Nucleic Acid Therapeutics, 24(6), 374-387 (2014)). This resistance has been found in both DNA and RNA and can be implemented in a wide range of nucleotide-based therapeutic modalities, including both ASO and RNAi therapies. In addition, phosphorothioate linkages can increase bioavailability of the modulator in certain cases by improving overall cellular uptake of the modulator.

Other embodiments of backbone modifications include the use of other phosphorothioate-based linkages, such as chiral phosphorothioate linkages, phosphorodithioate linkages, and phophorotrithioate linkages; phosphotriesters and alkylphosphotriesters; phosphonates, such as chiral phosphonates, 3′- and 5′-alkylene phosphonates, methylphosphonates (including 5′-O-methylphosphonate and 3′-O-methylphosphonate), hydroxyphosphonates, thionoalkylphosphonates, and other alkyl phosphonates; phosphonoacetate and thiophosphonoacetate linkages; phosphoroamidates, such as N3′-P5′ phosphoramidates, cationic phosphoramidates, methoxyethyl phosphoramidates, aminoalkylphosphoramidates, thiophosphoramidates, dithiophosphoramidates, thionophosphoramidates, and methanesulfonyl phosphoramidates; phosphonoamidate and phosphonothioate linkages; other thio-linked backbones; other phosphate backbones, such as 3′-5′ linked and 2′-5′-linked selenophosphates and boranophosphates, as well as those phosphates with inverted polarity with 3′-3′, 5′-5′, or 2′-2′ linkages; phosphinate linkages; acetyl- and thioacetyl-based linkages; other sulfur-containing backbones, such as sulfonate, sulfamide, sulfamate, sulfone, sulfoxide, and sulfide; silicon-containing backbones, such as siloxane and silyl; other heteroatom backbones such as those backbones containing N, O, or S; linkages with mixed heteroatoms; CH₂-containing linkages; carbonate-modified linkages; carboxymethyl-modified linkages; carbamate-modified linkages; short chain alkyl or cycloalkyl linkages; alkene- and azide-containing linkages; formacetal thioformacetal, methylene formacetal, and methylene thioformacetal linkages; methyleneimino linkages; hydrazino- and methylhydrazino-based linkages; methylene methylimino linkages; 5′-5′ linkages exposing two 3′ ends, as described by Bhagat et al. (Bhagat L et al., Journal of Medicinal Chemistry, 54(8): 3027-3036 (2011)), which, along with its references, is incorporated herein in its entirety; and other backbones known to those skilled in the art. In one embodiment, the modulator includes one of the backbone modifications described herein or other backbone modifications as known by a person skilled in the art.

Modifications to an antisense modulator can be made by modifying the sugar component of the modulator molecule. This sugar component is referred to as ribose or deoxyribose of RNA and DNA, respectively, but can also include other sugar components in other nucleotides. In one embodiment the sugar component of at least one nucleotide of the modulator is modified to include a 2′-O-methoxyethyl group (also referred herein as 2′-MOE). The 2′-MOE modification has been demonstrated to enhance nuclease resistance, as well as to lower cell toxicity and increase binding affinity with the desired modulator target. In one embodiment, the 2′-MOE modification includes a 2′, 4′-constrained 2′-MOE modification, as described by Pallan et al. (Pallan P S et al., Chemical Communications, 48(66): 8195-8197 (2012)), which, along with its references, is incorporated herein in its entirety. In one embodiment, the 2′ OH-group of at least one nucleotide in the modulator is replaced by at least one of H, SH, F, Cl, Br, I, NH₂, or ON. In another embodiment the 2′ OH-group of at least one nucleotide in the modulator is replaced by at least one of R, SR, NHR, or NR₂. R is defined, in this case, as one of C₁-C₆ alkyl, alkenyl, or alkynyl. In another embodiment, the sugar modification of the modulator is a 2′-O-methyl (2′-OMe) modification. In one specific embodiment, a 2′-OMe or other 2′-O-alkyl group modification is made to sugar groups of the modulator located at the two nucleotides closest to the 5′ end of the sequence. In the case of an RNAi modulator, this modification can be made on the 5′ end of both the sense and anti-sense strands, as described by U.S. Pat. No. 7,834,171, which, along with its references, is incorporated herein in its entirety. In certain embodiments, the sugar component of at least one nucleotide of the modulator is modified to include a bicyclic sugar, such as a 4′-CH(R)—O-2′ or 4′ (CH₂)₂—O-2′, wherein R is independently selected from H, C₁-C₁₂ alkyl, or a protecting group. In one embodiment, the sugar component of at least one nucleotide of the modulator is modified to include a bicyclic sugar consisting of a 4′-CH(CH₃)—O-2′ bridge or constrained ethyl (cEt) modification. A cEt modification can improve the effectiveness and allele selectivity of the antisense modulator and is further described by Pallan et al. In another embodiment, the modification of at least one nucleotide includes a tetrahydropyran-modified nucleoside. Other embodiments include the modification of at least one nucleotide of the antisense molecule to include a 2′-dimethylaminooxyethoxy group or 2′-dimethylaminoethoxyethoxyl group or other moieties obvious to those skilled in the arts.

Modifications to an antisense modulator can be made by modifying the nucleobase component of the modulator molecule. The nucleobase component refers to the nitrogen-containing compounds that, along with the sugar component, form nucleosides. Nucleobases found in nucleotides and elsewhere include adenine, guanine, cytosine, thymine, uracil, and their isomers. Examples of other nucleobases or modifications to nucleobases can include 5-methylcytosine, 5-hydroxymethyl cytosine, xanthine, hypoxanthine, methylhypoxanthine, 1-methylcytosine, 2-O-methylcytosine, 2,6-diaminopurine, 6-methyl, 2-propyl, and other alkyl derivatives of adenine and guanine, 2-thiouracil, 2-thiothymine, 2-thiocytosine, 2-F-adenine, 2-aminoadenine, 2-aminopyridine, 2-amino-6-hydroxyaminopurine, 2-deoxyuridine, 3-ethylcytosine, 3 methylcytosine, 6-hydroxyaminopurine, 6-hydroxymethyladenine, 2-pyridone, 5-halo, including 5-bromo, 5-trifluoromethyl, 5-chloro, 5-fluoro, and other 5-halo, uracils and cytosines, 5-propynyl uracil and cytosine and other alkynyl-modified pyrimidine bases, 6-azouracil, 6-azocytosine, 6-azothymine, 5-uracil, 4-thiouracil, 3-deazaguanine, 3-deazaadenine tricyclic pyrimidines, and O- and N-alkylation, including N6-methyladenosine and N6-carbamoylmethyladenine, 3,N4-ethenocytosine, 1N2-ethenoguanine, N2,3-ethenoguanine, N2, N2-dimethylguanosine, carboxylethylguanine, 4-methylcytosine, 5-carboxylcytosine, 5-formylcytosine, 5-hydroxymethylcytosine, 5-glucosylmethylcytosine, methylthymine, 5,6-dihydrothymine, 5-hydroxymethyluracil, formyluracil, carboxyluracil, tricyclic pyrimidines, phenothiazine cytidine, G-clamps, carbazole cytidine, and pyridoindole, cytidine, 7-methylguanine, 7-methyladenine, 7-deazaguanine, 7-amido-7-deazaguanine, 7-deazaadenine, 8-halo, 8-amino, 8-thiol, 8-thioalkyl, 8-hydroxyl, and other 8-substituted adenines and guanines, 8-azaguanine, 8-azaadenine, inosine, 8-oxoguanine. In one embodiment, at least one nucleobase in at least one nucleotide of the antisense modulator is modified or replaced with at least one of the nucleobases or modified nucleobases mentioned above. In one embodiment the modification of the antisense modulator is a substitution of at least one cytosine nucleobase with 5-methylcytosine. In one embodiment, all cytosine nucleobases of the antisense modulator are substituted with 5-methylcytosine. The 5-methylcytosine substitution can lower the immunogenicity of antisense modulators and is a modification that is preferred in many forms of antisense compounds. In another embodiment, the antisense modulator is modified to have both at least one 5-methylcytosine substitution and at least one 2′-MOE sugar modification.

In one embodiment, the antisense modulator is modified to comprise phosphorodiamidate morpholino oligonucleotides (also referred to herein as PMOs or morpholinos). A PMO can be constructed with at least one nucleoside comprising a sugar component, such as ribose or deoxyribose, that has been substituted with a methylenemorpholine ring. This modified nucleoside can be linked to others via a phosphorodiamidate linkage, in place of a phosphodiester linkage. PMOs have been demonstrated to be resistant to a variety of enzymes, including nucleases, esterases, and proteases. PMOs, as uncharged molecules, also present limited interactions with charged molecules, such as proteins. These advantages can make PMOs a suitable modification for use in antisense applications, in which increased stability in vitro and in vivo and high target specificity can be important factors for consideration. Descriptions of morpholinos, as well as their properties and variations, included in some of the embodiments herein, can be found in U.S. Pat. Nos. 9,469,664 and 10,202,602, which, along with their references, are incorporated herein in their entirety.

In one embodiment, the antisense modulator is modified to comprise locked nucleic acids (LNA). LNAs are nucleotides that have a sugar component modified to comprise a 2′ O to 4′ C methylene linkage. These modified nucleotides provide higher resistance to cleavage by digestive enzymes such as nucleases, as well as present much higher binding affinities to complementary or near-complementary nucleic acid-based targets. In another embodiment, the antisense modulator is modified to comprise other bridged nucleic acids (BNA), such as the 2′, 4′-BNA^{NC}[N-Me] modification. Descriptions of LNAs, as well as their properties and variations, included in some of the embodiments herein can be found in U.S. Pat. No. 9,428,534, which, along with its references, is incorporated herein in its entirety.

In one embodiment, the antisense modulator is modified to comprise tricyclo-DNA (tcDNA). tcDNA modifications can provide several benefits, including improved nuclease resistance, binding stability and improved targeting. The structure and other pertinent information of tc-DNA included in some of the embodiments herein are described by Ittig et al. (Ittig, D et al., Artificial DNA PNA & XNA 1(1): 9-16 (2010)) and U.S. Pat. No. 10,465,191, which, along with their references, are incorporated herein in their entirety.

In one embodiment, the antisense modulator is a modified oligonucleotide containing a gap segment (also referred to herein as “gapped sequences”, and otherwise known as “gapmers”). These gapped sequences comprise a sequence of unmodified or modified oligonucleotides (also referred to herein as the “central sequence”) flanked on at least one end by at least one sequence of either unmodified or modified oligonucleotides (also referred to herein as the “wing sequence”). In one embodiment, the central sequence comprises unmodified DNA nucleotides, which when hybridized to a target RNA, allows for endonucleases such as RNase H to cleave the target RNA. In another embodiment, the central sequence comprises a mix of modified and unmodified nucleotides, which when hybridized to a target RNA, allows for RNase H cleavage. In one embodiment, the antisense modulator comprises a central sequence flanked by wing sequences at both the 5′ and 3′ ends of the central sequence, wherein at least one nucleoside of the wing sequences comprises a modified sugar. In one embodiment, the wing sequence is a combination of modified and unmodified nucleosides. In one embodiment, the wing sequences comprise nucleosides wherein each nucleoside comprises a modified sugar. In certain embodiments, the central sequence is chosen to consist of 8, 9, 10, 11 or 12 linked nucleosides. In certain embodiments, the central sequence is chosen to consist of 6, 7, 13, 14 15, 16, 17, or 18 linked nucleosides. In certain embodiments, the wing sequences are each independently chosen to consist of 4, 5, or 6 linked nucleosides. In certain embodiments, the wing sequences are each independently chosen to consist of 3 linked nucleosides. In one embodiment, the central sequence consists of 10 linked nucleosides flanked by wing sequences at the 5′ and 3′ ends of the central sequence, wherein each wing sequence consists of 5 linked nucleosides. In one embodiment, the central sequence consists of 10 linked nucleosides flanked by wing sequences at the 5′ and 3′ ends of the central sequence, wherein each wing sequence consists of 4 linked nucleosides. In one embodiment, the central sequence consists of 10 linked nucleosides flanked by wing sequences at the 5′ and 3′ ends of the central sequence, wherein each wing sequence consists of 6 linked nucleosides. Gapped sequences can increase resistance to enzymes, such as nucleases, and, in some cases, reduce the need for phosphorothioate modifications. In one embodiment, the gapped sequence comprises a central sequence flanked by wing sequences containing at least one LNA or BNA. In another embodiment, the gapped sequence comprises a central sequence flanked by wing sequences comprising at least one nucleoside consisting of a 2′-MOE modified sugar. In one embodiment, the gapped sequence comprises a central sequence flanked by wing sequences comprising nucleosides wherein each nucleoside consists of a 2′-MOE modified sugar. In another embodiment, the gapped sequence comprises a central sequence flanked by wing sequences comprising at least one nucleoside consisting of tcDNA. In another embodiment, the gapped sequence comprises a central sequence flanked by wing sequences comprising at least one nucleoside consisting of a cEt modification. In one embodiment, the wing sequences can be one or more of a combination of the aforementioned modified sequences. Other gapped sequences included in the embodiments herein are described in U.S. Pat. Nos. 7,015,215 and 10,017,764, which are incorporated, along with their references, herein in their entirety.

Gapped sequences containing modified nucleosides in the central sequence can reduce cellular protein-binding and improve the therapeutic index of the antisense modulator, as described by Shen et al. (Shen et al., Nature Biotechnology, 37(6): 640-650 (2019)), which, along with its references, is incorporated herein in its entirety. In some embodiments, the central sequence of the gapped sequence comprises at least one nucleoside consisting of a modified sugar. In some embodiments, the second nucleoside of the central sequence from the 5′ end of the gapped sequence consists of a modified sugar, such as a cEt or 2′-OMe modified sugar. In another embodiment, the antisense oligonucleotide comprises a gapped sequence consisting of a central sequence of deoxynucleotides, which are flanked on both sides by wing sequences consisting of 2′-MOE modified nucleotides, and wherein the second nucleotide of the central sequence from the 5′ end of the oligonucleotide contains a 2′-OMe sugar modification.

In one embodiment, the antisense modulator comprises a peptide nucleic acid (PNA). PNAs are modified nucleic acids that can be created through the substitution of the nucleotide backbone for a pseudopeptide backbone (e.g., N-(2-aminoethyl)-glycine), which links nucleosides together via peptide bonds. The lack of charged groups in the backbone of PNAs provide for higher affinity and specificity between the modified antisense modulator and its complementary or near-complementary oligonucleotide target. In one embodiment, the PNA comprises a GripNA compound (Active Motif, Inc., Carlsbad, Calif.). In another embodiment, the PNA is a phosphono-PNA molecule comprising an additional phosphate group, as described by Efimov et al. (Efimov, V A et al., Nucleic Acids Research, 26(2): 566-575 (1998)). In another embodiment, the PNA molecule contains charged groups to promote intracellular delivery (e.g., Efimov, V A et al., Nucleosides, Nucleotides & Nucleic Acids, 24(10-12): 1853-1874 (2005)). Certain types of PNAs included in the embodiments herein are described in U.S. Pat. No. 6,962,906 and Montazersaheb et al. (Montazersaheb S et al., Advanced Pharmaceutical Bulletin, 8(4): 551-563 (2018)), which are incorporated, along with their references, herein in their entirety.

In one embodiment, the antisense modulator includes at least one bifacial nucleotide, also known as a Janus base. A Janus base comprises two binding sites to a complementary nucleotide, which can be used to simultaneously bind to both sense and antisense strands of a target oligonucleotide through Watson-Crick bonding. Benefits of using a Janus base modification include potentially higher target specificity and higher levels of target deactivation or efficacy. Examples and descriptions of Janus bases can be found in Thadke et al. (Tadke S A, Communications Chemistry, 1(79) (2018)) and Asadi et al. (Asadi A, The Journal of Organic Chemistry, 72(2): 466-475 (2007)), both of which are incorporated herein, along with their references, in their entirety.

In one embodiment, the antisense modulator forms a chimeric compound. Chimeric compounds can comprise one of many configurations, including, but not limited, to RNA-DNA, PNA-DNA, PNA-RNA, and other modified or unmodified oligonucleotide analogues bound to other modified or unmodified oligonucleotides. Certain uses of chimeric compounds, for example, can include providing one region that confers improved nuclease resistance, while another region increases specificity of binding or binding stability to a complementary or near-complementary target. Other chimeric compounds can offer other combinations of benefits, including any of the benefits specified for the modifications mentioned above.

These modifications described herein and other modifications, including modifications to internucleoside backbones, sugars and nucleobases, as well as conjugates and methods of delivery described below, are described in part by Shen and Corey (Shen X and Corey D R, Nucleic Acids Research, 46(4): 1584-1600 (2018)), Smith and Rula (Smith CIE and Zain R, Annual Review of Pharmacology and Toxicology, 59: 605-630 (2019)), Khvorova and Watts (Khvorova A and Watts J K, Nature Biotechnology, 35(3): 238-248 (2017)), Manoharan (Manoharan M, Biochimica et Biophysica Acta, 1489(1): 117-130 (1999)), Chawla et al., (Chawla M et al., Nucleic Acids Research, 43(14): 6714-6729 (2015)), and U.S. Pat. Nos. 9,399,774 and 8,101,743, 7,407,943, all of which, along with each of their references, are incorporated herein in their entirety. Other modifications are known to those skilled in the art and are considered to be included in the embodiments herein.

### Conjugates, Complexes, and Structures

Certain embodiments of the invention comprise antisense modulators conjugated or bonded to at least one other molecule, such as a peptide or polypeptide, lipid, sugar, nucleotide or oligonucleotide, other polymer, cleavage agent, transport agent, intercalating agent, molecular beacon, hybridization-triggered crosslinking agent, lipophilic agent, and hydrophilic agent. These conjugated or bonded complexes can provide a number of benefits to the antisense modulator, including, but not limited to, increased effectiveness or activity, improved delivery to specific tissues or cells, enhanced cellular uptake, lowered toxicity, resistance to nuclease degradation, increased half-life or residence time, enhanced pharmacodynamic or pharmacokinetic properties, and improved selective targeting of alleles, genes or other targets. Certain embodiments can include a combination of one or more of the complexes mentioned below.

In one embodiment, the antisense modulator is conjugated to a protein or other polyamide, amine, or similar molecule. In another embodiment, the antisense modulator is conjugated to a lipid, phospholipid, cholesterol or thiocholesterol, cholic acid, aliphatic chain, hexylamino-carbonyl-oxycholesterol, or other similar molecule. In another embodiment, the antisense modulator is conjugated to another organic molecule, such as an ether or thioether, steroid, biotin, phenazine, folate, phenanthridine, anthraquinone, acridine, adamantine acetic acid, palmityl, fluorescein, rhodamine, coumarin, dye or other marker molecule, or other polymer, such as polyethylene glycol. In another embodiment, the antisense modulator is conjugated to another drug or pharmaceutical agent used to treat, prevent or ameliorate the symptoms of ALS or other degenerative disease, such as edaravone, riluzole, dextromethorphan, quinidine sulfate, dexpramipexole, or baclofen in the case of ALS. In some embodiments, the antisense modulator is conjugated to another drug or pharmaceutical agent used to treat, prevent, or ameliorate cancer. In certain embodiments, the antisense modulator is conjugated to another drug or pharmaceutical agent used to treat, prevent, or ameliorate oxidative stress, or obesity. In other embodiments, the antisense modulator is conjugated to another drug or pharmaceutical agent used to treat, prevent, or ameliorate VHL disease, BHD syndrome, or spontaneous pneumothorax. In other embodiments, the antisense modulator is conjugated to another drug, such as for example, another drug alleviating pain or other symptoms or improving uptake or delivery, such as blood thinners (e.g., aspirin, warfarin), anti-inflammatory and pain relief drugs (e.g., COX inhibitors, phenylbutazone, ibuprofen, suprofen, fenbufen, ketoprofen, pranoprofen, carprofen, indomethacin, folinic acid, taiprofenic acid, diclofenac, niflumic acid, diazepines or benzodiazepines, barbiturate); or antibacterial, antiviral, antibiotic, or other drug that promotes at least one benefit in a therapeutic setting, including treatment efficacy, symptom alleviation, drug tolerance, or side effect mediation. In another embodiment, the antisense modulator is conjugated to another agent promoting transport across cell membranes, such as those described in Letsinger et al. (Letsinger et al., PNAS, 86(17): 6553-6556 (1989)), Zhao et al. (Zhao et al., Current Opinion in Biomedical Engineering, 13: 76-83 (2020)) or another agent promoting transport across the blood-brain barrier, as described in PCT Patent WO89/10134. All three references, along with the references described therein, are incorporated herein in their entirety. In another embodiment, the antisense modulator is conjugated to one or more GalNAc residues, which are recognized by the asialoglycoprotein receptor resulting in efficient uptake into cells. In one embodiment, the antisense modulator is conjugated to a G-quadruplex, as described by PCT Patent WO2017188898, which, along with its references, is incorporated herein in its entirety. In another embodiment, the antisense modulator is conjugated to another compound with special electromagnetic or optical properties, such as a photo-labile protecting group, as described by PCT Patent WO2017157950, which, along with its references, is incorporated herein in its entirety. In yet another embodiment, the antisense modulator is conjugated on at least one terminus to at least one stabilizing group to enhance properties such as, for example, nuclease stability. In one embodiment, the stabilizing groups are cap structures such as, for example, inverted deoxy abasic caps. Other stabilizing groups that can be used to cap one or both ends of an antisense modulator to impart nuclease stability are described in PCT Patent WO2003004602, which, along with its references, is incorporated herein in its entirety. Other conjugates, included in the embodiments herein, are described by Benizri et al. (Benizri et al., Bioconjugate Chemistry, 30: 366-383 (2019)), which, along with its references, is incorporated herein in its entirety.

### Methods of Delivery

In certain embodiments, provided herein are methods and compositions for the delivery of antisense modulators into a cell, an animal, or human subject, which are capable of reducing or inhibiting the expression or activity of FLCN. Many of these methods and compositions are known to those skilled in the art. In other embodiments, the methods and compositions for the delivery of antisense modulators described herein are also applied, with suitable modifications in some cases, to the delivery of other types of modulators, such as for example, other oligonucleotide modulators, antibody modulators, peptide modulators, or small molecule modulators.

In one embodiment, the method of delivery of antisense modulators includes direct introduction, or transfection, into a cell, an animal, or a human subject, via a transfection reagent, such as a liposomal-based or amine-based transfection reagent. This method of delivery can be used with or without aforementioned modifications or conjugations to the antisense modulator. Certain modifications and conjugations can improve the rate of introduction or stability of the antisense modulator and are included in these embodiments. For example, implementing any of the aforementioned modifications that imbue the antisense modulator with increased nuclease resistance can increase stability of the antisense modulator during and after introduction into a cell, an animal, or a human subject. Another example is the conjugation of the antisense modulator with a charged molecule or amphiphilic molecule to promote delivery across the cell membrane. In another embodiment, the method of delivery is electroporation or permeabilization. In another embodiment, the method of delivery includes the use of a liposome. In another embodiment, the method includes other forms of lipid-mediated transport. In another embodiment, the method of delivery involves the use of membrane fusion. In another embodiment, the method of delivery includes the use of colloids containing polymeric particles or solutions of nanoparticles. Nanoparticles can include certain properties that assist in targeting certain areas for delivery or otherwise promote delivery, such as electromagnetic properties. In another embodiment, the method of delivery includes the use of chemical-mediated transport, including the use of calcium phosphate. In another embodiment, the method of delivery includes peptide-mediated transport, including the use of polylysine. In another embodiment, the method of delivery includes the use of endocytosis. In other embodiments, the method of delivery can include microinjections directly into cells. In one embodiment, antisense modulators are delivered naked without transfection reagents. Other methods and examples of methods are described by Dokka and Rojanasakul (Dokka and Rojanasakul, Advanced Drug Delivery Reviews, 44(1): 35-49 (2000)), Lochmann et al. (Lochmann et al., European Journal of Pharmaceutics and Biopharmaceutics, 58: 237-251 (2004)), Dong et al. (Dong et al., Advanced Drug Delivery Reviews, 144: 133-147 (2019)), and Juliano (Juliano, Nucleic Acids Research, 44(14): 6518-6548 (2016)), all of which are incorporated herein, along with each of their references, in their entirety.

In one embodiment, the method of delivery includes one or more of the common delivery methods used to deliver drugs, including, but not limited to, injections that are vascular, extravascular, into cerebral spinal fluid, into the blood or lymph, intrathecal, oral uptake, nasal delivery or otherwise as an inhalant, and transdermal uptake.

### Nucleic Acid Vector

In certain embodiments, the method of delivery of antisense modulators described herein into a cell, an animal, or human subject, involves the use of nucleic acid vectors. Nucleic acid vectors in various embodiments are biological vehicles used for the transmission of genetic material from one location to another and can be in any format well known to a person skilled in the art. Genetic material can include, but is not limited to, DNA, RNA, mRNA, siRNA, miRNA, lncRNA, guide RNA (gRNA), or antisense oligonucleotides. These and other forms of genetic material are well known to a person of ordinary skill in the art, and are included in some embodiments herein. A nucleic acid vector can be in the form of a plasmid, cosmid, artificial chromosome, DNA or other cassette, phagemid, and the like. The genetic sequence contained within vectors can be created by DNA/RNA synthesis, and/or modified via DNA/RNA editing or splicing techniques that are known to a person who is skilled in the art. These vectors can be specific or nonspecific to a certain type of cell. In one embodiment, the nucleic acid vector is non-integrating, and otherwise known as an episomal vector (i.e., it does not integrate into the genome of the cell). In one embodiment, non-integrating vectors are useful for targeting post-mitotic cells that are no longer undergoing cell division, since as long as the episomal vector can be stably maintained, the modulator can be stably expressed. In another embodiment, the nucleic acid vector is an integrating vector. In one embodiment, integrating vectors are useful for targeting stem cells that are actively undergoing cell division, since genome integration ensures that the encoded modulator is not lost during cell division. A nucleic acid vector can be classified as a viral vector or non-viral vector, which allows for different methods of delivery into a target cell, both of which are included in some embodiments.

A viral vector comprises a sequence of nucleotides that can be delivered into a target cell via a genetically modified virus, such as a retrovirus, adenovirus, adeno-associated virus, lentivirus, pox virus, alphavirus, or herpes virus. In some embodiments, the virus can be encapsulated or attached to liposomes, polymersomes, dynamic polyconjugates, nanoparticle complexes and the like. Once introduced, the virus delivers the nucleic acid vector to the host cell as part of its natural replication cycle. The complete nucleic acid vector can be integrated into the genome of the host cell. In certain embodiments, the virus directly inserts particular nucleic acid sequences contained within the nucleic acid vector into the genome of the host cell. In other embodiments, non-integrating viral vectors can be used to introduce nucleic acids into a host cell that are not subsequently integrated into the genome.

Several methods can be used to facilitate the successful delivery of viral vectors in gene therapy, such as pseudotyping, adaptor targeting, genetic systems targeting, and others known in the art. In general, viral vectors need to be modified to facilitate successful transduction into target cells. In pseudotyping, a viral attachment protein that is compatible with the target cell but that is produced by a different virus is integrated into the viral vector. In adaptor targeting, a small molecule is developed that has strong binding affinity to both the vector and the target cell. In genetic systems targeting, the genetic incorporation of a protein or polypeptide into the vector facilitates the binding of the vector to the target cell. Viral vectors for gene therapy are further described by Waehler et al. (Waehler et al., Nature Reviews Genetics, 8:573-587 (2007)), which along with references cited therein, is incorporated by reference in its entirety and are known to a person of ordinary skill in the art.

Non-viral vectors can be delivered by any one of a number of non-viral delivery methods. Vectors modified with lipids such as phospholipid phosphatidylserine, DOTMA, DOSPA, DOTAP, DMRIE, cholesterol, DOPE, or any combination thereof, can be used to form liposomes to deliver genetic material. Vectors modified with polymers, such as poly(L-lysine), polyethylenimine, PEG, or any combination thereof, can also be used to deliver genetic material in polymersomes. In certain cases, the vector can be modified with a combination of lipid or polymer or other molecule that is known to a person of ordinary skill in the art. Other methods of delivery of nucleic acid vectors include but is not limited to the use of cell-penetrating peptides, physiologically compatible nanoparticles, dynamic polyconjugates, GalNAc, or stable nucleic acid-lipid particle formulations. In certain cases, unmodified nucleic acid vectors are taken up using natural processes for the uptake of nucleic acids by a cell, such as endocytosis. Non-viral vectors for gene therapy and certain methods of delivery are described further by Yin et al. (2014) (Yin et al., Nature Reviews Genetics, 15:541-555 (2014)), which along with reference cited therein, are incorporated by reference in its entirety and are known to a person of ordinary skill in the art.

### Analysis of Antisense Modulator Activity

The antisense modulators disclosed herein can have variable activity, for example, as defined by percent reduction of target nucleic acid (e.g., RNA) levels, percent reduction of levels of proteins encoded by target nucleic acids, or percent reduction of the activity of proteins encoded by target nucleic acids. In certain embodiments, reductions in FLCN RNA levels, which include, but are not limited to, RNA involved in the transcription of the FLCN genes and FLCN protein translation, are indicative of inhibition of FLCN expression. In particular embodiments, reductions in levels of one or more FLCN transcripts disclosed by a SEQ ID NO herein, is indicative of inhibition of FLCN expression. In some embodiments, reductions in levels of FLCN protein is indicative of inhibition of FLCN expression. In particular embodiments, reductions in levels of one or more FLCN proteins that are translation products of one or more FLCN transcripts disclosed by a SEQ ID NO herein, is indicative of inhibition of FLCN expression. In other embodiments, reductions in activity of FLCN proteins that are translation products of one or more FLCN transcripts disclosed by a SEQ ID NO herein is indicative of inhibition of FLCN expression. Activity of FLCN refers to one or more activities that are normally carried out by FLCN transcripts or proteins, such as, for example, regulation of autophagy, shuttling of TDP-43 from the nucleus to the cytoplasm, regulation of cell-cell adhesion, regulation of nutrient sensing pathways, regulation of the mTOR pathway, regulation of the AMPK pathway, regulation of cell cycle, or regulation of other signaling or metabolic pathways. In certain embodiments, the antisense modulators disclosed herein can selectively target and reduce the levels or activity of one or more particular FLCN transcript variants and the proteins encoded by them, such reductions in levels or activity of one or more FLCN transcript variants or proteins being indicative of inhibition of FLCN expression. In yet other embodiments, certain phenotypic changes produced as a result of administration of an antisense modulator to cells, animals, or human subjects, can be indicative of inhibition of FLCN expression, for example, increased cell survival, or decreased levels of TDP-43 aggregates in the cytoplasm. In certain embodiments, the methods and compositions described herein for the analysis of the activity of an antisense modulator are applied directly or in modified form to the analysis of the activity of other types of modulators, such as, for example, other oligonucleotide modulators, antibody modulators, peptide modulators, and small molecule modulators.

In certain embodiments, the inhibition of expression or activity of FLCN transcripts or proteins can lead to changes in the expression or activity of other genes, mRNA, proteins, or pathways in the cell, wherein such changes are indicative of inhibition of FLCN expression or activity. FLCN regulates the mTORC1 and AMPK pathways in a cell type-dependent manner, which is described by Khabibullin et al. (Khabibullin et al., Physiol Rep, 2(8): e12107 (2014)), which along with reference cited therein, are incorporated herein by reference in its entirety. In some embodiments, an increase in mTOR signaling or activation of the mTOR pathway in certain cell types, for example SAEC cells, is indicative of inhibition of FLCN expression. In some embodiments, a decrease in mTOR signaling or inhibition of the mTOR pathway in certain cell types, for example HBE cells, is indicative of inhibition of FLCN expression. In some embodiments, an increase in AMPK signaling or activity of the AMPK pathway in certain cell types is indicative of inhibition of FLCN expression. In other embodiments, a decrease in AMPK signaling or activity of the AMPK pathway in certain cell types is indicative of inhibition of FLCN expression. FLCN is a negative regulator of PPARGC1A/PGC1α and mitochondrial biogenesis, as described by Hasumi et al. (Hasumi et al., Hum Mol Genet., 23(21): 5706-19 (2014)), which along with reference cited therein, are incorporated herein by reference in its entirety. Thus, in some embodiments, an increase in expression or activity of PPARGC1A/PGC1a or an increase in mitochondrial biogenesis, are indicative of inhibition of FLCN expression. FLCN inhibits the activity of TFEB and TFE3, which are transcription factors for autophagy genes, as described in Petit et al. (Petit et al., J Cell Biol., 202(7): 1107-22 (2013)), which along with reference cited therein, are incorporated herein by reference in its entirety. Thus, in some embodiments, an increase in activity of TFEB or TFE3 are indicative of inhibition of FLCN expression. Furthermore, FLCN can inhibit the induction of autophagy by inhibiting the accumulation of LC3B, and promoting the accumulation of LC3C, as described by Bastola et al. (Bastola et al., PLoS ONE 8(7), e70030 (2013)), which along with reference cited therein, are incorporated herein by reference in its entirety. Thus, in some embodiments, an increase in levels of LC3B, or a decrease in levels of LC3C, or an increase in autophagy activity, can be indicative of inhibition of FLCN expression. In some cases, FLCN can promote autophagy by interaction with GABARAP and ULK1, as described in Dunlop et al. (Dunlop et al., Autophagy, 10(10): 1749-1760 (2014)), which along with reference cited therein, are incorporated herein by reference in its entirety. Thus, in some embodiments, a decrease in autophagic flux can be indicative of inhibition of FLCN expression. Changes to other genes, mRNA, proteins, or pathways in the cell that result from inhibiting the expression or activity of FLCN are well known to a person skilled in the art and are incorporated herein as indicative of inhibition of FLCN expression or activity.

### Analysis of RNA Levels

In certain embodiments, the inhibition of FLCN expression by a modulator, such as an antisense modulator, can be assessed by measuring the decrease in levels of FLCN RNA transcripts. RNA analysis can be carried out on poly(A)+ mRNA or total cellular RNA. Methods of RNA isolation are well known in the art and include, for example, using the TRIZOL Reagent (Invitrogen, Carlsbad, Calif.) according to the manufacturer's recommended protocols, or using an RNA extraction kit (Qiagen) etc. The target RNA levels can be quantified using methods well known in the art and include, for example, Northern blot analysis, competitive polymerase chain reaction (PCR), or reverse transcription followed by quantitative real-time PCR using the ABI PRISM 7600, 7700, or 7900 Sequence Detection System (PE-Applied Biosystems, Foster City, Calif.) according to the manufacturer's instructions.

Prior to quantitative real-time PCR, the isolated RNA first undergoes a reverse transcription reaction to produce complementary DNA (cDNA), which is then used as the substrate for the real-time PCR amplification reaction. Reagents for reverse transcription and real-time PCR can be obtained commercially (e.g., Invitrogen, Carlsbad, Calif.). The reverse transcription reaction and real-time PCR reactions can be performed sequentially in the same sample well or in different sample wells. The levels of a target gene or RNA that are obtained by real-time PCR can be normalized using either total RNA levels quantified by, for example, RIBOGREEN (Invitrogen, Carlsbad, Calif.), or normalized using the expression level of a gene whose expression in the cell is more or less stable, such as cyclophilin A. Methods of RNA quantification using RIBOGREEN are described in Jones et al. (Jones et al., Analytical Biochemistry, 265: 368-374 (1998)), which together with the references cited therein, are incorporated herein in its entirety. A CYTOFLUOR 4000 instrument (PE Applied Biosystems) can be used to measure RIBOGREEN fluorescence. The expression levels of cyclophilin A can be quantified by real-time PCR within the same well as that used for quantifying the levels of target RNA (i.e., by performing a multiplex reaction) or by running it in a separate well.

Probes and primers that hybridize to a target nucleic acid encoding FLCN can be designed using methods that are well known in the art, and can include the use of software, such as, for example, PRIMER EXPRESS Software (Applied Biosystems, Foster City, Calif.).

### Analysis of Protein Levels

In certain embodiments, the inhibition of FLCN expression by a modulator, such as an antisense modulator, can be assessed by measuring the decrease in levels of FLCN protein. Several methods for quantifying or measuring protein levels of FLCN are well known in the art, such as Western blot analysis, enzyme-linked immunosorbent assay (ELISA), immunoprecipitation, immunocytochemistry, fluorescence activated cell sorting (FACS), immunohistochemistry, protein activity assays, quantitative protein assays, bicinchoninic acid assay (BCA assay) also known as the Smith assay, and the like. Antibodies that are specific for a target protein, such as FLCN, can be generated using conventional monoclonal or polyclonal antibody generation methods well known in the art, or identified and obtained commercially from a variety of sources, such as the MSRS catalog of antibodies (Aerie Corporation, Birmingham, Mich.). Antibodies for the detection of mouse, rat, monkey, and human FLCN are available commercially.

### In Vitro Testing of Antisense Modulators

In certain embodiments, the antisense modulators described herein can be administered to cultured cells in vitro to evaluate their effects on the expression of target gene(s) or other phenotypes. In certain embodiments, the antisense modulators described herein can be administered to cultured cells in vitro to evaluate the effects of antisense modulators on FLCN expression or activity. In certain embodiments, the antisense modulators provided herein can be administered to cultured cells in vitro to evaluate their effects on one or more phenotypes, such as, for example, cell survival, cell morphology, or levels of TDP-43 aggregates in the cytoplasm. In certain embodiments, other modulators described herein, such as, for example, other oligonucleotide modulators, antibody modulators, peptide modulators, or small molecule modulators can be administered to cultured cells in vitro to evaluate their effects on FLCN expression or activity, or one or more phenotypes, such as, for example, cell survival, cell morphology, or levels of TDP-43 aggregates in the cytoplasm.

In some embodiments, the cultured cells can have an animal origin, for example, Sf9 insect cells, Chinese hamster ovary (CHO) cells, rat cell lines, mouse cell lines, or non-human primate cell lines etc. In other embodiments, the cultured cells can have a human origin, for example, human embryonic kidney-derived epithelial cells (HEK293 or HEK293T), HeLa cells, human neural cell lines, ReN-VM cells, human fibroblast cell lines, HepG2 cells, Hep3B cells, and primary hepatocytes etc. Examples of cultured cells include those that are described in the catalogs of commercial vendors, such as, for example, Clonetics Corporation, Walkersville, Md.; American Type Culture Collection, Manassas, Va.; Zen-Bio, Inc., Research Triangle Park, N.C. etc., and are incorporated by reference herein. Such cells are cultured according to the vendor's instructions using commercially available reagents (e.g. Invitrogen Life Technologies, Carlsbad, Calif.). Cells can be cultured and tested in multi-well plates, for example, 24-well, 48-well, 96-well, 384-well plates etc.

In certain embodiments, the cultured cells are human induced pluripotent stem cell (iPSC) lines or other human stem cell lines. In certain embodiments, the cultured cells are differentiated cells that are derived from iPSC or other stem cell lines using methods that are well known in the art. Such differentiated cells include but are not limited to motor neuron cells, upper motor neuron cells, lower motor neuron cells, astrocyte cells, glial cells, microglial cells, corticol neuron cells, endothelial cells, dopaminergic neuron cells, neural stem cells, oligodendrocyte cells, other brain cells, cardiomyocytes, other cardiac cells, skeletal muscle cells, vascular endothelial or smooth muscle cells, hepatocytes, other liver cells, pancreatic (3-cells, other kidney cells, lung cells etc. In some embodiments, the modulators, including antisense modulators, described herein are administered to more than one cell type that are cultured together (i.e., co-culture).

In one embodiment, the human iPSCs or other stem cells are derived from healthy individuals. In another embodiment, the human iPSCs or other stem cells are derived from individuals that are at risk of, or suspected to be afflicted with, or diagnosed with a neuromuscular or neurodegenerative disease, including but not limited to ALS, Alzheimer's disease, retinal degeneration diseases such as age-related macular degeneration (AMD), and other TDP-43 proteinopathies disclosed herein etc. In some embodiments, the human iPSCs or other stem cells are derived from individuals with familial ALS, including but not limited to individuals with known pathogenic mutations in ALS genes such as C9orf72, SOD1, STMN2, NEK1, TARDBP, FUS, VCP, OPTN, SQSTM1, UBQLN2, hnRNPA1, MATR3 etc. In some embodiments, the human iPSCs or other stem cells are derived from individuals with sporadic ALS, including those with and without known ALS-causing mutations. In yet other embodiments, human iPSCs or other stem cells can be modified in the lab to reproduce or mimic the diseased condition, for example, by the introduction of disease-causing mutations in known disease-relevant genes using genetic engineering techniques such as homologous recombination, CRISPR/Cas9, TALENs or zinc-finger nucleases, etc. Such iPSC or stem cell lines are termed isogenic disease cell lines. In the case of ALS, examples of isogenic cell lines include but are not limited to those containing known disease causing mutations in ALS genes such as SOD1, TARDBP, and others (Hor et al., bioRxiv 713651 (2019)).

In other embodiments, the human iPSCs or other stem cells are derived from individuals that are at risk of, or suspected to be afflicted with, or diagnosed with one or more diseases, including but not limited to oxidative stress, obesity, anemia, ischemic disease, inflammatory disease, VHL disease, BHD syndrome, spontaneous pneumothorax, or cancer.

The use of cultured cells of human origin to evaluate the effects of modulators, including antisense modulators, produces results with higher relevance to the human condition compared to the use of animal models, which often fail to recapitulate important aspects of the disease due to a lack of conservation of gene targets, pathways, physiology and systems between the animal model and humans (van Damme et al., Disease Models & Mechanisms, 10, 537-549 (2017)). Consequently, promising therapies that show a positive effect in preclinical studies using ALS animal models (e.g., SOD1 transgenic mice) have all failed to translate to success in human clinical trials (Mitsumoto et al., Lancet Neurology, 13: 1127-38 (2014)). Moreover, the use of human iPSCs or other stem cells derived from disease patients with different profiles and backgrounds (e.g., sex, age, ethnicity, disease sub-type, presence or absence of known ALS mutations, etc.) allows for the evaluation of the effects of modulators, including antisense modulators, described herein on a broad spectrum of patients.

Described herein and in the Examples are methods for the administration of antisense modulators to cultured cells, which can be modified appropriately for the administration of other modulators such as, for example, other oligonucleotide modulators, antibody modulators, peptide modulators, or small molecule modulators. In general, antisense modulators are administered to cultured cells when the cells are approximately 60-80% confluent. Transfection reagents that are commonly used to introduce antisense modulators into cultured cells are well known in the art and include, for example, LIPOFECTAMINE or LIPOFECTIN (Invitrogen, Carlsbad, Calif.). Antisense modulators are mixed with LIPOFECTAMINE or LIPOFECTIN in OPTI-MEM 1 reduced serum medium (Invitrogen, Carlsbad, Calif.) to achieve the desired concentration of antisense modulator and a LIPOFECTAMINE or LIPOFECTIN concentration that typically ranges from 2-12 μg/mL per 100 nM of antisense modulator. Another technique that is commonly used to introduce antisense modulators into cultured cells includes electroporation. When LIPOFECTAMINE or LIPOFECTIN is used, the typical concentration range of antisense modulators administered to cultured cells is 1 nM-300 nM. When electroporation is used, the typical concentration range of antisense modulators administered to cultured cells is 625 nM-20,000 nM.

Following administration of antisense modulators to cultured cells, the cells are typically assayed 16-72 hours post-treatment. The cultured cells can be fixed, stained with antibodies and observed by microscopy to measure phenotypes such as cell survival, cell morphology and the levels of TDP-43 aggregates in the cytoplasm. The cultured cells can also be harvested to measure the levels of RNA or protein levels of target nucleic acids using methods known in the art and described herein. In general, when treatments are performed in multiple replicates, the data are presented as the average of the replicate treatments. The concentration of antisense modulator used can vary from cell line to cell line. Methods to determine the optimal concentrations of an antisense modulator for a particular cell line are well known in the art.

Methods for in vitro testing of modulators, including antisense modulators, are described in U.S. Pat. No. 10,577,604, which along with references cited therein, are incorporated herein in their entirety. Other modifications are known to those skilled in the art and are considered to be included in the embodiments herein.

### In Vivo Testing of Antisense Modulators

In certain embodiments, the antisense modulators described herein can be administered to animals (all of references of which can include humans) in vivo to evaluate their safety, effects on the expression or activity of target gene(s), and effects on other phenotypes, such as, for example, survival, motor function, respiration, behavior, body weight, etc. In certain embodiments, the antisense modulators described herein can be administered to animals in vivo to evaluate the effects of antisense modulators on FLCN expression or activity. In certain embodiments, the antisense modulators provided herein can be administered to animals in vivo to evaluate the safety of the compounds. In certain embodiments, the antisense modulators can be administered to animals in vivo to evaluate the effects of antisense modulators on one or more phenotypes, such as, for example, survival, motor function, respiration, behavior, body weight, etc. In certain embodiments, other modulators described herein, such as, for example, other oligonucleotide modulators, antibody modulators, peptide modulators, or small molecule modulators can be administered to animals in vivo to evaluate their effects on FLCN expression or activity, or on one or more phenotypes, such as, for example, survival, motor function, respiration, behavior, body weight, etc. Methods to measure motor function are well known in the art and include, for example, the grip strength assay, rotarod assay, walking initiation analysis, balance beam test, pole climb assay, open field performance, and hindpaw footprint tests. Methods to measure respiration are well known in the art and include, for example, whole body plethysmograph, invasive resistance, and compliance measurements in the animal. In some embodiments, testing can be performed in healthy animals. In other embodiments, testing can be performed in disease animals.

In certain embodiments, modulators described herein, including antisense modulators, are formulated in a pharmaceutically acceptable diluent, such as phosphate-buffered saline, for administration to animals. Administration includes parenteral routes of administration, such as, for example, intrathecal, intraperitoneal, intravenous, and subcutaneous, etc. Methods to calculate appropriate dosages of antisense modulators and dosing frequency are well known in the art and depends upon factors such as animal body weight and route of administration. Following the administration of modulators, including antisense modulators, to the animal, the animals are monitored at defined timepoints for the expression levels of target gene(s) such as FLCN, and effects on other phenotypes, such as, for example, survival, motor function, respiration, behavior, body weight, etc. The levels of FLCN RNA or FLCN protein can be measured in different tissues from the animal, such as, for example, the CSF, plasma, brain, spinal cord, lung, liver, kidney etc., using methods known in the art and described herein.

Methods for in vivo testing of modulators, including antisense modulators, are described in U.S. Pat. No. 10,577,604, which along with each of the references cited therein, are incorporated herein in their entirety. Other modifications are known to those skilled in the art and are considered to be included in the embodiments herein.

### Other Modulators

In certain embodiments, provided herein are modulators other than antisense modulators, for example other oligonucleotide modulators (e.g., ribozyme, deoxyribozyme, or aptamers), antibody modulators, peptide modulators, small molecule modulators, and nucleic acid vectors, which can be administered to a cell, an animal, or a human subject, to modulate the expression or activity of a target polypeptide or nucleic acid. In one embodiment, provided herein are modulators other than antisense modulators, for example other oligonucleotide modulators (e.g., ribozyme, deoxyribozyme, or aptamers), antibody modulators, peptide modulators, small molecule modulators, and nucleic acid vectors, which can be administered to a cell, an animal, or a human subject, to reduce or inhibit the expression or activity of FLCN, in order to treat, prevent or ameliorate a disease such as ALS or other TDP-43 proteinopathies.

### Ribozyme or Deoxyribozyme

In one embodiment, the modulator can be a therapeutic ribozyme or deoxyribozyme.

A ribozyme or deoxyribozyme in various embodiments can be in any format well known to a person skilled in the art. Ribozymes and deoxyribozymes are sequences of nucleotides (e.g., RNA and DNA sequences, respectively) with enzymatic properties. Ribozymes and deoxyribozymes have been developed to target other molecules, such as RNA introduced by viruses. The enzymatic properties of ribozymes and deoxyribozymes can be used to catalyze, for example, the ligation or cleavage of RNA or DNA via hydrolysis or transesterification of the phosphate groups of the RNA or DNA molecule. Other uses of ribozymes and deoxyribozymes can include catalysis of peptide bonds, as is commonly found within ribosomes. In some cases, the ribozyme or deoxyribozyme activity can be catalyzed by the presence of one or more metal ions. In certain cases, ribozymes or deoxyribozymes can have the ability to self-synthesize or self-splice. A more detailed description of ribozymes and their therapeutic applications is given by Mulhbacher et al. (Mulhbacher et al., Current Opinion in Pharmacology, 10:551-556 (2010)). The Mulhbacher et al. reference, and references cited therein, are incorporated herein by reference in its entirety.

In one embodiment, a ribozyme or deoxyribozyme modulator is used to modulate the translation of mRNA of a target genetic sequence, such as FLCN. In one embodiment, a ribozyme or deoxyribozyme modulator is used to inhibit or reduce the expression or activity of FLCN.

In another embodiment, a ribozyme or deoxyribozyme modulator is chemically attached to a large molecule or scaffold, creating a modulator-scaffold molecular complex. The modulator-scaffold molecular complex can enable additional functionality such as increased activity or efficacy of the modulator's enzymatic activity, improve the modulator's half-life and stability, detection or tracing, or other diagnostic or therapeutic functionality.

In another embodiment, a ribozyme or deoxyribozyme modulator is chemically modified to increase activity or efficacy of the modulator's enzymatic activity, improve the modulator's half-life and stability, detection or tracing, or other diagnostic or therapeutic functionality. In one embodiment, a ribozyme or deoxyribozyme modulator is chemically modified in the 2′ position of its constituent ribose.

Ribozymes or deoxyribozymes can be produced by any number of methods known to a person who is skilled in the art (see below for specific methods).

### Aptamer

In one embodiment, the modulator can be a therapeutic aptamer. A therapeutic aptamer can be an oligonucleotide aptamer, an oligopeptide aptamer, or a polypeptide aptamer.

Aptamers in various embodiments can be in any format well known to a person skilled in the art. Aptamers are oligonucleotide, oligopeptide, or polypeptide molecules engineered to have binding specificity to a target molecule of choice, often through the influence of higher-level structural factors.

In one embodiment, the aptamer modulator is integrated into a larger nucleotide or peptide scaffold. The scaffold can enable additional functionality such as increased activity or efficacy of the modulator, promoting an immunological response, detection or tracing, increased half-life and stability, or other diagnostic or therapeutic functionality. In one such embodiment, the scaffold is a peptide comprising one of the following: a monobody, an anticalin, a polypeptide with a Kunitz domain, an avimer, a knottin, a fynomer, or an atrimer.

In one embodiment, the aptamer modulator is integrated into a ribozyme, deoxyribozyme, or enzyme to form an aptamer-zyme complex. The aptamer-zyme complex can have additional functionality or specificity towards a targeted polypeptide or nucleotide sequence.

In one embodiment, the aptamer is an oligonucleotide chemically modified to increase activity or efficacy of the modulator's enzymatic activity, improve the modulator's half-life and stability, detection, or tracing, or other diagnostic or therapeutic functionality. A list of common chemical modifications in oligonucleotide aptamers is described by Dunn et al. (Dunn et al., Nature Reviews Chemistry, 1(10):0076 (2017)). The Dunn et al. reference, and references cited therein, is incorporated by reference in its entirety and is known to a person of ordinary skill in the art.

In one embodiment, a therapeutic aptamer is used to modulate the expression or activity of a target genetic sequence or protein, such as FLCN. In one embodiment, a therapeutic aptamer is used to inhibit or reduce the expression or activity of FLCN. Aptamers can be produced by any number of methods known to a person who is skilled in the art (see below for specific methods, such as in vitro selection).

### Antibody and Related Protein

In one embodiment, the modulator can be a therapeutic protein or polypeptide. A therapeutic protein can be an antibody, antibody fragment, or monobody. The terms “peptide”, “protein” and “polypeptide” herein are used interchangeably

Antibodies can be in any format well known to a person skilled in the art. Antibodies are heteromultimeric glycoproteins consisting of two larger polypeptide heavy chains and two smaller polypeptide light chains. Each of the heavy chains and light chains comprise a variable region and constant regions. The variable region of the light chain is aligned with the variable region of the heavy chain, and the constant regions of the light chain is aligned with the constant regions of the heavy chain. Each light chain is bound together to a heavy chain via a disulfide covalent bond, and the two heavy chains are bound together by disulfide covalent bonds that can vary in quantity depending on the type of antibody. Antibodies are typically grouped into five different isotypes in mammals: IgA, IgD, IgE, IgG, and IgM. These isotypes are determined by the amino acid sequence of the constant regions of the heavy chains, wherein within each isotype the constant regions of the heavy chains are identical. Light chains are grouped into two different type in mammals: kappa and lambda. Only one type of light chain is present in each antibody, except in the case when the antibody is bispecific. The variable region of the heavy and light chains of the antibody confer the antibody's ability to bind to specific antigens, and are otherwise known as the complementarity-determining region (CDR). The CDR is defined by Dondelinger et al. (Dondelinger et al., Frontiers in Immunology, 9: 2278 (2018)), which along with references cited therein, are incorporated herein in its entirety. The different isotypes determined by the constant regions enable different crystallizable fragments to bind to the antibody or antibody-antigen complex. These crystallizable fragments are associated with different pathways in immunological response and other physiological effects such as clearance rate, cell-mediated cytotoxicity, phagocytosis through agglutination or precipitation, and complement-dependent cytotoxicity.

In a preferred embodiment, the antibody modulator contains a constant region of the IgG isotype derived from human sources. In another embodiment, the antibody can be humanized or chimeric. In a preferred embodiment, the antibody is monoclonal. In another embodiment, the antibody modulator is used in conjunction with other antibody modulators of other polypeptides or nucleotide sequence of interest to inhibit the function of multiple polypeptides and/or multiple nucleotide sequences within one or more functional pathways. In yet another embodiment, the antibody is bispecific, being capable of binding to two separate polypeptides or nucleotide sequences or any combination thereof of interest.

In certain embodiments, the antibody can comprise additional polypeptide chains or functional groups that confer additional properties to the antibody, such as enhanced immune response, enhanced antigen specificity, or stability.

In certain embodiments, the modulator is an antibody fragment, such as a single-chain variable fragment or antigen binding fragment, which is able to bind to and inhibit the function of the polypeptide or nucleotide sequence of interest.

In other embodiments, the modulator is a monobody which is able to bind to and inhibit the function of the polypeptide or nucleotide sequence of interest. Monobodies in various embodiments can be in any format well known to a person skilled in the art. Monobodies are proteins that are smaller and less complex than antibodies, and that are engineered to have antigen-binding properties similar to that of antibodies. Monobodies are created with a fibronectin type III scaffold in which certain sections of its amino acid sequence is varied to create variable specificity to antigens of choice.

In certain embodiments, there is provided a modulator comprising an antibody, antibody fragment, or monobody that binds to the polypeptide or nucleotide sequence of interest, thus modulating the expression or activity of the target polypeptide or nucleotide sequence of interest. In other embodiments, there is provided a modulator comprising an antibody, antibody fragment, or monobody that binds to the polypeptide or nucleotide sequence of interest, thus modulating the interaction of the polypeptide or nucleotide sequence of interest with other molecules in the targeted functional pathway. In one embodiment, the modulator comprises an antibody, antibody fragment, monobody or other compound that binds to the FLCN protein, thus inhibiting or reducing the expression or activity of FLCN. In one embodiment, the modulator comprises an antibody, antibody fragment, or monobody that disrupts the activity of FLCN, preventing or reducing its ability to shuttle TDP-43 from the nucleus into the cytoplasm. In one embodiment, the modulator comprises an antibody, antibody fragment, or monobody that disrupts the interaction between FLCN and TDP-43. In one embodiment, the antibody, antibody fragment, or monobody targets FLCN and blocks its interaction with TDP-43. In another embodiment, the antibody, antibody fragment, or monobody targets the region between amino acids 202-299 of FLCN, thereby blocking its interaction with TDP-43. In one embodiment, the antibody, antibody fragment, or monobody targets TDP-43 and blocks its interaction with FLCN, leading to either a decrease in levels of TDP-43 aggregates in the cytoplasm, or an increase in levels of functional TDP-43 in the nucleus, or a combination thereof. In one embodiment, the antibody, antibody fragment, or monobody targets the RRM1 and RRM2 domains of TDP-43, thereby blocking its interaction with FLCN. Antibody modulators that target FLCN are provided in Example 9.

In one embodiment, antibody modulators that are capable of targeting FLCN can be obtained commercially from a variety of sources, such as the MSRS catalog of antibodies (Aerie Corporation, Birmingham, Mich.). In some embodiments, provided herein are antibodies for the detection of mouse, rat, monkey, and human FLCN, which are available from commercial sources, examples of which are provided in Table 16. In some embodiments, provided herein are antibodies, antibody fragments, monobodies, or other peptide modulator that binds to the same epitope as at least one antibody described in Table 16. In other embodiments, the antibody, antibody fragment, monobody, or peptide modulator binds to a different epitope as that of the modulators described in Table 16. In some embodiments, the antibody, antibody fragment, monobody, or peptide modulator comprises a CDR that is at least 30%, 40%, 50%, 60%, 70%, 75%, 80%, 85%, 90%, 95%, 96%, 97%, 98%, 99%, or 100% similar to the CDR of at least one antibody described in Table 16, as assessed by sequence alignment or other scoring methods known in the art. Such protein sequence alignment or scoring methods can take into account the 3D structure or conformation of the CDR region. Protein sequence alignment and scoring methods are described by Wang et al. (Wang et al., BMC Bioinformatics, 19: 529 (2018)) and Kunik et al. (Kunik et al., PLoS Computational Biology, 8(2): e1002388 (2012)), which together with references cited therein, are incorporated herein in their entirety.

Antibodies, antibody fragments, and monobodies can be produced by any number of methods known to a person who is skilled in the art (see below for specific methods).

### Small Molecule

In one embodiment, the modulator is a small molecule.

A small molecule in various embodiments can be in any format well known to a person skilled in the art. A small molecule is generally referred to as a molecule with molecular weight less than 3000 Daltons that specifically targets a molecule of interest.

In certain embodiments, there is provided a modulator comprising a small molecule that binds to a polypeptide or nucleotide sequence of interest, thus modulating the expression or activity of the target polypeptide or nucleotide sequence of interest. In other embodiments, there is provided a modulator comprising a small molecule that binds to a polypeptide or nucleotide sequence of interest, thus modulating the interaction of the polypeptide or nucleotide sequence of interest with other molecules in the targeted functional pathway. In one embodiment, the modulator comprises a small molecule that binds to a nucleotide sequence encoding FLCN, thus inhibiting or reducing the expression of FLCN. A nucleotide sequence encoding FLCN refers to a nucleic acid encoding FLCN. In one embodiment, the small molecule modulator targets a nucleic acid encoding FLCN and inhibits its transcription. In one embodiment, the small molecule modulator targets a nucleic acid encoding FLCN and degrades or destabilizes it. In one embodiment, the small molecule modulator targets a nucleic acid encoding FLCN and inhibits its translation. In yet another embodiment, the small molecule modulator targets a nucleic acid encoding FLCN and modulates its splicing, thereby decreasing the expression or activity of FLCN. In some embodiments, the small molecule modulator is a bivalent compound that is capable of binding to both FLCN RNA and a ribonuclease such as RNase L to induce degradation of the FLCN RNA. Such bivalent compounds are known as RIBOTACs (ribonuclease-targeting chimeras) and are described by Dey et al. (Dey et al., Cell Chemical Biology, 26(8): 1047-1049 (2019)), which together with references cited therein, are incorporated herein in their entirety. In one embodiment, the modulator comprises a small molecule that binds to a FLCN protein, thus inhibiting or reducing the activity of FLCN. In one embodiment, the modulator comprises a small molecule that disrupts the activity of FLCN, thus preventing or reducing its ability to shuttle TDP-43 from the nucleus into the cytoplasm. In one embodiment, the modulator comprises a small molecule that disrupts the interaction between FLCN and TDP-43. In one embodiment, the small molecule targets TDP-43 and blocks its interaction with FLCN, leading to a decrease in levels of TDP-43 aggregates in the cytoplasm. In one embodiment, the small molecule binds to the RRM1 and RRM2 domains of TDP-43, thereby blocking its interaction with FLCN. In one embodiment, the small molecule targets FLCN and blocks its interaction with TDP-43. In another embodiment, the small molecule targets the region between amino acids 202-299 of FLCN, thereby blocking its interaction with TDP-43. In certain embodiments, the small molecule modulator reduces or inhibits the expression or activity of FLCN protein by targeting it for degradation in the cell. In some embodiments, the small molecule modulator is a bivalent compound that is capable of binding to both FLCN protein and an E3 ubiquitin ligase to induce ubiquitination of FLCN and its subsequent degradation by the proteasome. Such bivalent compounds are known as PROTACS (proteolysis-targeting chimeras) and are described by Toure et al. (Toure et al., Angew. Chem. Int. Ed., 55: 1966-1973 (2016)), which together with references cited therein, are incorporated herein in their entirety. Small molecule modulators that target the FLCN protein are provided in Example 8. In certain embodiments, provided herein are small molecule modulators comprising at least one exemplar described in Table 15. In certain embodiments, the small molecule modulator comprises at least one scaffold described in Table 15. In some embodiments, the small molecule modulators, or part thereof, have a Tanimoto index of at least 0.20, 0.30, 0.40, 0.50, 0.60, 0.70, 0.75, 0.80, 0.85, 0.90, 0.95, 0.96, 0.97, 0.98, 0.99, or 1.00 compared to at least one exemplar or scaffold described in Table 15.

In other embodiments, the small molecule modulators bind to a nucleotide sequence encoding FLCN, thus increasing the expression of FLCN. In one embodiment, the small molecule modulator targets a nucleic acid encoding FLCN and increases its transcription. In one embodiment, the small molecule modulator targets a nucleic acid encoding FLCN and stabilizes it. In one embodiment, the small molecule modulator targets a nucleic acid encoding FLCN and increases its translation. In yet another embodiment, the small molecule modulator targets a nucleic acid encoding FLCN and modulates its splicing, thereby increasing the expression or activity of FLCN. In one embodiment, the modulator comprises a small molecule that binds to a FLCN protein, thus increasing the activity of FLCN. In some embodiments, the small molecule modulator targets the longin and/or DENN domains of FLCN, thereby promoting its interaction with FNIP1 and/or FNIP2, thus increasing the formation of the FLCN-FNIP1 complex and/or FLCN-FNIP2 complex respectively. In another embodiment, provided herein are bivalent small molecule modulators that are capable of increasing the interaction of FLCN with FNIP1 or FNIP2, thereby leading to an increase in the levels or activity of the FLCN-FNIP1 and/or FLCN-FNIP2 complex respectively.

Small molecules can be produced by any number of methods known to a person who is skilled in the art (see below for specific methods).

### Nucleic Acid Vector

In one embodiment, the modulator comprises a nucleic acid vector. In one embodiment, the nucleic acid vector can encode for the modulator of choice, or a nucleobase sequence that includes the modulator of choice, or a nucleobase sequence complementary to the modulator of choice, wherein the modulator of choice is, for example, a siRNA, miRNA, lncRNA, gRNA, an antisense oligonucleotide, or a gene. The modulator, or a nucleobase sequence containing the modulator, or a nucleobase sequence complementary to the modulator, can be expressed following delivery of the nucleic acid vector into a target cell. The terms vector and nucleic acid vector herein are used interchangeably.

In some embodiments, the nucleic acid vector encodes for one or more functional copies of the gene of interest, such as FLCN. The additional copies of FLCN are expressed in the cell to increase the levels of FLCN in the cell. In certain embodiments, the nucleic acid vector encodes for a modulator that is expressed to form an activator. The activator targets FLCN nucleic acids or polypeptides to increase the expression or activity of FLCN. In some embodiments, the activator targets DNA sequences encoding FLCN, or DNA sequences that regulate the expression of FLCN, and increases transcription of the FLCN gene to mRNA. In another embodiment, the activator targets the FLCN mRNA transcribed from the gene and increases translation of FLCN mRNA into the polypeptide. In one embodiment, the nucleic acid vector encodes for a modulator that is expressed to form an inhibitor. The inhibitor targets the polypeptide of interest, such as FLCN, and inhibits or reduces the interaction of FLCN with other molecules in the targeted functional pathway, such as TDP-43, thereby leading to a reduction in TDP-43 aggregation in the cytoplasm. In another embodiment, the inhibitor targets DNA sequences encoding FLCN or that regulate the expression of FLCN, and inhibits or reduces transcription of the FLCN gene to mRNA. In another embodiment the inhibitor targets the FLCN mRNA transcribed from the gene and inhibits or reduces translation of FLCN mRNA into the polypeptide.

In another embodiment, the nucleic acid vector encodes for any of the modulator embodiments above. In a certain embodiment, the nucleic acid vector is modified to enable a viral delivery method, such as pseudotyping, adaptor targeting, or genetic systems targeting, described herein. In other embodiments, the nucleic acid vector is modified to enable a non-viral delivery method described herein. In one embodiment, the nucleic acid vector for non-viral delivery is a minimized DNA vector. In one embodiment, the minimized DNA vector lacks antibiotic resistance genes that are typically present in plasmid DNA vectors. Minimized DNA vectors have the advantages of high transfection efficiency and high production yields over regular plasmid DNA. Furthermore, the lack of antibiotic resistance genes helps to reduce the risk of spread of antibiotic resistance genes in the environment. In one embodiment, the minimized DNA vector is a minicircle, wherein sequences of bacterial origin such as the origin of replication are removed. In one embodiment, the minimized DNA vector is a minivector, which can be smaller than a minicircle. Various advances in the design of non-viral minimized DNA vectors, as well as methods of production and use are described in Hardee et al. (Hardee et al., Genes, 8, 65 (2017)), which together with references cited therein, are incorporated herein in their entirety. Nucleic acid vectors and their methods of delivery (e.g., modified virus) can be produced by any number of methods known to a person who is skilled in the art (see below for specific methods).

### Gene Therapy

In certain embodiments, provided herein are compositions and methods involving transfer of genetic material to a cell, an animal, or a human subject, in order to treat, prevent, or ameliorate a disease, otherwise referred to as gene therapy. The genetic material can be integrated into the genome (e.g., via an integrating vector or by homology-directed repair). In certain embodiments, the genetic material is not integrated into the genome (e.g., carried on a non-integrating vector). The genetic material can be administered in vivo (directly into the patient), or the genetic material can be administered ex vivo (to cultured cells taken from the patient that are subsequently transplanted back into the patient). In certain embodiments, lentiviral vectors are used for ex vivo transfer of genetic material into hematopoietic and other stem cells. In other embodiments, adeno-associated viral (AAV) vectors are used for in vivo transfer of genetic material into postmitotic cell types. In some embodiments, AAV2 or AAV9 vectors are used for in vivo transfer of genetic material into the central nervous system (CNS). Compositions and methods of gene therapy as a therapeutic method are described by Anguela and High (Anguela and High, Annual Review of Medicine, 70, 273-288 (2019)), which along with references cited therein, are incorporated herein by reference in their entirety.

One possible goal of gene therapy is gene augmentation, which seeks to restore normal cellular function by increasing the expression of a gene. For example, if a mutation in a gene leads to a loss-of-function of the polypeptide or nucleotide sequence encoded by the gene, which leads to the disease, a modulator or additional normal functional copies of the gene can be supplied to the cell to increase the expression of the gene. In some embodiments, even if the disease is not caused by, or associated with, a loss-of-function of the polypeptide or nucleotide sequence encoded by the gene, gene augmentation can still be useful to treat, prevent or ameliorate the disease. Another possible goal of gene therapy is gene suppression, which seeks to restore cellular function by reducing the expression of a gene. For example, if a mutation in a gene leads to a gain-of-function of the polypeptide or nucleotide sequence encoded by the gene, which leads to the disease, a modulator to suppress the expression of the gene can be supplied to the cell. In some embodiments, even if the disease is not caused by, or associated with, a gain-of-function of the polypeptide or nucleotide sequence encoded by the gene, gene suppression can still be useful to treat, prevent or ameliorate the disease.

In certain embodiments, gene therapy comprises genome editing, which is the modification of the genome of the cell, for example, by removing pathogenic mutations or introducing beneficial mutations to one or more genetic features, in order to restore normal cellular function. In one embodiment, genome editing relies on an enzyme or enzyme complex, such as TALENs, CRISPR/Cas, zinc-finger nucleases (ZFNs), meganucleases, or other endonuclease system. The enzyme or enzyme complexes used for genome editing described here are non-exhaustive and other enzyme or enzyme complexes used for genome editing are well known to a person of ordinary skill in the art and are included in various embodiments. For example, genome editing as a therapeutic approach is described by Ho et al. (Ho B. X. et al. International Journal of Molecular Sciences 19: 2721 (2018)), which along with references cited therein, are incorporated herein by reference in their entirety.

The typical modus operandi of genome editing involves the inserting, replacing, or deleting of certain nucleotide sequences in the genome of an organism, often through the introduction of an enzyme or enzyme complex and an exogenous nucleotide sequence. In some embodiments, the enzyme or enzyme complex facilitates the genome editing process by creating site-specific double-stranded breaks in the genome. In certain embodiments, homology directed repair, such as homologous recombination, is used by the cell to replace parts of the genome using an exogenous sequence of nucleotides, which is often introduced into the cell via a nucleic acid vector. Homology directed repair uses the cells natural enzymatic mechanisms to repair double-stranded breaks in the genome through the use of a homologous template. By introducing an exogenous sequence of nucleotides comprising the desired modified nucleotide sequence flanked by nucleotide sequences that are homologous to the genomic nucleic acid sequence at or around the double-stranded break, the cell can utilize this exogenous sequence of nucleotides as the homologous template for the homology directed repair process, thereby inserting, modifying, or deleting the original sequence of nucleotides present at or around the double-stranded break. In other embodiments, non-homologous end-joining is used by the cell to directly repair double-stranded breaks in the genome without using a homologous template. During the non-homologous end-joining process, insertions or deletions are introduced into the genome, which in the case of protein-coding genes often leads to a change in the reading frame or introduction of a premature stop codon, thus rendering the gene non-functional.

In one embodiment, two double-stranded breaks are introduced at the region where genome editing is desired to increase the efficiency of homology-directed repair or non-homologous end-joining. In another embodiment, more than one genomic region is targeted for homology-directed repair or non-homologous end-joining by introducing more than one double-stranded break simultaneously at different genomic locations in the cell. In yet another embodiment, a single-stranded break (that is similarly capable of stimulating homology-directed repair or non-homologous end-joining) is introduced in the genome using an engineered endonuclease, instead of a double-stranded break, in order to reduce toxicity to the cell. In certain embodiments, genome editing can be achieved without single-stranded breaks, double-stranded breaks or using a homologous template. This can be achieved by using a catalytically impaired endonuclease (e.g., Cas9) fused to an engineered reverse transcriptase, which is programmed with a prime editing guide RNA (pegRNA) that both encodes the desired edits and specifies the target site, otherwise known as prime editing. Prime editing is described by Anzalone et al. (Anzalone et al., Nature, 576 (7785), 149-157 (2019)), which along with references cited therein, are incorporated herein by reference in their entirety.

In certain embodiments, genome editing involves making integrative changes (e.g., insertions, deletions, or modifications) to the DNA sequence in the chromosome of the cell. This can be achieved by using an endonuclease that can recognize and cleave DNA sequences, for example, Cas9 and Cas12a (also known as Cpf1). In other embodiments, genome editing involves making non-integrative changes (e.g., insertions, deletions, or modifications) to the RNA sequence in the cell. This can be achieved by using an endonuclease that can recognize and cleave RNA sequences, for example, Cas13. RNA-targeting CRISPR-Cas endonucleases and systems are described by Burmistrz et al. (Burmistrz et al., Int. J. Mol. Sci. 21, 1122 (2020)), which along with references cited therein, are incorporated herein by reference in their entirety.

In another embodiment, genome editing can comprise the correction of at least one point mutation using an engineered endonuclease that is catalytically inactive but able to recognize and bind to a specific sequence of DNA containing the point mutation, wherein the engineered endonuclease is coupled to a base editing enzyme, and correcting the point mutation via the activity of the coupled base editor. In one embodiment, the catalytically inactive endonuclease does not introduce a double-stranded break. Base editing can be used to introduce any of the four transition mutations, C to T, G to A, A to G, and T to C. Specifically, for DNA, the cytosine base editor (CBE) can alter a C-G base pair into a T-A base pair, while the adenine base editor (ABE) can alter an A-T base pair into a G-C pair. In another embodiment, genome editing can comprise the correction of at least one point mutation using an engineered endonuclease that is catalytically inactive but able to recognize and bind to a specific sequence of RNA containing the point mutation. For RNA, the RNA base editor (RBE) can convert adenine (A) to inosine (I). CRISPR/Cas-mediated base editing is described by Molla and Yang (Molla K. A. and Yang Y., Trends in Biotechnology, 37(10), 1121-1142, (2019)), which along with references cited therein, are incorporated herein by reference in their entirety.

In yet another embodiment, genome editing can comprise using a catalytically inactive endonuclease linked to at least one epigenetic modification enzyme to effect a change to the epigenetic state of the genome. For example, catalytically inactive Cas9 (CRISPR endonuclease) can be coupled to epigenetic modification enzymes including but not limited to KRAB, DNMT3A, LSD1, p300, TET, VP64, SunTag-epieffector, SAM-epieffector, and the like. In one embodiment, the catalytically inactive endonuclease is coupled to activator and/or repressor domains to effect a change in the expression of at least one target gene. Some examples that are known to a skilled person in the art include CRISPR interference (CRISPRi) and CRISPR activation (CRISPRa). CRISPR/Cas-mediated epigenetic methods are described in Xie et al. (Xie et al., Stem Cells International, Article ID 7834175 (2018)), which along with references cited therein, are incorporated herein by reference in their entirety.

In certain embodiments, provided herein are compositions and methods of gene therapy to modulate the expression or activity of FLCN. In one embodiment, provided herein are compositions and methods of gene therapy to reduce or inhibit the expression or activity of FLCN, in order to either reduce the levels of TDP-43 aggregates in the cytoplasm, or increase the levels of functional TDP-43 in the nucleus, or achieve a combination of both, thereby treating, preventing or ameliorating a disease such as ALS, or other TDP-43 proteinopathy. In one embodiment, genome editing is used to insert, delete, or modify DNA sequences associated with FLCN, such as sequences described by SEQ ID NOs: 1-15. In one embodiment, genome editing is used to insert, delete or modify RNA sequences associated with FLCN, such as sequences described by SEQ ID NOs: 1-15. Genome editing via enzymes or enzyme complexes and their methods of delivery can be produced by any number of methods known to a person who is skilled in the art, which are incorporated herein (see below for specific methods).

### Pharmaceutical Compositions

A set of embodiments provides a pharmaceutical composition comprising at least one modulator together with a pharmaceutically acceptable carrier or diluent. The carrier or diluent of the pharmaceutical composition must be “acceptable” in the sense of being compatible with the other ingredients of the composition and not deleterious to the recipients thereof. The pharmaceutical composition may be in unitary dosage form suitable, in particular, for administration orally, rectally, percutaneously, by parenteral injection or by inhalation. In some cases, administration can be via intravenous injection. For example, in preparing the composition in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable solutions, for example, may be prepared in which the carrier comprises saline solution, glucose solution or a mixture of saline and glucose solution. Injectable solutions containing the modulators described herein may be formulated in oil for prolonged action. Appropriate oils for this purpose are, for example, peanut oil, sesame oil, cottonseed oil, corn oil, soybean oil, synthetic glycerol esters of long chain fatty acids and mixtures of these and other oils. Injectable suspensions may also be prepared in which case appropriate liquid carriers, suspending agents and the like may be employed. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired composition. The composition may be administered in various ways, e.g., as a transdermal patch, as a spot-on, as an ointment.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

In one embodiment, the pharmaceutical composition can be administered to a cell, an animal or a human subject. In one embodiment, the pharmaceutical composition can be used to treat, prevent, or ameliorate ALS. In another embodiment, the pharmaceutical composition can be used to treat, prevent, or ameliorate other diseases, particularly neuromuscular or neurodegenerative diseases and other diseases that are associated with TDP-43 proteinopathy.

In another embodiment, the pharmaceutical composition can be used to treat, prevent, or ameliorate other diseases, particularly oxidative stress, obesity, anemia, or ischemic diseases, such as cardiovascular disease, myocardial ischemia, and peripheral vascular disease.

Pharmaceutical compositions can be created using standard practices that are known to a person who is skilled in the art. Pharmaceutical compositions can be designed for administration in one of a number of various methods that are known to a person who is skilled in the art. A more detailed list of common practices is described by Wu & Chen (US 2018/0112272 A1), which along with references cited therein, are incorporated herein in its entirety.

In one embodiment, the pharmaceutical composition is for parenteral administration. In certain embodiments, compositions for parenteral administration can be sterile solutions, emulsions or suspensions that can be prepared from a solid or lyophilized form prior to administration. In other embodiments, the composition can contain certain adjuvants, anesthetics, buffering agents, or wetting agents that promote more effective distribution of the composition, facilitate ease of administration of the composition, or improve patient response or wellbeing. In one embodiment, the pharmaceutical composition is for intrathecal administration. In other embodiments, the pharmaceutical composition is for intramuscular, intracerebral, intracerebroventricular, intravenous, intravitreal or intraocular administration.

In another embodiment, the pharmaceutical composition is for gastrointestinal or enteric administration. In one embodiment, the pharmaceutical composition can be administered orally. In certain embodiments, compositions for gastrointestinal or oral administration can be a tablet, powder, capsule, or liquid. Such compositions can be formulated with a solid or liquid physiologically compatible carrier, including but not limited to, mannitol, lactose, starch, magnesium stearate, sodium saccharine, talcum, cellulose, glucose, sucrose, magnesium, carbonate. In some embodiments, compositions can be formulated with disintegrants, including but not limited to starches, clays, celluloses, aligns, gums, and polymers, to facilitate the dissolution of solids. In other embodiments, compositions can also be formulated with lubricants, including but not limited to silicon dioxide, talc, or stearic acids, to facilitate the effective manufacturing of the composition.

In another embodiment, the pharmaceutical composition is administered transdermally or topically, such as in the form of an ointment, cream, or gel. In another embodiment, the pharmaceutical composition is administered transmucosally, such as in the form of a spray or a suppository.

In one embodiment, the pharmaceutical composition can be administered by nasal administration, including but not limited to, an inhalant, or delivered in an aerosol delivery device, such as an atomizer, nebulizer, or vaporizer. The aerosol delivery devices mentioned herein and other aerosol delivery devices are well known to a person of ordinary skill in the art and are included in various embodiments herein.

In another embodiment, the pharmaceutical composition is delivered via a targeted method that introduces or directs the pharmaceutical composition directly to the affected cells. Manish and Vimukta (Manish and Vimukta, Research Journal of Chemical Sciences, 1(2), 135-138 (2011)) describe common methods for targeted drug delivery, which along with references cited therein, are incorporated herein by reference in its entirety.

### Treatment Methods

In certain embodiments, methods of treatment comprising administration of the pharmaceutical compositions to a cell, an animal or a human subject can vary in terms of composition, quantity of doses, and scheduling of doses. A unit dose is a pre-determined therapeutically effective amount of pharmaceutical composition that is administered. Unit doses can vary depending on various factors, including but not limited to, weight, age, gender, severity of symptoms, medical history, and aggressiveness of treatment. A schedule is the frequency of administration of unit doses. The size of a unit dose and the schedule of administration of the pharmaceutical composition can be determined by a person of ordinary skill in the art, and are incorporated in certain embodiments herein.

In certain embodiments, one or more pharmaceutical compositions described herein are co-administered with one or more other pharmaceutical agents. In one embodiment, the one or more other pharmaceutical agents are designed to treat a different disease, disorder, symptom, or condition compared to the one or more pharmaceutical compositions described herein. In another embodiment, the one or more other pharmaceutical agents are designed to treat the same disease, disorder, symptom, or condition as the one or more pharmaceutical compositions described herein. In certain embodiments, the one or more other pharmaceutical agents are co-administered with one or more pharmaceutical compositions described herein to produce an additive effect. In certain embodiments, the one or more other pharmaceutical agents are co-administered with one or more pharmaceutical compositions described herein to produce a synergistic or supra-additive effect, wherein the co-administration of the pharmaceutical composition and agent results in an effect that is greater than the sum of the effects of administering either pharmaceutical composition or agent alone. In another embodiment, the one or more other pharmaceutical agents are co-administered with one or more pharmaceutical compositions described herein to treat an undesired side effect of one or more pharmaceutical compositions described herein. In certain embodiments, one or more pharmaceutical compositions described herein are co-administered with one or more other pharmaceutical agents to treat an undesired side effect of the one or more other pharmaceutical agents. In certain embodiments, one or more pharmaceutical compositions described herein are co-administered with one or more other pharmaceutical agents to prevent or delay the onset of symptoms, slow disease progression, improve the therapeutic efficacy of the one or more pharmaceutical compositions, or to otherwise improve patient outcomes.

In some embodiments, one or more pharmaceutical compositions described herein and one or more other pharmaceutical agents are prepared together in a single formulation. In other embodiments, one or more pharmaceutical compositions described herein and one or more other pharmaceutical agents are prepared separately. In certain embodiments, the one or more pharmaceutical agents are administered following administration of one or more pharmaceutical compositions described herein. In certain embodiments, the one or more pharmaceutical agents are administered prior to administration of one or more pharmaceutical composition described herein. In certain embodiments, the co-administered pharmaceutical agent is administered at the same time as a pharmaceutical composition described herein.

In certain embodiments, the one or more pharmaceutical composition described herein and the one or more other pharmaceutical agent are antisense modulators. In certain embodiments, the one or more pharmaceutical composition described herein is an antisense modulator, and the one or more other pharmaceutical agent is a small molecule modulator. In other embodiments, the one or more pharmaceutical composition described herein and the one or more other pharmaceutical agent can independently comprise modulators such as antisense modulators, other oligonucleotide modulators (e.g., ribozyme, deoxyribozyme, or aptamers), antibody modulators, peptide modulators, small molecule modulators, and/or nucleic acid vectors. In certain embodiments, one or more pharmaceutical agents that can be co-administered with one or more pharmaceutical compositions described herein include Riluzole (Rilutek), Dexpramipexole, Edaravone, Tofersen, Baclofen (Lioresal), or other drug that is typically administered to treat or ameliorate symptoms in ALS. In certain embodiments, one or more pharmaceutical agents that can be co-administered with one or more pharmaceutical compositions described herein include drugs that alleviate pain, inflammation or other symptoms (e.g., COX inhibitors, phenylbutazone, ibuprofen, suprofen, fenbufen, ketoprofen, pranoprofen, carprofen, indomethacin, folinic acid, tiaprofenic acid, diclofenac, niflumic acid, diazepines or benzodiazepines (e.g., Diazepam), barbiturate); or drugs that improve uptake or delivery, such as blood thinners (e.g., aspirin, warfarin); or antibacterial, antiviral, antibiotic; or other drug that provides at least one benefit, including treatment efficacy, symptom alleviation, drug tolerance, or side effect mediation, in a therapeutic setting.

In other embodiments, a pharmaceutical composition described herein is co-administered with one or more pharmaceutical agents or other drug that is typically administered to treat, ameliorate, or manage symptoms in oxidative stress, obesity, anemia or ischemic diseases; as well as inflammatory diseases, von Hippel-Lindau (VHL) disease, Birt-Hogg-Dube (BHD) syndrome, spontaneous pneumothorax, B cell deficiency, cardiomyopathy, and cancers.

In certain embodiments, one or more pharmaceutical agents that can be co-administered with a pharmaceutical composition to reduce or inhibit the expression or activity of FLCN described herein include, but are not limited to, an additional FLCN modulator, or other modulator that can reduce the levels of pathological TDP-43 aggregates in the cytoplasm, or increase the levels of functional TDP-43 in the nucleus, or achieve a combination of both. In certain embodiments, the dose of a co-administered pharmaceutical agent is lower than the dose that would be administered if the co-administered pharmaceutical agent was administered alone. In certain embodiments the dose of a co-administered pharmaceutical agent is higher than the dose that would be administered if the co-administered pharmaceutical agent was administered alone. In certain embodiments the dose of a co-administered pharmaceutical agent is the same as the dose that would be administered if the co-administered pharmaceutical agent was administered alone.

### Certain Indications

In certain embodiments, provided herein are methods of treatment of a human subject diagnosed with a neuromuscular or neurodegenerative disease, such as, for example, ALS, FTLD, Alzheimer's disease, retinal degeneration diseases such as age-related macular degeneration (AMD), or other TDP-43 proteinopathies described herein, comprising administering one or more pharmaceutical compositions described herein to the human individual. In certain embodiments, provided herein are methods for prophylactically reducing or inhibiting FLCN expression or activity in a human subject, wherein the human subject is at risk for developing a neuromuscular or neurodegenerative disease, including but not limited to, ALS, or other TDP-43 proteinopathies described herein.

In some embodiments, provided herein are methods of treatment of a human subject diagnosed with oxidative stress, obesity, anemia or ischemic disease, such as, for example, chronic anemia, cardiovascular disease, myocardial ischemia or peripheral vascular disease, comprising administering one or more pharmaceutical compositions described herein to the human individual. In certain embodiments, provided herein are methods for prophylactically reducing or inhibiting FLCN expression or activity in a human subject, wherein the human subject is at risk for developing oxidative stress, obesity, anemia, or ischemic diseases, such as cardiovascular disease, myocardial ischemia, or peripheral vascular disease.

In certain embodiments, provided herein are methods of treatment of a human subject diagnosed with a disease, such as Birt-Hogg-Dube (BHD) syndrome, fibrofolliculomas, lung cysts, spontaneous pneumothorax, kidney tumors, and other diseases that are linked to loss-of-function of FLCN described herein, comprising administering one or more pharmaceutical compositions described herein to the human individual. In certain embodiments, provided herein are methods for prophylactically increasing FLCN expression or activity in a human subject, wherein the human subject is at risk for developing Birt-Hogg-Dube (BHD) syndrome, fibrofolliculomas, lung cysts, spontaneous pneumothorax, kidney tumors, and other diseases that are linked to loss-of-function of FLCN described herein. In certain embodiments, provided herein are methods of treatment of a human subject diagnosed with a disease, such as inflammatory diseases, von Hippel-Lindau (VHL) disease, B cell deficiency, cardiomyopathy, as well as other cancers described herein, comprising administering one or more pharmaceutical compositions described herein to the human individual. In certain embodiments, provided herein are methods for prophylactically increasing FLCN expression or activity in a human subject, wherein the human subject is at risk for developing inflammatory diseases, von Hippel-Lindau (VHL) disease, B cell deficiency, cardiomyopathy, as well as other cancers described herein.

In certain embodiments, provided herein are methods of treatment of a human subject in need thereof by administering to the human individual a therapeutically effective amount of an antisense modulator targeting one or more FLCN nucleic acids disclosed by SEQ ID NOs: 1-15 herein. In one embodiment, administration of a therapeutically effective amount of an antisense modulator targeted to a FLCN nucleic acid disclosed by SEQ ID NOs: 1-15 herein is accompanied by monitoring of FLCN levels in the human individual, to determine the individual's response to administration of the antisense modulator. In certain embodiments, provided herein are methods of treatment of a human subject in need thereof by administering to the human individual a therapeutically effective amount of a modulator described herein, such as, for example, other oligonucleotide modulator, antibody modulator, peptide modulator, or small molecule modulator, that targets the expression or activity of FLCN. Other examples of a modulator include a nucleic acid vector and gene therapy. In one embodiment, administration of a therapeutically effective amount of a modulator described herein, such as, for example, other oligonucleotide modulator, antibody modulator, peptide modulator, or small molecule modulator, is accompanied by monitoring of FLCN levels in the human individual to determine the individual's response to administration of the modulator. Other examples of a modulator include a nucleic acid vector and gene therapy. A human subject's response to administration of the antisense or other modulator can be used by a physician to determine the dose, schedule, and duration of therapeutic intervention.

In certain embodiments, administration of an antisense modulator targeted to a FLCN nucleic acid disclosed by SEQ ID NOs: 1-15 herein, results in reduction of FLCN mRNA or protein expression by at least 5, 10, 20, 30, 40, 50, 60, 70, 80, 85, 90, 95, 96, 97, 98, or 99%, or a range defined by any two of these values. In other embodiments, administration of an antisense modulator targeted to a FLCN nucleic acid disclosed by SEQ ID NOs: 1-15 herein, results in an increase of FLCN mRNA or protein levels by at least 10, 50, 100, 200, 300, 400, 500, 600, 700, 800, 900, 1900, 2900, 3900, 4900, 5900, 6900, 7900, 8900, or 9900%, or a range defined by any two of these values. In certain embodiments, administration of an antisense modulator targeted to a FLCN nucleic acid disclosed by SEQ ID NOs: 1-15 herein, results in improved motor function and respiration in a human subject. In certain embodiments, administration of a FLCN antisense modulator improves motor function and respiration by at least 5, 10, 20, 30, 40, 50, 60, 70, 80, 85, 90, 95, 96, 97, 98, or 99%, or a range defined by any two of these values. In certain embodiments, administration of a modulator, such as an oligonucleotide modulator, antibody modulator, peptide modulator, or small molecule modulator, results in reduction of FLCN mRNA or protein expression by at least 5, 10, 20, 30, 40, 50, 60, 70, 80, 85, 90, 95, 96, 97, 98, or 99%, or a range defined by any two of these values. In other embodiments, administration of a modulator, such as a nucleic acid vector, gene therapy, oligonucleotide modulator, antibody modulator, peptide modulator, or small molecule modulator targeted to a FLCN nucleic acid disclosed by SEQ ID NOs: 1-15 herein, results in an increase of FLCN mRNA or protein levels by at least 10, 50, 100, 200, 300, 400, 500, 600, 700, 800, 900, 1900, 2900, 3900, 4900, 5900, 6900, 7900, 8900, or 9900%, or a range defined by any two of these values. In certain embodiments, administration of a modulator, such as an oligonucleotide modulator, antibody modulator, peptide modulator, or small molecule modulator, results in improved motor function and respiration in a human subject. In certain embodiments, administration of a modulator, such as an oligonucleotide modulator, antibody modulator, peptide modulator, or small molecule modulator, improves motor function and respiration by at least 5, 10, 20, 30, 40, 50, 60, 70, 80, 85, 90, 95, 96, 97, 98, or 99%, or a range defined by any two of these values.

In certain embodiments, pharmaceutical compositions comprising an antisense modulator targeted to FLCN are used for the preparation of a medicament for treating a patient diagnosed with or susceptible to a disease, in particular neuromuscular or neurodegenerative disease, such as, for example ALS, FTLD, or other TDP-43 proteinopathies described herein. In other embodiments, pharmaceutical compositions comprising a modulator described herein, such as an oligonucleotide modulator, antibody modulator, peptide modulator, or small molecule modulator, are used for the preparation of a medicament for treating a patient diagnosed with or susceptible to a disease, in particular neuromuscular or neurodegenerative disease, such as, for example ALS, FTLD, or other TDP-43 proteinopathies described herein.

In other embodiments, pharmaceutical compositions comprising a modulator described herein, such as an antisense modulator, oligonucleotide modulator, nucleic acid vector, gene therapy, antibody modulator, peptide modulator, or small molecule modulator, are used for the preparation of a medicament for treating a patient diagnosed with or susceptible to a disease, such as oxidative stress, obesity, anemia and ischemic diseases, such as cardiovascular disease, myocardial ischemia and peripheral vascular disease described herein. In other embodiments, pharmaceutical compositions comprising a modulator described herein, such as an antisense modulator, nucleic acid vector, oligonucleotide modulator, nucleic acid vector, gene therapy, antibody modulator, peptide modulator, or small molecule modulator, wherein the modulator is capable of increasing the expression or activity of FLCN, are used for the preparation of a medicament for treating a patient diagnosed with or susceptible to a disease, particularly inflammatory diseases, von Hippel-Lindau (VHL) disease, Birt-Hogg-Dube (BHD) syndrome, spontaneous pneumothorax, B cell deficiency, cardiomyopathy, as well as cancers described herein.

### Overexpression of FLCN

In certain embodiments, provided herein are compositions, systems, and methods for increasing or upregulating the expression or activity of FLCN in a cell, animal, or human subject. Compositions, systems, and methods disclosed herein can be used to prevent, ameliorate, or treat diseases, particularly Birt-Hogg-Dube (BHD) syndrome, fibrofolliculomas, lung cysts, spontaneous pneumothorax, kidney tumors, and other diseases that are linked to loss-of-function of FLCN.

In some embodiments, provided herein are compositions, systems, and methods for increasing or upregulating the expression or activity of FLCN in a cell, animal, or human subject, which can be used to treat, prevent or ameliorate diseases such as inflammatory diseases, von Hippel-Lindau (VHL) disease, B cell deficiency, cardiomyopathy, as well as other cancers.

In certain embodiments, the modulators provided herein can be utilized to increase or upregulate, the expression or activity of FLCN in a cell, animal, or human subject, in order to treat, prevent or ameliorate diseases such as BHD syndrome, fibrofolliculomas, lung cysts, spontaneous pneumothorax, kidney tumors, and other diseases that are linked to loss-of-function of FLCN. In certain embodiments, the modulators provided herein can be utilized to increase or upregulate, the expression or activity of FLCN in a cell, animal, or human subject, in order to treat, prevent or ameliorate diseases such as inflammatory diseases, von Hippel-Lindau (VHL) disease, B cell deficiency, cardiomyopathy, as well as other cancers. In one embodiment, antisense modulators described herein can increase or upregulate the expression of a particular gene, such as FLCN. In one embodiment, those antisense modulators comprise antisense oligonucleotides (ASOs) and can include the characteristics, lengths, modifications, complexes, and conjugates described herein. In one embodiment, the antisense modulator targets and decreases the levels of a natural antisense transcript (NAT) that is responsible for downregulating a particular gene, thereby increasing the expression of the particular gene. In one embodiment, the antisense modulator targets and blocks a miRNA binding site present on the mRNA transcript of a particular gene that is responsible for downregulating the particular gene, thereby increasing the expression of the particular gene. In one embodiment, the antisense modulator targets and decreases the levels of a miRNA that is responsible for downregulating the expression of a particular gene, thereby increasing the expression of the particular gene. In one embodiment, the antisense modulator targets a destabilizing motif present on the mRNA transcript of a particular gene, thereby increasing the stability of the mRNA and leading to increased expression of the particular gene. In one embodiment, the antisense modulator targets a polyadenylation signal motif on the mRNA transcript of a particular gene, thereby increasing the stability of the mRNA and leading to increased expression of the particular gene. In one embodiment, the antisense modulator targets an upstream open reading frame, thereby leading to increased expression of the particular gene. In certain embodiments, methods and compositions for the delivery of antisense modulators into a cell, an animal, or a human subject described herein can be utilized to increase or upregulate the expression or activity of FLCN.

In other embodiments, modulators other than antisense modulators, for example other oligonucleotide modulators (e.g., ribozyme, deoxyribozyme, or aptamers), antibody modulators, peptide modulators, small molecule modulators, and nucleic acid vectors, described herein can be utilized to increase or upregulate the expression or activity of FLCN in a cell, an animal, or human subject. Oligonucleotide modulators also include RNAi oligonucleotide modulators, such as miRNA, siRNA, or shRNA, which can be utilized to increase or upregulate the expression or activity of FLCN in a cell, an animal, or human subject.

In certain embodiments, compositions and methods of gene therapy described herein can be utilized to increase or upregulate the expression or activity of FLCN, in order to correct for loss-of-function of FLCN, thereby treating, preventing or ameliorating a disease such as BHD syndrome, fibrofolliculomas, lung cysts, spontaneous pneumothorax, kidney tumors, and other diseases that are linked to loss-of-function of FLCN. In certain embodiments, compositions and methods of gene therapy described herein can be utilized to increase or upregulate, the expression or activity of FLCN in a cell, animal, or human subject, in order to treat, prevent or ameliorate diseases such as inflammatory diseases, von Hippel-Lindau (VHL) disease, B cell deficiency, cardiomyopathy, as well as other cancers. In one embodiment, genome editing is used to insert, delete or modify DNA sequences associated with FLCN, such as sequences described by SEQ ID NOs: 1-15. In one embodiment, genome editing is used to insert, delete, or modify RNA sequences associated with FLCN, such as sequences described by SEQ ID NOs: 1-15.

One set of embodiments provide for pharmaceutical compositions, comprising a modulator and a pharmaceutically acceptable carrier or diluent, mentioned herein which can be administered to a cell, an animal, or a human subject to increase or upregulate, the expression or activity of FLCN in the cell, animal or human subject. Such treatment methods can be used to treat, ameliorate, or prevent diseases such as BHD syndrome, fibrofolliculomas, lung cysts, spontaneous pneumothorax, kidney tumors, and other diseases that are linked to loss-of-function of FLCN. Such pharmaceutical compositions and treatment methods can also be used to treat, ameliorate, or prevent diseases, particularly inflammatory diseases, von Hippel-Lindau (VHL) disease, Birt-Hogg-Dube (BHD) syndrome, spontaneous pneumothorax, B cell deficiency, and cardiomyopathy; as well as cancers. In one embodiment, a pharmaceutical composition described herein is co-administered with one or more other pharmaceutical agents, or other drug that is typically administered to treat, ameliorate, or manage symptoms in diseases such as BHD syndrome, fibrofolliculomas, lung cysts, spontaneous pneumothorax, kidney tumors, and other diseases that are linked to loss-of-function of FLCN.

### Methods of Development

In certain embodiments, provided herein are methods of development of a modulator, such as an antisense modulator, oligonucleotide modulator, antibody modulator, peptide modulator, or small molecule modulator, which are capable of reducing or inhibiting the expression or activity of FLCN. In certain embodiments, the modulator is alternatively capable of increasing the expression or activity of FLCN. In certain embodiments, the methods of development of a modulator are entirely computational. In some embodiments, the methods of development of a modulator involve biochemical methods, such as screens and selections. In some embodiments, the methods of development of a modulator involve a combination of biochemical and computational methods. Such methods of development involve standard practices that are known to a person who is skilled in the art and are incorporated in certain embodiments herein.

In some embodiments, computational methods can involve artificial intelligence or machine learning software, rely on large molecular databases, utilize high throughput analysis, or any combination thereof. In one embodiment, a computational method can be used to screen existing drug candidates, which can be repurposed for use as modulators herein. Certain methods for computational drug repurposing are described by Li et al. (Li et al., Briefings in Bioinformatics, 17(1): 2-12 (2016)) and Hodos et al. (Hodos et al., Wiley Interdisciplinary Reviews: Systems Biology and Medicine, 8(3): 186-210 (2016)), which, along with the references cited therein, are incorporated by reference herein in their entirety. Other computational methods are well known to a person of ordinary skill in the art and are included in various embodiments herein.

### Antisense Modulator

In certain embodiments, provided herein are methods of development of antisense modulators that are capable of targeting one or more transcripts of FLCN described by SEQ ID NOs: 1-15, or its associated genes or pathways, thereby reducing or inhibiting the expression or activity of FLCN. In some embodiments, the antisense modulator can instead increase the expression or activity of FLCN. Antisense modulators include compounds that do not act through the RNAi pathway, such as antisense oligonucleotides, as well as compounds that act through the RNAi pathway, such as siRNA, shRNA, or miRNA. The design of an appropriate antisense modulator is critical to its safety and effectiveness as a therapy.

Several guiding principles can be used individually or in conjunction with one another to design antisense oligonucleotide modulators. Firstly, the sequence of the modulator should be antisense to the target genetic sequence of choice. However, mismatches or imperfect complementarity between the antisense oligonucleotide modulator and target sequence can be tolerated as described elsewhere herein. Secondly, the sequence of the antisense oligonucleotide modulator is ideally not more than 10, 12, 14, 16, 18, 20, 22, 24, 26, 28, 30, 32, 34, 36, 38, or 40 nucleotides in length. In a preferred embodiment, the antisense oligonucleotide modulator is between 12 to 30 nucleotides in length. Thirdly, the antisense oligonucleotide modulator ideally targets a region of the target nucleic acid that is accessible and does not contain stable secondary structures. Fourthly, the antisense oligonucleotide modulator should possess sufficient binding energy to the target nucleic acid molecule. The antisense oligonucleotide modulator can be modified to improve stability, delivery, and efficacy, as described elsewhere herein. Methods and procedures for designing and developing antisense oligonucleotides are well known in the art, for example, as described by Chan et al. (Chan et al., Clinical and Experimental Pharmacology and Physiology, 33:533-540 (2006)), which along with references cited therein, is incorporated by reference in its entirety. Delivery systems for antisense oligonucleotides are described herein and further described by Chan et al. and Zhao et al. (Zhao et al., Expert Opinion on Drug Delivery, 6(7): 673-686 (2009)), both of which along with references cited therein, are incorporated by reference in their entirety and are known to a person of ordinary skill in the art.

The methods detailed below are relevant to the screening and development of siRNA, shRNA, or miRNA, and any procedures detailed below for the screening of siRNA are also applicable to shRNA and miRNA. The methods detailed below are also relevant to the screening and development of antisense oligonucleotides, and any procedures detailed below for the screening of siRNA are also applicable to antisense oligonucleotide modulators.

The design of an appropriate siRNA sequence is critical to the performance of RNAi therapies. siRNA sequences are chosen based on several guiding principles that can be used individually or in conjunction with one another. Firstly, the siRNA should be antisense to the target genetic sequence of choice. Secondly, the siRNA sequence can ideally be chosen to be in the range of 19 to 29 nucleotides in length. Thirdly, the target area of the genetic sequence should be at least 100 nucleotides from the initiation codon and 50 nucleotides away from the stop codon. Fourthly, the introduction of 3′-d(TT) overhangs are recommended. Fifthly, structural considerations should be made to promote stability and effectiveness of the siRNA (e.g., the GC ratio limited to between 45% and 55%, or the elimination of poly-C or poly-G sequences). Certain tools (e.g., http://sirna.wi.mitedu/) can be used to assist in the development of appropriate siRNA designs. A BLAST search is recommended to be performed to eliminate siRNA candidates with low specificity to the targeted genetic sequence. In certain cases, the siRNA sequence or multiple siRNA sequences can be integrated into a strand of double-stranded RNA. The siRNA modulator can be modified to improve stability, delivery, and efficacy, as described elsewhere herein. Methods and procedures for developing oligonucleotides for RNAi treatment are further described by Duxbury and Whang (Duxbury and Whang, Journal of Surgical Research, 117:339-344 (2004)), which along with references cited therein, are incorporated by reference in its entirety and are known to a person of ordinary skill in the art. Delivery systems for RNAi treatments are further described by Deng et al. (Deng et al., Gene, 538(2):217-227 (2014)), which along with references cited therein, is incorporated by reference in its entirety and are known to a person of ordinary skill in the art.

### Small Molecule

In certain embodiments, provided herein are methods of development of small molecule modulators that are capable of targeting FLCN, or its associated genes or pathways, thereby reducing or inhibiting the expression or activity of FLCN. In some embodiments, the small molecule modulators can instead increase the expression or activity of FLCN. In some embodiments, methods of development of small molecule modulators include computational methods. Computational methods described by Mendez-Lucio et al. (Mendez-Lucio et al., Nature Communications, 11, 10 (2020)), Dallakyan and Olson (Dallakyan and Olson, Hempel et al. (ed.) Chemical Biology: Methods and Protocols, Chapter 19, Methods in Molecular Biology pg 243-250 (2015)), Zoete et al. (Zoete et al., Journal of Chemical Information and Modeling, 56: 1399-1404 (2016)), and Merk et al. (Merk et al., Molecular Informatics, 37: 1700153 (2018)) are representative of some of the computational methods available, and these reference along with references cited therein, are incorporated by reference herein. In one embodiment, a computational library of small molecule modulators is computationally docked individually with a target protein, such as FLCN. In one embodiment, a computational method is used to determine the binding energy of each small molecule modulator to a target protein, such as FLCN. In yet another embodiment, new small molecule modulators are created for computational screening from an amalgamation of existing molecules or atoms from one or more databases. In certain embodiments, small molecule modulators that are predicted to have favorable binding energies to the target protein are prioritized for further analysis and development.

In certain embodiments, methods of development of small molecule modulators include high throughput biochemical screening methods, which are known to a person of ordinary skill in the art. Physical libraries of small molecules are built or obtained from commercially available sources. These libraries are screened against a target molecule of choice, such as FLCN, by introducing the small molecules to the target molecule of choice and then implementing a washing or separating method to determine binding affinity and/or specificity. A biochemical or cell-based assay or a series of biochemical or cell-based assays can be used to determine structural and chemical properties of the small molecules or molecular complexes that are formed between the small molecule and the target molecule of choice. Controls or negative selection steps can be used to screen out small molecules with off-target binding activity to other molecules that are not the target molecule of choice. Further, screening at different concentrations of the small molecule against the target molecule of choice is used to determine other properties such as IC50, EC50, potency, and the like. Further description of methods and procedures for developing small molecule modulators via high-throughput screening are described by Cronk (Cronk, Drug Discovery and Development (Second Edition) Chapter 8, pp. 95-117 (2013)), which along with references cited therein, are incorporated by reference in its entirety and are known to a person of ordinary skill in the art. Other methods are well known to a person of ordinary skill in the art and are included in various embodiments herein.

In certain embodiments, methods of development of small molecule modulators include fragment-based discovery techniques that are known to a person of ordinary skill in the art. These methods involve screening of a library of small molecular fragments that contain one or more binding epitopes for binding affinity and/or specificity to a target molecule, such as FLCN. Typically, the small molecular fragments have a molecular mass of around 120-250 Daltons. In certain cases, these fragment-based discovery methods are combined with computational methods, some of which are described below. Examples of fragment-based discovery techniques include lead identification by fragment evolution, lead identification by fragment linking, lead identification by fragment self-assembly, and lead progression by fragment optimization, which are described herein. Often, assessment of target molecule binding sites, the development of fragment complexes, and subsequent determination of efficacy or specificity of binding is informed by structural, morphological, and chemical data acquired from assessment tools such as nuclear magnetic resonance spectroscopy, mass spectrometry, or X-ray crystallography.

In lead identification by fragment evolution, a library of fragments is applied to the target molecule of choice, and the strength and specificity of binding is determined. The fragments with higher binding specificity are then reacted or evolved with various other fragments to form fragment complexes that are screened for having even higher binding specificity.

In lead identification by fragment linking, fragment libraries are screened through multiple binding sites of a target molecule of choice for binding specificity. Two or more fragments with high binding specificity to two or more nearby binding sites on a target molecule of choice are chemically linked together.

In lead identification by fragment self-assembly, also termed combinatorial chemistry, a library of fragments capable of self-assembly is introduced to a target molecule of choice. The fragments are allowed to bind to the target molecule of choice in a manner that produces a complex that inhibits the expression or activity of the target molecule of choice. The various fragments are capable of assembling together while bound to the target molecule of choice via complementary reactive groups. Once assembled, these fragment complexes can then be isolated to assess their chemical and structural properties.

In lead progression by fragment optimization, a library of fragments is used to modify the properties of an existing modulator or fragment complex. Typically, this method is used to address the optimization of certain properties, such as selectivity, solubility, stability, or efficacy.

Examples and further discussion of methods and procedures for the development of small molecule modulators via fragment-based discovery are described by Rees et al. (Rees et al., Nature Reviews Drug Discovery, 3(8): 660 (2004)), Erlanson et al. (Erlanson et al., Journal of Medicinal Chemistry, 47(14): 3463-3482 (2004)), and Congreve et al. (Congreve et al., Journal of Medicinal Chemistry, 51(13): 3661-3680 (2008)), all of which along with references cited therein, are incorporated by reference in its entirety and are known to a person of ordinary skill in the art. Other methods are well known to a person of ordinary skill in the art and are included in various embodiments herein.

### Antibody and Related Protein

In certain embodiments, provided herein are methods of development of antibody and other related protein modulators that are capable of targeting FLCN, or its associated genes or pathways, thereby reducing or inhibiting the expression or activity of FLCN. In some embodiments, the antibody and related protein modulator can instead increase the expression or activity of FLCN. In some embodiments, computational methods are used to develop antibody and related protein modulators. Computational methods as described by Chevalier et al. (Chevalier et al., Nature, 550, 7674 (2017)) are representative of some of the computational methods available, and this reference along with references cited therein, are incorporated by reference herein. In other embodiments, biochemical methods are used to develop, screen and produce antibody or related protein modulators. Such methods use a common set of methods that are known to a person of ordinary skill in the art. Throughout this specification, the antibodies referred to include all forms of antibodies, including but not limited to, monoclonal antibodies, polyclonal antibodies, chimeric antibodies, humanized antibodies, antibody fragments, and monobodies.

In certain embodiments, antibodies are developed by introducing an antigen that activates an immunological response in an animal species, such as, but not limited to, rabbit, mouse, rat, hamster, guinea pig, goat, sheep, chicken, or humans, and harvesting the resulting antibodies from blood or, in some cases, eggs, tissue, or other fluids. In certain embodiments, an adjuvant is also used alongside the antigen to increase the level of immunological response. Examples of commonly used adjuvants include, but are not limited to, Freund's complete adjuvant, Freund's incomplete adjuvant, aluminum salts, Quil A, Iscoms, Montanide, TiterMax, and RIBI.

Methods of development or production of polyclonal antibodies are known to persons skilled in the art. One such process can include either single or multiple introductions of at least one antigen or antigen/adjuvant mixture into the animal species of choice, clinical monitoring of antibody levels, and antibody collection once sufficient antibody levels are reached.

Methods of development or production of monoclonal antibodies are known to persons skilled in the art. In one common embodiment, BALB/c mice are used as the animal species for antigen injection, although other species mentioned above can be used. One such process can include either single or multiple introductions of at least one antigen or antigen/adjuvant mixture into the animal species of choice and clinical monitoring of antibody levels. A final injection of just antigen with no adjuvant is typically administered prior to harvesting B cells from the animal. The B cells are fused with non-secreting myeloma cells to form hybridoma B cells, which are cloned and selected for antigen specificity to a target molecule using one of any number of screening methods known to persons skilled in the art. Typical production processes utilize antigen-specific hybridoma B cells to generate identical copies of the antibody. Other methods, such as in vitro display selection (see below), can be used for the development of monoclonal antibodies and are known to a person of ordinary skill in the art.

In certain embodiments, provided herein are methods to further increase production levels of monoclonal antibodies. In the ascites method, the chosen monoclonal antibody-producing hybridoma cells are isolated and injected into an animal species, such as mice, which instigates the growth of ascites in areas, such as the abdominal cavity. In certain cases, a priming agent, such as pristine, is first injected into the animal to suppress immune response, promote the secretion of serous fluid, and slow hybridoma cell clearance. After incubation for several days to weeks, antibodies can be extracted from the animal directly from the ascites. In in vitro methods for increasing production levels of monoclonal antibodies, the chosen monoclonal antibody-producing hybridoma cells are isolated and cultured, often in a nutrient medium and/or serum. Single-compartment culture systems, such as culture flasks, roller bottles, or gas-permeable bags can be used for smaller scale production. In larger scale production, double-compartment culture systems, such as hollow-fiber systems, fermenters, perfusion-tank systems, airlift reactors, or continuous-culture systems can be used.

In certain embodiments, provided herein are methods for the development or production of chimeric or humanized antibodies, which are known in the art. The development or production of chimeric or humanized antibodies can be accomplished through genetic engineering of an animal genome to contain human or human-like coding segments in the antibody. Further modification of antibodies can also be carried out by genetic engineering of the genome of B cells, hybridoma cells, or vectors.

A 1999 report by the National Research Council (Monoclonal Antibody Production: A Report of the Committee on Methods of Producing Monoclonal Antibodies, Institute for Laboratory Animal Research, National Research Council (1999)) and an article by Leenars and Hendriksen (Leenars and Hendriksen, Ilar Journal, 46(3): 269-279 (2005)) detail further certain procedures for synthesizing antibodies, both of which along with references cited therein, are incorporated by reference in their entirety and are known to a person of ordinary skill in the art. Cell-free antibody synthesis procedures are described by Stech and Kubick (Stech and Kubick, Antibodies, 4: 12-33 (2015)), which along with references cited therein, is incorporated by reference in its entirety and are known to a person of ordinary skill in the art.

In one embodiment, an antibody modulator is screened for and developed using an antibody development, screening, or production method, or any combination of methods thereof detailed above.

### Aptamer

In certain embodiments, provided herein are methods of development of aptamer modulators, including oligonucleotide, oligopeptide, or polypeptide aptamers, which are capable of targeting FLCN, or its associated genes or pathways, thereby reducing or inhibiting the expression or activity of FLCN. In some embodiments, the aptamer modulator can instead increase the expression or activity of FLCN. In certain embodiments, in vitro selection, also referred to as SELEX or in vitro evolution, can be used to develop and screen for aptamer modulators, such as oligonucleotide aptamers, which have strong binding affinities to a target molecule of choice, such as FLCN. In vitro selection methods are known to a person of ordinary skill in the art, and are incorporated in certain embodiments herein. In the first step, a large population of degenerate oligonucleotides is synthesized and amplified to create an oligonucleotide library using polymerase chain reaction. In the second step, the oligonucleotide library is treated with a change in temperature to renature the oligonucleotide library into stable secondary or tertiary structures. In the third step, the oligonucleotide library is introduced to the target molecule of choice, which has been immobilized to a substrate. In the fourth step, the oligonucleotides that are bound to the target molecule of choice are isolated from the rest of the oligonucleotide library via a washing method or other related method. In the fifth step, the oligonucleotides that are bound to the target molecule of choice are eluted from the target molecule of choice, typically using an oligonucleotide denaturing method such as high temperature or application of denaturing solutions, and isolated. In the sixth step, the oligonucleotides are further amplified by polymerase chain reaction and converted into the desired oligonucleotide format (e.g., single-stranded DNA, single-stranded RNA, or other oligonucleotide format). In the seventh step, the second step through the sixth step are repeatedly cycled until the desired specificity and/or binding affinity of the oligonucleotides to the target molecule of choice is reached. In certain instances, a negative selection step can be implemented between repeated cycles during which other target molecules can be used to bind to and remove oligonucleotides with specificity to those other target molecules. Other methods are well known to a person of ordinary skill in the art and are included in various embodiments.

In certain embodiments, provided herein are methods of in vitro display to develop and screen for oligopeptide and polypeptide aptamers that have strong binding affinities to a target molecule of choice, such as FLCN. In vitro display methods are known to a person of ordinary skill in the art and include, but are not limited to, phage display, cell surface display, ribosome display, mRNA display, DNA display, and in vitro compartmentalization.

Phage display can be used as a selection process that identifies and screens the binding affinity and specificity of a collection of oligopeptide or polypeptide aptamers to a target molecule of choice, using common strains of bacteriophage (e.g., M13, fd, or fl) as a means of surface peptide display. In the first step, a combinatorial library of oligopeptide or polypeptide fragments is identified, and a nucleotide library is built encoding the combinatorial library of peptide fragments. In the second step, the nucleotide sequences from the nucleotide library are coupled to nucleotide sequences encoding the major or minor coat proteins in the genome of the bacteriophage of choice. In the third step, the bacteriophages are introduced into a bacteria host (e.g., E. coli) to be replicated. The bacteriophages that are replicated express the various peptide fragments from the combinatorial library on the bacteriophage surface and contain the corresponding modified genome. In the fourth step, the bacteriophages are introduced to the target molecules of choice that have been immobilized on a substrate. In the fifth step, the bacteriophages that are bound to the target molecule of choice are isolated from the rest of the bacteriophages via a washing method or other related method. In the sixth step, the remaining, bound bacteriophages are eluted via a method such as increased temperature or a denaturing solution. In the seventh step, the eluted bacteriophages are cycled through the third through the sixth step until the desired level of binding affinity and/or specificity towards the target molecule of choice is achieved. In the eighth step, the genetic sequence of the polypeptide or oligopeptide aptamer with the desired level of specificity and/or binding affinity towards the target molecule of choice is isolated from the bacteriophage and can be used to produce additional copies of the polypeptide or oligopeptide aptamer. In certain instances, a negative selection step can be implemented between repeated cycles during which other target molecules can be used to bind to and remove polypeptide or oligopeptide aptamers with specificity to those other target molecules.

Cell surface display can be used as a selection process that identifies and screens the binding affinity and specificity of a collection of oligopeptide or polypeptide aptamers to a target molecule of choice using a cell or collection of cells (e.g., bacteria or yeast) as a means of surface peptide display. The selection process of aptamers using cell surface display is similar to that of phage display. In the first step, a combinatorial library of peptide fragments is identified, and the nucleotide library is built encoding the combinatorial library of peptide fragments. In the second step, the nucleotide sequences from the nucleotide library are coupled to nucleotide sequences encoding an outer membrane protein and introduced into the cell of choice, either through transformation, introduction via a vector, or other similar method known in the art. In the third step, the cell is allowed to replicate as well as to transcribe and translate the nucleotide sequence encoding the peptide fragment linked to an outer membrane protein such that the peptide fragment is displayed on the cell surface. In the fourth step, the cells are introduced to the target molecules of choice that have been immobilized on a substrate. In the fifth step, the cells that are bound to the target molecule of choice are isolated from the rest of the cells via a washing method or other related method. In the sixth step, the remaining, bound cells are eluted via a method such as increased temperature or a denaturing solution. In the seventh step, the eluted cells are cycled through the third through the sixth step until the desired level of specificity and/or binding affinity towards the target molecule of choice is achieved. In the eighth step, the genetic sequence of the polypeptide or oligopeptide aptamer with the desired level of specificity towards the target molecule of choice is isolated from the cell, which can be used to produce additional copies of the aptamer. In certain instances, a negative selection step can be implemented between repeated cycles during which other target molecules can be used to bind to and remove polypeptide or oligopeptide aptamers with specificity to those other target molecules.

Ribosome display can be used as a selection process that identifies and screens for polypeptide or oligopeptide aptamers that bind with high affinity and/or specificity to a target molecule of choice, such as FLCN. The ribosome display process involves a library of mRNA molecules that code for the library of aptamers identified for screening. These mRNA molecules are modified to have a ribosome binding site at the 5′ end and a spacer sequence with no stop codon on the 3′ end. In the first step, the modified mRNA molecules are prepared. In the second step, the modified mRNA molecules are translated in vitro, which causes an mRNA-peptide-ribosome complex to be formed. In the third step, the mRNA-peptide-ribosome complex is isolated and introduced to the target molecules of choice that have been immobilized on a substrate. In the fourth step, the complexes that bind to the target molecules of choice are sorted and isolated. In the fifth step, the isolated complexes bound to the target molecules of choice are eluted and dissociated, and the mRNA molecules that correspond to those complexes with preferential binding to the target molecules of choice are recovered. In the sixth step, those mRNA molecules are reverse transcribed and amplified via polymerase chain reaction to produce DNA sequences encoding aptamers with high specificity and/or binding affinity to the target molecules of choice. In the seventh step, the first step through the sixth step are repeated using the nucleotides obtained from the previous sixth step until the desired level of specificity and/or affinity towards the target molecules of choice is obtained. The genetic sequence of the aptamer can then be used to produce additional copies of the aptamer. In certain instances, a negative selection step can be implemented between repeated cycles during which other target molecules can be used to bind to and remove polypeptide or oligopeptide aptamers with specificity to those other target molecules.

mRNA display can be used as a selection process that identifies and screens for polypeptide or oligopeptide aptamers that bind with high affinity and/or specificity to a target molecule of choice, such as FLCN. The mRNA display process involves creating a library of mRNA molecules that code for the library of aptamers identified for screening. These mRNA molecules are modified to have a short DNA linker with puromycin at the 3′ end. In the first step, these modified mRNA molecules are prepared. In the second step, the modified mRNA molecules are translated in vitro, which causes an mRNA-peptide complex to be formed from the reaction between puromycin and the nascent polypeptide. In the third step, the mRNA-peptide complex is isolated and introduced to the target molecules of choice that have been immobilized on a substrate. In the fourth step, the complexes that bind to the target molecules of choice are sorted and isolated. In the fifth step, the isolated complexes bound to the target molecules of choice are eluted and dissociated, and the mRNA molecules that encode for oligopeptide or polypeptide aptamers with high affinity and/or specificity to the target molecules of choice are recovered. In the sixth step, those mRNA molecules are reverse transcribed and amplified via polymerase chain reaction to form DNA sequence encoding the aptamers. In the seventh step, the first step through the sixth step are repeated using the nucleotides obtained from the previous sixth step until the desired level of specificity and/or affinity towards the target molecules of choice is obtained. The genetic sequence of the aptamer can then be used to produce additional copies of the aptamer. In certain instances, a negative selection step can be implemented between repeated cycles during which other target molecules can be used to bind to and remove polypeptide or oligopeptide aptamers with specificity to those other target molecules.

DNA display can be used as a selection process that identifies and screens for polypeptide or oligopeptide aptamers that bind with high affinity and/or specificity to a target molecule of choice, such as FLCN. The DNA display process involves a library of DNA sequences that code for the library of aptamers identified for screening linked to DNA sequence encoding the protein streptavidin, which forms the conjugate. These DNA sequences are further labeled with biotin. In the first step, the modified DNA sequences are prepared and mRNA molecules are transcribed from these DNA sequences in separate compartments containing on average one member of the DNA library. In the second step, the mRNA molecules are translated in vitro, which produces a polypeptide or oligopeptide aptamer linked to streptavidin, which binds to the biotin-labeled DNA. In the third step, the DNA-peptide complex is isolated and introduced to the target molecules of choice that have been immobilized on a substrate. In the fourth step, the complexes that bind to the target molecules of choice are sorted and isolated. In the fifth step, the isolated complexes bound to the target molecules of choice are eluted and dissociated, and the DNA molecules that encode the aptamers with preferential binding to the target molecules of choice are recovered. In the sixth step, those DNA molecules are amplified via polymerase chain reaction to obtain the genetic sequence of aptamers that possess high binding affinity and/or specificity to the target molecules of choice. In the seventh step, the first step through the sixth step are repeated using the nucleotides obtained from the previous sixth step until the desired level of specificity and/or affinity towards the target molecules of choice is obtained. The genetic sequence of the aptamer can then be used to produce additional copies of the aptamer. In certain variations of this method, a different pair of molecules with high binding affinity to each other, besides streptavidin and biotin, can be used for both attachment to the DNA sequence and its corresponding conjugate that is translated from the DNA sequence that is linked to the aptamer. These are able to create similar DNA-peptide complexes described above. In other variations, puromycin or other similar protein can be attached to the DNA sequence. The puromycin can bind directly to the nascent protein without the need for a conjugate polypeptide strand to form a DNA-peptide complex, as described by Chen et al. (Chen et al., RSC Advances 3, 16251 (2013)). In certain instances, a negative selection step can be implemented between repeated cycles during which other target molecules can be used to bind to and remove polypeptide or oligopeptide aptamers with specificity to those other target molecules. Other methods are well known to a person of ordinary skill in the art and are included in various embodiments herein.

### Gene Therapy

In certain embodiments, provided herein are methods of development of molecules for gene therapy to reduce or inhibit the expression or activity of FLCN, thereby treating, preventing, or ameliorating a disease such as ALS, or other TDP-43 proteinopathy. In some embodiments, the gene therapy can instead increase the expression or activity of FLCN. Methods of development of molecules for gene therapy are known in the art and are included in various embodiments herein. In the case of developing gene therapy where the goal is gene augmentation, a nucleotide sequence encoding one or more functional copies of the gene of interest can be inserted into a nucleic acid vector. In one embodiment, functional copies of the gene are placed under control of appropriate regulatory elements, such as a tissue-specific promoter, wherein the gene is expressed at appropriate levels and/or at appropriate times and/or in appropriate tissues to rescue the defect caused by loss-of-function of the gene. In one embodiment, the promoter is a CNS-specific promoter. In another embodiment, a modulator that can increase the activity or expression of the gene of interest is inserted into a nucleic acid vector. In another embodiment, the nucleic acid vector carrying the gene of interest is designed to target a specific tissue, such as by selecting an appropriate viral vector that is specific to a particular tissue, or by any other means known in the art. In the case of developing gene therapy where the goal is gene suppression, a nucleotide sequence encoding at least one modulator that can suppress the expression, or the activity of the gene of interest, such as FLCN, is inserted into a nucleic acid vector. Methods of development of gene therapy for clinical applications are described in Sung et al. (Sung et al., Biomaterials Research, 23, Article number: 8 (2019)) and Kumar et al. (Kumar et al., Mol. Ther. Methods Clin. Dev., 3, 16034 (2016)), which together with the references cited therein, are incorporated herein in their entirety.

In the case of developing gene therapy where the goal is gene editing, the development method depends on the particular endonuclease system and desired effects on the cell. A key consideration is ensuring the specificity of targeting of the endonuclease to a specific DNA or RNA sequence, in order to reduce side effects from mis-targeting of the endonuclease. In the case of Transcription activator-like effector nucleases (TALEs/TALENs), DNA specificity is determined by a DNA Binding Domain (DBD) containing on average 1.5-33.5 tandem repeats of 34 amino acid sequences (termed monomers), wherein each monomer recognizes a specific nucleotide. Although the sequence of each monomer is highly conserved, they differ primarily in two positions (the 12th and 13th) named as repeat variable di-residues (RVDs). Recent reports have found that the identity of these two residues determines the nucleotide binding specificity of each TALE repeat in a simple cipher that specifies the target base of each RVD (NI=A, HD=C, NG=T, NN=G). Thus, each monomer targets one nucleotide, and the linear sequence of monomers in a TALE specifies the target DNA sequence in the 5′ to 3′ direction. In one embodiment, a computational method is employed to design an appropriate sequence of monomers in a TALE to target a specific DNA sequence. Methods for developing TALE/TALENs are described in Zhang et al. (Zhang et al., Molecular Therapy: Methods & Clinical Development, 13, 310-320 (2019)), which along with references cited therein, is incorporated by reference herein in their entirety. Although less modular than TALENs, Zinc-finger nucleases (ZFN) can be designed to target a specific DNA sequence through a combination of certain design rules coupled with in vitro selection techniques. Such development and screening methods for ZFNs are described in detail in Chandrasegaran and Carroll (Chandrasegaran and Carroll, Journal of Molecular Biology, 428(5), 963-989 (2016)), which along with the references cited therein, are incorporated by reference in its entirety herein.

In one embodiment of the CRISPR system, the specificity of targeting is determined by a gRNA, which directs the endonuclease to bind to a specific DNA or RNA sequence that is complementary to the gRNA. Several studies have established key rules for the design of gRNA to increase the specificity of targeting a nucleotide sequence. For example, in the case of the Cas9 nuclease, a canonical protospacer adjacent motif (PAM) site comprising the nucleotides NGG, where N is any nucleobase, must be present immediately 3′ to the sequence that is targeted by the gRNA. In the case of the Cpf1 (Cas12a) nuclease, the PAM comprises “TTTN” or “YTN”. In addition, key rules for optimizing gRNA design include avoiding poly-T sequences, limiting the GC content and avoiding a G immediately upstream of the PAM (i.e., a GNGG motif). Furthermore, it is important to check for potential off-target sites that are similar in sequence to the target site. Several computational tools have been developed for the design and/or screening of specific gRNAs and the prediction of off-target sites. Such development and screening methods for CRISPR-Cas9 are described in Wilson et al. (Wilson et al., Frontiers in Pharmacology, 9, 749 (2018)), which along with the references cited therein, are incorporated by reference herein in its entirety.

### Diagnostics and Testing

In some embodiments, measuring and detecting an increase in expression levels of FLCN, or measuring and detecting an increase in activity of FLCN, can be used to determine an increased risk for or increased susceptibility to ALS. In certain embodiments, measuring and detecting an increase in signaling through FLCN can be used to determine an increased risk for or increased susceptibility to ALS. In certain embodiments, measuring and detecting an increase in signaling through a pathway associated with FLCN, can be used to determine an increased risk for, or increased susceptibility to ALS. In some embodiments, measuring and detecting a decrease in expression levels of FLCN, or measuring and detecting a decrease in activity of FLCN, can be used to determine a decreased risk for, or decreased susceptibility to ALS. In certain embodiments, measuring and detecting a decrease in signaling through FLCN can be used to determine a decreased risk for, or decreased susceptibility to ALS. In certain embodiments, measuring and detecting a decrease in signaling through a pathway associated with FLCN, can be used to determine a decreased risk for, or decreased susceptibility to ALS.

In certain embodiments, measuring and detecting an increase in expression or activity of FLCN, or an increase in signaling through a pathway associated with FLCN, can be used to determine an increased risk for or increased susceptibility to neuromuscular or neurodegenerative diseases, such as FTLD, Alzheimer's disease, retinal degeneration diseases such as age-related macular degeneration (AMD), or other TDP-43 proteinopathies; as well as oxidative stress, obesity, anemia or ischemic diseases, such as cardiovascular disease, myocardial ischemia and peripheral vascular disease. In other embodiments, measuring and detecting a decrease in expression or activity of FLCN, or a decrease in signaling through a pathway associated with FLCN, can be used to determine an increased risk for or increased susceptibility to inflammatory diseases, von Hippel-Lindau (VHL) disease, Birt-Hogg-Dube (BHD) syndrome, spontaneous pneumothorax, B cell deficiency, cardiomyopathy, as well as cancers such as fibrofolliculomas, kidney tumors, clear cell renal cell carcinoma, multilocular clear cell renal carcinoma, chromophobe renal cell carcinoma, renal oncocytic hybrid carcinoma, bladder cancer, uterine corpus endometrioid cancer, interdigitating dendritic cell sarcoma, hemangioblastomas, pancreatic neuroendocrine tumors, pheochromocytomas, endolymphatic sac tumors, kidney cysts, and lung cysts.

In one embodiment, a diagnostic method for determining a subject's susceptibility to ALS comprises obtaining nucleic acid sequence data from that subject, detecting the presence or absence of at least one allele of at least one polymorphic marker (or markers in linkage disequilibrium therewith) present within at least one genomic region associated with FLCN, such as but not limited to genomic regions found in SEQ ID NOs: 1-15, wherein different alleles are associated with different susceptibilities to ALS, and determining a susceptibility to ALS from the nucleic acid sequence data. In one embodiment, the at least one allele associated with susceptibility to ALS is present within an exon of FLCN described in Tables 1-5 that encodes for the FLCN protein. In another embodiment, the at least one allele associated with susceptibility to ALS is located within a non-exonic (i.e. non-coding) region of FLCN that affects the expression of FLCN, such as, for example, a promoter, an enhancer, an intron, a 5′ UTR or a 3′ UTR. In another embodiment, a diagnostic method for determining a subject's susceptibility to ALS comprises obtaining a sample, including from tissue, fluids, or other sample containing cellular material, from a subject, analyzing the sample for concentration and/or polymorph(s) of FLCN protein, comparing to known and/or calibrated control samples, and determining a susceptibility to ALS. In yet another embodiment, a diagnostic method for determining a subject's susceptibility to ALS comprises obtaining nucleic acid sequence data from the subject, such as for example RNA-seq or cDNA data, detecting the expression levels of at least one transcript that is associated with a sequence described by SEQ ID NOs: 1-15, wherein different expression levels of the transcript(s) are associated with different susceptibilities to ALS, and determining a susceptibility to ALS from the nucleic acid sequence data.

In certain embodiments, the methods of determining risk or susceptibility to ALS, or methods of diagnosis of ALS stated above, can be applied to predict prognosis of a human individual diagnosed with, or experiencing symptoms associated with, ALS. In other embodiments, the methods of determining risk or susceptibility to ALS, or methods of diagnosis of ALS stated above, can be used to assess a human individual for a probability of a response to a therapeutic method and/or modulator used to treat, prevent or ameliorate symptoms associated with ALS. In one embodiment, such methods can be used to select a modulator used in treating a subject with ALS.

In yet other embodiments, the methods of determining risk or susceptibility to ALS, or methods of diagnosis of ALS, or methods of predicting prognosis of a human individual diagnosed with, or experiencing symptoms associated with ALS, or methods of assessing a human individual for a probability of a response to a therapeutic method and/or modulator used to treat, prevent or ameliorate symptoms associated with ALS, can be applied to other diseases, particularly neuromuscular or neurodegenerative diseases, such as, for example, FTLD, Alzheimer's Disease, retinal degeneration diseases such as age-related macular degeneration (AMD), and other TDP-43 proteinopathies disclosed herein. In yet other embodiments, the methods of determining risk or susceptibility to ALS, or methods of diagnosis of ALS, or methods of predicting prognosis of a human individual diagnosed with, or experiencing symptoms associated with ALS, or methods of assessing a human individual for a probability of a response to a therapeutic method and/or modulator used to treat, prevent or ameliorate symptoms associated with ALS, can be applied to other diseases, such as, for example, oxidative stress, obesity, anemia, ischemic disease, inflammatory disease, von Hippel-Lindau (VHL) disease, Birt-Hogg-Dube (BHD) syndrome, spontaneous pneumothorax, B cell deficiency, cardiomyopathy, or cancer described herein.

### Kits

Some embodiments also relate to kits and apparatuses for determining susceptibility of a human individual to ALS; or for diagnosing ALS; or predicting prognosis of a human individual diagnosed with, or experiencing symptoms associated with ALS; or assessing a human individual for a probability of a response to a therapeutic method and/or modulator used to treat, prevent or ameliorate symptoms associated with ALS.

Some embodiments also relate to kits and apparatuses for determining susceptibility of a human individual to a disease; or for diagnosing a disease; or predicting prognosis of a human individual diagnosed with, or experiencing symptoms associated with a disease; or assessing a human individual for a probability of a response to a therapeutic method and/or modulator used to treat, prevent or ameliorate symptoms associated with a disease, wherein the disease is a neuromuscular or neurodegenerative disease, such as, for example, FTLD, Alzheimer's Disease, retinal degeneration disease such as age-related macular degeneration (AMD), other TDP-43 proteinopathy, oxidative stress, obesity, anemia, ischemic disease, inflammatory disease, von Hippel-Lindau (VHL) disease, Birt-Hogg-Dube (BHD) syndrome, spontaneous pneumothorax, B cell deficiency, cardiomyopathy, or cancer described herein.

Kits that are useful in any of the methods described herein can comprise of any component that is useful in any of the methods described herein, including but not limited to probes (e.g., hybridization probes, allele-specific oligonucleotides), enzymes (e.g., for RFLP analysis, activity assays), reagents for amplification of nucleic acids, reagents for direct analysis of at least one allele of at least one polymorphic marker within or associated with FLCN, reagents for indirect analysis of at least one allele of at least one polymorphic marker within or associated with FLCN, etc. In one embodiment, the kit can include necessary buffers. In another embodiment, the kit can additionally provide reagents for other ALS diagnostic methods known in the art to be carried out in conjunction with the methods described herein.

In certain embodiments, the reagents in the kits include at least one contiguous oligonucleotide, such as for example described in SEQ ID NOs: 16-618, which is capable of hybridizing to a fragment of the genome of the individual containing at least one allele of at least one polymorphic marker within or associated with FLCN, or markers in linkage disequilibrium therewith. In another embodiment, the reagents in the kits comprise at least two oligonucleotide primers, such as for example described in SEQ ID NOs: 16-618, which are designed to amplify a fragment of the genome of the individual containing at least one allele of at least one polymorphic marker within or associated with FLCN, or markers in linkage disequilibrium therewith. Furthermore, the oligonucleotide(s) in the kits can contain mismatches to the fragment of the genome, as is well known to a skilled person in the art. In another embodiment, the kit comprises at least one or more labeled oligonucleotides and reagents for detection of the label. Suitable labels can include but are not limited to a radioisotope, a fluorescent label, an enzyme label, an enzyme co-factor label, a magnetic label, a spin label, or an epitope label.

In some embodiments, the kits and apparatuses include a collection of data comprising correlation data between the at least one allele of at least one polymorphic marker within or associated with FLCN that is selectively assessed by the kit and susceptibility to ALS, or prognosis for ALS, or response to at least one ALS therapy. In certain embodiments, the kits and apparatuses include a collection of data comprising correlation data between the expression levels of at least one transcript associated with a sequence described by SEQ ID NOs: 1-15 that is selectively assessed by the kit, and susceptibility to ALS, or prognosis for ALS, or response to at least one ALS therapy. Another set of embodiments relates to methods of use of at least one oligonucleotide probe in the manufacture of a diagnostic reagent for diagnosing and/or assessing the susceptibility to ALS in a human individual, wherein the probe is capable of hybridizing to a segment of a nucleic acid containing at least one allele of at least one polymorphic marker within or associated with FLCN, or markers in linkage disequilibrium therewith. In one embodiment, the segment is 15-500 nucleotides in length. In one embodiment, the kit further comprises a set of instructions for using the reagents comprising the kit. In another embodiment, the kit comprises a set of instructions or guidelines for interpreting the results of a test using the reagents comprising the kit.

A further set of embodiments provides for a kit (also referred to as a pharmaceutical pack and are used interchangeably) comprising a therapeutic modulator and a set of instructions for administration of the therapeutic modulator to a human. The therapeutic modulator can be an antisense modulator, antisense oligonucleotide, other oligonucleotide, a small molecule, an antibody, a peptide, a gene therapy, or other therapeutic modulator described herein. In one embodiment, an individual identified as a carrier of at least one allele of at least one polymorphic marker within or associated with FLCN, or markers in linkage disequilibrium therewith, is instructed to take a prescribed dose of the therapeutic modulator. In another embodiment, an individual identified as a homozygous carrier of at least one allele of at least one polymorphic marker within or associated with FLCN, or markers in linkage disequilibrium therewith, is instructed to take a prescribed dose of the therapeutic modulator. In another embodiment, an individual identified as a non-carrier of at least one allele of at least one polymorphic marker within or associated with FLCN, or markers in linkage disequilibrium therewith, is instructed to take a prescribed dose of the therapeutic agent.

### Computers-Readable Medium and Apparatuses

The materials, methods, and kits described herein can be implemented, in all or in part, as computer executable instructions on computer-readable media. As understood by a person skilled in the art, the various steps of the materials, methods and kits described herein can be implemented as various blocks, operations, routines, tools, modules and techniques, which in turn can be implemented in hardware, firmware, software, or any combination of hardware, firmware, and/or software. In certain embodiments, hardware implementations can include but are not limited to a custom integrated circuit (IC), an application specific integrated circuit (ASIC), a field programmable logic array (FPGA), a programmable logic array (PLA), etc. In other embodiments, when implemented as software, the software can be stored in any computer readable medium known in the art, including but not limited to a solid-state disk, a magnetic disk, an optical disk, or other storage medium, in a RAM or ROM or flash memory of a computer, processor, hard disk drive, thumb drive, optical disk drive, tape drive, etc. In one embodiment, the software can be delivered to a user or a computing system via any delivery method known in the art, including but not limited to over a communication channel such as the internet, a wireless connection, a satellite connection, a telephone line, a computer readable disk or other transportable computer storage mechanism.

One set of embodiments provides for a suitable computing system environment known in the art to implement the materials, methods and kits described herein, including but not limited to mobile phones, laptops, personal computers, server computers, multiprocessor systems, microprocessor-based systems, set top boxes, programmable consumer electronics, network PCs, minicomputers, mainframe computers, cloud computing environments, and distributed computing environments that include any of the above systems or devices, etc. In some embodiments, the steps of the materials, methods or kits described herein are implemented via computer-executable instructions such as program modules, including but not limited to routines, programs, objects, components, data structures, etc. that perform particular tasks or implement particular abstract data types. In one embodiment, the methods and apparatuses are practiced in a distributed computing environment, where tasks are performed by remote processing devices that are linked through a communications network. In one embodiment, the methods and apparatuses are practiced in an integrated computing environment. In both integrated and distributed computing environments, program modules can be located in both local and/or remote computer storage media, including memory storage devices.

Thus, one set of embodiments provides a computer-readable medium having computer executable instructions for determining the effect of administering a modulator to a cell an animal, or a human subject, the computer-readable medium comprising data indicative of the level of at least one protein, nucleotide, marker, or other phenotype, and a routine stored on the computer readable medium and adapted to be executed by a processor to determine the effect of administering the modulator from the data. In certain embodiments, the effect being determined is a change in levels of FLCN RNA, or a change in levels of FLCN protein, or a change in phenotype such as cell survival, cell morphology, levels of TDP-43 aggregates in the cytoplasm, survival of the organism, motor function, respiration, behavior or body weight etc. In certain embodiments, the modulator is an antisense modulator, an antisense oligonucleotide, other oligonucleotide modulator, antibody modulator, peptide modulator, or a small molecule modulator, etc. In one embodiment, the computer-readable medium is used to determine progression of ALS and its response to administration of a modulator described herein to a human subject. In another embodiment, the computer-readable medium is used to determine progression of a disease described herein, and its response to administration of a modulator described herein to a human subject.

Another set of embodiments provides for a computer-readable medium having computer executable instructions for developing a modulator using at least one computational method described herein, or other computational methods that are known to those skilled in the art, which are also included in the embodiments herein. The computer-readable medium can comprise data associated with a particular nucleotide sequence, SEQ ID NO, or oligonucleotide represented by a specific sample reference number (GI ID #), or portion thereof disclosed herein, as well as any resulting polypeptide sequences due to transcription and translation of said nucleotide sequences. The computer-readable medium can also be adapted to be executed by a processor to develop a modulator from said data.

Many modifications and variations can be made in the materials, methods, and kits described herein without departing from the spirit and scope of the invention. Accordingly, it should be understood that the materials, methods, and kits described herein are illustrative only and are not limiting upon the scope of the invention.

### EXAMPLES

The following examples are put forth so as to provide those of ordinary skill in the art with a complete disclosure and description of how to make and use the present invention, and are not intended to limit the scope of what the inventors regard as their invention, nor are the examples intended to represent or imply that the experiments below are all of or the only experiments performed. It will be appreciated by persons skilled in the art that numerous variations and/or modifications can be made to the invention as shown in the specific embodiments without departing from the spirit or scope of the invention as broadly described. It should also be appreciated that the examples provide enabling guidance on the use of the combined features of the disclosure to apply such compositions, methods, and systems to other uses. The present embodiments are, therefore, to be considered in all respects as illustrative and not restrictive.

The examples can be implemented in certain embodiments by computers or other processing devices incorporating and/or running software, where the methods and features, software, and processors utilize specialized methods to analyze data.

Efforts have been made to ensure accuracy with respect to numbers used (e.g., amounts, intensity, temperature, etc.) but some experimental errors and deviations should be accounted for.

### Example 1: Antisense Inhibition of Human FLCN in ReN-VM Cells

The antisense oligonucleotides listed in this example have all nucleosides linked by modified phosphorothioate backbones. Bolded nucleosides presented in Table 7 indicate nucleosides that are modified with a 2′-MOE sugar modification. Underlined nucleosides presented in Table 7 indicate non-modified DNA nucleosides. GI1, GI2, GI3, GI4, GI10 and GI12 are gapped sequences comprising a central sequence of unmodified DNA nucleosides flanked by wing sequences on both ends comprising nucleosides with a 2′-MOE sugar modification. GI11 comprises nucleosides wherein each nucleoside is modified with a 2′-MOE sugar modification.

The antisense oligonucleotides were tested in the human neural stem cell line ReN-VM. Human ReN-VM cells were seeded at 200,000 cells per well in a 24-well plate. After 1 day, 100 nM of antisense oligonucleotides were transfected into the cells using Lipofectamine. After a 3-day incubation period, the cells were harvested to obtain whole cell lysates. The whole cell lysates were analyzed by Western blot and probed with a FLCN-specific antibody (CST #3697). The intensity of Western blot bands were quantified by FIJI (Image J) and used to quantify the expression levels of FLCN. The relative expression values of FLCN for the different treatment groups were calculated by normalizing with their respective α-Tubulin loading control, and presented as a percentage to that of the untreated control (which in this case comprised cells not transfected with ASOs or Lipofectamine).

Antisense oligonucleotides listed with the reference number (GI ID #) GI1 and GI2 in Table 8 were observed to produce strong knockdown (≥80%) of the human FLCN protein, with GI2 producing the strongest knockdown of −94%.

### Example 2: Dose-dependent Antisense Inhibition of Human FLCN in ReN-VM Cells

Selected antisense oligonucleotides from Example 1 were tested at various doses in human ReN-VM cells. ReN-VM cells were plated at a density of 200,000 cells per well in a 24-well plate and after 24 hours, were transfected with the following concentrations of antisense oligonucleotide: 0 nM, 1 nM, 25 nM and 100 nM. Whole cell lysates were harvested 48 hours post-transfection for Western blot analysis using a FLCN-specific antibody (CST #3697). The relative expression values of FLCN for the different treatment groups were calculated by normalizing with their respective α-Tubulin loading control, and presented as a ratio to that of the untreated control (in this case transfected with Lipofectamine and no antisense oligonucleotide), which is set to unity (Table 9).

FLCN protein was reduced significantly in a dose-dependent manner for all antisense oligonucleotides tested. GI ID #GI2 was more potent than GI1, achieving knockdown levels of 46% compared to 6%, respectively, at a concentration of 1 nM (shown in Table 9).

### Example 3: Inhibition of Human FLCN in HEK293T Cells by RNAi

siRNAs designed to target various regions of the FLCN transcript produced from the FLCN gene (the reverse complement of RefSeq Accession No. NC_000017.11 truncated from nucleotides 17206900 to 17239000, incorporated herein as SEQ ID NO: 2) were synthesized commercially. Such constructs have been used previously to study the tumor suppressor role of FLCN in BHD syndrome and other renal and lung cancers (Takagi et al., Oncogene 27, 5339-5347 (2008); Hartman et al., Oncogene 28(13), 1594-1604 (2009); Bastola et al., PLoS ONE 8(7), e70030 (2013)). These references together with all the references cited therein, are incorporated herein in their entirety. The nucleotide sequence of the antisense region of the siRNA targeting FLCN as well as non-targeting controls, target start site, target stop site, target region, and description of each are specified in Table 10.

The siRNAs with antisense sequence corresponding to GI ID #GI13, GI14, GI15 and GI16 shown in Table 10 were pooled (these pooled antisense modulators also being referred to as si-FLCN) and tested in HEK293T cells. The siRNAs with antisense sequence corresponding to GI17, GI18, GI19 and GI20 shown in Table 10 were pooled (these pooled antisense modulators also being referred to as si-NT) and used as a non-targeting control. HEK293T cells were transfected with 10 nM of pooled siRNA using Lipofectamine. After 72 hours, the cells were harvested to obtain whole cell lysates. The whole cell lysates were analyzed by Western blot and probed with a FLCN-specific antibody (CST #3697). The intensity of Western blot bands were quantified by FIJI (Image J) and used to quantify the expression levels of FLCN. The relative expression values were calculated by normalizing the FLCN bands with its respective α-Tubulin loading control, and presented as a percentage to that of the si-NT control (Table 11).

The pool of siRNAs comprising antisense sequences corresponding to GI13, GI14, GI15 and GI16 was observed to achieve strong knockdown (86% compared to the si-NT control) of the human FLCN protein (Table 11). These results suggest that one, or any combination, of siRNAs containing antisense sequences matching closely GI13, GI14, GI15 or GI16, or SEQ ID NOs: 22-25 can be used to achieve knockdown of human FLCN. In addition, antisense oligonucleotides containing sequences that match closely to either one, or any combination of, GI13, GI14, GI15 or GI16, or SEQ ID NOs: 22-25 can also be used to achieve knockdown of human FLCN.

### Example 4: Human Stem Cell Models for Evaluating Therapies for Neurodegenerative Disease Such as ALS

In order to evaluate therapies for neurodegenerative diseases such as ALS, in particular to evaluate the inhibition of human FLCN as a therapy for ALS, several ALS induced pluripotent stem cell (iPSC) lines were obtained from various sources and propagated (Table 12). The cell lines GI-iPSC 2 and GI-iPSC 3, shown in Table 12, are isogenic ALS lines, which have been engineered to contain known disease-causing mutations in ALS genes such as the G298S mutation in TARDBP and the L144F mutation in SOD1 respectively (Hor et al., bioRxiv 713651 (2019)). GI-iPSC 4 through GI-iPSC 9 (commercially sourced), shown in Table 12, are cell lines that are derived from ALS patients with various known and unknown genetic mutations, including sporadic ALS with unknown genetic causes, C9orf72 repeat expansion, TARDBP or SOD1 mutations. GI-iPSC 1, GI-iPSC 10 and GI-iPSC 11, shown in Table 12, are healthy lines used as control.

### Example 5: Effect of Inhibition of Human FLCN by RNAi on Survival of Human ALS iPSC-Derived Motor Neurons

The assay described herein was performed to determine the effect of inhibition of human FLCN by antisense modulators such as RNAi on the survival of human ALS iPSC-derived motor neurons. Human ALS iPSCs were differentiated into motor neurons over a period of 28 days according to an established protocol (Hor et al., bioRxiv 713651 (2019)). On Day 28, motor neuron cells were transfected with 10 nM of siRNA that was either validated to inhibit human FLCN (si-FLCN from Example 3) or that served as the non-targeting control (si-NT from Example 3) using Lipofectamine. The motor neuron cells were fixed with 4% paraformaldehyde on Days 28, 31, and 35 for determination of motor neuron survival. Motor neuron cells that were analyzed on Day 35 underwent an additional round of transfection with 10 nM of siRNA on Day 32. The fixed motor neuron cells were stained with a specific antibody against the motor neuron marker ISL1, and cellular nuclei were counterstained with 4′,6-diamidino-2-phenylindole (DAPI) prior to imaging using the Opera Phenix High-content Screening System (Perkin Elmer). To calculate normalized survival index (NSI shown in Table 13) on Days 28, 31, and 35, the ratio of ISL1+ cells (those cells stained with the ISL1 antibody) to DAPI-stained nuclei was determined and normalized against the ratio of ISL1+ cells to DAPI-stained nuclei for the healthy control motor neurons (BJ-iPS) at Day 28 (Table 13). Results shown in Table 13 are the average of at least 5 technical replicates.

The results indicate that treatment with si-FLCN can increase the survival of human ALS iPSC-derived motor neurons on both Day 31 and Day 35, compared to treatment with the non-targeting control siRNA (si-NT) (Table 13). This suggests that inhibition of human FLCN (via e.g., RNAi, antisense oligonucleotides, antibodies, small molecules, and other modulators etc.) can promote the survival of human motor neuron cells. Furthermore, treatment with si-FLCN increases the survival of iPSC-derived motor neurons representing different ALS sub-types, including but not limited to the isogenic SOD1 (L144F), isogenic TDP-43 (G298S) and patient-derived sporadic ALS line (CS14isALS-Tn16) (Table 13). This finding suggests that inhibition of human FLCN via a modulator, such as those aforementioned modulators comprising RNAi, antisense oligonucleotide, peptide, antibody, or small molecule, can be an effective therapy for a broad subset of ALS patients, including, but not limited to, patients with mutations in known ALS genes such as SOD1 and TARDP43, as well as sporadic ALS patients with no known mutations in ALS genes.

### Example 6: Effect of Inhibition of Human FLCN by RNAi on the Levels of Phosphorylated TDP-43 in the Cytoplasm of Human ALS iPSC-Derived Motor Neurons

The assay described herein was performed to determine the effect of inhibition of human FLCN by antisense modulators such as RNAi on the levels of phosphorylated TDP-43 (pTDP-43) in the cytoplasm of human ALS iPSC-derived motor neurons. Human ALS iPSCs were differentiated into motor neurons over a period of 28 days according to an established protocol (Hor et al., bioRxiv 713651 (2019)). On Day 28, motor neuron cells were transfected with 10 nM of siRNA that was either validated to inhibit human FLCN (si-FLCN from Example 3) or that served as the non-targeting control (si-NT from Example 3) using Lipofectamine. The motor neuron cells were fixed with 4% paraformaldehyde on Days 28, 31, and 35 to determine the levels of pTDP-43 in the cytoplasm. Motor neuron cells that were analyzed on Day 35 underwent an additional round of transfection with 10 nM of siRNA on Day 32. The fixed motor neuron cells were stained with a specific antibody against the motor neuron marker ISL1, counterstained with DAPI, and also co-stained with a specific antibody against TDP-43 phosphorylated at Ser 409 or Ser 410, prior to imaging using the Opera Phenix High-content Screening System (Perkin Elmer). The number of pTDP-43 foci (spots) per unit area of cytoplasm were quantified and used as a proxy for the levels of cytoplasmic pTDP-43. The levels of pTDP-43 in the cytoplasm on Days 28, 31, and 35 for the different treatment groups were normalized against the cytoplasmic pTDP-43 levels in healthy control motor neurons (BJ-iPS) at Day 28 (Table 14). Results shown in Table 14 are the average of at least 5 technical replicates.

The results indicate that compared to the healthy control BJ-iPS, cytoplasmic pTDP-43 levels were elevated to over two-fold in all ALS iPSC motor neurons tested, including, but not limited to, the isogenic SOD1 (L144F), isogenic TDP-43 (G298S) and patient-derived sporadic ALS line (CS14isALS-Tn16). Over 97% of all ALS cases (both sporadic and familial) display TDP-43 positive aggregates in the cytoplasm of affected neurons and the cytoplasmic TDP-43 aggregates have been associated with ALS pathology (Prasad et al., 2019). This finding suggests that the ALS iPSC-derived motor neurons can replicate important pathological features of ALS and can serve as a relevant disease model. Treatment of the ALS iPSC-derived motor neurons with si-FLCN but not si-NT reduced the levels of cytoplasmic pTDP-43 aggregates in those cells (Table 14). This suggests that inhibition of human FLCN via a modulator, such as those aforementioned modulators comprising RNAi, antisense oligonucleotide, peptide, antibody, small molecule, or other modulator, can reduce the levels of pathological cytoplasmic pTDP-43 aggregates and potentially improve disease outcomes for ALS patients.

### Example 7: Identification of Small Molecule Modulators

Small molecule modulators that are capable of targeting FLCN were identified using methods of development described herein. Briefly, a high-throughput computational screen was performed to identify small molecule exemplars and small molecule scaffolds (otherwise known as pharmacophores) that can bind to the FLCN protein. An X-ray crystal structure of the FLCN protein was obtained from the Protein Data Bank (PDB). The cavities on the surface of FLCN that are amenable to small molecule binding were identified using established tools such as Autodock. Molecular docking was performed using a computational database of small molecule ligands, which include small molecule exemplars and scaffolds, to the identified cavities on the surface of the FLCN protein. Small molecule exemplars with excellent predicted binding scores to the FLCN protein were identified and listed in Table 15. In one embodiment, small molecule exemplars described in Table 15 can be used as modulators to inhibit the activity of FLCN. In another embodiment, small molecule modulators that possess similar scaffolds as that of the identified exemplars, which are described in Table 15, can be used as modulators to inhibit the activity of FLCN.

### Example 8: Identification of Antibody or Peptide Modulators

Antibody modulators that are capable of targeting FLCN were identified from commercial sources and disclosed in Table 16. Such antibodies can be capable of inhibiting the expression or activity of FLCN. Furthermore, antibodies, antibody fragments, monobodies, or other peptide modulators comprising a similar CDR to at least one antibody described in Table 16, can be capable of inhibiting the expression or activity of FLCN.

### Example 9: Antisense Inhibition of Human FLCN in Mammalian Cells

The antisense oligonucleotides listed in this example have all nucleosides linked by modified phosphorothioate backbones. Bolded nucleosides presented in Table 17 indicate nucleosides that are modified with a 2′-MOE sugar modification. Underlined nucleosides presented in Table 17 indicate non-modified DNA nucleosides (deoxynucleotides). Nucleosides that are italicized indicate nucleosides with a 2′-OMe sugar modification. GI81, GI84, GI85, GI86, GI88, GI89 and GI90 are gapped sequences comprising a central sequence of deoxynucleotides, which are flanked on both sides by wing sequences consisting of 2′-MOE modified nucleotides, wherein the second nucleotide of the central sequence from the 5′ end of the oligonucleotide contains a 2′-OMe sugar modification.

These antisense oligonucleotides were tested in the human cell line HEK293T. HEK293T cells were seeded at 400,000 cells per well in a 6-well plate. After 1 day, various concentrations of antisense oligonucleotides ranging from 0 nM to 100 nM were transfected into the cells using Lipofectamine. After a 2-day incubation period, the cells were harvested to obtain whole cell lysates. The whole cell lysates were analyzed by Western blot and probed with a FLCN-specific antibody (CST #3697). The intensity of Western blot bands were quantified by FIJI (Image J) and used to quantify the expression levels of FLCN. The relative expression values of FLCN for the different treatment groups were calculated by normalizing with their respective α-Tubulin loading control, and presented as a percentage to that of the untreated control (which in this case comprised cells not transfected with ASOs). The potencies of these antisense oligonucleotides are reported in the form of IC50 values in Table 17.

TABLE 1
Examples of Functional Segments
for NM_144997.7 (SEQ ID NO: 1)

| | | | Start site with | Stop site with |
|---|---|---|---|---|
| Exon | mRNA | mRNA | reference to | reference to |
| number | start site | stop site | SEQ ID NO: 2 | SEQ ID NO: 2 |
| 1 | 1 | 257 | 1833 | 2089 |
| 2 | 258 | 371 | 6100 | 6213 |
| 3 | 372 | 460 | 7119 | 7207 |
| 4 | 461 | 733 | 10840 | 11112 |
| 5 | 734 | 880 | 12679 | 12825 |
| 6 | 881 | 1102 | 14858 | 15079 |
| 7 | 1103 | 1263 | 16340 | 16500 |
| 8 | 1264 | 1355 | 17373 | 17464 |
| 9 | 1356 | 1546 | 19792 | 19982 |
| 10 | 1547 | 1660 | 21819 | 21932 |
| 11 | 1661 | 1784 | 22498 | 22621 |
| 12 | 1785 | 1916 | 23685 | 23816 |
| 13 | 1917 | 2022 | 23911 | 24016 |
| 14 | 2023 | 3667 | 25145 | 26789 |

TABLE 2
Examples of Functional Segments
for NM_144606.7 (SEQ ID NO: 3)

| | | | Start site with | Stop site with |
|---|---|---|---|---|
| Exon | mRNA | mRNA | reference to | reference to |
| number | start site | stop site | SEQ ID NO: 2 | SEQ ID NO: 2 |
| 1 | 1 | 257 | 1833 | 2089 |
| 2 | 258 | 371 | 6100 | 6213 |
| 3 | 372 | 460 | 7119 | 7207 |
| 4 | 461 | 733 | 10840 | 11112 |
| 5 | 734 | 880 | 12679 | 12825 |
| 6 | 881 | 1102 | 14858 | 15079 |
| 7 | 1103 | 1263 | 16340 | 16500 |
| 8B | 1264 | 3325 | 17373 | 19434 |

TABLE 3
Examples of Functional Segments
for NM_001353229.2 (SEQ ID NO: 4)

| | | | Start site with | Stop site with |
|---|---|---|---|---|
| Exon | mRNA | mRNA | reference to | reference to |
| number | start site | stop site | SEQ ID NO: 2 | SEQ ID NO: 2 |
| 1 | 1 | 257 | 1833 | 2089 |
| Intron 1 | 258 | 465 | 2318 | 2525 |
| 2 | 466 | 579 | 6100 | 6213 |
| 3 | 580 | 668 | 7119 | 7207 |
| 4 | 669 | 941 | 10840 | 11112 |
| 5 | 942 | 1088 | 12679 | 12825 |
| Intron 5 | 1089 | 1142 | 14490 | 14543 |
| 6 | 1143 | 1364 | 14858 | 15079 |
| 7 | 1365 | 1525 | 16340 | 16500 |
| 8 | 1526 | 1617 | 17373 | 17464 |
| 9 | 1618 | 1808 | 19792 | 19982 |
| 10 | 1809 | 1922 | 21819 | 21932 |
| 11 | 1923 | 2046 | 22498 | 22621 |
| 12 | 2047 | 2178 | 23685 | 23816 |
| 13 | 2179 | 2284 | 23911 | 24016 |
| 14 | 2285 | 3929 | 25145 | 26789 |

TABLE 4
Examples of Functional Segments
for NM_001353230.2 (SEQ ID NO: 5)

| | | | Start site with | Stop site with |
|---|---|---|---|---|
| Exon | mRNA | mRNA | reference to | reference to |
| number | start site | stop site | SEQ ID NO: 2 | SEQ ID NO: 2 |
| 1 | 1 | 257 | 1833 | 2089 |
| 2 | 258 | 371 | 6100 | 6213 |
| 3 | 372 | 460 | 7119 | 7207 |
| Intron 3 | 461 | 743 | 10096 | 10378 |
| 4 | 744 | 1016 | 10840 | 11112 |
| 5 | 1017 | 1163 | 12679 | 12825 |
| 6 | 1164 | 1385 | 14858 | 15079 |
| 7 | 1386 | 1546 | 16340 | 16500 |
| 8 | 1547 | 1638 | 17373 | 17464 |
| 9 | 1639 | 1829 | 19792 | 19982 |
| 10 | 1830 | 1943 | 21819 | 21932 |
| 11 | 1944 | 2067 | 22498 | 22621 |
| 12 | 2068 | 2199 | 23685 | 23816 |
| 13 | 2200 | 2305 | 23911 | 24016 |
| 14 | 2306 | 3950 | 25145 | 26789 |

TABLE 5
Examples of Functional Segments
for NM_001353231.2 (SEQ ID NO: 6)

| | | | Start site with | Stop site with |
|---|---|---|---|---|
| Exon | mRNA | mRNA | reference to | reference to |
| number | start site | stop site | SEQ ID NO: 2 | SEQ ID NO: 2 |
| 1 | 1 | 257 | 1833 | 2089 |
| 2 | 258 | 371 | 6100 | 6213 |
| 3 | 372 | 460 | 7119 | 7207 |
| Intron 3 | 461 | 659 | 10180 | 10378 |
| 4 | 660 | 932 | 10840 | 11112 |
| 5 | 933 | 1079 | 12679 | 12825 |
| 6 | 1080 | 1301 | 14858 | 15079 |
| 7 | 1302 | 1462 | 16340 | 16500 |
| 8 | 1463 | 1554 | 17373 | 17464 |
| 9 | 1555 | 1745 | 19792 | 19982 |
| 10 | 1746 | 1859 | 21819 | 21932 |
| 11 | 1860 | 1983 | 22498 | 22621 |
| 12 | 1984 | 2115 | 23685 | 23816 |
| 13 | 2116 | 2221 | 23911 | 24016 |
| 14 | 2222 | 3866 | 25145 | 26789 |

TABLE 6
Antisense Modulators
Antisense modulators targeting SEQ ID NO 2

| | | Target | Target | | Binding | SEQ |
|---|---|---|---|---|---|---|
| NO | Sequence | start site | stop site | Target region | Score | ID NO |
| 1 | CATGGCTGATATATCCCGGG | 12705 | 12724 | Exon 5 | −25.6 | 16 |
| 2 | CAGCCAGCGTTAATAACGGG | 14741 | 14760 | Intron 5 | −29.1 | 17 |
| 3 | TCCTCTGGTGTAGGAATGGCGT | 16400 | 16421 | Exon 7 | −31.3 | 18 |
| 4 | CAGCCAGCGTTAATAACG | 14743 | 14760 | Intron 5 | −25.5 | 19 |
| 5 | CTGGTGTAGGAATGGCGT | 16400 | 16417 | Exon 7 | −27.3 | 20 |
| 6 | TGGCTGATATATCCCGGG | 12705 | 12722 | Exon 5 | −24.5 | 21 |
| 7 | ATTTAATGGAGGTCTCTTT | 12727 | 12745 | Exon 5 | −24.3 | 22 |
| 8 | TCATGGCTGATATATCCCG | 12707 | 12725 | Exon 5 | −23.3 | 23 |
| 9 | AATGGCGTGAAGGCTGTGT | 16389 | 16407 | Exon 7 | −24.8 | 24 |
| 10 | TCTGGACCAAGGTATCCTC | 17431 | 17449 | Exon 8 | −18.2 | 25 |
| 11 | ATGGTGATGATGCTGTACC | 14967 | 14985 | Exon 6 | −16.2 | 26 |
| 12 | ATCATGGCTGATATATCCCGG | 12706 | 12726 | Exon 5 | −26.1 | 27 |
| 13 | ATGAGGTGTGACTTGTAGGTC | 25283 | 25303 | Exon 14 | −28.6 | 28 |
| 14 | AATCTTATTCAGGATGGTGGG | 23916 | 23936 | Exon 13 | −31.4 | 29 |
| 15 | AAAAAAAAAAAGGGCCAGGC | 26491 | 26510 | Exon 14 | −21.5 | 30 |
| 16 | TGAAATTGTCTTCAATCACC | 26171 | 26190 | Exon 14 | −22.8 | 31 |
| 17 | AACTGTAACCAAACGTAAAT | 26134 | 26153 | Exon 14 | −21 | 32 |
| 18 | TGAAAGCAGCAATAAAGACA | 26112 | 26131 | Exon 14 | −25.5 | 33 |
| 19 | GGACTCCATCATTAAGCCCC | 25985 | 26004 | Exon 14 | −28.7 | 34 |
| 20 | ACCTTCACCAGCACCTGCAG | 25960 | 25979 | Exon 14 | −31.3 | 35 |
| 21 | CACATTTCAGAGGACCAAAA | 25901 | 25920 | Exon 14 | −23.8 | 36 |
| 22 | GATGAGGAAGAGATCCACTT | 25881 | 25900 | Exon 14 | −28.2 | 37 |
| 23 | CTTCACTCCCCAAAGTCTCT | 25864 | 25883 | Exon 14 | −25.5 | 38 |
| 24 | GCGGAGCCCTAACTCAATCA | 25843 | 25862 | Exon 14 | −24.3 | 39 |
| 25 | GTACTGAATATTCACAACTG | 25800 | 25819 | Exon 14 | −23.1 | 40 |
| 26 | GCTTGAATGTTAACCTCGGG | 25719 | 25738 | Exon 14 | −24.6 | 41 |
| 27 | AACCTCGGGAGCAGACATGT | 25708 | 25727 | Exon 14 | −29.2 | 42 |
| 28 | CATGTTATTGCGACTGCATA | 25693 | 25712 | Exon 14 | −22.2 | 43 |
| 29 | CCTGTTTCTCCTGCGGGTTT | 25663 | 25682 | Exon 14 | −28.8 | 44 |
| 30 | AGAGAGAGCCCATCATCCCT | 25572 | 25591 | Exon 14 | −24.8 | 45 |
| 31 | GCGATTCCAACGGCTGGAGG | 25530 | 25549 | Exon 14 | −27.6 | 46 |
| 32 | AAACCTGACAGGGCCGAGCC | 25484 | 25503 | Exon 14 | −24.9 | 47 |
| 33 | GACACAGCTCCTTCCAGCAG | 25435 | 25454 | Exon 14 | −21.3 | 48 |
| 34 | GTGGACAGCCATCCCTGTCT | 25369 | 25388 | Exon 14 | −20.3 | 49 |
| 35 | TGCGGACCGTGGACATGAGG | 25298 | 25317 | Exon 14 | −32.2 | 50 |
| 36 | GACTTGTAGGTCTTGCTCAG | 25275 | 25294 | Exon 14 | −26 | 51 |
| 37 | CAATCCCTCGAGCCCTGGTC | 25086 | 25105 | Intron 13 | −29.5 | 52 |
| 38 | AGGCTTGGCCCCTGAACTGC | 24984 | 25003 | Intron 13 | −26.9 | 53 |
| 39 | GGAAGCTGTCGGGGCTGGGC | 24936 | 24955 | Intron 13 | −36.7 | 54 |
| 40 | CTCGGCCCTCTGTCGGGTAT | 24917 | 24936 | Intron 13 | −28.4 | 55 |
| 41 | GTATGGGTATGGGGCCTGTG | 24901 | 24920 | Intron 13 | −24.3 | 56 |
| 42 | TCAAGCTGTTGTCAAGACGC | 24863 | 24882 | Intron 13 | −29.1 | 57 |
| 43 | CGTGGAAGACACTGGTTGCT | 24814 | 24833 | Intron 13 | −33.4 | 58 |
| 44 | TACCAAGAATCGAGGCAGGA | 24729 | 24748 | Intron 13 | −26.3 | 59 |
| 45 | AAAACCCGGGGGGCCAGGTG | 24679 | 24698 | Intron 13 | −26.3 | 60 |
| 46 | TGGGCGGATCACGAAGTCAG | 24616 | 24635 | Intron 13 | −23.9 | 61 |
| 47 | CACGAAGTCAGGAGTTCAAG | 24607 | 24626 | Intron 13 | −23.9 | 62 |
| 48 | GTGGTGAGCCAAGATCATGC | 24449 | 24468 | Intron 13 | −31.8 | 63 |
| 49 | GGGGCATGGGTTACTGGGCT | 24343 | 24362 | Intron 13 | −23.7 | 64 |
| 50 | CCTCAGTCGCTCTCCAAGGC | 24241 | 24260 | Intron 13 | −25.8 | 65 |
| 51 | CTCTCCAAGGCAGGCGCCAC | 24232 | 24251 | Intron 13 | −24.6 | 66 |
| 52 | GAGGCCACCTGAGCTTTGCA | 24163 | 24182 | Intron 13 | −27.6 | 67 |
| 53 | GTGGCGGACGTGGAGTTGGA | 24143 | 24162 | Intron 13 | −26.4 | 68 |
| 54 | GGAGTTGGAACCCCGCCCCC | 24132 | 24151 | Intron 13 | −28.2 | 69 |
| 55 | AGGGGCAGAGCAAGGGCAGG | 23877 | 23896 | Intron 12 | −39.8 | 70 |
| 56 | CCTCACCTCCCCTGCGCTAG | 23622 | 23641 | Intron 11 | −34.4 | 71 |
| 57 | CCCTGCGCTAGCCCACCGTG | 23613 | 23632 | Intron 11 | −32.5 | 72 |
| 58 | CTCGTAGTGGTAAGAATTTG | 23318 | 23337 | Intron 11 | −29.2 | 73 |
| 59 | CCAATGCTACCCGGAAAAAA | 23269 | 23288 | Intron 11 | −24.3 | 74 |
| 60 | GCCTCGCAGGGAGTCAGGAC | 23243 | 23262 | Intron 11 | −32.8 | 75 |
| 61 | TTGGGGGCTCAGGGTAGCTC | 23207 | 23226 | Intron 11 | −31.4 | 76 |
| 62 | CCACTCGGACCATGAGTCAG | 23188 | 23207 | Intron 11 | −25.7 | 77 |
| 63 | GTTTGCTGAGCCCTGACCAC | 23081 | 23100 | Intron 11 | −25.4 | 78 |
| 64 | CACACCAGTGAAGGCCGGGA | 23064 | 23083 | Intron 11 | −30.9 | 79 |
| 65 | CGGGAAGACCTCGGGCACAT | 23049 | 23068 | Intron 11 | −27.6 | 80 |
| 66 | GCATTCAGCAGGCCTCCATC | 22925 | 22944 | Intron 11 | −23.1 | 81 |
| 67 | AGTCCCTGCCAGCCAACCTT | 22813 | 22832 | Intron 11 | −29.6 | 82 |
| 68 | CCAACCTTCCCATCAGGTCC | 22801 | 22820 | Intron 11 | −29.2 | 83 |
| 69 | GCACGCACCTGAGGAGAGCA | 22610 | 22629 | Exon 11-Intron | −31 | 84 |
| | | | | 11 junction | | |
| 70 | ACGTGGGGGGGGATCTGCAC | 22591 | 22610 | Exon 11 | −27.5 | 85 |
| 71 | GAGCCCCAGGAAGTTGCACC | 22562 | 22581 | Exon 11 | −35.4 | 86 |
| 72 | CTCCACCACCAGGTCCCTAA | 22307 | 22326 | Intron 10 | −25.9 | 87 |
| 73 | TCCCTAACTCTTGTCTGGAC | 22294 | 22313 | Intron 10 | −24.4 | 88 |
| 74 | CCGCCAGTCACACACCAGCT | 22275 | 22294 | Intron 10 | −30.1 | 89 |
| 75 | AGCTTGTACCGCCCCTCGCT | 22259 | 22278 | Intron 10 | −29.6 | 90 |
| 76 | GCTTTTACCAAGACTGTGTC | 22171 | 22190 | Intron 10 | −25.5 | 91 |
| 77 | TATGCATTTTTGTTCCCTCT | 22150 | 22169 | Intron 10 | −23.3 | 92 |
| 78 | CCCCCAGTGGAGACCGTGTG | 22045 | 22064 | Intron 10 | −28.3 | 93 |
| 79 | CAGCGGTTCTGTGCTGGGCA | 22020 | 22039 | Intron 10 | −24.3 | 94 |
| 80 | AAGATGTTCTCACCCGAAGT | 21926 | 21945 | Exon 10-Intron | −25.8 | 95 |
| | | | | 10 junction | | |
| 81 | CTTCAAAAGCTGACTGGACG | 21905 | 21924 | Exon 10 | −29.7 | 96 |
| 82 | GAGGTCCACGTCTCTGCTTT | 21886 | 21905 | Exon 10 | −24.3 | 97 |
| 83 | CCAGGCCAGCATGCGGAAAG | 21835 | 21854 | Exon 10 | −30.9 | 98 |
| 84 | GGAAAGAAGGGGCACCCAGG | 21821 | 21840 | Exon 10 | −30.4 | 99 |
| 85 | GCACCCAGGACCTAAACAAG | 21810 | 21829 | Exon 10-Intron | −26.7 | 100 |
| | | | | 9 junction | | |
| 86 | AGTGCTTTCAGCGTGACTAG | 21783 | 21802 | Intron 9 | −27.2 | 101 |
| 87 | GGCTCAGGAGAAAGACACTT | 21692 | 21711 | Intron 9 | −27.5 | 102 |
| 88 | ATCTTCTCAGGGAGGCGGGT | 21672 | 21691 | Intron 9 | −26.9 | 103 |
| 89 | AGGGTTAAAGGGGCAGAGAG | 21643 | 21662 | Intron 9 | −28 | 104 |
| 90 | GAGAGAAGTTCCAGGTTTGC | 21626 | 21645 | Intron 9 | −24.2 | 105 |
| 91 | TGCACATAAATGCTAGATTC | 21609 | 21628 | Intron 9 | −20.5 | 106 |
| 92 | CCAAGATCTTTCTCCCAGTT | 21576 | 21595 | Intron 9 | −21.8 | 107 |
| 93 | TATTCACCTTCTCTCTACAG | 21512 | 21531 | Intron 9 | −21.4 | 108 |
| 94 | TTTCTGCTGAACCATTTGAA | 21488 | 21507 | Intron 9 | −24.8 | 109 |
| 95 | ATTTGAAAGCAAGCTGTAGG | 21475 | 21494 | Intron 9 | −26.2 | 110 |
| 96 | GGCCCTCGACATGGCAGCAC | 21456 | 21475 | Intron 9 | −29.9 | 111 |
| 97 | TACTACAGGACCCATCATGG | 21295 | 21314 | Intron 9 | −23.5 | 112 |
| 98 | CAGCCTCTCCCCACACCCCA | 21239 | 21258 | Intron 9 | −32 | 113 |
| 99 | TCCAGCCTGCTGTCTCACGG | 21180 | 21199 | Intron 9 | −27.6 | 114 |
| 100 | TTCTAAGAAGCCAAACCCAG | 21159 | 21178 | Intron 9 | −21 | 115 |
| 101 | AGAGGCCACAATACTATGTC | 21118 | 21137 | Intron 9 | −24.5 | 116 |
| 102 | CAAAGTAGTCCCACCTCCAG | 21078 | 21097 | Intron 9 | −31.8 | 117 |
| 103 | GGAATTCCAGACCCCTTCCA | 21057 | 21076 | Intron 9 | −25.5 | 118 |
| 104 | CAACCTTCATCAACATAAGC | 21039 | 21058 | Intron 9 | −20.6 | 119 |
| 105 | TAAGCAACGGACAAGGAAGG | 21024 | 21043 | Intron 9 | −23.8 | 120 |
| 106 | CAAGGAAGGTTTCAGCCAAA | 21013 | 21032 | Intron 9 | −24.5 | 121 |
| 107 | TCAGCCAAATGCAGGATTTT | 21002 | 21021 | Intron 9 | −21.6 | 122 |
| 108 | GTGTTTACATCCAGAAGCCC | 20972 | 20991 | Intron 9 | −21.8 | 123 |
| 109 | AAGCCCCAAGGACATCTGCC | 20958 | 20977 | Intron 9 | −23.7 | 124 |
| 110 | ATTTTATAAGAAGGCAGCCT | 20854 | 20873 | Intron 9 | −20.4 | 125 |
| 111 | GCAGCCTGCCCTTGTAATCC | 20841 | 20860 | Intron 9 | −26.2 | 126 |
| 112 | CCAGCAACCCACACCCCTCT | 20823 | 20842 | Intron 9 | −28.8 | 127 |
| 113 | TCTCTCCCGGCATCTGACTC | 20806 | 20825 | Intron 9 | −25.6 | 128 |
| 114 | GGAGACAACACTGTTAGATT | 20777 | 20796 | Intron 9 | −22.2 | 129 |
| 115 | CACCCTGAACACACAGTGCC | 20723 | 20742 | Intron 9 | −25 | 130 |
| 116 | ACACAGTGCCTGCTTTATTT | 20713 | 20732 | Intron 9 | −20.4 | 131 |
| 117 | CTTCTAATTAATCATATTCC | 20693 | 20712 | Intron 9 | −21.2 | 132 |
| 118 | TATTCCATGGTTTGGCTACT | 20679 | 20698 | Intron 9 | −25.6 | 133 |
| 119 | GTTTACTAGTATGGTTTGAT | 20659 | 20678 | Intron 9 | −21.7 | 134 |
| 120 | GTTCCTAATTTTTTGTAAGA | 20630 | 20649 | Intron 9 | −20.8 | 135 |
| 121 | CTCAGCCTCCAAGTAGCTGG | 20482 | 20501 | Intron 9 | −24.6 | 136 |
| 122 | CAGCCAGACCATCACTTACA | 20306 | 20325 | Intron 9 | −22.6 | 137 |
| 123 | CCAGGCTGCATCCAAACCAA | 20275 | 20294 | Intron 9 | −29.1 | 138 |
| 124 | GCCTTTAGGAGCTTCTTAGA | 20238 | 20257 | Intron 9 | −32.1 | 139 |
| 125 | CTATCTGGGCTTTTCTTTGA | 20193 | 20212 | Intron 9 | −25.8 | 140 |
| 126 | GAAGATACTTAGGTCATCAC | 20175 | 20194 | Intron 9 | −21.9 | 141 |
| 127 | GAGCACTGATCCTATGGCTA | 20147 | 20166 | Intron 9 | −26.5 | 142 |
| 128 | GGCTAATACCCCCAACGCCC | 20132 | 20151 | Intron 9 | −22.5 | 143 |
| 129 | CAGCCACCCACCGAGGAGGC | 20104 | 20123 | Intron 9 | −20.1 | 144 |
| 130 | CTGCAGGGTTTTGAAGGTGG | 20071 | 20090 | Intron 9 | −34 | 145 |
| 131 | CAGAGGCAAGGCGTGTGGGC | 20045 | 20064 | Intron 9 | −22.2 | 146 |
| 132 | CCTGAGCTCCTGATGCGCTG | 19999 | 20018 | Intron 9 | −26.6 | 147 |
| 133 | CTACCTGCCTCATGTGCCGG | 19967 | 19986 | Exon 9-Intron | −27 | 148 |
| | | | | 9 junction | | |
| 134 | GCCGGAGGGACTTGAAGACT | 19952 | 19971 | Exon 9 | −27.9 | 149 |
| 135 | TCAGCTCCCGCCCTTCTGTA | 19868 | 19887 | Exon 9 | −27.4 | 150 |
| 136 | CTCTCTGGCAACACAGGGGC | 19848 | 19867 | Exon 9 | −20.4 | 151 |
| 137 | CCTCCTCTTCAGCCTCAGAG | 19823 | 19842 | Exon 9 | −25.9 | 152 |
| 138 | CCTCAGAGTTGTCCCAGCTT | 19811 | 19830 | Exon 9 | −23.4 | 153 |
| 139 | CCCAGCTTTCTGATTCCTCT | 19799 | 19818 | Exon 9 | −22.7 | 154 |
| 140 | TGACAAGGACAGTTACAGAT | 19760 | 19779 | Intron 8 | −23.9 | 155 |
| 141 | ACAGATACAAACAGTCTCAT | 19746 | 19765 | Intron 8 | −21.2 | 156 |
| 142 | TGAGCCGGTCAGTGTAGATT | 19694 | 19713 | Intron 8 | −22.4 | 157 |
| 143 | GTGTAGATTCCTGGCTGCGG | 19683 | 19702 | Intron 8 | −23.1 | 158 |
| 144 | GGCCAGGTCCTATGCTCCCT | 19665 | 19684 | Intron 8 | −24.7 | 159 |
| 145 | GCTGTCTACTTAGCCCGGGG | 19598 | 19617 | Intron 8 | −22.9 | 160 |
| 146 | GCCAACAGCGTGTCTACGGA | 19493 | 19512 | Intron 8 | −22.8 | 161 |
| 147 | GGATCTACACGTTGTTTCTT | 19446 | 19465 | Intron 8 | −22.5 | 162 |
| 148 | GTTGTTTCTTTTTTTTCCAC | 19436 | 19455 | Intron 8 | −20.3 | 163 |
| 149 | CGATTCTTCTGCCCCAGCCT | 19310 | 19329 | Intron 8 | −25.7 | 164 |
| 150 | TGTCCGGCTAACTTTTTTTG | 19256 | 19275 | Intron 8 | −20.4 | 165 |
| 151 | CCGGCTAACTTTTTTTGGTA | 19253 | 19272 | Intron 8 | −20.4 | 166 |
| 152 | ACCTCGGCCTCCCGGTCCAC | 19162 | 19181 | Intron 8 | −28.9 | 167 |
| 153 | CTTGAGGGCGTACGTGTGCG | 19131 | 19150 | Intron 8 | −26.4 | 168 |
| 154 | GTACGTGTGCGGCTTTGTTA | 19122 | 19141 | Intron 8 | −25.2 | 169 |
| 155 | TGGTGCTAAAGTTTGGGCTT | 19087 | 19106 | Intron 8 | −20.7 | 170 |
| 156 | ATCCAATAGAAAGCATTTCA | 19012 | 19031 | Intron 8 | −20.6 | 171 |
| 157 | AAGCATTTCAGCCCTTCCTC | 19002 | 19021 | Intron 8 | −21.2 | 172 |
| 158 | CAAGTGATCCTCCCAACGAA | 18838 | 18857 | Intron 8 | −25.9 | 173 |
| 159 | CAACGAAGCCTTCCAAGTAG | 18825 | 18844 | Intron 8 | −23.6 | 174 |
| 160 | TTCCAGGCGTGCACCAGTGC | 18666 | 18685 | Intron 8 | −28.3 | 175 |
| 161 | TGCACCAGTGCCTGACTTCG | 18657 | 18676 | Intron 8 | −25 | 176 |
| 162 | TTATGGCTACATCCAACATC | 18633 | 18652 | Intron 8 | −24.1 | 177 |
| 163 | TTGTCATTTCAACGAAACTT | 18540 | 18559 | Intron 8 | −20.2 | 178 |
| 164 | AACGAAACTTCATGGAGCCC | 18530 | 18549 | Intron 8 | −24.5 | 179 |
| 165 | CCCTCACAAATGACAACATC | 18513 | 18532 | Intron 8 | −21.9 | 180 |
| 166 | CAACATCTCCATTTCACATC | 18500 | 18519 | Intron 8 | −22.9 | 181 |
| 167 | GAGACCCAAAGGGAAGGGTG | 18478 | 18497 | Intron 8 | −28.2 | 182 |
| 168 | GAAGGGTGCACGTCAGAAGC | 18466 | 18485 | Intron 8 | −26.2 | 183 |
| 169 | GAAGCAAATCCAGGATGCGA | 18451 | 18470 | Intron 8 | −27.6 | 184 |
| 170 | CTTCGTGCTAAAAAGCTCAC | 18320 | 18339 | Intron 8 | −20.4 | 185 |
| 171 | GCTCACCAAGACACTGCCCT | 18306 | 18325 | Intron 8 | −29.1 | 186 |
| 172 | GCCATCTGCACCTGCCTAGA | 18285 | 18304 | Intron 8 | −27.7 | 187 |
| 173 | TGCCTAGAAACATGATTAGC | 18273 | 18292 | Intron 8 | −24.3 | 188 |
| 174 | TATAATAAATGGCTCCAGGG | 18251 | 18270 | Intron 8 | −20.7 | 189 |
| 175 | CAGGGCCAGCCAACCTGCAG | 18236 | 18255 | Intron 8 | −24.5 | 190 |
| 176 | CTGCAGGGCATGGGGGTGGA | 18222 | 18241 | Intron 8 | −32.6 | 191 |
| 177 | CTTTGTCTTTCCTGACCAGG | 18194 | 18213 | Intron 8 | −26.1 | 192 |
| 178 | CTGAGACTCCATTGGCAAAG | 18163 | 18182 | Intron 8 | −20.7 | 193 |
| 179 | GCATGGGAAGCATTATTAAT | 18056 | 18075 | Intron 8 | −20.8 | 194 |
| 180 | TTAATGACATGTGGTGTGGA | 18041 | 18060 | Intron 8 | −25.4 | 195 |
| 181 | GGTGTGGAACAATGGTCGTA | 18029 | 18048 | Intron 8 | −24.3 | 196 |
| 182 | ACCACAGACCTGTAGCACCA | 18010 | 18029 | Intron 8 | −26.2 | 197 |
| 183 | TGGTGCACACAGTGCCACGA | 17934 | 17953 | Intron 8 | −20.2 | 198 |
| 184 | AAACACAGGGCCCCAAGCCC | 17909 | 17928 | Intron 8 | −22.2 | 199 |
| 185 | CAAGCCCCAGATCAGGAACC | 17896 | 17915 | Intron 8 | −24.4 | 200 |
| 186 | CCTGGGAGGTCAGGGAACCA | 17860 | 17879 | Intron 8 | −29.7 | 201 |
| 187 | GAACACTTTATTTGTAAAGG | 17804 | 17823 | Intron 8 | −21.3 | 202 |
| 188 | TCGACCAGGCCAAGCACTTG | 17773 | 17792 | Intron 8 | −26.4 | 203 |
| 189 | CCAAGCACTTGGCTATCACA | 17764 | 17783 | Intron 8 | −24.1 | 204 |
| 190 | GCCTTTAATCAGCCAGTTCT | 17729 | 17748 | Intron 8 | −22.6 | 205 |
| 191 | CTACAGACAGCCCACCTGTG | 17707 | 17726 | Intron 8 | −23.1 | 206 |
| 192 | CTTTCACATGGCGGTCAAGG | 17657 | 17676 | Intron 8 | −22.5 | 207 |
| 193 | CAAGGCAAACGAGACAGGAA | 17642 | 17661 | Intron 8 | −25 | 208 |
| 194 | AGGAAATCACAACAATCACA | 17627 | 17646 | Intron 8 | −20.1 | 209 |
| 195 | CACAACAATCACACCGAGAT | 17611 | 17630 | Intron 8 | −21.9 | 210 |
| 196 | GAGATCGGAGGGTGAGCTTC | 17596 | 17615 | Intron 8 | −23.7 | 211 |
| 197 | GAAGGCTCGTTCTGGGCTGA | 17574 | 17593 | Intron 8 | −20.4 | 212 |
| 198 | CTGATTCAGAGCCGCGTTTC | 17558 | 17577 | Intron 8 | −21.3 | 213 |
| 199 | CCTCAGCGATTCCTGCCAGG | 17534 | 17553 | Intron 8 | −30.2 | 214 |
| 200 | GTACCGCCCCACGGCCATCC | 17500 | 17519 | Intron 8 | −22.2 | 215 |
| 201 | TCACCAGCGAGCTTCTCCAT | 17449 | 17468 | Exon 8-Intron | −23.4 | 216 |
| | | | | 8 junction | | |
| 202 | CTGGACCAAGGTATCCTCGG | 17429 | 17448 | Exon 8 | −22.9 | 217 |
| 203 | GAGCTTCTCGGTCAGCCGGC | 17393 | 17412 | Exon 8 | −27.2 | 218 |
| 204 | GCCGGCTGCCACACGCCTTC | 17379 | 17398 | Exon 8 | −23.5 | 219 |
| 205 | CACACGCCTTCAGGAGCCTG | 17370 | 17389 | Exon 8-Intron | −24.4 | 220 |
| | | | | 7 junction | | |
| 206 | GGAGAACACAGCACCAGCTA | 17351 | 17370 | Intron 7 | −20.6 | 221 |
| 207 | TGAGCGTTCTCGCCAAAGGA | 17331 | 17350 | Intron 7 | −21.9 | 222 |
| 208 | ACCTGACGCTCACCCAGCCC | 17303 | 17322 | Intron 7 | −22.2 | 223 |
| 209 | GGCACAACCAGCCAGATCCC | 17234 | 17253 | Intron 7 | −21.8 | 224 |
| 210 | GCCAGATCCCGCATGCAGGC | 17224 | 17243 | Intron 7 | −23.2 | 225 |
| 211 | GCTCCGATGCCAATGCAGTC | 17097 | 17116 | Intron 7 | −24.1 | 226 |
| 212 | AGGTAAACTGCACAAGGGCT | 17063 | 17082 | Intron 7 | −22.6 | 227 |
| 213 | GCAGCCTGGAGAACACTCAG | 17042 | 17061 | Intron 7 | −26 | 228 |
| 214 | GTGATTATATTTATGGCTGG | 16986 | 17005 | Intron 7 | −21.1 | 229 |
| 215 | ATGGCTGGAATGATACGACT | 16974 | 16993 | Intron 7 | −23.4 | 230 |
| 216 | CGACTCAGATTTGCTTTAAA | 16959 | 16978 | Intron 7 | −22.6 | 231 |
| 217 | AAAAAAAAAGGCTGGGCGCG | 16936 | 16955 | Intron 7 | −25.7 | 232 |
| 218 | GAAGAAGTGAGTTCCACATC | 16718 | 16737 | Intron 7 | −24.4 | 233 |
| 219 | TCCACATCAATATGTTTTCA | 16706 | 16725 | Intron 7 | −21.7 | 234 |
| 220 | TTCAGCACACTCAGGGGAAG | 16690 | 16709 | Intron 7 | −28.2 | 235 |
| 221 | CCTACAGAACAAGGACTCCC | 16667 | 16686 | Intron 7 | −24.4 | 236 |
| 222 | CCAGATCTGTGCTCACTGAC | 16644 | 16663 | Intron 7 | −25.7 | 237 |
| 223 | CTCACTGACAAGTGCCCACA | 16633 | 16652 | Intron 7 | −21.5 | 238 |
| 224 | CCAGTGCTCCTCACAGAGGC | 16611 | 16630 | Intron 7 | −25.6 | 239 |
| 225 | CAGAGGCAGCAAGCAAACAC | 16598 | 16617 | Intron 7 | −24.2 | 240 |
| 226 | GCTAAGGACTGTTCTCCCAA | 16577 | 16596 | Intron 7 | −24.9 | 241 |
| 227 | CCAAATCCATGGACAAGCCA | 16561 | 16580 | Intron 7 | −20.8 | 242 |
| 228 | GACTGCTCTATCCTAACAGA | 16528 | 16547 | Intron 7 | −21.4 | 243 |
| 229 | CAAAAGCAGAGACGCCCGTT | 16503 | 16522 | Intron 7 | −21.5 | 244 |
| 230 | CCCGTTACCAGGCAAAGGAG | 16489 | 16508 | Exon 7-Intron | −21.8 | 245 |
| | | | | 7 junction | | |
| 231 | AGCGATGTCAGCGAGCGGGC | 16433 | 16452 | Exon 7 | −22 | 246 |
| 232 | GGCGTTGCCGTTCCTCTGGT | 16413 | 16432 | Exon 7 | −27.3 | 247 |
| 233 | CATCCTCTGAGCACGCTGTG | 16368 | 16387 | Exon 7 | −21.7 | 248 |
| 234 | AGCAGCTCGGACCCCTACTC | 16275 | 16294 | Intron 6 | −26 | 249 |
| 235 | CTCGTTCACAGCCAACTCCA | 16258 | 16277 | Intron 6 | −25.1 | 250 |
| 236 | CAACTCCAGGACCTGACTCC | 16246 | 16265 | Intron 6 | −21.7 | 251 |
| 237 | GACTCCTGGAGGATCAGTCC | 16232 | 16251 | Intron 6 | −25.5 | 252 |
| 238 | GAGCACACAAAGTGCCACCA | 16192 | 16211 | Intron 6 | −21.4 | 253 |
| 239 | ATTCCCAACAGCATAGCTCT | 16164 | 16183 | Intron 6 | −25.5 | 254 |
| 240 | GCTCTGCCCCGGCTACATCA | 16149 | 16168 | Intron 6 | −24 | 255 |
| 241 | ACATCAGGAGCTATCCGAGA | 16135 | 16154 | Intron 6 | −20.5 | 256 |
| 242 | GCTATCCGAGAACAGAAATC | 16126 | 16145 | Intron 6 | −24.5 | 257 |
| 243 | GAGCTTCTCCTGTCCCCTTC | 16102 | 16121 | Intron 6 | −27 | 258 |
| 244 | TTCCTAATGAGTGACCCAAG | 16085 | 16104 | Intron 6 | −20.4 | 259 |
| 245 | TTCTCGTGAGTGGCCATCTG | 16052 | 16071 | Intron 6 | −22.4 | 260 |
| 246 | TGGCCATCTGCGCTGCTCCC | 16042 | 16061 | Intron 6 | −22.7 | 261 |
| 247 | GTCTCCACCTCCCCTTCTGG | 16017 | 16036 | Intron 6 | −32.1 | 262 |
| 248 | GACTGGCTCTGGTCAATGAG | 15971 | 15990 | Intron 6 | −25.9 | 263 |
| 249 | GGTCAATGAGCTGAGACAAA | 15961 | 15980 | Intron 6 | −22.5 | 264 |
| 250 | TTCAAGCGTTTCTCCTGCCT | 15792 | 15811 | Intron 6 | −21.6 | 265 |
| 251 | TATTTCTGGAAGAGACGGGG | 15716 | 15735 | Intron 6 | −22.8 | 266 |
| 252 | AGCATTTTCATATGGCTGCC | 15582 | 15601 | Intron 6 | −20.9 | 267 |
| 253 | CTGCCTGCCAGGACGACCAC | 15559 | 15578 | Intron 6 | −21.7 | 268 |
| 254 | GGACGACCACAGCAAGCAGA | 15549 | 15568 | Intron 6 | −26.2 | 269 |
| 255 | CCCCACTCTCCTGCAATGCA | 15528 | 15547 | Intron 6 | −26.2 | 270 |
| 256 | CACACCATGAGTAAGAAACA | 15508 | 15527 | Intron 6 | −22 | 271 |
| 257 | GCTTGAAGGTGCTGAGATCC | 15479 | 15498 | Intron 6 | −25.7 | 272 |
| 258 | GTTACCAGAGCACGACCTGG | 15453 | 15472 | Intron 6 | −21.3 | 273 |
| 259 | CTGGCCTGAGCTGCATCTCC | 15427 | 15446 | Intron 6 | −25 | 274 |
| 260 | AGCTGGAGGCCTACATTCGG | 15405 | 15424 | Intron 6 | −24.3 | 275 |
| 261 | ACATTCGGTGGTGCAACTCA | 15393 | 15412 | Intron 6 | −25.9 | 276 |
| 262 | GAGATGAAGGCCAGAGGAAA | 15352 | 15371 | Intron 6 | −26.8 | 277 |
| 263 | GCCAGAGGAAACAAAGACAC | 15343 | 15362 | Intron 6 | −25.5 | 278 |
| 264 | GAAACCAGAGCTGAGCGAGA | 15302 | 15321 | Intron 6 | −22.6 | 279 |
| 265 | GAGCGAGAACCAGGACAACG | 15290 | 15309 | Intron 6 | −27.5 | 280 |
| 266 | GACAACGGGCACAAGAGAGA | 15277 | 15296 | Intron 6 | −20.7 | 281 |
| 267 | GAAGACGCCACGCGGTCTCC | 15157 | 15176 | Intron 6 | −22.2 | 282 |
| 268 | TGAATTCACCTTGAGCGCCT | 15069 | 15088 | Exon 6-Intron | −20.6 | 283 |
| | | | | 6 junction | | |
| 269 | GCCCTGGAGCTCATCGATGA | 15048 | 15067 | Exon 6 | −22.9 | 284 |
| 270 | TTGATGAGGTAGATCCGGTC | 14993 | 15012 | Exon 6 | −20.5 | 285 |
| 271 | AGATCCGGTCCATCATGATG | 14983 | 15002 | Exon 6 | −20.8 | 286 |
| 272 | GATGATGCTGTACCAGCGCT | 14961 | 14980 | Exon 6 | −22 | 287 |
| 273 | CCAGGCTGTCCTTGATGAAG | 14929 | 14948 | Exon 6 | −20.4 | 288 |
| 274 | CTTCACGGCCAGGGCAGACC | 14857 | 14876 | Exon 6-Intron | −21.9 | 289 |
| | | | | 5 junction | | |
| 275 | CAGGGCAGACCTGGAGGGAC | 14848 | 14867 | Exon 6-Intron | −23.6 | 290 |
| | | | | 5 junction | | |
| 276 | CTGGAGGGACACCGGCGACT | 14838 | 14857 | Intron 5 | −24.9 | 291 |
| 277 | CAGACAGCCCTTTCCTCGCT | 14818 | 14837 | Intron 5 | −21.7 | 292 |
| 278 | TTTCCTCGCTTAGTGACACC | 14808 | 14827 | Intron 5 | −22.6 | 293 |
| 279 | ACCAAATCAAAGCCTCTTCT | 14791 | 14810 | Intron 5 | −21.5 | 294 |
| 280 | TCTTCAGACTTTTCAGAGTC | 14774 | 14793 | Intron 5 | −20.7 | 295 |
| 281 | TCAGCTGGCACAAATCAGCC | 14756 | 14775 | Intron 5 | −20.2 | 296 |
| 282 | GCGTTAATAACGGGGAGAGT | 14735 | 14754 | Intron 5 | −20.8 | 297 |
| 283 | GAGTGGCACAGTGGGGGCCA | 14719 | 14738 | Intron 5 | −25.6 | 298 |
| 284 | CAGACTCAAATGTACCAGCT | 14551 | 14570 | Intron 5 | −23.4 | 299 |
| 285 | CTTACTCACCTGGAGCATGC | 14533 | 14552 | Intron 5 | −21.8 | 300 |
| 286 | CAGAAGAACCAGCAGGTCAG | 14514 | 14533 | Intron 5 | −23.4 | 301 |
| 287 | CCACGCCTGGCTAATTTTTT | 14293 | 14312 | Intron 5 | −29.7 | 302 |
| 288 | AGCCCCACGTACTTTATTCT | 14148 | 14167 | Intron 5 | −21.4 | 303 |
| 289 | AAGAGGATTTCAGTCCCTCC | 14125 | 14144 | Intron 5 | −22.8 | 304 |
| 290 | GAGAGGACAGTTGAGGGCAG | 14018 | 14037 | Intron 5 | −29.1 | 305 |
| 291 | GCCACACTGAATGAGAACAG | 13991 | 14010 | Intron 5 | −26.8 | 306 |
| 292 | GAGAACAGTAAGGGAGCTTG | 13979 | 13998 | Intron 5 | −30.3 | 307 |
| 293 | TGTCGAACTGGGTGGACACC | 13956 | 13975 | Intron 5 | −29.39 | 308 |
| 294 | GACACCAATGACAAGGAAGT | 13942 | 13961 | Intron 5 | −23.4 | 309 |
| 295 | GACAAGGAAGTTTAGTAACT | 13933 | 13952 | Intron 5 | −22.4 | 310 |
| 296 | GTAACTGAACGACGAATTCT | 13919 | 13938 | Intron 5 | −20 | 311 |
| 297 | GTAACAAAGACCTGAGCTCA | 13897 | 13916 | Intron 5 | −27.3 | 312 |
| 298 | CTCAGAGTCAAGAGCAACAG | 13881 | 13900 | Intron 5 | −23.61 | 313 |
| 299 | ATCCACACCTCAGCAGGCTG | 13833 | 13852 | Intron 5 | −26.8 | 314 |
| 300 | GCAGGCTGAGAGCAGCACGG | 13821 | 13840 | Intron 5 | −29.7 | 315 |
| 301 | TGAGACTTGGAATGAGGACT | 13792 | 13811 | Intron 5 | −28.49 | 316 |
| 302 | GGACTGAGATAATAAAAAAA | 13777 | 13796 | Intron 5 | −20.59 | 317 |
| 303 | GTGATGTTGATGCAATGAAC | 13756 | 13775 | Intron 5 | −20.6 | 318 |
| 304 | CATGGCGGCTCACACCTGTA | 13670 | 13689 | Intron 5 | −28.3 | 319 |
| 305 | GAGGATGAGCCAGAGGATTG | 13635 | 13654 | Intron 5 | −30.31 | 320 |
| 306 | CCAGAGGATTGCTTGAGTTC | 13626 | 13645 | Intron 5 | −29.8 | 321 |
| 307 | AGTTCGAGACCAACCTGGGC | 13611 | 13630 | Intron 5 | −26.5 | 322 |
| 308 | GGCCGAGGCGGGCAAATCAC | 13507 | 13526 | Intron 5 | −29.3 | 323 |
| 309 | CAAAAAACAAACCCCAGTTG | 13263 | 13282 | Intron 5 | −22 | 324 |
| 310 | ACCTGGGAGGCTAAGGCGGG | 13212 | 13231 | Intron 5 | −32.5 | 325 |
| 311 | CAGAGCGAGACCCTGACACA | 13120 | 13139 | Intron 5 | −27.9 | 326 |
| 312 | CCTGACACAAAAGAAGGAAA | 13109 | 13128 | Intron 5 | −22.62 | 327 |
| 313 | GAAAATGCTCAGCAAGTCCA | 13012 | 13031 | Intron 5 | −22.42 | 328 |
| 314 | CAGCAAGTCCAACATGACTC | 13003 | 13022 | Intron 5 | −21.22 | 329 |
| 315 | TCCTCCCGCAATTCTGGACA | 12985 | 13004 | Intron 5 | −20.12 | 330 |
| 316 | AGGCGTCCTGTACCCTGTGC | 12961 | 12980 | Intron 5 | −23.82 | 331 |
| 317 | CGGGTCCGCCCTGAGAGAGG | 12929 | 12948 | Intron 5 | −27.92 | 332 |
| 318 | AGGACCAGTGCCTGCCTCCC | 12912 | 12931 | Intron 5 | −23.9 | 333 |
| 319 | CTGCCTCCCTGTGCAATGCT | 12901 | 12920 | Intron 5 | −29.9 | 334 |
| 320 | GTGCAATGCTGGCTCCGAGC | 12891 | 12910 | Intron 5 | −34.5 | 335 |
| 321 | GCTCCGAGCCCACCCAGAGC | 12880 | 12899 | Intron 5 | −25.4 | 336 |
| 322 | GGCCACAAGGCTCACCTCAC | 12821 | 12840 | Exon 5-Intron | −23.6 | 337 |
| | | | | 5 junction | | |
| 323 | GCTCAGGCTCCGGACACAGG | 12800 | 12819 | Exon 5 | −22.4 | 338 |
| 324 | GGCCTGGCGGACAATGCTGA | 12782 | 12801 | Exon 5 | −23.6 | 339 |
| 325 | AAGAGCTGGGGGTGGCTGGG | 12763 | 12782 | Exon 5 | −28.5 | 340 |
| 326 | GGTGCTGGTGGCTGACGTAT | 12744 | 12763 | Exon 5 | −26 | 341 |
| 327 | TATCATGGCTGATATATCCC | 12708 | 12727 | Exon 5 | −22 | 342 |
| 328 | GGTGCCCTGCAGCAAGTGAC | 12687 | 12706 | Exon 5 | −28.3 | 343 |
| 329 | CATGAAAAGGAAAAGTAAAT | 12654 | 12673 | Intron 4 | −22.1 | 344 |
| 330 | GTAAATCTGTTAGTTGGGAA | 12640 | 12659 | Intron 4 | −22 | 345 |
| 331 | GACAAACTCTCTTAGGTTTA | 12613 | 12632 | Intron 4 | −21.5 | 346 |
| 332 | CAGAACACAGAGATCGCAGA | 12491 | 12510 | Intron 4 | −24.7 | 347 |
| 333 | GGTTGTTCCTAACAGGTATT | 12461 | 12480 | Intron 4 | −23 | 348 |
| 334 | GAGCACTTACGAAGTAGGTA | 12437 | 12456 | Intron 4 | −20.1 | 349 |
| 335 | AGGTAACATGAAAAGGGCCC | 12422 | 12441 | Intron 4 | −21 | 350 |
| 336 | CTGCATGCTCCATCTCATTC | 12403 | 12422 | Intron 4 | −27.9 | 351 |
| 337 | CCCTCTGAGATAATGTCAGT | 12369 | 12388 | Intron 4 | −21.5 | 352 |
| 338 | TCTACAGAAGACAAAACTGA | 12337 | 12356 | Intron 4 | −20 | 353 |
| 339 | GAAGTAGCTGGCCCGAGGGT | 12306 | 12325 | Intron 4 | −30.6 | 354 |
| 340 | GTAACACTACCCACTGCCCC | 12270 | 12289 | Intron 4 | −24.4 | 355 |
| 341 | CTCCATCATTGGTTCTGAAC | 12246 | 12265 | Intron 4 | −23.7 | 356 |
| 342 | ACCTGGAAGGGGCTTCAGCC | 12222 | 12241 | Intron 4 | −27.3 | 357 |
| 343 | AGCCACAGGCGAGAGGCCCA | 12206 | 12225 | Intron 4 | −29.8 | 358 |
| 344 | TAGGGCGAGCACCAAGCTCC | 12181 | 12200 | Intron 4 | −20.3 | 359 |
| 345 | AGCCACTCCGATGCTGAGGC | 12107 | 12126 | Intron 4 | −24.1 | 360 |
| 346 | GAGGCTGTGCGCCCTGGAGC | 12092 | 12111 | Intron 4 | −23 | 361 |
| 347 | GCTGCTCCTACCCCAGGACT | 12074 | 12093 | Intron 4 | −20.3 | 362 |
| 348 | CTGTGCGGCAGTGCGTCAGC | 12056 | 12075 | Intron 4 | −21.4 | 363 |
| 349 | AGACTCTCCCCACAGTGTCG | 12035 | 12054 | Intron 4 | −24.7 | 364 |
| 350 | CCTCCATGGGCAACCCCAAA | 12008 | 12027 | Intron 4 | −21.2 | 365 |
| 351 | AACCAGCTCAAGGATGCCCC | 11989 | 12008 | Intron 4 | −26.3 | 366 |
| 352 | GCAGCCTGGCGGTGCCCTCC | 11968 | 11987 | Intron 4 | −31.9 | 367 |
| 353 | CCCTCCTTCCCAGGTCTGAG | 11954 | 11973 | Intron 4 | −25.3 | 368 |
| 354 | TGCTCCCCATCCCCCATTCC | 11933 | 11952 | Intron 4 | −25.9 | 369 |
| 355 | CCCCATTCCTTACAGGCAAT | 11922 | 11941 | Intron 4 | −24.1 | 370 |
| 356 | TCCAGCAATGGATGCAGCTC | 11902 | 11921 | Intron 4 | −23.6 | 371 |
| 357 | GCTCTTACACGTCCTCTGCT | 11886 | 11905 | Intron 4 | −21.5 | 372 |
| 358 | GAACAAGCTTGGGTTCCCGT | 11848 | 11867 | Intron 4 | −20.6 | 373 |
| 359 | GGCAGGTCCAACACCCACCC | 11816 | 11835 | Intron 4 | −22.9 | 374 |
| 360 | GATTTTGGGCCCTGACCCAG | 11794 | 11813 | Intron 4 | −23.3 | 375 |
| 361 | GACCCAGGTCCTCCACAAAA | 11781 | 11800 | Intron 4 | −20.3 | 376 |
| 362 | CACTAATTCCCGAGAGTTCA | 11735 | 11754 | Intron 4 | −21.2 | 377 |
| 363 | GGAGTGACCCACCGAGAACA | 11706 | 11725 | Intron 4 | −23 | 378 |
| 364 | GGGAGCAGGAAACTCCACTG | 11675 | 11694 | Intron 4 | −23.9 | 379 |
| 365 | GACCCGCCTCCAGGACAGGC | 11637 | 11656 | Intron 4 | −28.6 | 380 |
| 366 | CCAGGACAGGCTGGAGTGTG | 11628 | 11647 | Intron 4 | −25.8 | 381 |
| 367 | ACAAACATGAAGGCCACGGC | 11603 | 11622 | Intron 4 | −27.6 | 382 |
| 368 | CCACGGCAGGGAGGCAGAGT | 11590 | 11609 | Intron 4 | −22.8 | 383 |
| 369 | GTTAAAGAAGTTAACATAGG | 11568 | 11587 | Intron 4 | −20.7 | 384 |
| 370 | GTTAACATAGGCTGGGTGTG | 11559 | 11578 | Intron 4 | −30.2 | 385 |
| 371 | CGTGGTGGCAGGCAAGTGTA | 11409 | 11428 | Intron 4 | −34.5 | 386 |
| 372 | AATTCGTTATTCGGGAGGCT | 11390 | 11409 | Intron 4 | −27.4 | 387 |
| 373 | TTGCCTCGCACATGTCCGAC | 11097 | 11116 | Exon 4-Intron | −20.1 | 388 |
| | | | | 4 junction | | |
| 374 | TGTCCGACTTTTTGGGCCCC | 11085 | 11104 | Exon 4 | −22.2 | 389 |
| 375 | CCCCGGGCTGCTGGACTCGA | 11069 | 11088 | Exon 4 | −25.3 | 390 |
| 376 | ACGCTGGCCCCCTCTGCGGG | 11050 | 11069 | Exon 4 | −27 | 391 |
| 377 | CACCTCCGTGCAGAAGAGAG | 10916 | 10935 | Exon 4 | −23.8 | 392 |
| 378 | GAGAGTGCGGGGGCCGTGGA | 10901 | 10920 | Exon 4 | −20 | 393 |
| 379 | GGGCCGTGGAGCTCGCAGAA | 10891 | 10910 | Exon 4 | −27.3 | 394 |
| 380 | GGACAGGGGACATGTCAGCT | 10813 | 10832 | Intron 3 | −25.8 | 395 |
| 381 | GGTGCCATGGACTTCCTGCC | 10732 | 10751 | Intron 3 | −27.7 | 396 |
| 382 | AGAGGCCACCCGCCAGTTCC | 10708 | 10727 | Intron 3 | −25.4 | 397 |
| 383 | CAGTTCCCCAGCCCCCTGCC | 10695 | 10714 | Intron 3 | −32.8 | 398 |
| 384 | CTGCTTTGCCCCAGTGCGAG | 10671 | 10690 | Intron 3 | −25.6 | 399 |
| 385 | AGATAGGATCTTCCCATGGG | 10643 | 10662 | Intron 3 | −24.6 | 400 |
| 386 | AGTCACAGCAAATAGGGGAG | 10623 | 10642 | Intron 3 | −21.9 | 401 |
| 387 | GGGGAGGGGACAATGTGGAC | 10609 | 10628 | Intron 3 | −31.1 | 402 |
| 388 | ACCAATGATAGCACAGCTGG | 10591 | 10610 | Intron 3 | −21.4 | 403 |
| 389 | GCCACAAAGCCAACGGCGCT | 10553 | 10572 | Intron 3 | −20.3 | 404 |
| 390 | CGGCGCTGGAAAGGAATGTC | 10540 | 10559 | Intron 3 | −27 | 405 |
| 391 | GAACTGCAGCCAAACCTGGC | 10508 | 10527 | Intron 3 | −21.3 | 406 |
| 392 | ACAAATAGATAAAAGGCCAC | 10475 | 10494 | Intron 3 | −20.5 | 407 |
| 393 | GAGGGTCCTCCACCCAATAC | 10450 | 10469 | Intron 3 | −27 | 408 |
| 394 | ACCCAATACATATCTGCTTG | 10439 | 10458 | Intron 3 | −21.8 | 409 |
| 395 | CTTACGGGGCCAAGCAAGAG | 10262 | 10281 | Intron 3 | −22.9 | 410 |
| 396 | GCAAGAGTTGAAGGGCAGGG | 10249 | 10268 | Intron 3 | −31.3 | 411 |
| 397 | GCAGGGTGGGTGCTGTACTG | 10235 | 10254 | Intron 3 | −31.9 | 412 |
| 398 | GAGCAGCAGCAGACAGGGTG | 10184 | 10203 | Intron 3 | −28.8 | 413 |
| 399 | GAGGCTGTAGGAAGCAGATC | 10164 | 10183 | Intron 3 | −26 | 414 |
| 400 | AGCAGATCTTATAGCTCCTC | 10152 | 10171 | Intron 3 | −22.3 | 415 |
| 401 | TCCAGGCCACCGAAAGACCC | 10134 | 10153 | Intron 3 | −23.3 | 416 |
| 402 | GGTTCTGATGAGACATGAGA | 10080 | 10099 | Intron 3 | −26 | 417 |
| 403 | GAGACATGAGAAAATGTGAA | 10071 | 10090 | Intron 3 | −23.3 | 418 |
| 404 | TGAATTTCACTTGAATAGGT | 10055 | 10074 | Intron 3 | −20.3 | 419 |
| 405 | GGTTCACTCATGCCCATGTG | 10038 | 10057 | Intron 3 | −21.8 | 420 |
| 406 | TGGCAGGGGGCCAGGGTGGA | 10009 | 10028 | Intron 3 | −33.4 | 421 |
| 407 | GGGGAACAAGCTGAGGCACC | 9986 | 10005 | Intron 3 | −29.5 | 422 |
| 408 | GATCTGAGCAGGAAGGGACA | 9957 | 9976 | Intron 3 | −29.9 | 423 |
| 409 | CAGGCCAGGCAGGGTGAAGA | 9931 | 9950 | Intron 3 | −34.2 | 424 |
| 410 | AGGGTGAAGACGCACAGTGC | 9921 | 9940 | Intron 3 | −24.4 | 425 |
| 411 | GTGCGGGTGGGGTGCAGAGT | 9905 | 9924 | Intron 3 | −30.4 | 426 |
| 412 | GAGAAGGCCCAGTGAGTGCC | 9867 | 9886 | Intron 3 | −29.3 | 427 |
| 413 | GAGTGCCTGCTGAGCCTTCA | 9854 | 9873 | Intron 3 | −24.5 | 428 |
| 414 | GGACGGGACACAGCAGCAGA | 9831 | 9850 | Intron 3 | −33.4 | 429 |
| 415 | AGCAGCAGAGCCAAGCGCTG | 9820 | 9839 | Intron 3 | −26.2 | 430 |
| 416 | CGGTCGGGGCTGACGCATGG | 9800 | 9819 | Intron 3 | −21.6 | 431 |
| 417 | ATGGGACCTCCGTGGAGTGC | 9784 | 9803 | Intron 3 | −26.2 | 432 |
| 418 | AGCCCTTGTAGACTCAGAGG | 9764 | 9783 | Intron 3 | −21 | 433 |
| 419 | AGGGAGGACAGCTAGCCAAC | 9738 | 9757 | Intron 3 | −29.9 | 434 |
| 420 | GCTAGCCAACATGACCAAGC | 9728 | 9747 | Intron 3 | −23.6 | 435 |
| 421 | CCAAGCACAGGCAGTGAGGC | 9714 | 9733 | Intron 3 | −24.9 | 436 |
| 422 | GTGAGGCCAGAGGGGAGCTC | 9701 | 9720 | Intron 3 | −26.7 | 437 |
| 423 | GCTTTAGCAAGGTGGGACTG | 9653 | 9672 | Intron 3 | −26.2 | 438 |
| 424 | GTCACATGGCCTTAAAAAGG | 9629 | 9648 | Intron 3 | −23.3 | 439 |
| 425 | GCCACAAGCTACGGCACGGC | 9598 | 9617 | Intron 3 | −20.9 | 440 |
| 426 | GGCACGGCTGAAACTGAAGG | 9586 | 9605 | Intron 3 | −21.5 | 441 |
| 427 | GAAGGACATTACACGAAATG | 9571 | 9590 | Intron 3 | −21.7 | 442 |
| 428 | CCACTTAAATAGGAGGTACC | 9513 | 9532 | Intron 3 | −22.8 | 443 |
| 429 | GTCCTCAAAGCCATAGAGAC | 9489 | 9508 | Intron 3 | −23.3 | 444 |
| 430 | AGAGAGCAGAATGGCAGCTG | 9469 | 9488 | Intron 3 | −31.5 | 445 |
| 431 | TGCCGGGGACTGGGAGGGAA | 9451 | 9470 | Intron 3 | −37.5 | 446 |
| 432 | GGGGCTCAGTTCTGGAGACG | 9427 | 9446 | Intron 3 | −33.2 | 447 |
| 433 | GACGGAAGGTGGCGATGGCC | 9411 | 9430 | Intron 3 | −34.8 | 448 |
| 434 | GCGATGGCCGCACAGCACTG | 9400 | 9419 | Intron 3 | −23.2 | 449 |
| 435 | GTGAACATACTTCATGCCAC | 9381 | 9400 | Intron 3 | −22.8 | 450 |
| 436 | GGCACTGCACGCCGAACAGT | 9361 | 9380 | Intron 3 | −21.3 | 451 |
| 437 | CAGTGGTTCAAATGGCGCAT | 9345 | 9364 | Intron 3 | −20.6 | 452 |
| 438 | CATTTTAGGTCATGTATTAT | 9328 | 9347 | Intron 3 | −20 | 453 |
| 439 | AATCTTTTTAAAAGGGGGAG | 9300 | 9319 | Intron 3 | −20.3 | 454 |
| 440 | GGAGTGGTAACCTGCAGAAC | 9284 | 9303 | Intron 3 | −26.8 | 455 |
| 441 | CAAAGGTGACCTTAACACAG | 9253 | 9272 | Intron 3 | −22.1 | 456 |
| 442 | GAAAAAGCTCACAGGAGTGG | 9222 | 9241 | Intron 3 | −23.3 | 457 |
| 443 | GTTTCCGTGGCAGAGCCAGC | 9181 | 9200 | Intron 3 | −24.5 | 458 |
| 444 | CAGAGCCAGCAATGGGACAC | 9171 | 9190 | Intron 3 | −25.8 | 459 |
| 445 | GGGGACATGGAGTCAAGGAA | 9146 | 9165 | Intron 3 | −32.5 | 460 |
| 446 | TGGCCACAGTACAGCTGCGA | 9115 | 9134 | Intron 3 | −21.5 | 461 |
| 447 | ACCATGTGAAGGCCAGGGGC | 9085 | 9104 | Intron 3 | −30.8 | 462 |
| 448 | CAGCTGCAGGCACAAAGTCT | 9066 | 9085 | Intron 3 | −28.8 | 463 |
| 449 | AGCACTCATTGCAGAGGGAT | 9035 | 9054 | Intron 3 | −26.1 | 464 |
| 450 | CCGCGGCAGCATGAGGCAGT | 9013 | 9032 | Intron 3 | −29.8 | 465 |
| 451 | ACGAATGCAGGAAAGCCAGA | 8982 | 9001 | Intron 3 | −23 | 466 |
| 452 | GAAGGCTTTAAAATGGGGGT | 8962 | 8981 | Intron 3 | −24.5 | 467 |
| 453 | GTTCTCACTGGCAGAGAAGT | 8932 | 8951 | Intron 3 | −23 | 468 |
| 454 | GAAGTTCTGGAAACGAGACC | 8917 | 8936 | Intron 3 | −21.7 | 469 |
| 455 | CCTGACACATGCAGCCAGAC | 8838 | 8857 | Intron 3 | −22.6 | 470 |
| 456 | CATTGTCCATACGGTGGCCA | 8815 | 8834 | Intron 3 | −23.8 | 471 |
| 457 | GGCCACAGCTATGTGTGACA | 8800 | 8819 | Intron 3 | −23.8 | 472 |
| 458 | AATTTAAATTAGCTAGGAGA | 8768 | 8787 | Intron 3 | −20.5 | 473 |
| 459 | TAGGAGAGGCCGGCACAGTG | 8755 | 8774 | Intron 3 | −34 | 474 |
| 460 | TCCCATGAGCCTGTAATCCC | 8721 | 8740 | Intron 3 | −20.6 | 475 |
| 461 | CACTTGAGGTCAGGAGTTTG | 8672 | 8691 | Intron 3 | −31.8 | 476 |
| 462 | TGGCCGACGCAGTGAAACCC | 8643 | 8662 | Intron 3 | −24.6 | 477 |
| 463 | CACTTGAACCTGGGAGGCAA | 8537 | 8556 | Intron 3 | −36 | 478 |
| 464 | GATCGCACCACGGCACTCGA | 8501 | 8520 | Intron 3 | −29 | 479 |
| 465 | CCAAAAACAACAAAAACAAA | 8444 | 8463 | Intron 3 | −20.6 | 480 |
| 466 | ACAAAATAAAACAAAAACCT | 8424 | 8443 | Intron 3 | −21.9 | 481 |
| 467 | ACAAAAACCTTAGCTGGGCG | 8414 | 8433 | Intron 3 | −21.7 | 482 |
| 468 | GCGTGTTGGCGGTCACCTAT | 8397 | 8416 | Intron 3 | −21.1 | 483 |
| 469 | ATCACTTGAACCTGGGAGGC | 8344 | 8363 | Intron 3 | −35.2 | 484 |
| 470 | GCCTTTATGACAGGGCGAGA | 8285 | 8304 | Intron 3 | −21.8 | 485 |
| 471 | AAAAAAAGTAGCCTGGAGTG | 8245 | 8264 | Intron 3 | −23.8 | 486 |
| 472 | CCTGGAGTGGTGGCGCACGC | 8234 | 8253 | Intron 3 | −38.5 | 487 |
| 473 | GCTGAGTGCGGTTGTTCATG | 8066 | 8085 | Intron 3 | −21.9 | 488 |
| 474 | ATATTAGCTAGGACAAAAGC | 7756 | 7775 | Intron 3 | −22 | 489 |
| 475 | TCACCGGCCATGTCCTGAGT | 7722 | 7741 | Intron 3 | −21.1 | 490 |
| 476 | TGCTAGGCACACACGACTCA | 7699 | 7718 | Intron 3 | −29.8 | 491 |
| 477 | AGCCCAGGTCACAGAATATT | 7663 | 7682 | Intron 3 | −25.6 | 492 |
| 478 | GTGTGGCTGGCAGGGAATGG | 7610 | 7629 | Intron 3 | −31.6 | 493 |
| 479 | AGGGCTGGTGCTCAGTAGGT | 7590 | 7609 | Intron 3 | −24.6 | 494 |
| 480 | TCCGAGAGGGAGGCAGACAG | 7566 | 7585 | Intron 3 | −32.3 | 495 |
| 481 | CAGAGTTTCAATAAAGGGCC | 7530 | 7549 | Intron 3 | −26 | 496 |
| 482 | CTTGCTTGGGACGATGAAGA | 7501 | 7520 | Intron 3 | −23.9 | 497 |
| 483 | TCATGTAAGTTTCTGAATGT | 7480 | 7499 | Intron 3 | −22.3 | 498 |
| 484 | CTGAGTAGTGGACACTGGCC | 7456 | 7475 | Intron 3 | −29.6 | 499 |
| 485 | TCTTCTCTTCTGGGGAGCCC | 7423 | 7442 | Intron 3 | −24.8 | 500 |
| 486 | CCCCACCCCTGTTACGGATC | 7405 | 7424 | Intron 3 | −21.1 | 501 |
| 487 | TGTCTTCGCTCCTGACAGGC | 7369 | 7388 | Intron 3 | −24 | 502 |
| 488 | GTGAACACAAACAAGGCATG | 7344 | 7363 | Intron 3 | −24.8 | 503 |
| 489 | CCTGCCAAAGCCGCTAACTC | 7323 | 7342 | Intron 3 | −20.6 | 504 |
| 490 | TCTAGGAAGAAGCAAAGGAC | 7305 | 7324 | Intron 3 | −21.8 | 505 |
| 491 | CGATGTGCATGGTGGTGGGG | 7286 | 7305 | Intron 3 | −25.7 | 506 |
| 492 | GCAGCCAGCCTCTCTGCCGG | 7260 | 7279 | Intron 3 | −27 | 507 |
| 493 | CAGCATGCCTGCGAATGCAC | 7215 | 7234 | Intron 3 | −20.2 | 508 |
| 494 | GCACCACCCACCTGACGTTT | 7199 | 7218 | Exon 3-Intron | −27.4 | 509 |
| | | | | 3 junction | | |
| 495 | GACGTTTCTGCAGTTGGCTG | 7186 | 7205 | Exon 3 | −25.4 | 510 |
| 496 | AGCTGCACGAGCTGCTCCGG | 7163 | 7182 | Exon 3 | −22.7 | 511 |
| 497 | GCACGAGTGGTCACACTGGG | 7114 | 7133 | Exon 3-Intron | −20.9 | 512 |
| | | | | 2 junction | | |
| 498 | GGGCTGGCAGTGAGGCAGGT | 7047 | 7066 | Intron 2 | −26 | 513 |
| 499 | GCAGGTGTGGGAGTCAGAGA | 7033 | 7052 | Intron 2 | −30.3 | 514 |
| 500 | GAGAGGGAGACCCTGGGTGT | 7017 | 7036 | Intron 2 | −30 | 515 |
| 501 | GGGTGTGGCTGGCAGGGAAT | 7003 | 7022 | Intron 2 | −30.5 | 516 |
| 502 | TGTAGGTGCACTCCGAGAGG | 6968 | 6987 | Intron 2 | −23.9 | 517 |
| 503 | TGCTCAGAGTACCCCTTGCC | 6755 | 6774 | Intron 2 | −28.4 | 518 |
| 504 | CACCAGAAGAGCTCCCCTCC | 6731 | 6750 | Intron 2 | −21.1 | 519 |
| 505 | CTTTGAGTCGTAGCGCTCCT | 6687 | 6706 | Intron 2 | −21.5 | 520 |
| 506 | CTCCTCATCGCAGAACTGCG | 6672 | 6691 | Intron 2 | −22.7 | 521 |
| 507 | GAACTGCGCCTCCAAAGTGC | 6660 | 6679 | Intron 2 | −22.9 | 522 |
| 508 | CGGAATCTAGGGGCGGCAGA | 6542 | 6561 | Intron 2 | −25 | 523 |
| 509 | GAACCAAACAGACAGTTTCA | 6469 | 6488 | Intron 2 | −24.1 | 524 |
| 510 | ACAGTTTCAATAAAGGGCCC | 6458 | 6477 | Intron 2 | −22.9 | 525 |
| 511 | CCCACAGGGTTCACTATGAT | 6441 | 6460 | Intron 2 | −23.2 | 526 |
| 512 | GAGTGGCTTAAAAATCTACT | 6421 | 6440 | Intron 2 | −22.4 | 527 |
| 513 | CAGGAGATGGGGCGACCACT | 6395 | 6414 | Intron 2 | −25.5 | 528 |
| 514 | GTAAGGGGTAGCAGGACCCA | 6369 | 6388 | Intron 2 | −21.9 | 529 |
| 515 | GCGAAAAGGAAGGCCACGCC | 6325 | 6344 | Intron 2 | −24.7 | 530 |
| 516 | GCCACGCCTTTCTCTGAGAG | 6313 | 6332 | Intron 2 | −25 | 531 |
| 517 | TAAGGTTCCTTCTAGCAAGA | 6275 | 6294 | Intron 2 | −23.3 | 532 |
| 518 | TCTAGCAAGACATGCACAGG | 6265 | 6284 | Intron 2 | −21.8 | 533 |
| 519 | AAGTCTCAGCCCACCTGCAG | 6229 | 6248 | Intron 2 | −26.1 | 534 |
| 520 | CACGCTTCCACCGTCCACAC | 6177 | 6196 | Exon 2 | −20.3 | 535 |
| 521 | GACACCCTTGAAGACCTGGC | 6105 | 6124 | Exon 2 | −28.6 | 536 |
| 522 | GGCGTTTTCTAGGTAAGAGA | 6088 | 6107 | Exon 2-Intron | −24 | 537 |
| | | | | 1 junction | | |
| 523 | AAATAATGGGAGCAGCTTAC | 6019 | 6038 | Intron 1 | −25 | 538 |
| 524 | GTAGACACTCCCCAGGGTTC | 5964 | 5983 | Intron 1 | −27.5 | 539 |
| 525 | GGGTTCTGAATGGCTTAGCA | 5950 | 5969 | Intron 1 | −28.1 | 540 |
| 526 | TTAGCTGAACTCTTGCACCT | 5898 | 5917 | Intron 1 | −24.3 | 541 |
| 527 | TCAAGGATCTCTGTATTTAT | 5852 | 5871 | Intron 1 | −23.7 | 542 |
| 528 | GTCTTGAGGCCAGCTCAGTG | 5817 | 5836 | Intron 1 | −21.1 | 543 |
| 529 | AAATACAAAAAAAATCAGCC | 5546 | 5565 | Intron 1 | −20 | 544 |
| 530 | TCAGCCAGGCATGGTGACAG | 5532 | 5551 | Intron 1 | −27.5 | 545 |
| 531 | TGAGACGCAGAGCTTGCAGT | 5459 | 5478 | Intron 1 | −27.8 | 546 |
| 532 | AGTAGCTGGGATTACAGGTG | 5132 | 5151 | Intron 1 | −31 | 547 |
| 533 | TGCATCCAGCCAAGTCCCCA | 4970 | 4989 | Intron 1 | −20 | 548 |
| 534 | GAGGCAACGCTGACAAGCCA | 4838 | 4857 | Intron 1 | −21.63 | 549 |
| 535 | CCACTTCCCAGCACCCAGAG | 4821 | 4840 | Intron 1 | −26.43 | 550 |
| 536 | CAACATGGGCTACACTGTTT | 4800 | 4819 | Intron 1 | −26.4 | 551 |
| 537 | TTTTTTTTTGCGGAAGGGTC | 4761 | 4780 | Intron 1 | −20.2 | 552 |
| 538 | TGCAGCAATCTCGGATCACT | 4713 | 4732 | Intron 1 | −22.6 | 553 |
| 539 | GTAGCTGGAATTATGGCCGT | 4643 | 4662 | Intron 1 | −20.77 | 554 |
| 540 | CGTGCGCCACCAGGACCAGC | 4626 | 4645 | Intron 1 | −28.7 | 555 |
| 541 | AGTTTGTGACCAGCCAGGCT | 4345 | 4364 | Intron 1 | −28.6 | 556 |
| 542 | CCAGGCTAACATGGTGAAAC | 4332 | 4351 | Intron 1 | −28.4 | 557 |
| 543 | AGGCAGGCGGACCACGAGGT | 4237 | 4256 | Intron 1 | −31.8 | 558 |
| 544 | AAATTAGGCTGGGTGCGGTG | 4158 | 4177 | Intron 1 | −26.6 | 559 |
| 545 | CATGGTGGCGGGGGCCTGTA | 4010 | 4029 | Intron 1 | −31.4 | 560 |
| 546 | AGGCACAGCTTGCAGTGAGC | 3942 | 3961 | Intron 1 | −31.6 | 561 |
| 547 | GCTTGAACCTGGGAGACGGA | 3787 | 3806 | Intron 1 | −35.9 | 562 |
| 548 | GGGAGACGGAGGTTGCAGTG | 3777 | 3796 | Intron 1 | −32.1 | 563 |
| 549 | CTGTAGTGCCAGCTACTCAG | 3656 | 3675 | Intron 1 | −28.6 | 564 |
| 550 | TGTAGTGCCAGCTACTCAGG | 3655 | 3674 | Intron 1 | −28.6 | 565 |
| 551 | TTTGAGCCTGGAAGCGGAGG | 3615 | 3634 | Intron 1 | −26.1 | 566 |
| 552 | TTGTCAGACGAGAGCTCTGA | 3495 | 3514 | Intron 1 | −20.3 | 567 |
| 553 | GAGAGCTCTGACTCTGACTC | 3486 | 3505 | Intron 1 | −22 | 568 |
| 554 | ACTCCATGGATCATGTGCAC | 3470 | 3489 | Intron 1 | −24.1 | 569 |
| 555 | GCACCAGAGGCCCAGGCAGC | 3454 | 3473 | Intron 1 | −30.2 | 570 |
| 556 | CAGCCCCTGAAAGAGAGGGA | 3438 | 3457 | Intron 1 | −23 | 571 |
| 557 | GAGAGGGAGGTGCCCTACAG | 3426 | 3445 | Intron 1 | −24.7 | 572 |
| 558 | GAGCACTTAGCTTTGCGGTC | 3407 | 3426 | Intron 1 | −22 | 573 |
| 559 | TGCCCTCACCAGCCAGGTGA | 3378 | 3397 | Intron 1 | −27.5 | 574 |
| 560 | GACCTCTCTGAATACCACCC | 3343 | 3362 | Intron 1 | −25 | 575 |
| 561 | CTGGGCAGCGTGAGGTCATT | 3268 | 3287 | Intron 1 | −29.5 | 576 |
| 562 | AATCTCTACCAGGTGTTTGC | 3227 | 3246 | Intron 1 | −24.9 | 577 |
| 563 | TGAGTGTGTCTCACGAGTCT | 3188 | 3207 | Intron 1 | −23.2 | 578 |
| 564 | ACAAGCTTTCCAGAACCCAC | 3161 | 3180 | Intron 1 | −24.2 | 579 |
| 565 | TAGGAAGCCGAAATGCCCTG | 3140 | 3159 | Intron 1 | −21.1 | 580 |
| 566 | CCCTGATGTGGTAGAGTGTC | 3075 | 3094 | Intron 1 | −24.3 | 581 |
| 567 | TGTCTGTTCATGTCCCCCTC | 3059 | 3078 | Intron 1 | −30.5 | 582 |
| 568 | TGGCTGAGTCCCAGACCTGG | 3033 | 3052 | Intron 1 | −22.7 | 583 |
| 569 | GACCTCACCACCACCCGTGG | 3008 | 3027 | Intron 1 | −28.1 | 584 |
| 570 | CTGGCCCTATTTCAACCCTA | 2982 | 3001 | Intron 1 | −22.4 | 585 |
| 571 | CCTACTTCCTATGGTGTTCT | 2966 | 2985 | Intron 1 | −25 | 586 |
| 572 | TGTTCTCATAGCACCGTGGA | 2952 | 2971 | Intron 1 | −28.6 | 587 |
| 573 | CACCGTGGAGAATTAAAGAG | 2941 | 2960 | Intron 1 | −23 | 588 |
| 574 | CACTAAGTAAGGGCACTCTA | 2919 | 2938 | Intron 1 | −22.6 | 589 |
| 575 | CAAAGCACTATAACGGATCT | 2893 | 2912 | Intron 1 | −20.1 | 590 |
| 576 | AACCAGCAGTGGACACACAG | 2872 | 2891 | Intron 1 | −20.6 | 591 |
| 577 | AACAAGCCCAGGGTGAAAAG | 2797 | 2816 | Intron 1 | −22.9 | 592 |
| 578 | GTGAAAAGTTCTGCAGGGGA | 2785 | 2804 | Intron 1 | −30.9 | 593 |
| 579 | AGGGGATGACTGCCAGTTCA | 2771 | 2790 | Intron 1 | −22.9 | 594 |
| 580 | TCTCACTGTGGCTCTATTTC | 2744 | 2763 | Intron 1 | −23.4 | 595 |
| 581 | TGACATAGGACACTAGAAGG | 2689 | 2708 | Intron 1 | −20.2 | 596 |
| 582 | CTAGAAGGGCAAACGCAGTG | 2677 | 2696 | Intron 1 | −20.9 | 597 |
| 583 | GTGTCACCTGCCACTATTAC | 2660 | 2679 | Intron 1 | −23.5 | 598 |
| 584 | TACTCAATTACTTGATGACC | 2643 | 2662 | Intron 1 | −20.1 | 599 |
| 585 | TGGCACTAAACTGGCTGTGG | 2611 | 2630 | Intron 1 | −21.9 | 600 |
| 586 | GTGGCCAAAGGCTAGAGAGC | 2595 | 2614 | Intron 1 | −27.6 | 601 |
| 587 | AGCAAGCTCCTCCTTGCCCT | 2561 | 2580 | Intron 1 | −26.2 | 602 |
| 588 | CCCACTTGAAGAGACTGCTA | 2536 | 2555 | Intron 1 | −22.5 | 603 |
| 589 | CACCTTGGACTTCAATCCAC | 2509 | 2528 | Intron 1 | −20.7 | 604 |
| 590 | CTGGAGAGAAGCGCGGTCTT | 2437 | 2456 | Intron 1 | −22.8 | 605 |
| 591 | CTCCCTGGAGGACACGGTGC | 2412 | 2431 | Intron 1 | −30.3 | 606 |
| 592 | GAACGCCAAACTGGGAGAAG | 2362 | 2381 | Intron 1 | −21.4 | 607 |
| 593 | CTCCCCCATTACTCGTCCAC | 2148 | 2167 | Intron 1 | −24.6 | 608 |
| 594 | TCAGACCCCCGCCTACCTGC | 2086 | 2105 | Exon 1-Intron | −30.3 | 609 |
| | | | | 1 junction | | |
| 595 | CCCCGCCCCTGACTGCGCTG | 2003 | 2022 | Exon 1 | −24.9 | 610 |
| 596 | GCGCTGGGTAGGTGGTCGCT | 1989 | 2008 | Exon 1 | −26 | 611 |
| 597 | GGACCGGCGGAATCACACCC | 1960 | 1979 | Exon 1 | −24.4 | 612 |

TABLE 7
Antisense oligonucleotides targeting SEQ ID NO: 2

| GI | | Target | Target | Target | SEQ |
|---|---|---|---|---|---|
| ID# | Sequence | Start Site | Stop Site | Region | ID NO |
| GI1 | CATGGCTGATATATCCCGGG | 12705 | 12724 | Exon 5 | 16 |
| GI2 | CAGCCAGCGTTAATAACGGG | 14741 | 14760 | Intron 5 | 17 |
| GI3 | TCCTCTGGTGTAGGAATGGCGT | 16400 | 16421 | Exon 7 | 18 |
| GI4 | TGGCACACTGCAACAGTACCAC | — | — | Non-targeting | — |
| | | | | control | |
| GI10 | CAGCCAGCGTTAATAACG | 14743 | 14760 | Intron 5 | 19 |
| GI11 | CTGGTGTAGGAATGGCGT | 16400 | 16417 | Exon 7 | 20 |
| GI12 | TGGCTGATATATCCCGGG | 12705 | 12722 | Exon 5 | 21 |

TABLE 8
Antisense oligonucleotides targeting SEQ ID NO: 2

| | Percent expression of human FLCN protein |
|---|---|
| GI ID# | relative to untreated sample |
| GI1 | 18 |
| GI2 | 6 |

TABLE 9
Dose response assay for antisense oligonucleotides
targeting SEQ ID NO: 2

| GI ID# | 1 nM | 25 nM | 100 nM |
|---|---|---|---|
| GI1 | 0.94 | 0.73 | 0.45 |
| GI2 | 0.54 | 0.44 | 0.30 |

TABLE 10
Antisense modulators targeting SEQ ID NO: 2

| | | Target | Target | | SEQ |
|---|---|---|---|---|---|
| GI | | Start | Stop | | ID |
| ID# | Sequence | Site | Site | Target Region | NO |
| GI13 | ATTTAATGGAGGTCTCTTT | 12727 | 12745 | Exon 5 | 22 |
| GI14 | TCATGGCTGATATATCCCG | 12707 | 12725 | Exon 5 | 23 |
| GI15 | AATGGCGTGAAGGCTGTGT | 16389 | 16407 | Exon 7 | 24 |
| GI16 | TCTGGACCAAGGTATCCTC | 17431 | 17449 | Exon 8 | 25 |
| GI17 | TTAGTCGACATGTAAACCA | — | — | Non-targeting | — |
| | | | | control | |
| GI18 | TCACACAACATGTAAACCA | — | — | Non-targeting | — |
| | | | | control | |
| GI19 | TCAGAAAACATGTAAACCA | — | — | Non-targeting | — |
| | | | | control | |
| GI20 | TAGGAAAACATGTAAACCA | — | — | Non-targeting | — |
| | | | | control | |

TABLE 11
siRNA targeting SEQ ID NO: 2

| | Percent expression of human FLCN protein |
|---|---|
| GI ID# | relative to si-NT |
| si-NT | 100 |
| si-FLCN | 14 |

TABLE 12
Human iPSC Lines for Evaluating Therapies
for Neurodegenerative Disease such as ALS

| Name | Type | ALS Subtype/Mutation |
|---|---|---|
| GI-iPSC 1 | Healthy | NA |
| GI-iPSC 2 | Isogenic | TARDBP (G298S) |
| GI-iPSC 3 | Isogenic | SOD1 (L144F) |
| GI-iPSC 4 | Patient | Sporadic |
| GI-iPSC 5 | Patient | C9ORF72 |
| GI-iPSC 6 | Patient | TARDBP |
| GI-iPSC 7 | Patient | Sporadic |
| GI-iPSC 8 | Patient | C9ORF72 |
| GI-iPSC 9 | Patient | SOD1 (A4V) |
| GI-iPSC 10 | Healthy | NA |
| GI-iPSC 11 | Healthy | NA |

TABLE 13
Normalized Survival Index (NSI) of Human ALS iPSC-derived Motor Neurons

| | | Day 28 | | Day 31 | | Day 35 | |
|---|---|---|---|---|---|---|---|
| Cell line | Treatment | NSI | STDEV | NSI | STDEV | NSI | STDEV |
| BJ-iPS | si-NT | 1.00 | 0.08 | 0.98 | 0.03 | 0.94 | 0.04 |
| | si-FLCN | | | 0.98 | 0.02 | 0.91 | 0.01 |
| BJ-SOD1 | si-NT | 0.96 | 0.04 | 0.69 | 0.03 | 0.41 | 0.05 |
| (L144F) | si-FLCN | | | 0.92 | 0.02 | 0.82 | 0.02 |
| BJ-TDP43 | si-NT | 0.95 | 0.04 | 0.79 | 0.04 | 0.42 | 0.02 |
| (G298S) | si-FLCN | | | 0.90 | 0.03 | 0.77 | 0.02 |
| CS14isALS- | si-NT | 0.94 | 0.03 | 0.77 | 0.06 | 0.58 | 0.04 |
| Tn16 | si-FLCN | | | 0.87 | 0.02 | 0.79 | 0.05 |

TABLE 14
Phosphorylated TDP-43 (pTDP43) levels in Human Motor Neurons

| | | Day 28 | | Day 31 | | Day 35 | |
|---|---|---|---|---|---|---|---|
| Cell line | Treatment | pTDP43 | STDEV | pTDP43 | STDEV | pTDP43 | STDEV |
| BJ-iPS | si-NT | 1.00 | 0.13 | 1.01 | 0.08 | 1.00 | 0.12 |
| | si-FLCN | | | 1.03 | 0.09 | 1.03 | 0.10 |
| BJ-SOD1 | si-NT | 2.32 | 0.40 | 2.60 | 0.08 | 2.92 | 0.17 |
| (L144F) | si-FLCN | | | 1.69 | 0.15 | 1.88 | 0.16 |
| BJ-TDP43 | si-NT | 2.51 | 0.46 | 2.36 | 0.22 | 2.95 | 0.24 |
| (G298S) | si-FLCN | | | 1.68 | 0.22 | 2.00 | 0.18 |
| CS14isALS- | si-NT | 2.49 | 0.45 | 2.67 | 0.17 | 2.88 | 0.32 |
| Tn16 | si-FLCN | | | 1.76 | 0.29 | 2.00 | 0.15 |

TABLE 15
Small molecule modulators

| | | | | Exemplar |
|---|---|---|---|---|
| SM | Exemplar | Exemplar | | Binding |
| NO | Name | SMILES | Exemplar Image | Score |
| SM1 | 1,9,10,11-tetrahydro-8H- benzo[h]pyrrolo[3,4- b]quinolin-8-one | O=C1NCC2=C1C=c1ccc3c(c1N2)CC=CC=3 | 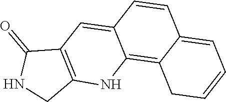 [SMILES: O=C1NCC2=C1C=c1ccc3c(c1N2)CC=CC=3] | 3.42E−07 |
| SM2 | 8-Methylbenzo[f]quinazoline- 1,3-diamine | Cc1ccc2c(ccc3nc(N)nc(N)c32)c1 | 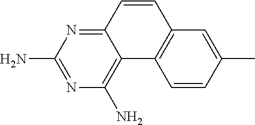 [SMILES: Cc1ccc2c(ccc3nc(N)nc(N)c32)c1] | 5.56E−07 |
| SM3 | 8,9-dimethyl-3,4,5,6- tetrahydrobenzo[f]quinazoline- 1,3-diamine | Cc1cc2c(cc1C)C1=C(CC2)NC(N)N=C1N | 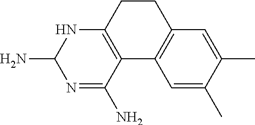 [SMILES: Cc1cc2c(cc1C)C1=C(CC2)NC(N)N=C1N] | 5.56E−07 |
| SM4 | 3-amino-2H- benzo[f]quinazolin-1-one | Nc1nc2ccc3ccccc3c2c(=O)[nH]1 | 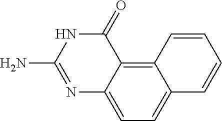 [SMILES: Nc1nc2ccc3ccccc3c2c(=O)[nH]1] | 5.56E−07 |
| SM5 | 3-amino-1-chloro-5H- phenanthridin-6-one | Nc1cc(CI)c2c(c1)[nH]c(=O)c1ccccc12 | 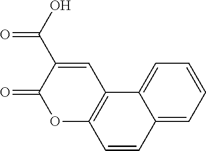 [SMILES: O=C(O)c1cc2c(ccc3ccccc32)oc1=O] | 7.68E−07 |
| SM6 | (5,7-dimethyl-3-oxo-4H- quinoxalin-2-yl)urea | Cc1cc(C)c2[nH]c(=O)c(NC(N)=O)nc2c1 | 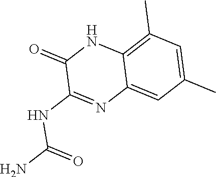 [SMILES: Cc1cc(C)c2[nH]c(=O)c(NC(N)=O)nc2c1] | 7.68E−07 |
| SM7 | 2,3,6,7,8,9, 10,11- octahydrocycloocta[g] phthalazine-1,4-dione | O=c1[nH][nH]c(=O)c2cc3c(cc12)CCCCCC3 | 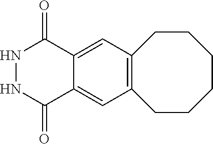 [SMILES: O=c1[nH][nH]c(=O)c2cc3c(cc12)CCCCCC3] | 7.68E−07 |
| SM8 | 2-amino-4-naphthalen-1-yl-1H- pyrimidin-6-one | Nc1nc(-c2cccc3ccccc23)cc(=O)[nH]1 | 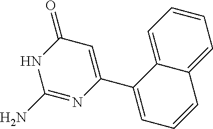 [SMILES: Nc1nc(-c2cccc3ccccc23)cc(=O)[nH]1] | 7.68E−07 |
| SM9 | 9-Amino-7a,8,10- triazacyclopenta[a]phenalen-7- one | Nc1nc2c3cccc4cccc(c(=O)n2n1)c43 | 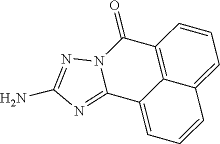 [SMILES: Nc1nc2c3cccc4cccc(c(=O)n2n1)c43] | 9.03E−07 |
| SM10 | 3-aminobenzo[g]quinoxalin- 2(1H)-one | Nc1nc2cc3ccccc3cc2[nH]c1=O | 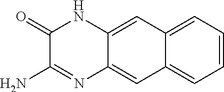 [SMILES: Nc1nc2cc3ccccc3cc2[nH]c1=O] | 9.03E−07 |
| SM11 | 2-[(Z)-1-naphthalen-1- ylethylideneamino]guanidine | C/C(=N\NC(=N)N)c1cccc2ccccc12 | 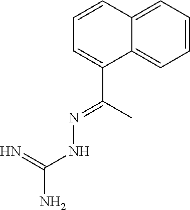 [SMILES: C/C(=N\NC(=N)N)c1cccc2ccccc12] | 9.03E−07 |
| SM12 | 2-(4,6,8-trimethylquinazolin-2- yl)guanidine | Cc1cc(C)c2nc(NC(=N)N)nc(C)c2c1 | 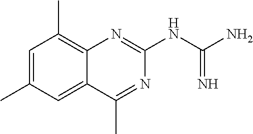 [SMILES: Cc1cc(C)c2nc(NC(=N)N)nc(C)c2c1] | 9.03E−07 |
| SM13 | 4,7-diamino-9-azofluorene | NNC1=c2cc(N)ccc2=C2C(N)C=CC=C12 | 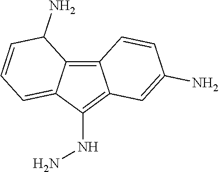 [SMILES: NNC1=c2cc(N)ccc2=C2C1=CC=CC2N] | 1.06E−06 |
| SM14 | 1-methyl-2,5-dihydro-1H- pyrido[4,3-b]indol-3-amine | CC1NC(N)=Cc2[nH]c3ccccc3c21 | 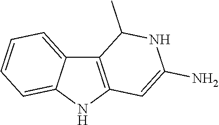 | 1.06E−06 |
| SM15 | 4-methyl-6-(5-methyl-2H- indazol-6-yl)piperidin-3-amine | CC1CC(c2cc3n[nH]cc3cc2C)NCC1N | 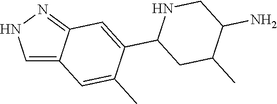 [SMILES: Cc1cc2c[nH]nc2cc1C1CC(C)C(N)CN1] | 1.06E−06 |
| SM16 | 5-(3H-benzimidazol-5-yl)- 1,2,3,4-tetrahydroisoquinoline | C1Cc2c(cccc2-c2ccc3[nH]cnc3c2)CN1 | 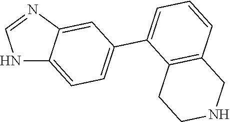 [SMILES: c1cc2c(c(-c3ccc4[nH]cnc4c3)c1)CCNC2] | 1.25E−06 |
| SM17 | (6,8-dimethyl-5H- [1,2,4]triazino[5,6-b]indol-3- yl)hydrazine | Cc1cc(C)c2c(c1)C1=N[N]C(NN)=NC1=N2 | 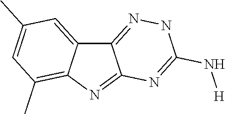 [SMILES: Cc1cc(C)c2nc3nc(N)[nH]nc-3c2c1] | 1.25E−06 |
| SM18 | 2-amino-5-chloro-3,9- dihydropyrimido[4,5-b]indol-4- one | Nc1nc2[nH]c3cccc(CI)c3c2c(=O)[nH]1 | 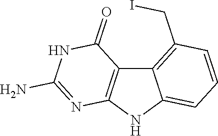 [SMILES: Nc1nc2[nH]c3cccc(CI)c3c2c(=O)[nH]1] | 1.25E−06 |
| SM19 | 8aH-fluorene-8- carboximidamide | N=C(N)C1=CC=CC2=c3ccccc3=CC12 | 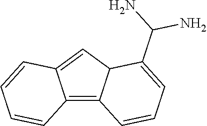 [SMILES: NC(N)C1=CC=CC2=c3ccccc3=CC12] | 1.47E−06 |
| SM20 | (1-(1H-fluoren-7- yl)ethyl)hydrazine | CC(NN)c1ccc2c(c1)C=C1CC=CC=C12 | 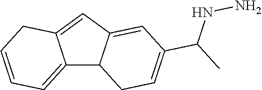 [SMILES: CC(NN)C1=CCC2C(=C1)C=C1CC=CC=C12] | 1.47E−06 |
| SM21 | 6,7-Dimethyl-1H-pyrazolo[3,4- b]quinolin-3-ylamine | Cc1cc2cc3c(N)n[nH]c3nc2cc1C | 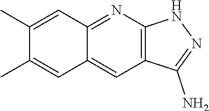 [SMILES: Cc1cc2cc3c(N)n[nH]c3nc2cc1C] | 1.47E−06 |
| SM22 | 2-(acenaphthylen-5- yl)acetamide | NC(=O)Cc1ccc2c3c(cccc13)C=C2 | 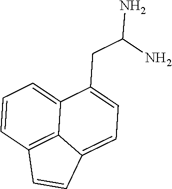 [SMILES: NC(N)Cc1ccc2c3c(cccc13)C=C2] | 1.72E−06 |
| SM23 | 2-amino-1H-indeno[6,7,1- def]isoquinoline-1,3(2H)-dione | NN1C(=O)c2ccc3c4c(ccc(c24)C1=O)C=C3 | 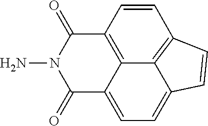 [SMILES: NN1C(=O)c2ccc3c4c(ccc(c24)C1=O)C=C3] | 1.72E−06 |
| SM24 | 7-(3-methylphenyl)-3,5- dihydro-4H-pyrrolo[3,2- d]pyrimidin-4-one | Cc1cccc(-c2c[nH]c3c(=O)[nH]cnc23)c1 | 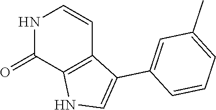 [SMILES: Cc1cccc(-c2c[nH]c3c(=O)[nH]ccc23)c1] | 1.72E−06 |
| SM25 | 5-naphthalen-1-yl-1H-pyrazol- 3-amine | Nc1cc(-c2cccc3ccccc23)[nH]n1 | 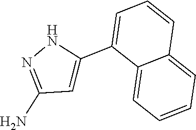 [SMILES: Nc1cc(-c2cccc3ccccc23)[nH]n1] | 2.03E−06 |
| SM26 | 11,15-dioxa-16-azatetracyclo [8.7.0.02,7.013,17]heptadeca- 1(10),2,4,6,8-pentaene | C1ONC2C1COc1ccc3ccccc3c12 | 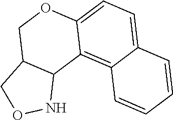 [SMILES: c1ccc2c3c(ccc2c1)OCC1CONC31] | 2.03E−06 |
| SM27 | Benz[cd]indole-3- carboxamide, 1,3,4,5- tetrahydro- | NC(=O)C1CCc2cccc3[nH]cc1c23 | 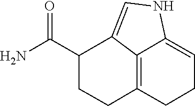 [SMILES: NC(=O)C1CCC2=c3c1c[nH]c3=CCC2] | 2.03E−06 |
| SM28 | (2R)-spiro[1H-acenaphthylene- 2,5′-imidazolidine]-2′,4′-dione | O=C1NC(=O)[C@]12(Cc3cccc4cccc2c34)N1 | 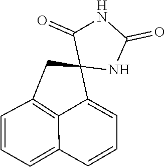 [SMILES: O=C1NC(=O)[C@]2(Cc3cccc4cccc2c34)N1] | 2.03E−06 |
| SM29 | Indeno[6,7,1-cde]indol-2(1H)- one | O=C1Nc2ccc3c4c(ccc1c24)C=C3 | 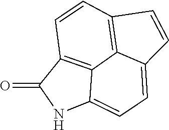 [SMILES: O=C1Nc2ccc3c4c(ccc1c24)C=C3] | 2.03E−06 |
| SM30 | 9-hydroxy-4- methylbenzo[h]chromen-2-one | Cc1cc(=O)oc2c1ccc1ccc(O)cc12 | 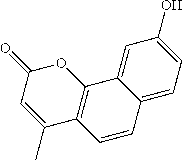 [SMILES: Cc1cc(=O)oc2c1ccc1ccc(O)cc12] | 2.38E−06 |
| SM31 | 7-methyl-1,2,3,4- tetrahydrocyclopenta[b]indol- 3-ol | Cc1ccc2[nH]c3c(c2c1)CCC3O | 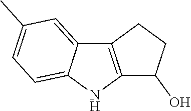 | 2.38E−06 |
| SM32 | 7,9-dimethyl-1H- [1,2]oxazolo[4,3-c]quinolin-3- one | Cc1cc(C)c2c3noc(=O)c-3c[nH]c2c1 | 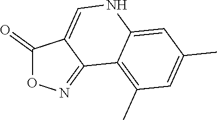 [SMILES: Cc1cc(C)c2c3noc(=O)c-3c[nH]c2c1] | 2.38E−06 |
| SM33 | 2-amino-5,5-dimethyl- 4,6,7,8,9,9a-hexahydro-3aH- benzo[f]isoindole-1,3-dione | CC1(C)CCCC2=C1CC1C(C2)C(=O)N(N)C1=O | 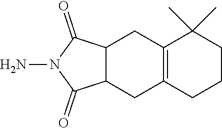 [SMILES: CC1(C)CCCC2=C1CC1C(=O)N(N)C(=O)C1C2] | 2.38E−06 |
| SM34 | 4,4a,5,6-tetrahydro-2H- benzo[h]cinnolin-3-one | O=C1CC2CCc3ccccc3C2=NN1 | 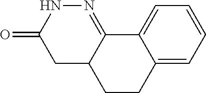 [SMILES: O=C1CC2CCc3ccccc3C2=NN1] | 2.80E−06 |
| SM35 | N-methyl-2H-fluorene-1- carboxamide | CNC(=O)C1=C2C=c3ccccc3=C2C=CC1 | 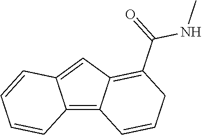 [SMILES: CNC(=O)C1=C2C=c3ccccc3=C2C=CC1] | 2.80E−06 |
| SM36 | 2-amino-6-(4-fluorophenyl)- 3,7-dihydropyrrolo[2,3- d]pyrimidin-4-one | Nc1nc2[nH]c(-c3ccc(F)cc3)cc2c(=O)[nH]1 | 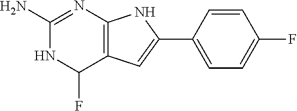 [SMILES: NC1=Nc2[nH]c(-c3ccc(F)cc3)cc2C(F)N1] | 2.80E−06 |
| SM37 | 8-fluoro-6-methyl-5,9-dihydro- 1\|2-pyrazolo[4,5-c]quinolin- 3(2H)-one | CC1=C2NC=C3C(=O)N[N]C3=C2CC(F)=C1 | 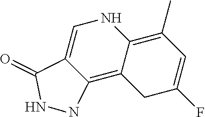 [SMILES: CC1=C2NC=c3c(=O)[nH][nH]c3=C2CC(F)=C1] | 2.80E−06 |
| SM38 | Benzo[g]quinazolin-4(3H)-one | O=c1[nH]cnc2cc3ccccc3cc12 | 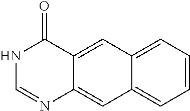 [SMILES: O=c1[nH]cnc2cc3ccccc3cc12] | 2.80E−06 |
| SM39 | 2,3,4,9-tetrahydro-1h- carbazole-6-carboxamide | NC(=O)c1ccc2[nH]c3c(c2c1)CCCC3 | 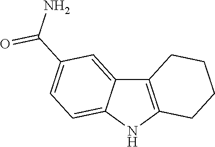 [SMILES: NC(=O)c1ccc2[nH]c3c(c2c1)CCCC3] | 2.80E−06 |
| SM40 | (3R)-3-methyl-1,2- dihydrobenzo[f]chromen-3-ol | C[C@]1(O)CCc2c(ccc3ccccc23)O1 | 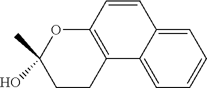 [SMILES: C[C@]1(O)CCc2c(ccc3ccccc23)O1] | 2.80E−06 |
| SM41 | Anthracen-9- ylmethylhydrazine | NNCc1c2ccccc2cc2ccccc12 | 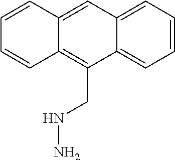 [SMILES: NNCc1c2ccccc2cc2ccccc12] | 2.80E−06 |
| SM42 | 5-(4-Methylphenyl)-1H-indole- 2,3-dione | Cc1ccc(-c2ccc3c(c2)C(=O)C(=O)N3)cc1 | 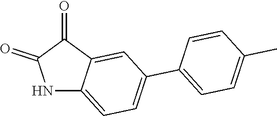 [SMILES: Cc1ccc(-c2ccc3c(c2)C(=O)C(=O)N3)cc1] | 2.80E−06 |
| SM43 | 6-oxo-8,8a,9,10-tetrahydro-7H- phenanthrene-9-carboxylic acid | [O]C(=O)C1Cc2ccccc2C2=CC(=O)CCC12 | 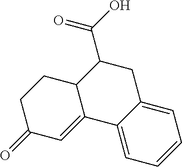 [SMILES: O=C1C=C2c3ccccc3CC(C(=O)O)C2CC1] | 2.80E−06 |
| SM44 | 3-oxo-3H-benzo[f]chromene-2- carboxamide | NC(=O)c1cc2c(ccc3ccccc32)oc1=O | 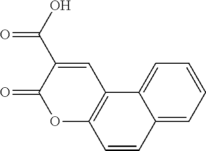 [SMILES: O=C(O)c1cc2c(ccc3ccccc32)oc1=O] | 3.29E−06 |
| SM45 | 4-(1,2,3,4- tetrahydroisoquinolin-1- yl)phenol | Oc1ccc(C2NCCc3ccccc32)cc1 | 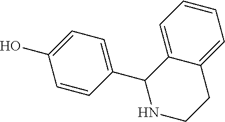 [SMILES: Oc1ccc(C2NCCc3ccccc32)cc1] | 3.29E−06 |
| SM46 | 5-(1-methylindol-2-yl)-1H- pyrazol-3-amine | Cn1c(-c2cc(N)n[nH]2)cc2ccccc21 | 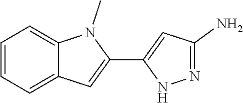 [SMILES: Cn1c(-c2cc(N)n[nH]2)cc2ccccc21] | 3.29E−06 |
| SM47 | 8-fluoro-4-hydrazineyl-5,9- dihydro-3H-pyrimido[5,4- b]indole | NNC1=c2[nH]c3c(c2N=CN1)CC(F)=CC=3 | 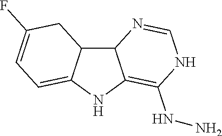 [SMILES: NNC1=C2NC3=CC=C(F)CC3C2N=CN1] | 3.29E−06 |
| SM48 | 6-isopropyl-5,5a-dihydro-1\|2- pyrazolo[4,5-c]quinolin-3(2H)- one | CC(C)C1=CC=CC2=C3[N]NC(=O)C3=CNC12 | 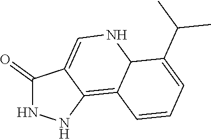 [SMILES: CC(C)C1=CC=CC2=c3[nH][nH]c(=O)c3=CNC12] | 3.29E−06 |
| SM49 | 5-pyridin-3-yl-1H-indazol-3- amine | Nc1n[nH]c2ccc(-c3cccnc3)cc12 | 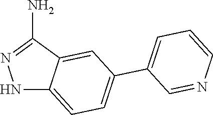 [SMILES: Nc1n[nH]c2ccc(-c3cccnc3)cc12] | 3.29E−06 |
| SM50 | Acenaphthenequinone Dioxime | O/N=C1C(=N\O)\c2cccc3cccc\1c23 | 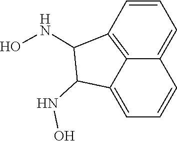 [SMILES: ONC1c2cccc3cccc(c23)C1NO] | 3.87E−06 |
| SM51 | 7,10-dimethyl-2,3,4,5- tetrahydro-[1,4]oxazepino[7,6- b]quinoline | Cc1ccc(C)c2nc3c(cc12)CNCCO3 | 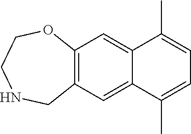 [SMILES: Cc1ccc(C)c2cc3c(cc12)CNCCO3] | 3.87E−06 |
| SM52 | 3-(6-hydroxy-1,2,3,4- tetrahydronaphthalen-2- yl)cyclopentan-1-one | O=C1CCC(C2CCc3cc(O)ccc3C2)C1 | 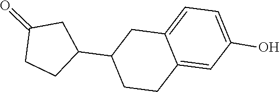 [SMILES: O=C1CCC(C2CCc3cc(O)ccc3C2)C1] | 3.87E−06 |
| SM53 | 2,4-dimethyl-2,3,5,9- tetrahydrofuro[3,2-c]quinolin- 8-yl)(\|1-oxidaneyl)methanone | CC1CC2=C(C)NC3=CC=C(C([O])=O)CC3=C2O1 | 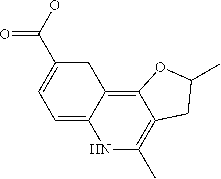 [SMILES: CC1=C2CC(C)OC2=C2CC(C(=O)O)=CC=C2N1] | 3.87E−06 |
| SM54 | 4,7-dimethyl-2H-pyrano[3,2- c]quinoline-2,5(6H)-dione | Cc1cccc2c1[nH]c(=O)c1c(C)cc(=O)oc12 | 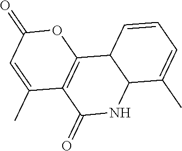 [SMILES: CC1=CC=CC2c3oc(=O)cc(C)c3C(=O)NC12] | 3.87E−06 |
| SM55 | 7,8-dihydro-6H- [1,3]benzodioxolo[5,6- b]quinolin-9-one | O=C1CCCc2nc3cc4c(cc3cc21)OCO4 | 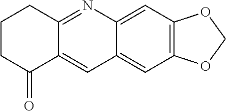 [SMILES: O=C1CCCc2nc3cc4c(cc3cc21)OCO4] | 4.55E−06 |
| SM56 | 5-naphthalen-1- ylimidazolidine-2,4-dione | O=C1NC(c2cccc3ccccc23)C(=O)N1 | 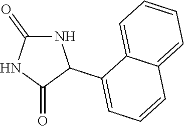 [SMILES: O=C1NC(=O)C(c2cccc3ccccc23)N1] | 4.55E−06 |
| SM57 | 1H-benzo[f]benzimidazol-2- ylmethanol | OCc1nc2cc3ccccc3cc2[nH]1 | 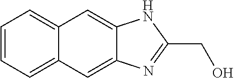 [SMILES: OCc1nc2cc3ccccc3cc2[nH]1] | 4.55E−06 |
| SM58 | N-methyl-2-naphthalen-1- ylcyclopropane-1-carboxamide | CNC(=O)C1CC1c1cccc2ccccc12 | 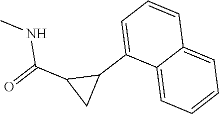 [SMILES: CNC(=O)C1CC1c1cccc2ccccc12] | 4.55E−06 |
| SM59 | 3-oxo-3H-pyrano[3,2- f]quinoline-1-carboxylic acid | [O]C(=O)c1cc(=O)oc2ccc3ncccc3c12 | 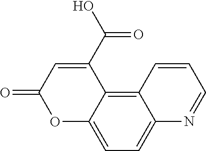 [SMILES: O=C(O)c1cc(=O)oc2ccc3ncccc3c12] | 4.55E−06 |

| SM | Scaffold | Scaffold | |
|---|---|---|---|
| NO | Name | SMILES | Scaffold Image |
| SM1 | 1,9,10,11-tetrahydro-8H- benzo[h]pyrrolo[3,4- b]quinolin-8-one | O=C1NCC2=C1C=c1ccc3c(c1N2)CC=CC=3 | 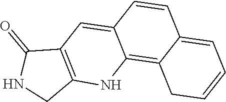 [SMILES: O=C1NCC2=C1C=c1ccc3c(c1N2)CC=CC=3] |
| SM2 | Benzo[f]quinazoline | c1ccc2c(c1)ccc1ncncc12 | 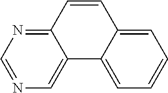 [SMILES: c1ccc2c(c1)ccc1ncncc12] |
| SM3 | Tetrahydrobenzo[f] quinazoline | C1=NCNC2=C1c1ccccc1CC2 | 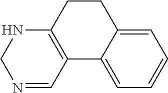 [SMILES: C1=NCNC2=C1c1ccccc1CC2] |
| SM4 | Benzo[f]quinazolin-1(2H)-one | O=c1[nH]cnc2ccc3ccccc3c12 | 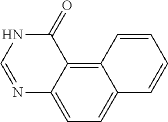 [SMILES: O=c1[nH]cnc2ccc3ccccc3c12] |
| SM5 | 6(5H)-Phenanthridinone | O=c1[nH]c2ccccc2c2ccccc12 | 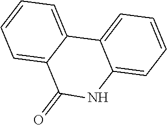 [SMILES: O=c1[nH]c2ccccc2c2ccccc12] |
| SM6 | 2-Hydroxyquinoxaline | O=c1cnc2ccccc2[nH]1 | 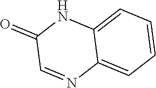 [SMILES: O=c1cnc2ccccc2[nH]1] |
| SM7 | 2,3,6,7,8,9,10,11- octahydrocycloocta[g] phthalazine-1,4-dione | O=c1[nH][nH]c(=O)c2cc3c(cc12)CCCCCC3 | 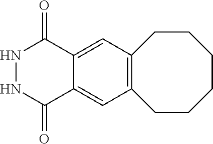 [SMILES: O=c1[nH][nH]c(=O)c2cc3c(cc12)CCCCCC3] |
| SM8 | 4-naphthalen-1-yl-1H- pyrimidin-6-one | O=c1cc(-c2cccc3ccccc23)nc[nH]1 | 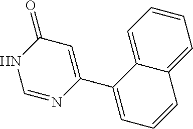 [SMILES: O=c1cc(-c2cccc3ccccc23)nc[nH]1] |
| SM9 | 11,13,14-triazatetracyclo [7.6.1.05,16.010,14]hexadeca- 1,3,5(16),6,8,10,12-heptaen- 15-one | O=c1c2cccc3cccc(c32)c2ncnn12 | 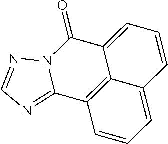 [SMILES: O=c1c2cccc3cccc(c32)c2ncnn12] |
| SM10 | benzo[g]quinoxalin-2(1h)-one | O=c1cnc2cc3ccccc3cc2[nH]1 | 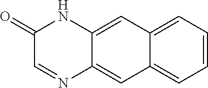 [SMILES: O=c1cnc2cc3ccccc3cc2[nH]1] |
| SM11 | Naphthalene | c1ccc2ccccc2c1 | 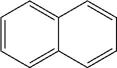 |
| SM12 | Quinazoline | c1ccc2ncncc2c1 | 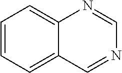 |
| SM13 | 4H-fluorene | C1=CCC2=c3ccccc3=CC2=C1 | 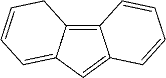 [SMILES: C1=CCC2=c3ccccc3=CC2=C1] |
| SM14 | 2,5-dihydro-1H-pyrido[4,3- b]indole | C1=Cc2[nH]c3ccccc3c2CN1 | 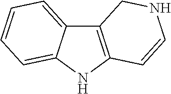 [SMILES: C1=Cc2[nH]c3ccccc3c2CN1] |
| SM15 | 6-(piperidin-2-yl)-2H- indazole | c1cc2c[nH]nc2cc1C1CCCCN1 | 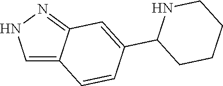 [SMILES: c1cc2c[nH]nc2cc1C1CCCCN1] |
| SM16 | 6-phenyl-1H-benzimidazole | c1ccc(-c2ccc3[nH]cnc3c2)cc1 | 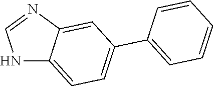 [SMILES: c1ccc(-c2ccc3[nH]cnc3c2)cc1] |
| SM17 | 5H-[1,2,4]triazino[5,6- b]indole | C1=NC2=Nc3ccccc3C2=N[N]1 | 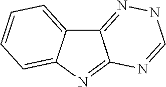 [SMILES: c1ccc2c3n[nH]cnc-3nc2c1] |
| SM18 | 3H-pyrimido[4,5-b]indol- 4(9h)-one | O=c1[nH]cnc2[nH]c3ccccc3c12 | 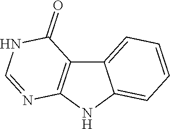 [SMILES: O=c1[nH]cnc2[nH]c3ccccc3c12] |
| SM19 | 8aH-fluorene | C1=CC2=c3ccccc3=CC2C=C1 | 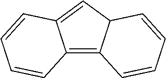 [SMILES: C1=CC2=c3ccccc3=CC2C=C1] |
| SM20 | 1H-fluorene | C1=CCC2=Cc3ccccc3C2=C1 | 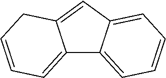 [SMILES: C1=CCC2=Cc3ccccc3C2=C1] |
| SM21 | 1H-Pyrazolo[3,4-b]quinoline | c1ccc2nc3[nH]ncc3cc2c1 | 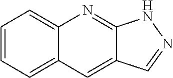 [SMILES: c1ccc2nc3[nH]ncc3cc2c1] |
| SM22 | Acenaphthylene | C1=Cc2cccc3cccc1c23 | 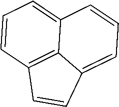 [SMILES: C1=Cc2cccc3cccc1c23] |
| SM23 | 1,8-Naphthalimide | O=C1NC(=O)c2cccc3cccc1c23 | 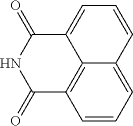 [SMILES: O=C1NC(=O)c2cccc3cccc1c23] |
| SM24 | 7-phenyl-3,5- dihydropyrrolo[3,2- d]pyrimidin-4-one | O=c1[nH]cnc2c(-c3ccccc3)c[nH]c12 | 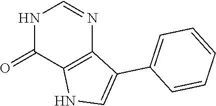 [SMILES: O=c1[nH]cnc2c(-c3ccccc3)c[nH]c12] |
| SM25 | 3-(naphthalen-1-yl)-1H- pyrazole | c1ccc2c(-c3ccn[nH]3)cccc2c1 | 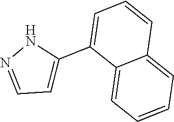 [SMILES: c1ccc2c(-c3ccn[nH]3)cccc2c1] |
| SM26 | 11,15-dioxa-16-azatetracyclo [8.7.0.02,7.013,7]heptadeca- 1(10),2,4,6,8-pentaene | c1ccc2c3c(ccc2c1)OCC1CONC31 | 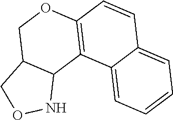 [SMILES: c1ccc2c3c(ccc2c1)OCC1CONC31] |
| SM27 | 1,3,4,5- tetrahydrobenzo[cd]indole | c1cc2c3c(c[nH]c3c1)CCC2 | 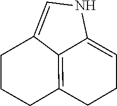 [SMILES: C1=c2[nH]cc3c2=C(CC1)CCC3] |
| SM28 | Acenaphthene | c1cc2c3c(cccc3c1)CC2 | 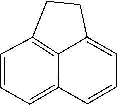 [SMILES: c1cc2c3c(cccc3c1)CC2] |
| SM29 | Benz[cd]indol-2(1H)-one | O=C1Nc2cccc3cccc1c23 | 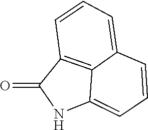 [SMILES: O=C1Nc2cccc3cccc1c23] |
| SM30 | Benzochromenone | O=c1ccc2ccc3ccccc3c2o1 | 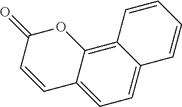 [SMILES: O=c1ccc2ccc3ccccc3c2o1] |
| SM31 | 1,2,3,4- tetrahydrocyclopenta[b]indole | c1ccc2c3c([nH]c2c1)CCC3 | 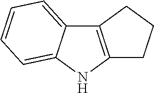 [SMILES: c1ccc2c3c([nH]c2c1)CCC3] |
| SM32 | 1H-[1,2]oxazolo[4,3- c]quinolin-3-one | O=c1onc2c3ccccc3[nH]cc1-2 | 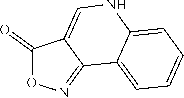 [SMILES: O=c1onc2c3ccccc3[nH]cc1-2] |
| SM33 | 3a,4,5,6,7,8,9,9a-octahydro- 1H-benzo[f]isoindole- 1,3(2H)-dione | O=C1NC(=O)C2CC3=C(CCCC3)CC12 | 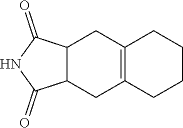 [SMILES: O=C1NC(=O)C2CC3=C(CCCC3)CC12] |
| SM34 | 4,4a,5,6-tetrahydro-2H- benzo[h]cinnolin-3-one | O=C1CC2CCc3ccccc3C2=NN1 | 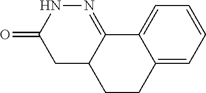 [SMILES: O=C1CC2CCc3ccccc3C2=NN1] |
| SM35 | 2H-fluorene | C1=CC2=c3ccccc3=CC2=CC1 | 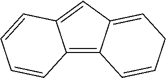 [SMILES: C1=CC2=c3ccccc3=CC2=CC1] |
| SM36 | 6-Phenyl-4-hydroxy-7H- pyrrolo[2,3-d]pyrimidine | O=c1[nH]cnc2[nH]c(-c3ccccc3)cc12 | 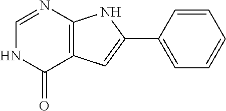 [SMILES: O=c1[nH]cnc2[nH]c(-c3ccccc3)cc12] |
| SM37 | 5,9-dihydro-1\|2-pyrazolo[4,5- c]quinolin-3(2H)-one | O=C1N[N]C2=C3CC=CC=C3NC=C12 | 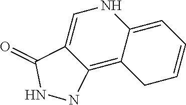 [SMILES: O=c1[nH][nH]c2c1=CNC1=CC=CCC=21] |
| SM38 | Benzo[g]quinazolin-4(3H)- one | O=c1[nH]cnc2cc3ccccc3cc12 | 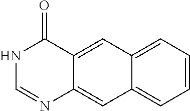 [SMILES: O=c1[nH]cnc2cc3ccccc3cc12] |
| SM39 | 1,2,3,4-Tetrahydrocarbazole | c1ccc2c3c([nH]c2c1)CCCC3 | 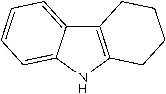 [SMILES: c1ccc2c3c([nH]c2c1)CCCC3] |
| SM40 | Dihydronaphthopyran | c1ccc2c3c(ccc2c1)OCCC3 | 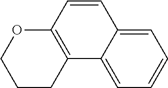 [SMILES: c1ccc2c3c(ccc2c1)OCCC3] |
| SM41 | Anthracene | c1ccc2cc3ccccc3cc2c1 | 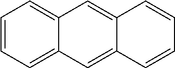 [SMILES: c1ccc2cc3ccccc3cc2c1] |
| SM42 | 5-Phenylisatin | O=C1Nc2ccc(-c3ccccc3)cc2C1=O | 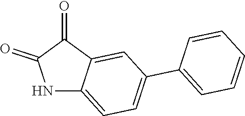 [SMILES: O=C1Nc2ccc(-c3ccccc3)cc2C1=O] |
| SM43 | 2,9,10,10a-tetrahydro-1H- phenanthren-3-one | O=C1C=C2c3ccccc3CCC2CC1 | 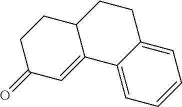 [SMILES: O=C1C=C2c3ccccc3CCC2CC1] |
| SM44 | 3H-Naphtho[2,1-b]pyran-3- one | O=c1ccc2c(ccc3ccccc32)o1 | 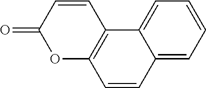 [SMILES: O=c1ccc2c(ccc3ccccc32)o1] |
| SM45 | 1-Phenyl-1,2,3,4- tetrahydroisoquinoline | c1ccc(C2NCCc3ccccc32)cc1 | 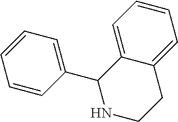 [SMILES: c1ccc(C2NCCc3ccccc32)cc1] |
| SM46 | 2-(1H-pyrazol-5-yl)-1H- indole | c1ccc2[nH]c(-c3ccn[nH]3)cc2c1 | 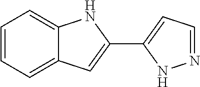 [SMILES: c1ccc2[nH]c(-c3ccn[nH]3)cc2c1] |
| SM47 | 5,9-dihydro-3H-pyrimido[5,4- b]indole | C1=CCc2c3c([nH]c2=C1)=CNC=N3 | 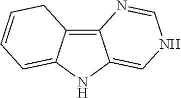 [SMILES: C1=CCc2c3c([nH]c2=C1)=CNC=N3] |
| SM48 | 5,5a-dihydro-1\|2- pyrazolo[4,5-c]quinolin- 3(2H)-one | O=C1N[N]C2=C3C=CC=CC3NC=C12 | 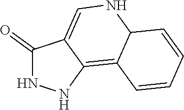 [SMILES: O=c1[nH][nH]c2c1=CNC1C=CC=CC=21] |
| SM49 | 5-pyridin-3-yl-1H-indazole | c1cncc(-c2ccc3[nH]ncc3c2)c1 | 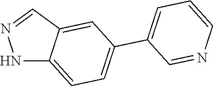 [SMILES: c1cncc(-c2ccc3[nH]ncc3c2)c1] |
| SM50 | Acenaphthene-1,2-diimine | N=C1C(=N)c2cccc3cccc1c23 | 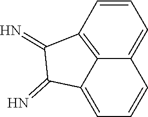 [SMILES: N=C1C(=N)c2cccc3cccc1c23] |
| SM51 | 2,3,4,5-tetrahydro- [1,4]oxazepino[7,6- b]quinoline | c1ccc2nc3c(cc2c1)CNCCO3 | 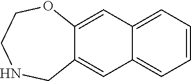 [SMILES: c1ccc2cc3c(cc2c1)CNCCO3] |
| SM52 | 3-(1,2,3,4- tetrahydronaphthalen-2- yl)cyclopentan-1-one | O=C1CCC(C2CCc3ccccc3C2)C1 | 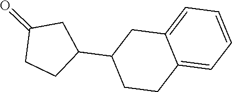 [SMILES: O=C1CCC(C2CCc3ccccc3C2)C1] |
| SM53 | 2,3,5,9-tetrahydrofuro[3,2- c]quinoline | C1=CCC2=C3OCCC3=CNC2=C1 | 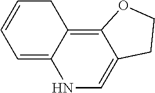 [SMILES: C1=CCC2=C3OCCC3=CNC2=C1] |
| SM54 | pyrano-(3,2-c)quinoline- 2,5(6H)-dione | O=c1ccc2c(=O)[nH]c3ccccc3c2o1 | 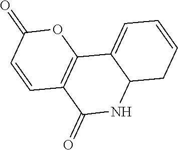 [SMILES: O=C1NC2CC=CC=C2c2oc(=O)ccc21] |
| SM55 | 1,3-Dioxolo[4,5-g]quinoline | c1cnc2cc3c(cc2c1)OCO3 | 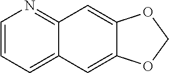 [SMILES: c1cnc2cc3c(cc2c1)OCO3] |
| SM56 | 5-naphthalen-1- ylimidazolidine-2,4-dione | O=C1NC(=O)C(c2cccc3ccccc23)N1 | 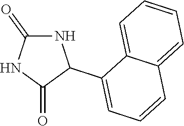 [SMILES: O=C1NC(=O)C(c2cccc3ccccc23)N1] |
| SM57 | 1H-naphth[2,3-d]imidazole | c1ccc2cc3[nH]cnc3cc2c1 | 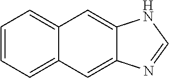 [SMILES: c1ccc2cc3[nH]cnc3cc2c1] |
| SM58 | 1-cyclopropylnaphthalene | c1ccc2c(C3CC3)cccc2c1 | 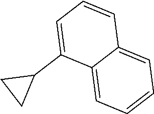 [SMILES: c1ccc2c(C3CC3)cccc2c1] |
| SM59 | 3H-Pyrano(3,2-f)quinolin-3- one | O=c1ccc2c(ccc3ncccc32)o1 | 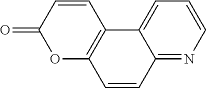 [SMILES: O=c1ccc2c(ccc3ncccc32)o1] |

TABLE 16
Commercial Antibodies or Peptides Targeting FLCN

| Catalog No. | Vendor/Source |
|---|---|
| abx004978 | Abbexa |
| abx034049 | Abbexa |
| abx100526 | Abbexa |
| abx112567 | Abbexa |
| 200154 | Abbiotec |
| ab124885 | Abcam |
| ab169224 | Abcam |
| ab176707 | Abcam |
| ab187311 | Abcam |
| ab87753 | Abcam |
| ab93196 | Abcam |
| AP8658b | Abgent |
| AP8658c | Abgent |
| R2692-2 | Abiocode, Inc. |
| Y055282 | ABM |
| H00201163-A01 | Abnova |
| H00201163-A02 | Abnova |
| H00201163-B01P | Abnova |
| H00201163-K | Abnova Corporation |
| AP18021CP-N | Acris Antibodies GmbH |
| AP18021PU-N | Acris Antibodies GmbH |
| AP18022CP-N | Acris Antibodies GmbH |
| AP18022PU-N | Acris Antibodies GmbH |
| DF12608 | Affinity Biosciences |
| ABIN1078048 | Antibodies-online.com |
| ABIN1086227 | Antibodies-online.com |
| ABIN1086228 | Antibodies-online.com |
| ABIN1176707 | Antibodies-online.com |
| ABIN1584472 | Antibodies-online.com |
| ABIN1715479 | Antibodies-online.com |
| ABIN1734396 | Antibodies-online.com |
| ABIN1812719 | Antibodies-online.com |
| ABIN1812720 | Antibodies-online.com |
| ABIN1896065 | Antibodies-online.com |
| ABIN1896066 | Antibodies-online.com |
| ABIN1896067 | Antibodies-online.com |
| ABIN1896068 | Antibodies-online.com |
| ABIN1896069 | Antibodies-online.com |
| ABIN1896070 | Antibodies-online.com |
| ABIN1896071 | Antibodies-online.com |
| ABIN1896072 | Antibodies-online.com |
| ABIN1896073 | Antibodies-online.com |
| ABIN1896074 | Antibodies-online.com |
| ABIN1896075 | Antibodies-online.com |
| ABIN1896076 | Antibodies-online.com |
| ABIN1896077 | Antibodies-online.com |
| ABIN1896078 | Antibodies-online.com |
| ABIN2030106 | Antibodies-online.com |
| ABIN2035376 | Antibodies-online.com |
| ABIN2141985 | Antibodies-online.com |
| ABIN2267936 | Antibodies-online.com |
| ABIN2267938 | Antibodies-online.com |
| ABIN2267939 | Antibodies-online.com |
| ABIN2267940 | Antibodies-online.com |
| ABIN2267941 | Antibodies-online.com |
| ABIN2267942 | Antibodies-online.com |
| ABIN2267943 | Antibodies-online.com |
| ABIN2267944 | Antibodies-online.com |
| ABIN2267945 | Antibodies-online.com |
| ABIN2267946 | Antibodies-online.com |
| ABIN2267947 | Antibodies-online.com |
| ABIN2267948 | Antibodies-online.com |
| ABIN2267949 | Antibodies-online.com |
| ABIN2423484 | Antibodies-online.com |
| ABIN2423485 | Antibodies-online.com |
| ABIN2430124 | Antibodies-online.com |
| ABIN2430125 | Antibodies-online.com |
| ABIN2436904 | Antibodies-online.com |
| ABIN2436905 | Antibodies-online.com |
| ABIN2562622 | Antibodies-online.com |
| ABIN2602234 | Antibodies-online.com |
| ABIN2602235 | Antibodies-online.com |
| ABIN2602236 | Antibodies-online.com |
| ABIN2602237 | Antibodies-online.com |
| ABIN2602238 | Antibodies-online.com |
| ABIN2602239 | Antibodies-online.com |
| ABIN2788829 | Antibodies-online.com |
| ABIN2788830 | Antibodies-online.com |
| ABIN2813329 | Antibodies-online.com |
| ABIN2842403 | Antibodies-online.com |
| ABIN2842404 | Antibodies-online.com |
| ABIN2881720 | Antibodies-online.com |
| ABIN2929574 | Antibodies-online.com |
| ABIN2969374 | Antibodies-online.com |
| ABIN2969375 | Antibodies-online.com |
| ABIN2977305 | Antibodies-online.com |
| ABIN3003855 | Antibodies-online.com |
| ABIN4312283 | Antibodies-online.com |
| ABIN4312284 | Antibodies-online.com |
| ABIN4312285 | Antibodies-online.com |
| ABIN453652 | Antibodies-online.com |
| ABIN4903684 | Antibodies-online.com |
| ABIN5002801 | Antibodies-online.com |
| ABIN5002802 | Antibodies-online.com |
| ABIN531278 | Antibodies-online.com |
| ABIN5539359 | Antibodies-online.com |
| ABIN5539360 | Antibodies-online.com |
| ABIN566940 | Antibodies-online.com |
| ABIN5706162 | Antibodies-online.com |
| ABIN5944719 | Antibodies-online.com |
| ABIN5944720 | Antibodies-online.com |
| ABIN5944721 | Antibodies-online.com |
| ABIN5973885 | Antibodies-online.com |
| ABIN5999318 | Antibodies-online.com |
| ABIN6041862 | Antibodies-online.com |
| ABIN6042938 | Antibodies-online.com |
| ABIN6133517 | Antibodies-online.com |
| ABIN6135168 | Antibodies-online.com |
| ABIN6140670 | Antibodies-online.com |
| ABIN6140671 | Antibodies-online.com |
| ABIN6140672 | Antibodies-online.com |
| ABIN6222156 | Antibodies-online.com |
| ABIN652601 | Antibodies-online.com |
| ABIN652602 | Antibodies-online.com |
| ABIN761081 | Antibodies-online.com |
| ABIN761083 | Antibodies-online.com |
| ABIN761084 | Antibodies-online.com |
| ABIN761085 | Antibodies-online.com |
| ABIN761086 | Antibodies-online.com |
| ABIN761087 | Antibodies-online.com |
| ABIN761088 | Antibodies-online.com |
| ABIN761090 | Antibodies-online.com |
| ABIN786437 | Antibodies-online.com |
| ABIN786438 | Antibodies-online.com |
| ABIN798440 | Antibodies-online.com |
| ABIN798661 | Antibodies-online.com |
| ABIN897351 | Antibodies-online.com |
| ABIN897352 | Antibodies-online.com |
| ABIN897354 | Antibodies-online.com |
| ABIN897355 | Antibodies-online.com |
| ABIN926754 | Antibodies-online.com |
| ABIN926755 | Antibodies-online.com |
| ABIN949906 | Antibodies-online.com |
| 20-5432 | ARP |
| 25-A6493 | ARP |
| HPA028760 | Atlas Antibodies |
| ARP61520_P050 | Aviva Systems Biology |
| ARP61521_P050 | Aviva Systems Biology |
| OAAB05252 | Aviva Systems Biology |
| OAAB05253 | Aviva Systems Biology |
| A302-408A | Bethyl Laboratories |
| IHC-00679 | Bethyl Laboratories |
| orb101471 | Biorbyt |
| orb102606 | Biorbyt |
| orb386876 | Biorbyt |
| orb39348 | Biorbyt |
| orb39349 | Biorbyt |
| orb446788 | Biorbyt |
| orb468575 | Biorbyt |
| orb484451 | Biorbyt |
| orb520866 | Biorbyt |
| orb520867 | Biorbyt |
| orb539494 | Biorbyt |
| orb539495 | Biorbyt |
| bs-6007R | Bioss |
| bs-6007R-A350 | Bioss |
| bs-6007R-A488 | Bioss |
| bs-6007R-A555 | Bioss |
| bs-6007R-A647 | Bioss |
| bs-6007R-Biotin | Bioss |
| bs-6007R-Cy3 | Bioss |
| bs-6007R-Cy5 | Bioss |
| bs-6007R-Cy5.5 | Bioss |
| bs-6007R-Cy7 | Bioss |
| bs-6007R-FITC | Bioss |
| bs-6007R-HRP | Bioss |
| BS8262 | Bioworld Technology, Inc |
| A00718 | Boster Biological Technology |
| 3697 | Cell Signaling Technology |
| CBMAB-1263-YC | Creative Biolabs |
| CBMAB-CP0716-LY | Creative Biolabs |
| CBMAB-F0915-CQ | Creative Biolabs |
| CBMAB-F2026-CQ | Creative Biolabs |
| MOR-1315 | Creative Biolabs |
| CPBT-34048RH | creative-diagnostics |
| CPBT-34049RH | creative-diagnostics |
| DCABH-11581 | creative-diagnostics |
| DPABH-07844 | creative-diagnostics |
| DPABH-12066 | creative-diagnostics |
| MABC288 | EMD Millipore |
| GTX33200 | GeneTex |
| GTX81112 | GeneTex |
| PA5-23016 | Invitrogen |
| PA5-55971 | Invitrogen |
| PA5-76954 | Invitrogen |
| PA5-100247 | Invitrogen Antibodies |
| PA5-72548 | Invitrogen Antibodies |
| APR06111G | Leading Biology |
| LS-C166173 | LifeSpan Biosciences |
| LS-C166174 | LifeSpan Biosciences |
| LS-C334752 | LifeSpan Biosciences |
| LS-B15471 | LifeSpan BioSciences, Inc. |
| LS-B15499 | LifeSpan BioSciences, Inc. |
| LS-B7753 | LifeSpan BioSciences, Inc. |
| LS-C145402 | LifeSpan BioSciences, Inc. |
| LS-C145628 | LifeSpan BioSciences, Inc. |
| LS-C244410 | LifeSpan BioSciences, Inc. |
| LS-C244411 | LifeSpan BioSciences, Inc. |
| LS-C244412 | LifeSpan BioSciences, Inc. |
| LS-C244413 | LifeSpan BioSciences, Inc. |
| LS-C244414 | LifeSpan BioSciences, Inc. |
| LS-C244415 | LifeSpan BioSciences, Inc. |
| LS-C244416 | LifeSpan BioSciences, Inc. |
| LS-C244417 | LifeSpan BioSciences, Inc. |
| LS-C244418 | LifeSpan BioSciences, Inc. |
| LS-C244419 | LifeSpan BioSciences, Inc. |
| LS-C247586 | LifeSpan BioSciences, Inc. |
| LS-C247587 | LifeSpan BioSciences, Inc. |
| LS-C286884 | LifeSpan BioSciences, Inc. |
| LS-C289155 | LifeSpan BioSciences, Inc. |
| LS-C293968 | LifeSpan BioSciences, Inc. |
| LS-C299346 | LifeSpan BioSciences, Inc. |
| LS-C305401 | LifeSpan BioSciences, Inc |
| LS-C327392 | LifeSpan BioSciences, Inc |
| LS-C327393 | LifeSpan BioSciences, Inc. |
| LS-C401689 | LifeSpan BioSciences, Inc. |
| LS-C404990 | LifeSpan BioSciences, Inc. |
| LS-C412394 | LifeSpan BioSciences, Inc. |
| LS-C413733 | LifeSpan BioSciences, Inc. |
| LS-C468751 | LifeSpan BioSciences, Inc. |
| LS-C468752 | LifeSpan BioSciences, Inc. |
| LS-C468753 | LifeSpan BioSciences, Inc. |
| LS-C468754 | LifeSpan BioSciences, Inc. |
| LS-C468755 | LifeSpan BioSciences, Inc. |
| LS-C468756 | LifeSpan BioSciences, Inc. |
| LS-C565158 | LifeSpan BioSciences, Inc. |
| LS-C565159 | LifeSpan BioSciences, Inc. |
| LS-C584811 | LifeSpan BioSciences, Inc. |
| LS-C584812 | LifeSpan BioSciences, Inc. |
| LS-C604463 | LifeSpan BioSciences, Inc. |
| LS-C604464 | LifeSpan BioSciences, Inc. |
| LS-C624115 | LifeSpan BioSciences, Inc. |
| LS-C624116 | LifeSpan BioSciences, Inc. |
| LS-C643764 | LifeSpan BioSciences, Inc. |
| LS-C643767 | LifeSpan BioSciences, Inc. |
| LS-C665616 | LifeSpan BioSciences, Inc. |
| LS-C749523 | LifeSpan BioSciences, Inc. |
| LS-C810110 | LifeSpan BioSciences, Inc. |
| MBS2520402 | My BioSource |
| MBS9205508 | My BioSource |
| MBS9210297 | My BioSource |
| 110686 | NovoPro Bioscience Inc |
| NBP1-44995 | Novus Biologicals |
| NBP1-89983 | Novus Biologicals |
| TA309661 | OriGene |
| TA349991 | OriGene |
| 63-573 | ProSci |
| 63-574 | ProSci |
| 11236-2-AP | Proteintech Group |
| 3929-1 | RabMAbs |
| T3335 | RabMAbs |
| sc-25168 | Santa Cruz Biotechnology |
| sc-25777 | Santa Cruz Biotechnology |
| sc-271558 | Santa Cruz Biotechnology |
| F4931 | Sigma-Aldrich |
| STJ113428 | St John's Laboratory |
| STJ116732 | St John's Laboratory |
| STJ28576 | St John's Laboratory |
| 140351 | US Biological Life Sciences |
| 218964 | US Biological Life Sciences |
| 222723 | US Biological Life Sciences |
| 246150 | US Biological Life Sciences |
| 316145 | US Biological Life Sciences |
| 316146 | US Biological Life Sciences |
| 316223 | US Biological Life Sciences |
| 389172 | US Biological Life Sciences |
| 389173 | US Biological Life Sciences |
| 481134 | US Biological Life Sciences |
| 481135 | US Biological Life Sciences |
| 509528 | US Biological Life Sciences |
| 218964-AP | US Biological Life Sciences |
| 218964-APC | US Biological Life Sciences |
| 218964-Biotin | US Biological Life Sciences |
| 218964-FITC | US Biological Life Sciences |
| 218964-HRP | US Biological Life Sciences |
| 218964-ML405 | US Biological Life Sciences |
| 218964-ML490 | US Biological Life Sciences |
| 218964-ML550 | US Biological Life Sciences |
| 218964-ML650 | US Biological Life Sciences |
| 218964-ML750 | US Biological Life Sciences |
| 218964-PE | US Biological Life Sciences |
| F5730-01 | US Biological Life Sciences |
| F5730-01-AP | US Biological Life Sciences |
| F5730-01-APC | US Biological Life Sciences |
| F5730-01-Biotin | US Biological Life Sciences |
| F5730-01-FITC | US Biological Life Sciences |
| F5730-01-HRP | US Biological Life Sciences |
| F5730-01-ML405 | US Biological Life Sciences |
| F5730-01-ML490 | US Biological Life Sciences |
| F5730-01-ML550 | US Biological Life Sciences |
| F5730-01-ML650 | US Biological Life Sciences |
| F5730-01-ML750 | US Biological Life Sciences |
| F5730-01-PE | US Biological Life Sciences |
| F5730-01A | US Biological Life Sciences |
| F5730-01A-AP | US Biological Life Sciences |
| F5730-01A-APC | US Biological Life Sciences |
| F5730-01A-Biotin | US Biological Life Sciences |
| F5730-01A-FITC | US Biological Life Sciences |
| F5730-01A-HRP | US Biological Life Sciences |
| F5730-01A-ML405 | US Biological Life Sciences |
| F5730-01A-ML490 | US Biological Life Sciences |
| F5730-01A-ML550 | US Biological Life Sciences |
| F5730-01A-ML650 | US Biological Life Sciences |
| F5730-01A-ML750 | US Biological Life Sciences |
| F5730-01A-PE | US Biological Life Sciences |

TABLE 17
Antisense oligonucleotides targeting SEQ ID NO: 2

| | | Target | Target | | | |
|---|---|---|---|---|---|---|
| GI | | Start | Stop | Target | SEQ | IC₅₀ |
| ID# | Sequence | Site | Site | Region | ID NO | (nM) |
| GI81 | TGGCTGATATATCCCGGG | 12705 | 12722 | Exon 5 | 21 | 14 |
| GI84 | GCGTTGCCGTTCCTCTGG | 16414 | 16431 | Exon 7 | 613 | — |
| GI85 | TGCGGACCGTGGACATGA | 25298 | 25315 | Exon 14 | 614 | — |
| GI86 | GCCGTGGAGCTCGCAGAA | 10893 | 10910 | Exon 4 | 615 | — |
| GI88 | GCCTGGCGGACAATGCTG | 12783 | 12800 | Exon 5 | 616 | 19 |
| GI89 | GTGCTGGTGGCTGACGTA | 12745 | 12762 | Exon 5 | 617 | — |
| GI90 | CGGGTGCCCTGCAGCAAG | 12685 | 12702 | Exon 5 | 618 | 35 |

1. A compound comprising a modified oligonucleotide consisting of 12 to 30 linked nucleosides and having a nucleobase sequence that is at least 70%, at least 80%, at least 85%, at least 90%, at least 95%, at least 98%, or 100% complementary to an equal length portion of nucleobases 1833-26789 of SEQ ID NO: 2,
wherein the thymine bases are optionally uracil bases, and
wherein the oligonucleotide comprises at least one modified sugar, at least one modified internucleoside linkage, and/or at least one modified nucleobase.

2. (canceled)

3. The compound of claim 1, wherein the modified oligonucleotide consists of at least 8 consecutive nucleobases with at least 70%, at least 80%, at least 85%, at least 90%, at least 95%, or 100% identity to any of the nucleobase sequences of SEQ ID NOs: 16-618.

4. (canceled)

5. The compound of claim 3, wherein the SEQ ID NO is one of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 613, SEQ ID NO: 614, SEQ ID NO: 615, SEQ ID NO: 616, SEQ ID NO: 617, or SEQ ID NO: 618.

6-10. (canceled)

11. The compound of claim 1, wherein the modified oligonucleotide comprises at least one 2′-O-methoxyethyl (2′-MOE) modified sugar.

12. The compound of claim 1, wherein the modified oligonucleotide comprises at least one 2′-O-methyl (2′-OMe) modified sugar.

13. The compound of claim 1, wherein the modified oligonucleotide comprises at least one bicyclic sugar.

14. The compound of claim 13, wherein each bicyclic sugar comprises a chemical bridge between the 4′ and 2′ positions of the sugar, wherein each chemical bridge is independently selected from: 4′-CH(R)—O-2′ and 4′-(CH₂)₂(CH2)2-O-2′, wherein R is independently selected from H, Ci-Cu alkyl, or a protecting group.

15. The compound of claim 1, wherein the modified oligonucleotide comprises at least one phosphorothioate internucleoside linkage.

16. The compound of claim 15, wherein each internucleoside linkage of the modified oligonucleotide is a phosphorothioate internucleoside linkage.

17. The compound of claim 15, wherein the modified oligonucleotide comprises at least one phosphodiester internucleoside linkage.

18. The compound of claim 1, wherein the modified oligonucleotide comprises at least one 5-methylcytosine modified nucleobase.

19. (canceled)

20. The compound of claim 1, wherein the modified oligonucleotide comprises a gapped sequence, which gapped sequence comprises:
a central sequence of linked deoxynucleosides; and
wing sequences flanking both the 5′ and the 3′ ends of the central sequence, wherein at least one nucleoside of the wing sequences comprises a modified sugar.

21. The compound of claim 20, wherein the central sequence is chosen to consist of 6, 7, 8, 9, 10, 11, or 12 linked nucleosides and the wing sequences are each independently chosen to consist of 3, 4, 5, or 6 linked nucleosides.

22. The compound of claim 20, wherein the central sequence consists of 10 linked nucleosides and the wing sequences each consist of 4 linked nucleosides.

23. The compound of claim 20, wherein the wing sequences comprise at least one nucleoside consisting of a 2′-O-methoxyethyl modified sugar.

24-46. (canceled)

47. A composition comprising the compound of claim 1 or a salt thereof, and a pharmaceutically acceptable carrier or diluent.

48. A method of inhibiting the expression of FLCN in cells or tissues comprising administering the compound of claim 1 or a salt thereof or a pharmaceutical composition comprising the compound of claim 1 or salt thereof to a cell, animal, or human.

49-50. (canceled)

51. A method to treat, prevent or ameliorate a TDP-43 proteinopathy, a neurodegenerative or neuromuscular disease in a subject, comprising administering the compound of claim 1 or a salt thereof or a pharmaceutical composition comprising the compound of claim 1 or salt thereof any one of claims 1-46 to the subject.

52. The method of claim 51, wherein the TDP-43 proteinopathy, the neurodegenerative or neuromuscular disease is amyotrophic lateral sclerosis, frontotemporal lobar degeneration, age-related macular degeneration, or Alzheimer's disease.

53. A method of inhibiting the expression of FLCN in cells or tissues to treat, prevent or ameliorate a TDP-43 proteinopathy in a subject, comprising administering the compound claim 1 or a salt thereof or a pharmaceutical composition comprising the compound of claim 1 or salt thereof to the subject, such that expression of FLCN is inhibited.

54-83. (canceled)
